SEQUENCE LISTING (186 sequences)

SEQ ID NO: 1 - DNA, 2649 bp, synthetic construct
  misc_feature 1..2649: note = OTHER INFORMATION: ITR2-PtrnQ:mRuby-TCAT-CAT-PCAT-ITR1 expression cassette
  source 1..2649: mol_type = other DNA
aacaggttggctgataagtccccggtctactgagtcccaccacacccgcccgaccgaactaagttaagacacgcctagcacgaagcccacagaagtacgttgacaccaactgatatgcaaagtcatctctttttcccattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatgaattccaaaggtgaagaactgatcaaagaaaacatgcgcatgaaagtggtgatggaaggtagcgtgaacggtcaccaattcaaatgcaccggcgagggcgaaggcaacccctacatgggcacccaaaccatgcggattaaggtgattgaaggcgggcccctccctttcgcattcgatattctcgcgacctcctttatgtacggctcgcgcacgtttatcaagtaccccaagggcatcccggatttcttcaagcagtcctttccagaaggtttcacgtgggaacgggttacccgttacgaggacggtggcgtcgttactgtcatgcaagacacttccctcgaagatggttgcctggtgtatcatgtgcaagtccgcggtgttaactttccgagtaacggccctgtgatgcaaaagaagaccaaaggttgggaaccgaataccgaaatgatgtatcccgctgacggtggcctgcgcggctatactcacatggcgttgaaagtggacggcggcggccatttgtcgtgcagtttcgtcactacttatcgttccaaaaagacggttggcaatatcaaaatgcccgggattcacgcagtcgatcatcgtttggagcgcctggaagaaagcgacaacgagatgttcgtcgtgcaacgtgaacacgctgtggccaaatttgcgggcttgggcggtggtatggatgagttgtataaataagcggccgcacatcatcatcaccatcactaagctttaagcgatgctgagcaggtatgcaaaaagagtttgtagaaactcctacggcgggctccgtgacgattaattaaccccacccgaagtcgcgcaaccaactaactttacagaagacataccttgagatctcgcgcgtttcggtgatgacggtgaaaacctctgacacatgcagctcccgtagacggtcacagcttgtctgtaagcggatgccgggagcagacaagcccgtcagggcgcgtcagcgggtgttggcgggtgtcggggctggcttaactatgcggcatcagagcagagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 2 - DNA, 5287 bp, synthetic construct
  misc_feature 1..5287: note = OTHER INFORMATION: ITR2-PsigX:Pz_porCA-TCAT-CAT-PCAT-ITR1 expression cassette
  source 1..5287: mol_type = other DNA
aacaggttggctgataagtccccggtctactgagtcccaccacacccgcccgaccgaactaagttaagacacgcctagcacgaagcccacagaagtacgttgacaccaagctatcgttaaaaatatcgtggaagcccacaacggatcaattactgtgcacagccgaatagataaaggaacaacattttctttttatattccgacaaaacggtaaaatcgagtctgaatttgccgaagaatcttgttccataagaaacacccgctgactgagcgggtgtttttttaatagccaacattaataaaatttaaggatatgttaatataaattcccttccaaattccagttactcgtaatatagttgtaatgtaacttttcaagctattcatacgacaaaaaagtgaacggaggggtttcagatgtcggtggtacaattaccgaaagtgaacgaacttgggtttttcgagatccgactcgaatccataggaggactcggcgcgaaccttgccggtaaaatgctggctgaagcaggggtagtaggcgcgaatttgaatggcgtcagcttctcgtcttatggttcggagaagaaaggttcgccggttaaagcgcatatccgtttctgcgatttgaatacgccgatccgcgatacttcgccggttgaacggccgcatgtcgttgccgtgttccacgaagccatgggcaagattgttaatgtaacaagcggcattaccgcagaaagcaccgtgctgatcaactcagcgaaatctcccgaagagctgaaggaaattcttggcgtaactgcgggcacaatcgctgttgtcgatgcgaccattatcgcgctggaagagaagaaccgcgtgaacatggcgatgctgggcggtctgttccgactctgtccgttccttgatcctaatacaatgaagaacgttatcgagaagtcgctcggcaagaaataccctcaagcggttcagtcggccatcacgactttcgaccgcggctacaatgaagtgaagttccaaacgtttgagctgccggaaggcgagagcatgccggaatttgtacggtccgacatttcgccgctcggatacgaaacccagccgattggcggcaccatcaccaatccgggaacaagcttcctgaaagatctcagcatttcccgttccgggcttgtgccggtatatgacaacgagtcctgcatcaactgcgcggagtgcgacacggtttgtccggatatgtgcttcgtttgggaagaaaaggtcaataaaaaaggcaaatcgatgatgttcctgaaagggatcgattatcaatactgtaaaggctgtctcaaatgcgtggaagcctgcccgacctccgcactctcccgttcgcgtgagaaggaaggcttcgcggaagctaatacggtccggcacacttttgatcttgtaaaccagatctaaatcccgagagaaaggtggaacactaatggctatcgattacgaaaaagaagtcggctccgccaaggtggagcagaagtttttatatgaatccggcaatgaaatggcggcttacgctgcgcatcaaatcaactatcatgtgatgggctatttcccgatctccccgtccacggaggttgcgcaattcctcgattccatgaaagccagcggacagcatgacatcatgctggtgccttctgacggcgagcacagctctgcgggcatctgttacggcgcatccaccgcaggcggccgcgtatttaacgcgacaagtgcccaaggctacctgtacatgctggagcagctcccggttcaatccggcacgcggatgccgatggtcatgaacctgatctgccgttccgtttccggaccgctgaacattcatggagatcactccgacctgtattatgcgctgaacaccggctggccgatcctgatgtgcccggatccacagtccgtatacgatatgaacatcatggcaatcaagctggccgagcatgccaaggttcgcctgcccgtgctggtagcgatggacggatactttacttctcaccagaagcgccgcgtccaagcgattgcgaaccgtgaagatgtccaaagctttatcggacctcaaccgcaaggttatgacgatacgctggaccgtgagaatccgatttccgtcggcccttatatgaacgaaccggactatatcaacaacaaataccaacaatccgtggcgatgtacaatgctgcggaagtattcgaagaaatcgccaaagaatatggcgagctgacaggccgccattacccgatcctgtcgcagtaccggatggaagatgccgaggttgcggtattcttgatgaactccgcttccgagatcatcaaggacgttgtcgatcagcttcgtgagaaaggcattaaagccggcgccatttcgccgaacatgatccgtccgttcccgcagaagcaaattgccgaagcgctgaagaacgttaaagccatcaccgtcggtgaccgcgcggattctttcggagcgcatggcggcaacatggttaacgaaatcaaggcggctctgttcacttacggcaatacaacaacgaaagtaatcagccggatttacggtgtcggcggcaaggacttctttgccgaagacggccacagcctgttcgagatggctattgaggcagcggaaaccggtgaagtgaaagtacctttcgattattacggccacaatccgggcgttgccgaaagcgcgccgaagcgtgtgatcaaaccgcttaaattcgagtctctcaagacaggcttgattactgtggacaaggatgaggaaacaggcaaactcaaggttaagattccgccaatgcgcagcttgaccgttaaaccgaagcgcttgtccccgggtcacggtacttgcccaggctgcggtattttctccggcctggagctgttcttcaaaggcatcgaaggagatgtcgttgctctgtaccacacaggctgcgcctttgttacgacgaccggctatccttattcgtcgcataaatcgacgttcatccataacctgttccaaagtggtgccgcaactatgtccggtgtggttgaaatgttctgggaacgcaaacgccgcggcgagcttgatcatctgaacctgaaagacgactttacctttgtcatggttaccggcgacggcggcatggacatcggtatggggccggcaatcggcgccgcgcttcgcggacataagatgatcgttctggaatatgacaatgagggctacatgaatacgggtgcacagcaatcgtactcgacgccgatcggtcaccgcacatcgacatccagcatcggcaagacgcaaaatggtaaggtgacgcagcataaagatactccgcaaattatggctgccaccaatattccttatgtgttcacaggctgcgaagcgttcccgcaggatttggtgaagaaagccgccaaagcccaatggtacgctcagaacgaagggcttgtatacggcaaaatcttgtctgcctgcccgctgaattggatgaccgaagacaatgtaggcaccaagctggtatccctggctgtcgattcctgcttcttcccgctgtatgaagtggagcaaggcgttaccacgattacgtacaatccggaagaaaaggaaaaacgcgtagacgttaccgaatggctcaaaggcatgggtaaaaccaagcatctgcttaaggaagagaacgaagctgcgctgaacggcttcaaggaagaagtagagcgccgctggagccgcctgaaagcgaagcacgagcatccggagctgtaagatccgcttgattgcttgaaaatggagctgtcccataagtagattttcaaagtcagagacagttcttcttcattctagaaaacgcaaaaagtcagcaaaacagcgattctccggttattggagaatcgctgttttgcgtttttggtccactgcggcttgttttagtacattatgggcaagggaaagccacccgacctcaagcgtcactttctccatgccgcgaagcagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 3 - DNA, 6912 bp, synthetic construct
  misc_feature 1..6912: note = OTHER INFORMATION: ITR2-PsigX:Pz_sufCDSUB-TCAT-CAT-PCAT-ITR1 expression cassette
  source 1..6912: mol_type = other DNA
aacaggttggctgataagtccccggtctactgagtcccaccacacccgcccgaccgaactaagttaagacacgcctagcacgaagcccacagaagtacgttgacaccaagctatcgttaaaaatatcgtggaagcccacaacggatcaattactgtgcacagccgaatagataaaggaacaacattttctttttatattccgacaaaacggtaaaatcgagtctgaatttgccgaagaatcttgttccataagaaacacccgctgactgagcgggtgtttttttaatagccaacattaataaaatttaaggatatgttaatataaattcccttccaaattccagttactcgtaatatagttgtaatgtaacttttcaagctattcatacgacaaaaaagtgaacggaggggtttcagatggcagcagaatttgtcattgagggtttgaaagcgacgattgaggggaaagagatcttgaagggatttaaccttcagatgaagggcggggaggttcatgccatcatgggcccgaacggcactggtaaaagtactctggcgtcttcgctgatgggtcatccgaaatatgaagtgacggaaggaaccgtaacccttgagggtgaagatgtgctggagatgtccgttgacgagcgtgcccgcgccggattattccttgctatgcagtatccaagcgagatcgccggcgtaacgaactccgactttctgcgcagtgcgatcaatgcgcgccgcgaagagggaagtgaaatttcgctgatcaagtttatccgccaaatggaaggcaagatgaaggatttggagatgaatccggagttcatgcaccgctatttgaatgaaggcttctccggcggcgagaagaaacgcaacgaaattttgcaaatgctgctgcttgaaccgaaaattgtgattttggatgaaatcgattcgggtctggatatcgacgcgctgagggttgtcgcgcagggtgtaaactccatgcgaagcgaagaccggggtttcctggttatcactcactaccaacgtctgcttaactacattaaacccgactttgttcatattatgatgcaaggcagaatcgtaaaatccggaggccctgagcttgccgagcgattggaagccgatggttatgaatgggtgaaagaagaactggggatcgaagatgaaactgtagggcaggaagcttaagaacgcttggaaggaggagaacataatgactacgcaaaccattcttccggtggatgccggccgattaagcgaactgtcgctgagaagcggcgagccggaatggctgaaggaaagccgtcttaaggcgctggagctggccgcggaactggaattgccgaagctggagaagactcggattgatcgctggaatattaacaattacggcagttataaagaaggcgcggcgatcgccgcattgaacgaagcgccctccgccattgccgcgctagtaggcagctcggaggcaggaagccttattatccaacggaattctggagcggtctatacacggcttgcacctgagatggcggatcagggggtaatcttcacggatctgcacactgcggttaaggagcacggagagctggtgcggcggtacctgcacactgcggtcaaaccggaggagcattcggttgcggctctgcacgcggcgctgtggaacggtggcgtattcctgtacattccgaaaaatgtagtggtcgatacaccggttcaggctgtattcctgacagatgacgcggaggcttcatttgctccccatgttctgatcgttgcggacagcaacagttcggtaacttatgttgacaactatgtatcgggcaagagcgaagccggacttcacaacggcgcggtggaagtgttcgtctgcgccggagcgaaggttcgctacgcgacagtgcatcaattcggcgctgatactacggatgtaacgtatcgccgcgcgatcgtggagaatgacggatcggtcgagtggattgtcggcgaaatgaacgacggcgatacagccagcgacacgaagtcggtgcttaagggcaacggggcaagctccgacgcaaaagtcatcgcggtgggctcaggctcgcagaagctcaactacacgacgcaggcgcagcatttcggcaagaacacgccaagcaatatgattacccgtgcggttatgcgtgagaacgccacgtcgattatcaacggcattaccaaaatcgagaaaggcgcaacgcgcgctgacggccagcaaacggaaaaagtactaatgcttagccctaaggcgcgcggcgacgccaatccgattctgcttatagacgaggatgatgtgaccgcaggtcatgccgcttccgtagggcaggttaatccggaacaaatctattatctgatgtcccgcggtatttcgcgttcggacgcggagaaactggttatttacggcttccttgcgccggttgtgtcgcaaattccacttgagggactgcgtaatcagcttaagtctcttgtggaaaggaagttgggccaatgaacagctctatccgggaactattccctatcctagaccagaatgttaacggtcacccgctcgtttatctggatagtgcggcgacatcgcagaagccgcggcaggtaattgaagcgatcaaggcttactacgagcatgataattccaacgttcaccgcggcgtccatacgctgggcagccgcgcaaccgatgcttatgaaggagccagagagaaggttgccagatttattaacgcccatagtaccaaagaaatcgtatttacccgcggtacaacgacggctcttaatctggtagcttcctcttacgggccttccgccgtcggcgagggcgatgagatcgtgattaccctgatggagcaccacagtaacctgattccgtggcagcagcttgccaagaagacgggagcgactcttaagttcattccgctgcaaaaagacggaaccattctgctggaggatgccgagaagacaattacggacaacaccaagatcgtatccattgcctatgtctccaatgtaatgggcgtcaccagcccggtaaaagaaattgcggccatcgctcaccgccacggcgcggtcatggtcgttgacggcgcgcagagcacgccgcatatgaaggtggatgttcaggatctggactgcgacttctatgctttgtccggccacaaaatgtgcggtccaaccggcatcggagccctttacggcaaaagagcgctgcttgaagccatggaaccggttgagtttggcggggaaatgattgatgacgtcggcctttacgattcgacctggaaggaactaccctggaagttcgagggcggcaccccgatcatcgcgggcgcagtcggcttgggagcggcgatcgacttccttcaagaggtcggtctggatgagattcaccggcatgagattaagctcgcggcctatgcggagcagcgtctggccgaagttgaaggaatatcgatttacggaccgcgtaaccgggaagtgggcgtcataaccttcaatttgggggatgttcacccgcatgacgtagccactgtgcttgatgcggaaggaatagcggtccgggcgggccaccactgctgccagccgctgatgcgctggctggaagttagttctacggcaagggccagcctctacttgtataacaccgaagaagacgttgaccggctggcctcggcactaatccggacaaaggagtatttcgccgatgcaattagatgatttgtacagacgtgtaatcatggatcattataagaatccccggaaccgcggcttgttcgaagacgatgctatgaaggtggaactgaacaaccctacctgcggcgaccgcattacactgcagcttaaagtggaagacgggatcgtgaaggaagcccggtatatcggtgaaggctgttcgatcagcatgtcctccgcttcgatgatgaccgaagtggttaaagggaagacagtagagcaggcgctggatttggccagccgattctctgcactgatgaagggcgaggacgtgacgttcgacgattacgaagatttagaggccctgtccggcgttaacaaatttccggcgcggatcaaatgtgcgactctggcctggaatgcgctgcgcaaaggcatcgacgaggaagagcggcaacatcactaaataaactgaaggaggcgtaccccatggccaaaaaagcgcctgaaattgaagagtacaaatatggttttcgggacgagcacaaggcggtatttcaaacgggtaaagggctgactccggaaattgtgaaggaaatctcggcgattaagaatgagccgcagtggatgctggacttccgtctgaaatcgctcgagcaattcggtaaaatgccgctgccgcgttggggcggaaaccttgacgaactggatttcaacgacattcagtactatgtaagaccatccgagaagcagggcaagacctgggaagaagttccttccgaaatcaaggaaacattcgataagctcggcattcccgaagcggaacaaaagttccttgcaggagtatccgcacaatacgaatccgaggtcgtctaccatagcatgcagcatgatcttgaagagcagggtgtaatatttacggacacggacacggctttgcgtgagcatcccgagatctttaagcagtatttcggcaccgtgattcctccgaccgacaacaagttcgcagcgctcaacagcgccgtttggtcgggtggaagctttatctacgttccaaaaggcgttaaatgcgaaataccgctacaggcttacttccggatcaactccgagaacatgggacaattcgaaagaacgctgatcattgccgatgaggacagcttcgtgcattacgtggaaggctgcaccgctccggtctacagcacgaattcgctgcacagcgcggtcgttgaaatcatctgtaaaaagaacgcgcgtgtccgctatacaaccattcagaactgggcgccgaacatctacaacctcgttaccaaacgcgcggttgcggaagaaaacgcgacgatggaatgggttgacggcaacatcggttccaagctgacgatgaaatacccggcggtcgttctgaaaggacgcggagccaagggttccgtactctctatcgcggtagccggtaaaggccagcatcaggatgccggcgccaagatgatccatctggcaccggacacgacgtccaccatcatttccaagtcgatcagtaagcacggcgggaaagttacttaccgcggactcgcttcctttggccgtcaggcggaaggcgcgaagtcaaatattaaatgcgatacgctcattatggataacgaatctacttcggatacaattccttataatgagattatgaacgacaatattacgctggagcatgaagcgaccgtatccaaggtatccgaggagcagctcttctacctgatgagccgcggtcttacggaagccgaagcgacccagatgatcgtaatgggcttcattgagccgttcaccaaggaactgccgatggaatacgccgtcgagatgaaccgtcttatcaaattcgagatggaaggaagcatcggctagccttgaggtgatcaagggttcccggcttatgtaacatgcaaatacgtgtccgcagtgatcttgacgcatttttgcccaaggcgctcgggcggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 4 - DNA, 2339 bp, synthetic construct
  misc_feature 1..2339: note = OTHER INFORMATION: ITR2-PsigX:Pz_fldA-TCAT-CAT-PCAT-ITR1 expression cassette
  source 1..2339: mol_type = other DNA
aacaggttggctgataagtccccggtctactgagtcccaccacacccgcccgaccgaactaagttaagacacgcctagcacgaagcccacagaagtacgttgacaccaagctatcgttaaaaatatcgtggaagcccacaacggatcaattactgtgcacagccgaatagataaaggaacaacattttctttttatattccgacaaaacggtaaaatcgagtctgaatttgccgaagaatcttgttccataagaaacacccgctgactgagcgggtgtttttttaatagccaacattaataaaatttaaggatatgttaatataaattcccttccaaattccagttactcgtaatatagttgtaatgtaacttttcaagctattcatacgacaaaaaagtgaacggaggggtttcagatgattaaaatattagtggtatatgccagcctgaccggcaatacagaggaaatagcggaactgatcgcggagggaatccgccaatcgggaggggagacagtcataaaatcggtcaccgactgcaacgccttcgatatcgcatcttatgatggtgcggtcttgggagcctacacctggggagatggagaactgccggatgaattcctcgatttctatgaggagctggatgaaatagacttagcaggcatgaaggccgccgtattcgggagcggagacaccgggtatgatatttactgcggtgcggttgacctgattgaagcaaagctgaaggaaagaggcgccgtcatggtacagacgagtctgaaaattgaatatgggccgactgctatggagaaagaggattgccgcaaattcggacagcgttttgccttggcatgcgtaggggtatcttaaggtgagtacggcatgcggcgctgccgctaatccggcctctcatttccccaaatttcacgactatatcgtagagcgtacgctgctggatatggcggaggcaagcggcggcggaaaagaactgcatttgtggcggcagcaggtacagtatacggtcggccacacgttaaatgatgttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 5 - DNA, 1992 bp, synthetic construct
  misc_feature 1..1992: note = OTHER INFORMATION: ITR2-PgroES:Pz_fer-TCAT-CAT-PCAT-ITR1 expression cassette
  source 1..1992: mol_type = other DNA
aacaggttggctgataagtccccggtctactgagtcccaccacacccgcccgaccgaactaagttaagacacgcctagcacgaagcccacagaagtacgttgacaccaactgcatgatgatatatgtcaaaataataaaccaatactgttttctcaaatggtatgtactcctttgttaagtgggtttcgttcatctacagctattgtaacataatcggtacgggggtgaaaaagctaacggaaaagggagcggaaaagaatgatgtaagcgtgaaaaattttttatcttatcacttgaaattggaagggagattctttattataagaattgtgttagcactctttagtgctgagtgctaaaattacatattcatactattgaggaggttatttcgatggctaaatacacttgggtagagaaagatacctgcatcgcatgcggagcctgtggcgctaccgctcctgacatttacgattatgacgacgaaggactggctgaagttatctacgagggagatgccaaccgcggcgtaacggacattccggaagatttgttagacgatctgcaagatgccgctgacggatgtccgacggattccatcaagattgcggatacgcctttcaacaagtaaggttaattcttttgtttcacgaatttattaattggcttatgaaaagacatcccggttcacagagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 6 - DNA, 5182 bp, synthetic construct
  misc_feature 1..5182: note = OTHER INFORMATION: ITR2-PtrnQ:Pz_porCA-TCAT-CAT-PCAT-ITR1 expression cassette
  source 1..5182: mol_type = other DNA
aacaggttggctgataagtccccggtctactgagtcccaccacacccgcccgaccgaactaagttaagacacgcctagcacgaagcccacagaagtacgttgacaccaactgatatgcaaagtcatctctttttcccattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatgtcggtggtacaattaccgaaagtgaacgaacttgggtttttcgagatccgactcgaatccataggaggactcggcgcgaaccttgccggtaaaatgctggctgaagcaggggtagtaggcgcgaatttgaatggcgtcagcttctcgtcttatggttcggagaagaaaggttcgccggttaaagcgcatatccgtttctgcgatttgaatacgccgatccgcgatacttcgccggttgaacggccgcatgtcgttgccgtgttccacgaagccatgggcaagattgttaatgtaacaagcggcattaccgcagaaagcaccgtgctgatcaactcagcgaaatctcccgaagagctgaaggaaattcttggcgtaactgcgggcacaatcgctgttgtcgatgcgaccattatcgcgctggaagagaagaaccgcgtgaacatggcgatgctgggcggtctgttccgactctgtccgttccttgatcctaatacaatgaagaacgttatcgagaagtcgctcggcaagaaataccctcaagcggttcagtcggccatcacgactttcgaccgcggctacaatgaagtgaagttccaaacgtttgagctgccggaaggcgagagcatgccggaatttgtacggtccgacatttcgccgctcggatacgaaacccagccgattggcggcaccatcaccaatccgggaacaagcttcctgaaagatctcagcatttcccgttccgggcttgtgccggtatatgacaacgagtcctgcatcaactgcgcggagtgcgacacggtttgtccggatatgtgcttcgtttgggaagaaaaggtcaataaaaaaggcaaatcgatgatgttcctgaaagggatcgattatcaatactgtaaaggctgtctcaaatgcgtggaagcctgcccgacctccgcactctcccgttcgcgtgagaaggaaggcttcgcggaagctaatacggtccggcacacttttgatcttgtaaaccagatctaaatcccgagagaaaggtggaacactaatggctatcgattacgaaaaagaagtcggctccgccaaggtggagcagaagtttttatatgaatccggcaatgaaatggcggcttacgctgcgcatcaaatcaactatcatgtgatgggctatttcccgatctccccgtccacggaggttgcgcaattcctcgattccatgaaagccagcggacagcatgacatcatgctggtgccttctgacggcgagcacagctctgcgggcatctgttacggcgcatccaccgcaggcggccgcgtatttaacgcgacaagtgcccaaggctacctgtacatgctggagcagctcccggttcaatccggcacgcggatgccgatggtcatgaacctgatctgccgttccgtttccggaccgctgaacattcatggagatcactccgacctgtattatgcgctgaacaccggctggccgatcctgatgtgcccggatccacagtccgtatacgatatgaacatcatggcaatcaagctggccgagcatgccaaggttcgcctgcccgtgctggtagcgatggacggatactttacttctcaccagaagcgccgcgtccaagcgattgcgaaccgtgaagatgtccaaagctttatcggacctcaaccgcaaggttatgacgatacgctggaccgtgagaatccgatttccgtcggcccttatatgaacgaaccggactatatcaacaacaaataccaacaatccgtggcgatgtacaatgctgcggaagtattcgaagaaatcgccaaagaatatggcgagctgacaggccgccattacccgatcctgtcgcagtaccggatggaagatgccgaggttgcggtattcttgatgaactccgcttccgagatcatcaaggacgttgtcgatcagcttcgtgagaaaggcattaaagccggcgccatttcgccgaacatgatccgtccgttcccgcagaagcaaattgccgaagcgctgaagaacgttaaagccatcaccgtcggtgaccgcgcggattctttcggagcgcatggcggcaacatggttaacgaaatcaaggcggctctgttcacttacggcaatacaacaacgaaagtaatcagccggatttacggtgtcggcggcaaggacttctttgccgaagacggccacagcctgttcgagatggctattgaggcagcggaaaccggtgaagtgaaagtacctttcgattattacggccacaatccgggcgttgccgaaagcgcgccgaagcgtgtgatcaaaccgcttaaattcgagtctctcaagacaggcttgattactgtggacaaggatgaggaaacaggcaaactcaaggttaagattccgccaatgcgcagcttgaccgttaaaccgaagcgcttgtccccgggtcacggtacttgcccaggctgcggtattttctccggcctggagctgttcttcaaaggcatcgaaggagatgtcgttgctctgtaccacacaggctgcgcctttgttacgacgaccggctatccttattcgtcgcataaatcgacgttcatccataacctgttccaaagtggtgccgcaactatgtccggtgtggttgaaatgttctgggaacgcaaacgccgcggcgagcttgatcatctgaacctgaaagacgactttacctttgtcatggttaccggcgacggcggcatggacatcggtatggggccggcaatcggcgccgcgcttcgcggacataagatgatcgttctggaatatgacaatgagggctacatgaatacgggtgcacagcaatcgtactcgacgccgatcggtcaccgcacatcgacatccagcatcggcaagacgcaaaatggtaaggtgacgcagcataaagatactccgcaaattatggctgccaccaatattccttatgtgttcacaggctgcgaagcgttcccgcaggatttggtgaagaaagccgccaaagcccaatggtacgctcagaacgaagggcttgtatacggcaaaatcttgtctgcctgcccgctgaattggatgaccgaagacaatgtaggcaccaagctggtatccctggctgtcgattcctgcttcttcccgctgtatgaagtggagcaaggcgttaccacgattacgtacaatccggaagaaaaggaaaaacgcgtagacgttaccgaatggctcaaaggcatgggtaaaaccaagcatctgcttaaggaagagaacgaagctgcgctgaacggcttcaaggaagaagtagagcgccgctggagccgcctgaaagcgaagcacgagcatccggagctgtaagatccgcttgattgcttgaaaatggagctgtcccataagtagattttcaaagtcagagacagttcttcttcattctagaaaacgcaaaaagtcagcaaaacagcgattctccggttattggagaatcgctgttttgcgtttttggtccactgcggcttgttttagtacattatgggcaagggaaagccacccgacctcaagcgtcactttctccatgccgcgaagcagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 7 - DNA, 6807 bp, synthetic construct
  misc_feature 1..6807: note = OTHER INFORMATION: ITR2-PtrnQ:Pz_sufCDSUB-TCAT-CAT-PCAT-ITR1 expression cassette
  source 1..6807: mol_type = other DNA
aacaggttggctgataagtccccggtctactgagtcccaccacacccgcccgaccgaactaagttaagacacgcctagcacgaagcccacagaagtacgttgacaccaactgatatgcaaagtcatctctttttcccattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatggcagcagaatttgtcattgagggtttgaaagcgacgattgaggggaaagagatcttgaagggatttaaccttcagatgaagggcggggaggttcatgccatcatgggcccgaacggcactggtaaaagtactctggcgtcttcgctgatgggtcatccgaaatatgaagtgacggaaggaaccgtaacccttgagggtgaagatgtgctggagatgtccgttgacgagcgtgcccgcgccggattattccttgctatgcagtatccaagcgagatcgccggcgtaacgaactccgactttctgcgcagtgcgatcaatgcgcgccgcgaagagggaagtgaaatttcgctgatcaagtttatccgccaaatggaaggcaagatgaaggatttggagatgaatccggagttcatgcaccgctatttgaatgaaggcttctccggcggcgagaagaaacgcaacgaaattttgcaaatgctgctgcttgaaccgaaaattgtgattttggatgaaatcgattcgggtctggatatcgacgcgctgagggttgtcgcgcagggtgtaaactccatgcgaagcgaagaccggggtttcctggttatcactcactaccaacgtctgcttaactacattaaacccgactttgttcatattatgatgcaaggcagaatcgtaaaatccggaggccctgagcttgccgagcgattggaagccgatggttatgaatgggtgaaagaagaactggggatcgaagatgaaactgtagggcaggaagcttaagaacgcttggaaggaggagaacataatgactacgcaaaccattcttccggtggatgccggccgattaagcgaactgtcgctgagaagcggcgagccggaatggctgaaggaaagccgtcttaaggcgctggagctggccgcggaactggaattgccgaagctggagaagactcggattgatcgctggaatattaacaattacggcagttataaagaaggcgcggcgatcgccgcattgaacgaagcgccctccgccattgccgcgctagtaggcagctcggaggcaggaagccttattatccaacggaattctggagcggtctatacacggcttgcacctgagatggcggatcagggggtaatcttcacggatctgcacactgcggttaaggagcacggagagctggtgcggcggtacctgcacactgcggtcaaaccggaggagcattcggttgcggctctgcacgcggcgctgtggaacggtggcgtattcctgtacattccgaaaaatgtagtggtcgatacaccggttcaggctgtattcctgacagatgacgcggaggcttcatttgctccccatgttctgatcgttgcggacagcaacagttcggtaacttatgttgacaactatgtatcgggcaagagcgaagccggacttcacaacggcgcggtggaagtgttcgtctgcgccggagcgaaggttcgctacgcgacagtgcatcaattcggcgctgatactacggatgtaacgtatcgccgcgcgatcgtggagaatgacggatcggtcgagtggattgtcggcgaaatgaacgacggcgatacagccagcgacacgaagtcggtgcttaagggcaacggggcaagctccgacgcaaaagtcatcgcggtgggctcaggctcgcagaagctcaactacacgacgcaggcgcagcatttcggcaagaacacgccaagcaatatgattacccgtgcggttatgcgtgagaacgccacgtcgattatcaacggcattaccaaaatcgagaaaggcgcaacgcgcgctgacggccagcaaacggaaaaagtactaatgcttagccctaaggcgcgcggcgacgccaatccgattctgcttatagacgaggatgatgtgaccgcaggtcatgccgcttccgtagggcaggttaatccggaacaaatctattatctgatgtcccgcggtatttcgcgttcggacgcggagaaactggttatttacggcttccttgcgccggttgtgtcgcaaattccacttgagggactgcgtaatcagcttaagtctcttgtggaaaggaagttgggccaatgaacagctctatccgggaactattccctatcctagaccagaatgttaacggtcacccgctcgtttatctggatagtgcggcgacatcgcagaagccgcggcaggtaattgaagcgatcaaggcttactacgagcatgataattccaacgttcaccgcggcgtccatacgctgggcagccgcgcaaccgatgcttatgaaggagccagagagaaggttgccagatttattaacgcccatagtaccaaagaaatcgtatttacccgcggtacaacgacggctcttaatctggtagcttcctcttacgggccttccgccgtcggcgagggcgatgagatcgtgattaccctgatggagcaccacagtaacctgattccgtggcagcagcttgccaagaagacgggagcgactcttaagttcattccgctgcaaaaagacggaaccattctgctggaggatgccgagaagacaattacggacaacaccaagatcgtatccattgcctatgtctccaatgtaatgggcgtcaccagcccggtaaaagaaattgcggccatcgctcaccgccacggcgcggtcatggtcgttgacggcgcgcagagcacgccgcatatgaaggtggatgttcaggatctggactgcgacttctatgctttgtccggccacaaaatgtgcggtccaaccggcatcggagccctttacggcaaaagagcgctgcttgaagccatggaaccggttgagtttggcggggaaatgattgatgacgtcggcctttacgattcgacctggaaggaactaccctggaagttcgagggcggcaccccgatcatcgcgggcgcagtcggcttgggagcggcgatcgacttccttcaagaggtcggtctggatgagattcaccggcatgagattaagctcgcggcctatgcggagcagcgtctggccgaagttgaaggaatatcgatttacggaccgcgtaaccgggaagtgggcgtcataaccttcaatttgggggatgttcacccgcatgacgtagccactgtgcttgatgcggaaggaatagcggtccgggcgggccaccactgctgccagccgctgatgcgctggctggaagttagttctacggcaagggccagcctctacttgtataacaccgaagaagacgttgaccggctggcctcggcactaatccggacaaaggagtatttcgccgatgcaattagatgatttgtacagacgtgtaatcatggatcattataagaatccccggaaccgcggcttgttcgaagacgatgctatgaaggtggaactgaacaaccctacctgcggcgaccgcattacactgcagcttaaagtggaagacgggatcgtgaaggaagcccggtatatcggtgaaggctgttcgatcagcatgtcctccgcttcgatgatgaccgaagtggttaaagggaagacagtagagcaggcgctggatttggccagccgattctctgcactgatgaagggcgaggacgtgacgttcgacgattacgaagatttagaggccctgtccggcgttaacaaatttccggcgcggatcaaatgtgcgactctggcctggaatgcgctgcgcaaaggcatcgacgaggaagagcggcaacatcactaaataaactgaaggaggcgtaccccatggccaaaaaagcgcctgaaattgaagagtacaaatatggttttcgggacgagcacaaggcggtatttcaaacgggtaaagggctgactccggaaattgtgaaggaaatctcggcgattaagaatgagccgcagtggatgctggacttccgtctgaaatcgctcgagcaattcggtaaaatgccgctgccgcgttggggcggaaaccttgacgaactggatttcaacgacattcagtactatgtaagaccatccgagaagcagggcaagacctgggaagaagttccttccgaaatcaaggaaacattcgataagctcggcattcccgaagcggaacaaaagttccttgcaggagtatccgcacaatacgaatccgaggtcgtctaccatagcatgcagcatgatcttgaagagcagggtgtaatatttacggacacggacacggctttgcgtgagcatcccgagatctttaagcagtatttcggcaccgtgattcctccgaccgacaacaagttcgcagcgctcaacagcgccgtttggtcgggtggaagctttatctacgttccaaaaggcgttaaatgcgaaataccgctacaggcttacttccggatcaactccgagaacatgggacaattcgaaagaacgctgatcattgccgatgaggacagcttcgtgcattacgtggaaggctgcaccgctccggtctacagcacgaattcgctgcacagcgcggtcgttgaaatcatctgtaaaaagaacgcgcgtgtccgctatacaaccattcagaactgggcgccgaacatctacaacctcgttaccaaacgcgcggttgcggaagaaaacgcgacgatggaatgggttgacggcaacatcggttccaagctgacgatgaaatacccggcggtcgttctgaaaggacgcggagccaagggttccgtactctctatcgcggtagccggtaaaggccagcatcaggatgccggcgccaagatgatccatctggcaccggacacgacgtccaccatcatttccaagtcgatcagtaagcacggcgggaaagttacttaccgcggactcgcttcctttggccgtcaggcggaaggcgcgaagtcaaatattaaatgcgatacgctcattatggataacgaatctacttcggatacaattccttataatgagattatgaacgacaatattacgctggagcatgaagcgaccgtatccaaggtatccgaggagcagctcttctacctgatgagccgcggtcttacggaagccgaagcgacccagatgatcgtaatgggcttcattgagccgttcaccaaggaactgccgatggaatacgccgtcgagatgaaccgtcttatcaaattcgagatggaaggaagcatcggctagccttgaggtgatcaagggttcccggcttatgtaacatgcaaatacgtgtccgcagtgatcttgacgcatttttgcccaaggcgctcgggcggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 8 - DNA, 1909 bp, synthetic construct
  misc_feature 1..1909: note = OTHER INFORMATION: ITR2-PtrnQ:Pz_fer-TCAT-CAT-PCAT-ITR1 expression cassette
  source 1..1909: mol_type = other DNA
aacaggttggctgataagtccccggtctactgagtcccaccacacccgcccgaccgaactaagttaagacacgcctagcacgaagcccacagaagtacgttgacaccaactgatatgcaaagtcatctctttttcccattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatggctaaatacacttgggtagagaaagatacctgcatcgcatgcggagcctgtggcgctaccgctcctgacatttacgattatgacgacgaaggactggctgaagttatctacgagggagatgccaaccgcggcgtaacggacattccggaagatttgttagacgatctgcaagatgccgctgacggatgtccgacggattccatcaagattgcggatacgcctttcaacaagtaaggttaattcttttgtttcacgaatttattaattggcttatgaaaagacatcccggttcacagagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 9 - DNA, 2234 bp, synthetic construct
  misc_feature 1..2234: note = OTHER INFORMATION: ITR2-PtrnQ:Pz_fldA-TCAT-CAT-PCAT-ITR1 expression cassette
  source 1..2234: mol_type = other DNA
aacaggttggctgataagtccccggtctactgagtcccaccacacccgcccgaccgaactaagttaagacacgcctagcacgaagcccacagaagtacgttgacaccaactgatatgcaaagtcatctctttttcccattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatgattaaaatattagtggtatatgccagcctgaccggcaatacagaggaaatagcggaactgatcgcggagggaatccgccaatcgggaggggagacagtcataaaatcggtcaccgactgcaacgccttcgatatcgcatcttatgatggtgcggtcttgggagcctacacctggggagatggagaactgccggatgaattcctcgatttctatgaggagctggatgaaatagacttagcaggcatgaaggccgccgtattcgggagcggagacaccgggtatgatatttactgcggtgcggttgacctgattgaagcaaagctgaaggaaagaggcgccgtcatggtacagacgagtctgaaaattgaatatgggccgactgctatggagaaagaggattgccgcaaattcggacagcgttttgccttggcatgcgtaggggtatcttaaggtgagtacggcatgcggcgctgccgctaatccggcctctcatttccccaaatttcacgactatatcgtagagcgtacgctgctggatatggcggaggcaagcggcggcggaaaagaactgcatttgtggcggcagcaggtacagtatacggtcggccacacgttaaatgatgttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 10 - DNA, 6935 bp, synthetic construct
  misc_feature 1..6935: note = OTHER INFORMATION: ITR2-PsigX:Pf_sufCBSUB-TCAT-CAT-PCAT-ITR1 expression cassette
  source 1..6935: mol_type = other DNA
aacaggttggctgataagtccccggtctactgagtcccaccacacccgcccgaccgaactaagttaagacacgcctagcacgaagcccacagaagtacgttgacaccaagctatcgttaaaaatatcgtggaagcccacaacggatcaattactgtgcacagccgaatagataaaggaacaacattttctttttatattccgacaaaacggtaaaatcgagtctgaatttgccgaagaatcttgttccataagaaacacccgctgactgagcgggtgtttttttaatagccaacattaataaaatttaaggatatgttaatataaattcccttccaaattccagttactcgtaatatagttgtaatgtaacttttcaagctattcatacgacaaaaaagtgaacggaggggtttcagatgaaagggatgaacgttatggcagcagaatttgtcattgagggtttgaaagcgacgattgaggggaaagagatcttgaaggggtttaaccttcaaatgaagggcggcgaggttcatgccatcatgggaccgaacggcaccggcaaaagtacgcttgcctcttcactgatgggccatccgaaatatgaagtaacggaaggaaccgtaacccttgaaggtcaggatgtgctggaaatgtccgtcgacgaacgcgcccgcgcgggattgttcctggccatgcagtatcccagcgaaatcgccggcgtaacgaactccgactttcttcgcagtgcgctcaacgcgcgccgcgaagaaggaagtgaaatttcgctgatcaagtttatccgccaaatggaaagcaagatgaaggatctggagatgaatccggagttcatgcaccgttatttgaatgaaggcttttccggcggggagaagaagcgcaacgaaattctgcaaatgatgctgctggaaccgaagattgtcatcctggatgagatcgactcgggccttgatatcgacgcgctgagagttgtggcgcagggcgtaaacgccatgcggagcgaagaccgcggtttcctcgttatcactcactaccaacgtctgctcaactacatcaaacccgactttgttcatattatgatgcaaggaagaatcgttaaatcgggcggtccggagcttgccgagcgactggaagccgacggctatgaatgggtgaaggaagagctggggattgaagatgaagtcgtaggacaggaagtttaataacgcctggaaggaggagaacattatgactacgcaaaccattcttccggtggacgccgggcgattgagcgaactgtcgctgcgaagcggcgagccggagtggctgaaggaaagccgcctgaaggcactggagctggccgctgaactggaactgccaaagctggagaagacccgtattgaccgctggaatatcaaccaatacggcagctataaagaaagcgcggcgatcgccgcattgaacgaagccccctcctccattgccgcgcttgttggcagctcggaggaaggcagcctcattattcaacggaattcgggagcggtctacacgcggctcgcgccggaactggctgagcagggtgtaatcttcacggatctgcacactgcggtcaagaatcacggagagctggtgcgccgttatttgcatacggcggtcaaaccggaggagcataaggtagccgccctgcacgcggcgctgtggaacggcggcgtattcctgtacattccgaagaatgtagtggtcgatacaccggttcaggctatattcctgacagacgacgccgaggcttcattcgctccccatatcctgattgtagcggacagcaacagttcggtaacctatgtcgacaactacgtttcgggcaagagcgaagccggacttcataacggcgcagtggaagtattcgcctgcgccggagcgaaggttcgctacgcaacggtgcatcaattcggagccgatacgacggatgttacgtaccgccgcgccatcgtggagaacgacggagcggttgaatggattgtcggcgaaatgaacgacggcgatacggccagcgacacgaagtcggtgcttaagggcaacggagcaagctccgacgccaaagtgatcgcggtaggctcgggctcgcagaagctcaactatacgacgcaggcgcagcattttggcaagaacacgccaagcaatatgattacccgcgcggtcatgcgcgagaacgccacgtcgattattaacggaattaccaagatcgagaaaggcgcgacgcgggctgacggccaacagacggaaaaagtgctgatgctgagcccgaaggcgcgcggcgacgccaatccgattctgcttatagatgaggacgatgttacagcagggcatgccgcttccgtagggcaggtcaatccggaacaaatctattatctcatgtcccgcggtatttcgcgttcggatgcggagaaactggttatttacggcttccttgcgccggttgtgtcgcaaattccacttgagggacttcgcaatcagcttaagtctcttgtggaaaggaagttaggccaatgaacagcgcgatccgggagctattccctatcctgaaccaaaatgttaacggtcatccgcttgtgtatctggacagcgccgcaacctcgcagaagccacggcaggtcattgaagcgatcaaggcatactacgagtacgataactccaacgttcaccgcggcgtccatacgctgggcagccgcgcaaccgatgcctatgaaggagccagagagaaggttgccagatttattaacgcccacagcaccaaagagatcgtgtttacacgcggtacgacgacggctttgaatctggtagcttcctcttacggtccttccgccgtcggagagggcgatgagattgtcactaccttgatggagcaccacagcaacctgattccttggcagcagctcgccaagaagacgggagcgacgctcaagttcattccgcttcagaaggacggaaccattctgctggaggatgccgagaagacgattacggacaatacgaagatcgtttccgtggcctatgtctccaatgtaatgggcgttaccagtccggtgaaggagattgcagctatcgcgcaccgtcacggcgcggtcatggtggtcgacggcgcgcagagcacgccgcatatgaaggttgacgttcaggatctggactgcgacttctatgccttgtccggccacaagatgtgcgggccgaccggtatcggcgccctgtacggcaagagagcgctgcttgaagccatggaaccggttgagtttggcggggaaatgatcgatgatgtcggcttgtacgattccacctggaaggagcttccctggaagttcgagggcggcacgccgatcatcgcgggcgcagtcggcctgggggcggcaattgactttcttcaagaggtcggtctggatgaaatccatcggcatgagatcaagcttgcggcctacgccgagcagcgtcttgccgaagttgaggggataacgatctatggacctcgcaaccggaaagtgggcgtcgtcaccttcaatctgggggacgttcacccccatgacgtagccactgtgctcgatgcggagggaatagcagtccgggcggggcatcactgctgccagccactgatgcgctggctggaagctagttcaacggcaagggccagcctgtatttgtacaacaccgaagaggatattgaccggctggccgtggcattaatccggacaaaggagtatttcgccgatgcaattggatgatttgtacagacgtgtcattatggatcattataagaatccccggaaccgcggcttgttcgaggatgacagcatgaaggtcgaattgaacaatccaacctgcggcgaccgtattaccttacagcttaaagtggaagacggaattgtgaaggaagcccggtatgtcggcgaaggctgctcgatcagcatgtcctccgcatcgatgatgaccgaagtcgttaaagggaagaccgtagagaaggcgctggatctggccagccgattctccgcactgatgaaaggcgaggacgtgacgttcgacgattacgaggatctggaggccctttccggcgtcaacaaatttccggcccggatcaagtgtgcgacactggcctggaatgcgctgcgcaagggcatcgacgaggaagagcggcaccaccactaaataaactgaaggaggcttacccctatggccaaaaaagcgcctgaaattgaagagtacaaatatggttttcgggacgagcacaaggcggtatttcaaacgggtaaagggctgactccggaaattgtcaaggaaatctcggcgatcaagaatgagccggagtggatgctgaacttccgtctgaaatcgctcgaacaattcggtaaaatgccgctgccgcgctggggcggaaaccttgacgagcttgatttcgacgatatacagtactacgtgaggccatccgagaagcagggcaagacctgggaagaggttccttccgaaattaaggaaaccttcgataagctcggtatccccgaagcggagcaaaaattccttgcgggtgtgtcggcgcagtacgaatccgaggttgtctatcatagcatgcagcaggatcttgaagagcagggcgttatatttacggatacggatacggctctgcgcgagcatccagagattttcaaggagtacttcggcaccgtgattcctccgacagacaacaagttcgctgcgctgaacagcgccgtttggtcgggcggaagctttatctacgttccgaagggtgtgaaatgcgaaattccgctgcaggcttacttccgcatcaactccgagaacatgggacaattcgaaagaacgctgatcattgcggatgaggacagctttgtgcactatgtggaaggctgtaccgctccggtctacagcacgaattcgctgcacagcgcggtcgttgaaatcatctgtaaaaagaacgcgcgcgtccgctataccaccattcagaactgggcaccgaacatctacaaccttgttaccaaacgcgcagttgcggaagaaaacgcgacgatggaatgggttgacggcaacatcggttccaagctgacgatgaaatacccggcagtcgttctgaaaggacgcggagccaaaggttccgtgctctccatcgcggtggccggcaaaggccagcaccaggatgccggcgccaagatgattcatctggcaccggacacaacgtcgactatcatttccaagtcgatcagcaagcacggcggaaaggtgacttaccgcggactcgcttccttcggccgccaggcggaaggcgcgaagtcgaatatcaaatgcgatacgctcattatggataacgaatctacttcggatacgattccttataatgagattatgaacgataatattacgctggagcatgaagcgaccgtttccaaggtatccgaggagcagctcttctacctgatgagccggggccttacggaagccgaagcgacccagatgatcgtcatgggcttcattgagccgttcaccaaggagctgccaatggaatacgccgtcgagatgaaccgtcttatcaagtttgagatggagggaagcatcggctagtcttgaacaatcaaggccgtccggcttgcctaacatgcaaatacgtgtccgcagcggtcttgacgcttttatgcccaaggcgctcggacacgtaggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 11 - DNA, 5097 bp, synthetic construct
  misc_feature 1..5097: note = OTHER INFORMATION: ITR2-PmreB:Pf_porCA-TCAT-CAT-PCAT-ITR1 expression cassette
  source 1..5097: mol_type = other DNA
aacaggttggctgataagtccccggtctactgagtcccaccacacccgcccgaccgaactaagttaagacacgcctagcacgaagcccacagaagtacgttgacaccaatagacgaatgaaactttgaaaaagtttcccgctttctataagaattacaagatattttttccataaaaccgaaaaatgtattacttctgttacacagttccttgattttttacaaaaacatttctttaccgacgcacacccccaaaaatcgcagtatttctgagaaactttaaatgtagaatcatattaaattaggatagagattgggtgaaaagatgtcggtggtacaattaccgaaagttaacgaactcgggtttttcgagatccggctcgaatccataggaggtcttggcgcgaacctggccggtaagatgctggctgaagcaggggttgtaggcgcgaatttgaatggcgtcagcttctcgtcttatggttcggagaagaaaggttctccggttaaagcgcatatccgcttctgcgatttgaatacgccgatccgcgatacttcgccggttgaacggccgcatgtcgttgccgtgttccatgaagctatgggcaagattgtgaatgttacaagcggtatttccgcaggaagcacggtactgataaactccgcgaagtctcccgaagagctgaaggaactgctcggtgttaaagcaggcacaatcgcggttgtcgatgcaaccatcatcgcgctcgaagagaagaaccgcgtgaatatggcgatgctcggcggactgttccggctctgcccgttccttgatcctaatacaatgaagagcgttattgagaagtcgctcggcaagaaatatccgcaggcggttcagtcggccatcacgactttcgaccgcggctacaacgaagtgaagttccagacatttgaactcccggaaggcgagagcatgccggaatacgtacggtccgacatttcgccgctcggatttgaaactcagccgattgggggcacgattaccaatccgggtacaaccttcctgaaggatctcagcatttcccgttccgggcttgtgccggtatatgacaacgagtcctgcatcaactgtgcggaatgcgacaccgtatgtccggacatgtgcttcgtttgggaagagcagatcaataaaaaaggcagatcgatgatgttcctgaaagggatcgactaccaatactgtaaaggctgtctcaaatgtgtagaagcctgcccgacctccgcactctcccgttcccgtgagaaggaaggctttgcggagtccaaagcggttcgtcacacgtttgaccttgtaaaccagatctaattcccgagagaaaggtggaactcaaatggctatagattacgaaaaggaagtcggctccgccaaggtggagcagaagtttttatatgaatccggcaacgaaatggcggcttatgctgcacatcaaatcaactatcatgtaatgggctatttcccgatctccccatctacggaggttgcacagttcctggacgggatgaaagccagcggccagcatgacatcatgcttgttccttccgacggcgagcacagttccgcaggtatttgctatggcgcgtctacagcgggcggccgcgtattcaacgcgacaagcgctcaaggctacctgtacatgctggagcagcttccggttcaatccggcacacggatgccgatggtcatgaatcttatctgccggtccgtttccggtccgctgaacattcacggcgatcactccgacctgtattatgcgctgaacaccggctggccgatcctgatgtgtccggacccgcaatctgtatatgatatgaacatcatggcaatcaagcttgccgagcatgcgaaggttcgcctgcctgtgctggtagccatggacggttacttcacttctcaccagaagcgccgcgtccaagcgattgcgagccgtgaggatgttcaaagctttatcgggccgcaaccaaagggttatgacgacacattggaccgcgagaatccgatttctgtcggcccttacatgaacgagcccgactatatcaacaacaaatatcaacaatccgtggctatgtacaacgccgaggaagtatttgaagaaatcgccaaagaatacggcgatctgacgggccgctattacccgatcctgtcccagtaccggatggaagacgcggaggttgcggtattcctgatgaactctgcttccgagatcatcaaggatgttgttgaccagctccgtgagaaaggcgtgaaagctggagctatttcgccgaacatgatccgtccgttcccgcagaagcaaattgctgaagcgctgaagaacgttaaagccatcaccgttggcgaccgcggagattcctacggagcatacggcggcaacatggttaacgaaatcaaggcggcgctgttcactcacggcaacacgacaacgaaggtaatcagccgcatctatggcgtcggaggcaaggacttctatgccgaggatggtcatgccctgttcgagctggctattgaagctgccgaaaccggaacagtcaaagttccttttgactactacggccacaatccgggcgtagccgaaagcgcgccgaagcgcgtgctcaagccgcttaaattcgagtcgctcaagactggcctgattactgtggacaaggatgaagagactggcaagctcaaggtcaaggttccgccgatgcgcagcttgactgtgaagccgaaacgtctgtcccctggtcacggcacttgtccgggttgcggtattttctccggtctggagctgttcttcaaaggcatcgaaggagatgtcgttgcgctgtaccatacaggctgcgcctttgttacaacgaccggttatccttattcttcccataaatcgactttcattcacaacctgttccaaagcggggccgctacaatgtccggtgtggttgaaatgttctgggaacgcaaacgccgcggagaacttgatgatcttggcctgaaggacgatttcactttcgttatggttaccggcgacggcggtatggacatcggtatgggaccggcaatcggcgccgcgcttcgcggacacaagatgatcgtgctggaatatgacaacgaaggctacatgaatacaggggctcaacaatcgtactccacgccaatcggtcaccgcacatcgacttccagcattggcaagatgcaaaaaggtaaagtgacgcagcataaagatactccgcaaattatggctgccaccaatattccttatgtgttcacaggctgcgaagcgttcccgcaggatttggtgaagaaagccgccaaagcccaatggtacgctcagaacgaaggccttgtatacggtaagatcctgtcggcttgcccgctgaactggatgaccgaggacaacgtaggcaccaagctggtgtctctggctgttgattcctgcttcttcccgctgtacgaagtggagcaaggcgttaccacgattacgtacaatccggaagacaaggagaaacgcatcgacgtaacggaatggctcaaaggtatgggtaaaaccaaacatctgctcaaggaagagaacgaagctgcgctgaacggcttcaaggaagaagtagagcgccgctggatccgtctgaaagcgaagcacgagcatccggaactgtaagaacaagagctgctttatagcttgaattgaacagcaagcctcctttatttggaggcttgctgttttgcattttgcctccgaaatgatcacagtatattcacgtttcagggattgaattttccggccctgggttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 12 - DNA, 2369 bp, synthetic construct
  misc_feature 1..2369: note = OTHER INFORMATION: ITR2-PsigX:Pf_fldA-TCAT-CAT-PCAT-ITR1 expression cassette
  source 1..2369: mol_type = other DNA
aacaggttggctgataagtccccggtctactgagtcccaccacacccgcccgaccgaactaagttaagacacgcctagcacgaagcccacagaagtacgttgacaccaagctatcgttaaaaatatcgtggaagcccacaacggatcaattactgtgcacagccgaatagataaaggaacaacattttctttttatattccgacaaaacggtaaaatcgagtctgaatttgccgaagaatcttgttccataagaaacacccgctgactgagcgggtgtttttttaatagccaacattaataaaatttaaggatatgttaatataaattcccttccaaattccagttactcgtaatatagttgtaatgtaacttttcaagctattcatacgacaaaaaagtgaacggaggggtttcagatggctaaaatattggtggtatatgccagcctgaccggcaatacagaggaaattgcggaactgatcgcggagggaatccgccagtcaggaggcgaggcggttctgaaattggtcactgactgcaacgccttcgatatcgcatcctatgacggggcggtactcggggcctacacctggggagatggagaactgccggatgaatttctcgatttctatgaggagctggatgaaatagacctggcaggaaccaaagccgccgtgttcggaagcggcgactccggatatgaaatctactgcggcgcggttgaccggattgaagaaaagctgaaagaaagaggcgcggccatagtacaggcgagtctcaagatcgaatatgggccgactgcggtggagaaagaggaatgccgcaagttcggacagcgatttgccgaggcctgcgtggcggtatcttaatatggatacgtcatgcggcgcagccgctaattcggcctcccgtcttcccaaatttcatgattatattgttgagcgggcgctgctggacatggcctagacgaacggaggtggaaatggactgcctctgtggcggcagtaggtgcagtatgcggtcggtcatacgttaaatgattactacagcttgacgcccggggtgcgcgcagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 13 - DNA, 5190 bp, synthetic construct
  misc_feature 1..5190: note = OTHER INFORMATION: ITR2-PsigX:Pf_porCA-TCAT-CAT-PCAT-ITR1 expression cassette
  source 1..5190: mol_type = other DNA
aacaggttggctgataagtccccggtctactgagtcccaccacacccgcccgaccgaactaagttaagacacgcctagcacgaagcccacagaagtacgttgacaccaagctatcgttaaaaatatcgtggaagcccacaacggatcaattactgtgcacagccgaatagataaaggaacaacattttctttttatattccgacaaaacggtaaaatcgagtctgaatttgccgaagaatcttgttccataagaaacacccgctgactgagcgggtgtttttttaatagccaacattaataaaatttaaggatatgttaatataaattcccttccaaattccagttactcgtaatatagttgtaatgtaacttttcaagctattcatacgacaaaaaagtgaacggaggggtttcagatgtcggtggtacaattaccgaaagttaacgaactcgggtttttcgagatccggctcgaatccataggaggtcttggcgcgaacctggccggtaagatgctggctgaagcaggggttgtaggcgcgaatttgaatggcgtcagcttctcgtcttatggttcggagaagaaaggttctccggttaaagcgcatatccgcttctgcgatttgaatacgccgatccgcgatacttcgccggttgaacggccgcatgtcgttgccgtgttccatgaagctatgggcaagattgtgaatgttacaagcggtatttccgcaggaagcacggtactgataaactccgcgaagtctcccgaagagctgaaggaactgctcggtgttaaagcaggcacaatcgcggttgtcgatgcaaccatcatcgcgctcgaagagaagaaccgcgtgaatatggcgatgctcggcggactgttccggctctgcccgttccttgatcctaatacaatgaagagcgttattgagaagtcgctcggcaagaaatatccgcaggcggttcagtcggccatcacgactttcgaccgcggctacaacgaagtgaagttccagacatttgaactcccggaaggcgagagcatgccggaatacgtacggtccgacatttcgccgctcggatttgaaactcagccgattgggggcacgattaccaatccgggtacaaccttcctgaaggatctcagcatttcccgttccgggcttgtgccggtatatgacaacgagtcctgcatcaactgtgcggaatgcgacaccgtatgtccggacatgtgcttcgtttgggaagagcagatcaataaaaaaggcagatcgatgatgttcctgaaagggatcgactaccaatactgtaaaggctgtctcaaatgtgtagaagcctgcccgacctccgcactctcccgttcccgtgagaaggaaggctttgcggagtccaaagcggttcgtcacacgtttgaccttgtaaaccagatctaattcccgagagaaaggtggaactcaaatggctatagattacgaaaaggaagtcggctccgccaaggtggagcagaagtttttatatgaatccggcaacgaaatggcggcttatgctgcacatcaaatcaactatcatgtaatgggctatttcccgatctccccatctacggaggttgcacagttcctggacgggatgaaagccagcggccagcatgacatcatgcttgttccttccgacggcgagcacagttccgcaggtatttgctatggcgcgtctacagcgggcggccgcgtattcaacgcgacaagcgctcaaggctacctgtacatgctggagcagcttccggttcaatccggcacacggatgccgatggtcatgaatcttatctgccggtccgtttccggtccgctgaacattcacggcgatcactccgacctgtattatgcgctgaacaccggctggccgatcctgatgtgtccggacccgcaatctgtatatgatatgaacatcatggcaatcaagcttgccgagcatgcgaaggttcgcctgcctgtgctggtagccatggacggttacttcacttctcaccagaagcgccgcgtccaagcgattgcgagccgtgaggatgttcaaagctttatcgggccgcaaccaaagggttatgacgacacattggaccgcgagaatccgatttctgtcggcccttacatgaacgagcccgactatatcaacaacaaatatcaacaatccgtggctatgtacaacgccgaggaagtatttgaagaaatcgccaaagaatacggcgatctgacgggccgctattacccgatcctgtcccagtaccggatggaagacgcggaggttgcggtattcctgatgaactctgcttccgagatcatcaaggatgttgttgaccagctccgtgagaaaggcgtgaaagctggagctatttcgccgaacatgatccgtccgttcccgcagaagcaaattgctgaagcgctgaagaacgttaaagccatcaccgttggcgaccgcggagattcctacggagcatacggcggcaacatggttaacgaaatcaaggcggcgctgttcactcacggcaacacgacaacgaaggtaatcagccgcatctatggcgtcggaggcaaggacttctatgccgaggatggtcatgccctgttcgagctggctattgaagctgccgaaaccggaacagtcaaagttccttttgactactacggccacaatccgggcgtagccgaaagcgcgccgaagcgcgtgctcaagccgcttaaattcgagtcgctcaagactggcctgattactgtggacaaggatgaagagactggcaagctcaaggtcaaggttccgccgatgcgcagcttgactgtgaagccgaaacgtctgtcccctggtcacggcacttgtccgggttgcggtattttctccggtctggagctgttcttcaaaggcatcgaaggagatgtcgttgcgctgtaccatacaggctgcgcctttgttacaacgaccggttatccttattcttcccataaatcgactttcattcacaacctgttccaaagcggggccgctacaatgtccggtgtggttgaaatgttctgggaacgcaaacgccgcggagaacttgatgatcttggcctgaaggacgatttcactttcgttatggttaccggcgacggcggtatggacatcggtatgggaccggcaatcggcgccgcgcttcgcggacacaagatgatcgtgctggaatatgacaacgaaggctacatgaatacaggggctcaacaatcgtactccacgccaatcggtcaccgcacatcgacttccagcattggcaagatgcaaaaaggtaaagtgacgcagcataaagatactccgcaaattatggctgccaccaatattccttatgtgttcacaggctgcgaagcgttcccgcaggatttggtgaagaaagccgccaaagcccaatggtacgctcagaacgaaggccttgtatacggtaagatcctgtcggcttgcccgctgaactggatgaccgaggacaacgtaggcaccaagctggtgtctctggctgttgattcctgcttcttcccgctgtacgaagtggagcaaggcgttaccacgattacgtacaatccggaagacaaggagaaacgcatcgacgtaacggaatggctcaaaggtatgggtaaaaccaaacatctgctcaaggaagagaacgaagctgcgctgaacggcttcaaggaagaagtagagcgccgctggatccgtctgaaagcgaagcacgagcatccggaactgtaagaacaagagctgctttatagcttgaattgaacagcaagcctcctttatttggaggcttgctgttttgcattttgcctccgaaatgatcacagtatattcacgtttcagggattgaattttccggccctgggttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 14 - DNA, 2246 bp, synthetic construct
  misc_feature 1..2246: note = OTHER INFORMATION: ITR2-PmreB:Pz_fldA-TCAT-CAT-PCAT-ITR1 expression cassette
  source 1..2246: mol_type = other DNA
aacaggttggctgataagtccccggtctactgagtcccaccacacccgcccgaccgaactaagttaagacacgcctagcacgaagcccacagaagtacgttgacaccaatagacgaatgaaactttgaaaaagtttcccgctttctataagaattacaagatattttttccataaaaccgaaaaatgtattacttctgttacacagttccttgattttttacaaaaacatttctttaccgacgcacacccccaaaaatcgcagtatttctgagaaactttaaatgtagaatcatattaaattaggatagagattgggtgaaaagatgattaaaatattagtggtatatgccagcctgaccggcaatacagaggaaatagcggaactgatcgcggagggaatccgccaatcgggaggggagacagtcataaaatcggtcaccgactgcaacgccttcgatatcgcatcttatgatggtgcggtcttgggagcctacacctggggagatggagaactgccggatgaattcctcgatttctatgaggagctggatgaaatagacttagcaggcatgaaggccgccgtattcgggagcggagacaccgggtatgatatttactgcggtgcggttgacctgattgaagcaaagctgaaggaaagaggcgccgtcatggtacagacgagtctgaaaattgaatatgggccgactgctatggagaaagaggattgccgcaaattcggacagcgttttgccttggcatgcgtaggggtatcttaaggtgagtacggcatgcggcgctgccgctaatccggcctctcatttccccaaatttcacgactatatcgtagagcgtacgctgctggatatggcggaggcaagcggcggcggaaaagaactgcatttgtggcggcagcaggtacagtatacggtcggccacacgttaaatgatgttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 15 - DNA, 6842 bp, synthetic construct
  misc_feature 1..6842: note = OTHER INFORMATION: ITR2-PmreB:Pf_sufCBSUB-TCAT-CAT-PCAT-ITR1 expression cassette
  source 1..6842: mol_type = other DNA
aacaggttggctgataagtccccggtctactgagtcccaccacacccgcccgaccgaactaagttaagacacgcctagcacgaagcccacagaagtacgttgacaccaatagacgaatgaaactttgaaaaagtttcccgctttctataagaattacaagatattttttccataaaaccgaaaaatgtattacttctgttacacagttccttgattttttacaaaaacatttctttaccgacgcacacccccaaaaatcgcagtatttctgagaaactttaaatgtagaatcatattaaattaggatagagattgggtgaaaagatgaaagggatgaacgttatggcagcagaatttgtcattgagggtttgaaagcgacgattgaggggaaagagatcttgaaggggtttaaccttcaaatgaagggcggcgaggttcatgccatcatgggaccgaacggcaccggcaaaagtacgcttgcctcttcactgatgggccatccgaaatatgaagtaacggaaggaaccgtaacccttgaaggtcaggatgtgctggaaatgtccgtcgacgaacgcgcccgcgcgggattgttcctggccatgcagtatcccagcgaaatcgccggcgtaacgaactccgactttcttcgcagtgcgctcaacgcgcgccgcgaagaaggaagtgaaatttcgctgatcaagtttatccgccaaatggaaagcaagatgaaggatctggagatgaatccggagttcatgcaccgttatttgaatgaaggcttttccggcggggagaagaagcgcaacgaaattctgcaaatgatgctgctggaaccgaagattgtcatcctggatgagatcgactcgggccttgatatcgacgcgctgagagttgtggcgcagggcgtaaacgccatgcggagcgaagaccgcggtttcctcgttatcactcactaccaacgtctgctcaactacatcaaacccgactttgttcatattatgatgcaaggaagaatcgttaaatcgggcggtccggagcttgccgagcgactggaagccgacggctatgaatgggtgaaggaagagctggggattgaagatgaagtcgtaggacaggaagtttaataacgcctggaaggaggagaacattatgactacgcaaaccattcttccggtggacgccgggcgattgagcgaactgtcgctgcgaagcggcgagccggagtggctgaaggaaagccgcctgaaggcactggagctggccgctgaactggaactgccaaagctggagaagacccgtattgaccgctggaatatcaaccaatacggcagctataaagaaagcgcggcgatcgccgcattgaacgaagccccctcctccattgccgcgcttgttggcagctcggaggaaggcagcctcattattcaacggaattcgggagcggtctacacgcggctcgcgccggaactggctgagcagggtgtaatcttcacggatctgcacactgcggtcaagaatcacggagagctggtgcgccgttatttgcatacggcggtcaaaccggaggagcataaggtagccgccctgcacgcggcgctgtggaacggcggcgtattcctgtacattccgaagaatgtagtggtcgatacaccggttcaggctatattcctgacagacgacgccgaggcttcattcgctccccatatcctgattgtagcggacagcaacagttcggtaacctatgtcgacaactacgtttcgggcaagagcgaagccggacttcataacggcgcagtggaagtattcgcctgcgccggagcgaaggttcgctacgcaacggtgcatcaattcggagccgatacgacggatgttacgtaccgccgcgccatcgtggagaacgacggagcggttgaatggattgtcggcgaaatgaacgacggcgatacggccagcgacacgaagtcggtgcttaagggcaacggagcaagctccgacgccaaagtgatcgcggtaggctcgggctcgcagaagctcaactatacgacgcaggcgcagcattttggcaagaacacgccaagcaatatgattacccgcgcggtcatgcgcgagaacgccacgtcgattattaacggaattaccaagatcgagaaaggcgcgacgcgggctgacggccaacagacggaaaaagtgctgatgctgagcccgaaggcgcgcggcgacgccaatccgattctgcttatagatgaggacgatgttacagcagggcatgccgcttccgtagggcaggtcaatccggaacaaatctattatctcatgtcccgcggtatttcgcgttcggatgcggagaaactggttatttacggcttccttgcgccggttgtgtcgcaaattccacttgagggacttcgcaatcagcttaagtctcttgtggaaaggaagttaggccaatgaacagcgcgatccgggagctattccctatcctgaaccaaaatgttaacggtcatccgcttgtgtatctggacagcgccgcaacctcgcagaagccacggcaggtcattgaagcgatcaaggcatactacgagtacgataactccaacgttcaccgcggcgtccatacgctgggcagccgcgcaaccgatgcctatgaaggagccagagagaaggttgccagatttattaacgcccacagcaccaaagagatcgtgtttacacgcggtacgacgacggctttgaatctggtagcttcctcttacggtccttccgccgtcggagagggcgatgagattgtcactaccttgatggagcaccacagcaacctgattccttggcagcagctcgccaagaagacgggagcgacgctcaagttcattccgcttcagaaggacggaaccattctgctggaggatgccgagaagacgattacggacaatacgaagatcgtttccgtggcctatgtctccaatgtaatgggcgttaccagtccggtgaaggagattgcagctatcgcgcaccgtcacggcgcggtcatggtggtcgacggcgcgcagagcacgccgcatatgaaggttgacgttcaggatctggactgcgacttctatgccttgtccggccacaagatgtgcgggccgaccggtatcggcgccctgtacggcaagagagcgctgcttgaagccatggaaccggttgagtttggcggggaaatgatcgatgatgtcggcttgtacgattccacctggaaggagcttccctggaagttcgagggcggcacgccgatcatcgcgggcgcagtcggcctgggggcggcaattgactttcttcaagaggtcggtctggatgaaatccatcggcatgagatcaagcttgcggcctacgccgagcagcgtcttgccgaagttgaggggataacgatctatggacctcgcaaccggaaagtgggcgtcgtcaccttcaatctgggggacgttcacccccatgacgtagccactgtgctcgatgcggagggaatagcagtccgggcggggcatcactgctgccagccactgatgcgctggctggaagctagttcaacggcaagggccagcctgtatttgtacaacaccgaagaggatattgaccggctggccgtggcattaatccggacaaaggagtatttcgccgatgcaattggatgatttgtacagacgtgtcattatggatcattataagaatccccggaaccgcggcttgttcgaggatgacagcatgaaggtcgaattgaacaatccaacctgcggcgaccgtattaccttacagcttaaagtggaagacggaattgtgaaggaagcccggtatgtcggcgaaggctgctcgatcagcatgtcctccgcatcgatgatgaccgaagtcgttaaagggaagaccgtagagaaggcgctggatctggccagccgattctccgcactgatgaaaggcgaggacgtgacgttcgacgattacgaggatctggaggccctttccggcgtcaacaaatttccggcccggatcaagtgtgcgacactggcctggaatgcgctgcgcaagggcatcgacgaggaagagcggcaccaccactaaataaactgaaggaggcttacccctatggccaaaaaagcgcctgaaattgaagagtacaaatatggttttcgggacgagcacaaggcggtatttcaaacgggtaaagggctgactccggaaattgtcaaggaaatctcggcgatcaagaatgagccggagtggatgctgaacttccgtctgaaatcgctcgaacaattcggtaaaatgccgctgccgcgctggggcggaaaccttgacgagcttgatttcgacgatatacagtactacgtgaggccatccgagaagcagggcaagacctgggaagaggttccttccgaaattaaggaaaccttcgataagctcggtatccccgaagcggagcaaaaattccttgcgggtgtgtcggcgcagtacgaatccgaggttgtctatcatagcatgcagcaggatcttgaagagcagggcgttatatttacggatacggatacggctctgcgcgagcatccagagattttcaaggagtacttcggcaccgtgattcctccgacagacaacaagttcgctgcgctgaacagcgccgtttggtcgggcggaagctttatctacgttccgaagggtgtgaaatgcgaaattccgctgcaggcttacttccgcatcaactccgagaacatgggacaattcgaaagaacgctgatcattgcggatgaggacagctttgtgcactatgtggaaggctgtaccgctccggtctacagcacgaattcgctgcacagcgcggtcgttgaaatcatctgtaaaaagaacgcgcgcgtccgctataccaccattcagaactgggcaccgaacatctacaaccttgttaccaaacgcgcagttgcggaagaaaacgcgacgatggaatgggttgacggcaacatcggttccaagctgacgatgaaatacccggcagtcgttctgaaaggacgcggagccaaaggttccgtgctctccatcgcggtggccggcaaaggccagcaccaggatgccggcgccaagatgattcatctggcaccggacacaacgtcgactatcatttccaagtcgatcagcaagcacggcggaaaggtgacttaccgcggactcgcttccttcggccgccaggcggaaggcgcgaagtcgaatatcaaatgcgatacgctcattatggataacgaatctacttcggatacgattccttataatgagattatgaacgataatattacgctggagcatgaagcgaccgtttccaaggtatccgaggagcagctcttctacctgatgagccggggccttacggaagccgaagcgacccagatgatcgtcatgggcttcattgagccgttcaccaaggagctgccaatggaatacgccgtcgagatgaaccgtcttatcaagtttgagatggagggaagcatcggctagtcttgaacaatcaaggccgtccggcttgcctaacatgcaaatacgtgtccgcagcggtcttgacgcttttatgcccaaggcgctcggacacgtaggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 16 - DNA, 5194 bp, synthetic construct
  misc_feature 1..5194: note = OTHER INFORMATION: ITR2-PmreB:Pz_porCA-TCAT-CAT-PCAT-ITR1 expression cassette
  source 1..5194: mol_type = other DNA
aacaggttggctgataagtccccggtctactgagtcccaccacacccgcccgaccgaactaagttaagacacgcctagcacgaagcccacagaagtacgttgacaccaatagacgaatgaaactttgaaaaagtttcccgctttctataagaattacaagatattttttccataaaaccgaaaaatgtattacttctgttacacagttccttgattttttacaaaaacatttctttaccgacgcacacccccaaaaatcgcagtatttctgagaaactttaaatgtagaatcatattaaattaggatagagattgggtgaaaagatgtcggtggtacaattaccgaaagtgaacgaacttgggtttttcgagatccgactcgaatccataggaggactcggcgcgaaccttgccggtaaaatgctggctgaagcaggggtagtaggcgcgaatttgaatggcgtcagcttctcgtcttatggttcggagaagaaaggttcgccggttaaagcgcatatccgtttctgcgatttgaatacgccgatccgcgatacttcgccggttgaacggccgcatgtcgttgccgtgttccacgaagccatgggcaagattgttaatgtaacaagcggcattaccgcagaaagcaccgtgctgatcaactcagcgaaatctcccgaagagctgaaggaaattcttggcgtaactgcgggcacaatcgctgttgtcgatgcgaccattatcgcgctggaagagaagaaccgcgtgaacatggcgatgctgggcggtctgttccgactctgtccgttccttgatcctaatacaatgaagaacgttatcgagaagtcgctcggcaagaaataccctcaagcggttcagtcggccatcacgactttcgaccgcggctacaatgaagtgaagttccaaacgtttgagctgccggaaggcgagagcatgccggaatttgtacggtccgacatttcgccgctcggatacgaaacccagccgattggcggcaccatcaccaatccgggaacaagcttcctgaaagatctcagcatttcccgttccgggcttgtgccggtatatgacaacgagtcctgcatcaactgcgcggagtgcgacacggtttgtccggatatgtgcttcgtttgggaagaaaaggtcaataaaaaaggcaaatcgatgatgttcctgaaagggatcgattatcaatactgtaaaggctgtctcaaatgcgtggaagcctgcccgacctccgcactctcccgttcgcgtgagaaggaaggcttcgcggaagctaatacggtccggcacacttttgatcttgtaaaccagatctaaatcccgagagaaaggtggaacactaatggctatcgattacgaaaaagaagtcggctccgccaaggtggagcagaagtttttatatgaatccggcaatgaaatggcggcttacgctgcgcatcaaatcaactatcatgtgatgggctatttcccgatctccccgtccacggaggttgcgcaattcctcgattccatgaaagccagcggacagcatgacatcatgctggtgccttctgacggcgagcacagctctgcgggcatctgttacggcgcatccaccgcaggcggccgcgtatttaacgcgacaagtgcccaaggctacctgtacatgctggagcagctcccggttcaatccggcacgcggatgccgatggtcatgaacctgatctgccgttccgtttccggaccgctgaacattcatggagatcactccgacctgtattatgcgctgaacaccggctggccgatcctgatgtgcccggatccacagtccgtatacgatatgaacatcatggcaatcaagctggccgagcatgccaaggttcgcctgcccgtgctggtagcgatggacggatactttacttctcaccagaagcgccgcgtccaagcgattgcgaaccgtgaagatgtccaaagctttatcggacctcaaccgcaaggttatgacgatacgctggaccgtgagaatccgatttccgtcggcccttatatgaacgaaccggactatatcaacaacaaataccaacaatccgtggcgatgtacaatgctgcggaagtattcgaagaaatcgccaaagaatatggcgagctgacaggccgccattacccgatcctgtcgcagtaccggatggaagatgccgaggttgcggtattcttgatgaactccgcttccgagatcatcaaggacgttgtcgatcagcttcgtgagaaaggcattaaagccggcgccatttcgccgaacatgatccgtccgttcccgcagaagcaaattgccgaagcgctgaagaacgttaaagccatcaccgtcggtgaccgcgcggattctttcggagcgcatggcggcaacatggttaacgaaatcaaggcggctctgttcacttacggcaatacaacaacgaaagtaatcagccggatttacggtgtcggcggcaaggacttctttgccgaagacggccacagcctgttcgagatggctattgaggcagcggaaaccggtgaagtgaaagtacctttcgattattacggccacaatccgggcgttgccgaaagcgcgccgaagcgtgtgatcaaaccgcttaaattcgagtctctcaagacaggcttgattactgtggacaaggatgaggaaacaggcaaactcaaggttaagattccgccaatgcgcagcttgaccgttaaaccgaagcgcttgtccccgggtcacggtacttgcccaggctgcggtattttctccggcctggagctgttcttcaaaggcatcgaaggagatgtcgttgctctgtaccacacaggctgcgcctttgttacgacgaccggctatccttattcgtcgcataaatcgacgttcatccataacctgttccaaagtggtgccgcaactatgtccggtgtggttgaaatgttctgggaacgcaaacgccgcggcgagcttgatcatctgaacctgaaagacgactttacctttgtcatggttaccggcgacggcggcatggacatcggtatggggccggcaatcggcgccgcgcttcgcggacataagatgatcgttctggaatatgacaatgagggctacatgaatacgggtgcacagcaatcgtactcgacgccgatcggtcaccgcacatcgacatccagcatcggcaagacgcaaaatggtaaggtgacgcagcataaagatactccgcaaattatggctgccaccaatattccttatgtgttcacaggctgcgaagcgttcccgcaggatttggtgaagaaagccgccaaagcccaatggtacgctcagaacgaagggcttgtatacggcaaaatcttgtctgcctgcccgctgaattggatgaccgaagacaatgtaggcaccaagctggtatccctggctgtcgattcctgcttcttcccgctgtatgaagtggagcaaggcgttaccacgattacgtacaatccggaagaaaaggaaaaacgcgtagacgttaccgaatggctcaaaggcatgggtaaaaccaagcatctgcttaaggaagagaacgaagctgcgctgaacggcttcaaggaagaagtagagcgccgctggagccgcctgaaagcgaagcacgagcatccggagctgtaagatccgcttgattgcttgaaaatggagctgtcccataagtagattttcaaagtcagagacagttcttcttcattctagaaaacgcaaaaagtcagcaaaacagcgattctccggttattggagaatcgctgttttgcgtttttggtccactgcggcttgttttagtacattatgggcaagggaaagccacccgacctcaagcgtcactttctccatgccgcgaagcagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 17 - DNA, 2347 bp, synthetic construct
  misc_feature 1..2347: note = OTHER INFORMATION: ITR2-PgroES:Pf_fldA-TCAT-CAT-PCAT-ITR1 expression cassette
  source 1..2347: mol_type = other DNA
aacaggttggctgataagtccccggtctactgagtcccaccacacccgcccgaccgaactaagttaagacacgcctagcacgaagcccacagaagtacgttgacaccaactgcatgatgatatatgtcaaaataataaaccaatactgttttctcaaatggtatgtactcctttgttaagtgggtttcgttcatctacagctattgtaacataatcggtacgggggtgaaaaagctaacggaaaagggagcggaaaagaatgatgtaagcgtgaaaaattttttatcttatcacttgaaattggaagggagattctttattataagaattgtgttagcactctttagtgctgagtgctaaaattacatattcatactattgaggaggttatttcgatggctaaaatattggtggtatatgccagcctgaccggcaatacagaggaaattgcggaactgatcgcggagggaatccgccagtcaggaggcgaggcggttctgaaattggtcactgactgcaacgccttcgatatcgcatcctatgacggggcggtactcggggcctacacctggggagatggagaactgccggatgaatttctcgatttctatgaggagctggatgaaatagacctggcaggaaccaaagccgccgtgttcggaagcggcgactccggatatgaaatctactgcggcgcggttgaccggattgaagaaaagctgaaagaaagaggcgcggccatagtacaggcgagtctcaagatcgaatatgggccgactgcggtggagaaagaggaatgccgcaagttcggacagcgatttgccgaggcctgcgtggcggtatcttaatatggatacgtcatgcggcgcagccgctaattcggcctcccgtcttcccaaatttcatgattatattgttgagcgggcgctgctggacatggcctagacgaacggaggtggaaatggactgcctctgtggcggcagtaggtgcagtatgcggtcggtcatacgttaaatgattactacagcttgacgcccggggtgcgcgcagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 18 - DNA, 1921 bp, synthetic construct
  misc_feature 1..1921: note = OTHER INFORMATION: ITR2-PmreB:Pz fer-TCAT-CAT-PCAT-ITR1 expression cassette
  source 1..1921: mol_type = other DNA
aacaggttggctgataagtccccggtctactgagtcccaccacacccgcccgaccgaactaagttaagacacgcctagcacgaagcccacagaagtacgttgacaccaatagacgaatgaaactttgaaaaagtttcccgctttctataagaattacaagatattttttccataaaaccgaaaaatgtattacttctgttacacagttccttgattttttacaaaaacatttctttaccgacgcacacccccaaaaatcgcagtatttctgagaaactttaaatgtagaatcatattaaattaggatagagattgggtgaaaagatggctaaatacacttgggtagagaaagatacctgcatcgcatgcggagcctgtggcgctaccgctcctgacatttacgattatgacgacgaaggactggctgaagttatctacgagggagatgccaaccgcggcgtaacggacattccggaagatttgttagacgatctgcaagatgccgctgacggatgtccgacggattccatcaagattgcggatacgcctttcaacaagtaaggttaattcttttgtttcacgaatttattaattggcttatgaaaagacatcccggttcacagagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 19 - DNA, 2354 bp, synthetic construct
  misc_feature 1..2354: note = OTHER INFORMATION: ITR2-PsigX:Pz DUF269-TCAT-CAT-PCAT-ITR1 expression cassette
  source 1..2354: mol_type = other DNA
aacaggttggctgataagtccccggtctactgagtcccaccacacccgcccgaccgaactaagttaagacacgcctagcacgaagcccacagaagtacgttgacaccaagctatcgttaaaaatatcgtggaagcccacaacggatcaattactgtgcacagccgaatagataaaggaacaacattttctttttatattccgacaaaacggtaaaatcgagtctgaatttgccgaagaatcttgttccataagaaacacccgctgactgagcgggtgtttttttaatagccaacattaataaaatttaaggatatgttaatataaattcccttccaaattccagttactcgtaatatagttgtaatgtaacttttcaagctattcatacgacaaaaaagtgaacggaggggtttcagatggatcaaatggctaatgaagtcgcatctgcgaacgaacagccggcggacacagctcaagtgcggccgaagcgccgttccaaaccggcgcccgatcctgaaaaggcggagcgtattgtccgccgtttaaatgatctgcttagcgcgagcgattattttggccgttatgcttcactgactcccgaggagaagattgcgaagctgttcctggcttcagcggaggataaggctaaatcggacatcaactgcacggcgtccgatgaagtccgccgccaggtgccggtactgtttcaggccatcgccggtgtgatggaggagaagagcggcttgatgattcagagctctgcggaaattaatgtggaagggttcggccgtggactgatctacagcggccgcatcattcttgtgatgaagagcctgcgcgcaggctttccatttccgttcacttctctggagaagacggttgcctatgcggttgcttgcttggatgagggccttgccttcttagataagtataaagaacagactgctgtgcacggattactcagtctggagggataaacttcattgtcaagaaacacctccgatcctatactatttgaaactattaaatcatgagcgataacggaggtataatgcggttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 20 - DNA, 2261 bp, synthetic construct
  misc_feature 1..2261: note = OTHER INFORMATION: ITR2-PmreB:Pz DUF269-TCAT-CAT-PCAT-ITR1 expression cassette
  source 1..2261: mol_type = other DNA
aacaggttggctgataagtccccggtctactgagtcccaccacacccgcccgaccgaactaagttaagacacgcctagcacgaagcccacagaagtacgttgacaccaatagacgaatgaaactttgaaaaagtttcccgctttctataagaattacaagatattttttccataaaaccgaaaaatgtattacttctgttacacagttccttgattttttacaaaaacatttctttaccgacgcacacccccaaaaatcgcagtatttctgagaaactttaaatgtagaatcatattaaattaggatagagattgggtgaaaagatggatcaaatggctaatgaagtcgcatctgcgaacgaacagccggcggacacagctcaagtgcggccgaagcgccgttccaaaccggcgcccgatcctgaaaaggcggagcgtattgtccgccgtttaaatgatctgcttagcgcgagcgattattttggccgttatgcttcactgactcccgaggagaagattgcgaagctgttcctggcttcagcggaggataaggctaaatcggacatcaactgcacggcgtccgatgaagtccgccgccaggtgccggtactgtttcaggccatcgccggtgtgatggaggagaagagcggcttgatgattcagagctctgcggaaattaatgtggaagggttcggccgtggactgatctacagcggccgcatcattcttgtgatgaagagcctgcgcgcaggctttccatttccgttcacttctctggagaagacggttgcctatgcggttgcttgcttggatgagggccttgccttcttagataagtataaagaacagactgctgtgcacggattactcagtctggagggataaacttcattgtcaagaaacacctccgatcctatactatttgaaactattaaatcatgagcgataacggaggtataatgcggttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgtt

SEQ ID NO: 21 - DNA, 10 bp, synthetic construct
  misc_feature 1..10: note = OTHER INFORMATION: sequence downstream of ITR1 in Pb8
  source 1..10: mol_type = other DNA
atttgttaat

SEQ ID NO: 22 - DNA, 11 bp, synthetic construct
  misc_feature 1..11: note = OTHER INFORMATION: sequence upstream of ITR2 in Pb8
  source 1..11: mol_type = other DNA
acgttccttgt

SEQ ID NO: 23 - DNA, 21 bp, synthetic construct
  misc_feature 1..21: note = OTHER INFORMATION: concatenated sequence from SEQ ID NOs: and
  source 1..21: mol_type = other DNA
acgttccttgtatttgttaat

SEQ ID NO: 24 - DNA, 200 bp, synthetic construct
  misc_feature 1..200: note = OTHER INFORMATION:200 bp upstream of insert in Pb8
  source 1..200: mol_type = other DNA
ttaatctaccttaacacccaagttccgaaaaaatccccgcatattctgcatataagagtcctgagtagcggaagcttccttcagtagagtttgcatactgttcataaatgcggaagccgaagtcgtatcagctttgtcaaaccgattctttaactgtgtgttatttaacagctcttccatctgtcccgtacgttccttgt

SEQ ID NO: 25 - DNA, 200 bp, synthetic construct
  misc_feature 1..200: note = OTHER INFORMATION:200 bp downstream of insert in Pb8
  source 1..200: mol_type = other DNA
atttgttaatccaatcagggttttgagggaccttttgcgcaaagcttaacagttggctcagtccatttcctccacccttaatgtcattcatgaatgccgcatattgttgaggatccgcctgcttcatcacttccgacagtttgacgtaatcctcaggagcacctaacgggcgatctgttggagtcgtgtagctaatatct

SEQ ID NO: 26 - DNA, 400 bp, synthetic construct
  misc_feature 1..400: note = OTHER INFORMATION: bp of wt PROKKA_03723 locus flanking the insertion site in Pb8; concatenation of SEQ ID NOs:24 and25
  source 1..400: mol_type = other DNA
ttaatctaccttaacacccaagttccgaaaaaatccccgcatattctgcatataagagtcctgagtagcggaagcttccttcagtagagtttgcatactgttcataaatgcggaagccgaagtcgtatcagctttgtcaaaccgattctttaactgtgtgttatttaacagctcttccatctgtcccgtacgttccttgtatttgttaatccaatcagggttttgagggaccttttgcgcaaagcttaacagttggctcagtccatttcctccacccttaatgtcattcatgaatgccgcatattgttgaggatccgcctgcttcatcacttccgacagtttgacgtaatcctcaggagcacctaacgggcgatctgttggagtcgtgtagctaatatct

SEQ ID NO: 27 - DNA, 882 bp, synthetic construct
  misc_feature 1..882: note = OTHER INFORMATION: wild type PROKKA_03723 sequence
  source 1..882: mol_type = other DNA
atgaattttgggcctcttagagcacaacaagcggttcttaactctagtagcttacggataaatccgtcaagcaagcaaggagttccatccccccagctacatgaagaaaaacaatctggattgatcgttcaggacactgtggagattagtgaggccggcagacagctggccgaaggagctattgtgtcacatgctgccaagtattttggtacaacacagatcaatgattcattaaaccgggttttggaaggtcaaccagaagaagtgtccaattcagtgtacaatatgattcaatccaattttattgtcgacgggacggtaacggatgagaatcagcgagcggtgttgctggagatgggagtttcccaatccaagtatattgcagatcattacatggaaggcgataaggccaaagaatttctggaaaccatgaaccagatagctggaattgctttgacacgatccattgattcagagaccggagatattagctacacgactccaacagatcgcccgttaggtgctcctgaggattacgtcaaactgtcggaagtgatgaagcaggcggatcctcaacaatatgcggcattcatgaatgacattaagggtggaggaaatggactgagccaactgttaagctttgcgcaaaaggtccctcaaaaccctgattggattaacaaatacaaggaacgtacgggacagatggaagagctgttaaataacacacagttaaagaatcggtttgacaaagctgatacgacttcggcttccgcatttatgaacagtatgcaaactctactgaaggaagcttccgctactcaggactcttatatgcagaatatgcggggattttttcggaacttgggtgttaaggtagattaa

SEQ ID NO: 28 - DNA, 3065 bp, synthetic construct
  misc_feature 1..3065: note = OTHER INFORMATION: PROKKA_03723 with integrated Pb8 insert
  source 1..3065: mol_type = other DNA
ttaatctaccttaacacccaagttccgaaaaaatccccgcatattctgcatataagagtcctgagtagcggaagcttccttcagtagagtttgcatactgttcataaatgcggaagccgaagtcgtatcagctttgtcaaaccgattctttaactgtgtgttatttaacagctcttccatctgtcccgtacgttccttgtaacaggttggctgataagtccccggtctactgagtcccaccacacccgcccgaccgaactaagttaagacacgcctagcacgaagcccacagaagtacgttgacaccaagctatcgttaaaaatatcgtggaagcccacaacggatcaattactgtgcacagccgaatagataaaggaacaacattttctttttatattccgacaaaacggtaaaatcgagtctgaatttgccgaagaatcttgttccataagaaacacccgctgactgagcgggtgtttttttaatagccaacattaataaaatttaaggatatgttaatataaattcccttccaaattccagttactcgtaatatagttgtaatgtaacttttcaagctattcatacgacaaaaaagtgaacggaggggtttcagatgattaaaatattagtggtatatgccagcctgaccggcaatacagaggaaatagcggaactgatcgcggagggaatccgccaatcgggaggggagacagtcataaaatcggtcaccgactgcaacgccttcgatatcgcatcttatgatggtgcggtcttgggagcctacacctggggagatggagaactgccggatgaattcctcgatttctatgaggagctggatgaaatagacttagcaggcatgaaggccgccgtattcgggagcggagacaccgggtatgatatttactgcggtgcggttgacctgattgaagcaaagctgaaggaaagaggcgccgtcatggtacagacgagtctgaaaattgaatatgggccgactgctatggagaaagaggattgccgcaaattcggacagcgttttgccttggcatgcgtaggggtatcttaaggtgagtacggcatgcggcgctgccgctaatccggcctctcatttccccaaatttcacgactatatcgtagagcgtacgctgctggatatggcggaggcaagcggcggcggaaaagaactgcatttgtggcggcagcaggtacagtatacggtcggccacacgttaaatgatgttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacattagaaaaccgactgtaaaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatatcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaagtcaatattactgtaacataaatatatattttaaaaatatcccactttatccaattttcgtttgttgaactaatgggtgctttagttgaagaataaaagaccacattaaaaaatgtggtcttttgtgtttttttaaaggatttgagcgtagcgacagaccggggacttatcatccaacctgttatttgttaatccaatcagggttttgagggaccttttgcgcaaagcttaacagttggctcagtccatttcctccacccttaatgtcattcatgaatgccgcatattgttgaggatccgcctgcttcatcacttccgacagtttgacgtaatcctcaggagcacctaacgggcgatctgttggagtcgtgtagctaatatctccggtctctgaatcaatggatcgtgtcaaagcaattccagctatctggttcatggtttccagaaattctttggccttatcgccttccatgtaatgatctgcaatatacttggattgggaaactcccatctccagcaacaccgctcgctgattctcatccgttaccgtcccgtcgacaataaaattggattgaatcatattgtacactgaattggacacttcttctggttgaccttccaaaacccggtttaatgaatcattgatctgtgttgtaccaaaatacttggcagcatgtgacacaatagctccttcggccagctgtctgcc

SEQ ID NO: 29 - DNA, 947 bp, synthetic construct
  misc_feature 1..947: note = OTHER INFORMATION: (spacer1)-PtrnQ-Pz_fer-Tfer-(spacers4&5)-TgyrB-(spacer6) expression cassette
  source 1..947: mol_type = other DNA
aacaggttggctgataagtccccggtctactgagtcccaccacacccgcccgaccgaactaagttaagacacgcctagcacgaagcccacagaagtacgttgacaccaactgatatgcaaagtcatctctttttcccattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatggctaaatacacttgggtagagaaagatacctgcatcgcatgcggagcctgtggcgctaccgctcctgacatttacgattatgacgacgaaggactggctgaagttatctacgagggagatgccaaccgcggcgtaacggacattccggaagatttgttagacgatctgcaagatgccgctgacggatgtccgacggattccatcaagattgcggatacgcctttcaacaagtaaggttaattcttttgtttcacgaatttattaattggcttatgaaaagacatcccggttcacagagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacatt

SEQ ID NO: 30 - DNA, 983 bp, synthetic construct
  misc_feature 1..983: note = OTHER INFORMATION: (spacer1')-PtrnQ-Pz_fer-Tfer-(spacers4&5)-TgyrB-(spacer6) expression cassette
  source 1..983: mol_type = other DNA
gcctttaccgctagcgtctaacggactgacatttttaacaggttggctgataagtccccggtctactgagtcccaccacacccgcccgaccgaactaagttaagacacgcctagcacgaagcccacagaagtacgttgacaccaactgatatgcaaagtcatctctttttcccattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatggctaaatacacttgggtagagaaagatacctgcatcgcatgcggagcctgtggcgctaccgctcctgacatttacgattatgacgacgaaggactggctgaagttatctacgagggagatgccaaccgcggcgtaacggacattccggaagatttgttagacgatctgcaagatgccgctgacggatgtccgacggattccatcaagattgcggatacgcctttcaacaagtaaggttaattcttttgtttcacgaatttattaattggcttatgaaaagacatcccggttcacagagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacatt

SEQ ID NO: 31 - DNA, 960 bp, synthetic construct
  misc_feature 1..960: note = OTHER INFORMATION: (spacer1')-PtrnQ-Pm_fer-Tfer-(spacers4&5)-TgyrB-(spacer6) expression cassette
  source 1..960: mol_type = other DNA
gcctttaccgctagcgtctaacggactgacattttactgagtcccaccacacccgcccgaccgaactaagttaagacacgcctagcacgaagcccacagaagtacgttgacaccaactgatatgcaaagtcatctctttttcccattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatgagcaaatttacgtgggttgaaaaggatacttgcattgcgtgtggtgcttgtggggcgacggccccagacatttacgactacgacgatgaaggtttagctgaggtcatttttgacggagatgccaacaaaggtatcaaagccatcccggatgatttattcgatgatatgcaggatgcgtgcgacggttgtccgacggacagcatcaaggtagccgatgagcctttcaacaaggaaggctaaggttaattcttttgtttcacgaatttattaattggcttatgaaaagacatcccggttcacagagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacatt

SEQ ID NO: 32 - DNA, 945 bp, synthetic construct
  misc_feature 1..945: note = OTHER INFORMATION: (spacer1')-PR'-Pm_fer-Tfer-(spacers4&5)-TgyrB-(spacer6) expression cassette
  source 1..945: mol_type = other DNA
gcctttaccgctagcgtctaacggactgacattttactgagtcccaccacacccgcccgaccgaactaagttaagacacgcctagcacgaagcccacagaagtacgttgacaccaactgatatgcaaagtcatctctagacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatgagcaaatttacgtgggttgaaaaggatacttgcattgcgtgtggtgcttgtggggcgacggccccagacatttacgactacgacgatgaaggtttagctgaggtcatttttgacggagatgccaacaaaggtatcaaagccatcccggatgatttattcgatgatatgcaggatgcgtgcgacggttgtccgacggacagcatcaaggtagccgatgagcctttcaacaaggaaggctaaggttaattcttttgtttcacgaatttattaattggcttatgaaaagacatcccggttcacagagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacatt

SEQ ID NO: 33 - DNA, 924 bp, synthetic construct
  misc_feature 1..924: note = OTHER INFORMATION: (spacer1')-P43-Pm_fer-Tfer-(spacers4&5)-TgyrB-(spacer6) expression cassette
  source 1..924: mol_type = other DNA
gcctttaccgctagcgtctaacggactgacattttactgagtcccaccacacccgcccgaccgaactaagttaagacacgcctagcacgaagcccacagaagtacgttgacaccaactgatatgcaaagtcatctctatttttttgccaaagctgtaatggctgaaaattcttacatttattttacatttttagaaatgggcgtgaaaaaaagcgcgcgattatgtaaaatataaagtgagctgacgttctcataacgcagtctatataaggaggtgggaaagatgagcaaatttacgtgggttgaaaaggatacttgcattgcgtgtggtgcttgtggggcgacggccccagacatttacgactacgacgatgaaggtttagctgaggtcatttttgacggagatgccaacaaaggtatcaaagccatcccggatgatttattcgatgatatgcaggatgcgtgcgacggttgtccgacggacagcatcaaggtagccgatgagcctttcaacaaggaaggctaaggttaattcttttgtttcacgaatttattaattggcttatgaaaagacatcccggttcacagagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaaaagcacggggcaggttagtgacatt

SEQ ID NO: 34 - DNA, 1544 bp, synthetic construct
  misc_feature 1..1544: note = OTHER INFORMATION: UP homology for P. brasilensis czcB
  source 1..1544: mol_type = other DNA
atgatgccattgtccacggtcggtatgaacgctgttcatccgagcaaggtaggcaatgcatctactgcatccaatctggtacgtactgttgcttcatctatggctatcgcggtgctgacctcgatcatgcaaagtcgttcggcagctcatgcccaacaaatttcggagactgttacaccggacgcggctcatacgctacaagcgacgttgggcaactcatggatgtcatccataagtagtttaattacactggatgccacatcgagaggaattggggatacattccttatttcatccataccgctgttttgcacgattccactcatctttttgtttattcagaggaagaaagcaaagacacagccggaagcttaataaagagaagtacaaaaatatcgactggatggcttacattatttccacggacagaaagaaagggaattatcatgaaaatattgagagtagaaagtacattagcggctttggtcctcagcggcgcagtgttggcaggctgctctagtaacagcggagcagcgcaggacatggttgttccggttaagatcagccaagcgcagcagggtatcatcggacaggggaacatttatacaggtacggtcacaccttcggagacggtaaatatcgtgcctaaagtaggcggtaaagtggttcaattacctgtagatattggctcggaggtcaaagaaggtcaagtgctgttcaagcttgatgataaggatatgcgcaacaatgttgcgaaagcccgtgcagcagcagcggcggcggctgccggggtaagcagtgctgaggcatcccatgagtcgtccttggttcaagccaactctggcgtagtacagtccaagagcggtatgattcaatcccaaagtgcaatcaatcaggcgcagggcgttattaaccaggccactacagcgttagaacaagcgacaaatggtgtctccacggcggccaatacagtaaaacaaacacaacaagccttggcagacgctcagaagaatgtgacacgtacacaaagcctgtttgatgcaggtgcaagcacccaagctcaactagaacaagcaaaaacagctgccgtgaacgcggaagcggcctacaaaaatgcacaaaatgcacaggctaatgctcaagatcagctggtagctgcacaaaaggcactcactacagcccgtaacgcctatgatacagcaaaaaatagctatagtaatgccaacaacggctacagcaatgcacaaaatcagcttaaggtatctcaaaatactgcggtcattgagtctagccagcaaactctcaagcaggctcagttgaatgtaagtattgctcaagatgcactgaacgatactgtagttacatcaccaatcaacggtattgttgggacaaaaaatgcagaggtaggcgaaatggtatctgctcaggctcctgcactggtcattgccaatctgagtacagtcaatacactgatctatgttccagctgaacagatcaacaatgttaaagtgggtgacaaggtacaggttcgtgtagcagcatcc

SEQ ID NO: 35 - DNA, 1230 bp, synthetic construct
  misc_feature 1..1230: note = OTHER INFORMATION: DOWN homology for P. brasilensis czcB
  source 1..1230: mol_type = other DNA
aaaaatgtcagtccgttagacgctagcggtaaaggctatcctgtaaaaatctcggtagcgaacccggatatgaaactgaaatccggcatgctggcagatatcagcttcgtcgctgccaatgcgcaagaaggcattgtcatccctaccgcagctgttcaaaaggagcaaggtaaagaatatgtatatgttgtaaacggagaccatgcaaaacgcaaggaagtgaaaaccgttcaaacgacaggctcacaaacactggttaccagtggcttgagcaatgatgagaatgtaattgtgaataacctggcattgctgtccgacaatacaccaattacaatcagccaataaattggtctagagactaagttttgtttttcgtactatatattcgtgctttataatagcgagatttatgaaaaagaatatgagcaggacagaccgcagctgtttatcggtctgtcctgcttttattttttcgcatattttacataggattaatgtcggaaagaggctatttttgacgaacattggtatgataggatagactgaattggaaatagatagtctactagatgggatttattacattctcagatgagcgatcggaggaattgtaaaaatggaagtgaaattagtagttgattttgatgcttatgaggatggccaaacgatggctaaatccattgaggagctggcagccaagccagagagtgctgaggttacggagctaattattggcgattggggcggagcctatgaaaattcgcctgaggattttatcccggtgctggtgcagcataaagatcaatttccgaagttgcgcaagctgtttatcggcgatatggaatatgatgagtgtgaggtttcatggataaatcaaacgaatctgtccccgttgttagaagcgtttccagcactggagtctttttatgtcaaaggaagccaggacttgagcttggagccgcttcgccatagccgccttcaggaattggttattatttgcggtggtctgccggtcagcgtccttcagcaaatatatcatgccgagctgccagagctgcgcaagctggagctgtatttgggtgtggatcaatacggttttgatggtagtctgaaagatgttttaccgtttttgaacaataatccatttcccaaacttacatacttggggctaaaggatagcgaaatacaggatgaaattgcgattgcagcagctaatgccccagtcatcga

SEQ ID NO: 36 - DNA, 1000 bp, synthetic construct
  misc_feature 1..1000: note = OTHER INFORMATION: Homology-Fer (Pp5-Pp12) UP
  source 1..1000: mol_type = other DNA
agccgcctaaggcgacgcccaagccgtttgatgtgccaggatttttactggcggcaacctgtgcaggaacgctcttgtacgcattgacaaacggtcagcgtgatggctggacttcttttggcattgtatcgctgctgtttatttctttctgggcacttgtttttctcatctatgtagaaagcggcaaggataatgctgtaattgaaatatcattattcaaaaattcaaaatacacgattagtgtaattgcctccagtctggtcatgatggggatgtacggaggaacgttccttacaccgttgtttttgcaaaatattcagcgtgtgtcaccgattgatactggtattatcctcattccacaggccattgctatggcagccatgatgcctattgccgggaaactatttgataagttcggcattgtgcctttaggacttgtgggcctaacgctaacgagctttatgacgtatcatcttcatcagcttacgccagatacttcgcatgcctggttggaaaccgtaatggcattacggggtctaggaattgggatctgtatgatgccgttatccacagtcggtatgaacgccgttcatccgagcaaggtagctaatgcgtctacagcgtctaatctggtgcgtactgtggctgcatctatggcgattgcggtactgaccgccattatgcaaagccgttcggctgctcatgcccagcaaatttcagagtccattaccccagactctgctcatgtgcttcaagcgacaatgggcagctcatggatgtcatccataagtagtttgatcacactggatgctacatcaagaggaattggggatacgttcctcatttcatccataccgctgttttgcacgattccactcatctttttgtttattcagaggaagaaagcaaagacacagccggaagcttaataaagagaaatacaaaaatatcgactggatggcttacattatttccacggacagaaagaaagggaattatc

SEQ ID NO: 37 - DNA, 1000 bp, synthetic construct
  misc_feature 1..1000: note = OTHER INFORMATION: Homology-Fer (Pp5-Pp12) DN
  source 1..1000: mol_type = other DNA
cttggttttaaacttgcattctttataatagtagaaatttattaaggatgtgccaagacagcccacttgcgggctgtcttgtttttatttcaatgtgcatatgttacataagattaatgtcgaactgggactttttttgacgaacattggtagaataggatagactaaattggaaatagttagtttacgagatttcagataagcgattggaggaatgggaagatggaagtgaaattggtagtggattatgatgcttatgagaatggtcaaaccatggccaagtctattgaggaactggcagccaagccggagagcgcacaaattacagatctaatcattggagattggggcggcgcctatgaaaattcaccagatgattttataccggtgttagtaaagcttaaagatcaatttccgaaagtatgcaagctgtttattggtgatatggaatatgacgagtgtgaggtgtcatggattaaccaaacgaatctctccccgttgttagaagcgtttccaaagcttgaatccttatatattaaaggaagccaggacctgagcctggaaccccttcgccataatcatcttcaggaattggttattatctgtggtggtctgcctgtgagcgttcttcagcaaatacgtaatgcagaactgcctgaactgcgcaagctggagctgtatttgggtgtcgaggactatggttttgacggtaaactggaagatgttttaccgtttttgaacagcaatcctttcccaaaacttacatacctgggactcaaggatagtgaaatacaggatgaaattgcaattgctgctgctaacgccccagtgattgaacaactggaagtgctggatctctcacaaggtacattgtctgatcaaggtgcagaagcattgctggctagtgaaagtataaaagggctgaagcatctggatttgagctatcattatatgtctagtgagatgatggaacgctggaaaaagtcaggtttatctgtaa

SEQ ID NO: 38 - DNA, 1153 bp, synthetic construct
  misc_feature 1..1153: note = OTHER INFORMATION: (spacerA)-PR'-Pd_Duf269-(spacerB) expression cassette
  source 1..1153: mol_type = other DNA
tactgagctaataacaggcctgctggtaatcgcaggcctttttatttggattagacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatggatcaaatggctaatgaagctgcatctgcgaacgaacagccggcggacacagctcaagtgcggccgaagcgccgttccaaaccggcgcccgatcctgaaaaggcggagcgtattgtccgccgcttaaatgatctgcttagcgcgagcgattattttggccgttatggtacactgactcccgaggaaaagattgcgaagctgttcctggcttcagccgaggataaggctaaatcggacatcaactgcacggcgtccgatgaagtccgcggccaggtgccggtattgtttcaggccatcgccggtgtgatggaggagaagagcggcttaatgattcaaagctctgcggaaattaatgtggaagggttcggacgtggactgatctacagcggccgcattattcttgtgatgaagagcctccgcgcaggctttccatttccgtttacttctctggagaagaccgttgcctatgcagttgcttgcttggatgaaggccttgccttcttagataagtacaaagaacagactgctgtgcacggactgctcagtctggagggataaggttaattcttttgtttcacgaatttattaattggcttatgaaaagacatcccggttcacagagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaa

SEQ ID NO: 39 - DNA, 1153 bp, synthetic construct
  misc_feature 1..1153: note = OTHER INFORMATION: (spacerA)-PR'-Pf_Duf269-(spacerB) expression cassette
  source 1..1153: mol_type = other DNA
tactgagctaataacaggcctgctggtaatcgcaggcctttttatttggattagacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatggatcaaatggctaacgaagccgcatcagcgagcgcgcagccggctgacggagctcaggcgcggccgaagcgccgctccaaaccggctcccgatcctgaaaaggcggagcgtcttgtccgccgtctcaacgatctgctcagcgcgagcgattattttggccgttatgcttccatgactcccgaggaaaagattgcgaagctgttcctggtatcggcagaggataaggctaaatcggacattaactgcacggcgaccgatgaagtccgccttcaggtgccggtattgtttcaggccatcgctggcgtaatggaggagaagagcggcctgatgattcaaagctctgcagaaattaatatcgaaggattcggccgcggactgatctacagcggccgaattatccttgtcatgaagagtctgcgcgcaggctttcccttcccgttcacttctctggagaagacggtggcctatggggttgcttgcttggatgaaggccttgccttcctggataaatataaggaacagactgctgttcacggattgctcagtctagagggataaggttaattcttttgtttcacgaatttattaattggcttatgaaaagacatcccggttcacagagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaa

SEQ ID NO: 40 - DNA, 1159 bp, synthetic construct
  misc_feature 1..1159: note = OTHER INFORMATION: (spacerA)-PR'-Pg_Duf269-(spacerB) expression cassette
  source 1..1159: mol_type = other DNA
tactgagctaataacaggcctgctggtaatcgcaggcctttttatttggattagacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatggaatcgtcggctaacaggaagatagcagtccaggctgggctttcccgggagggggcggcggcgaaaccccgccgccgcaaggttgtaaaggagctggaccccgatacggcggatgcctttgtccgccgtttgtgccatctgctcgatacggctgactttttcggacggcatgcctcgctgtctccgcaggagaagattgccaagctgtttctggcatcccctgaggacaaggatcaatcggacttcaactgcgccgtatctcccaaggtccgccagcaggttccgattttgttccaggcgatcgccggagtcatggaagagaagagcggagccatggtgcagagcactgctgagattaatgtggagggttttgggcgggggctgctgtataccgggcgcgttattctggtgctgaaggccctgcgggccggtgcccctttcccgttcacttcggaggagaagaccatccgttatggggtggggtgtgtggaggaaggtctggtttttttggagaagtataaggaattgacggcaggccacggtttgttcagccttgaaggataaggttaattcttttgtttcacgaatttattaattggcttatgaaaagacatcccggttcacagagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaa

SEQ ID NO: 41 - DNA, 1159 bp, synthetic construct
  misc_feature 1..1159: note = OTHER INFORMATION: (spacerA)-PR'-Pj_Duf269-(spacerB) expression cassette
  source 1..1159: mol_type = other DNA
tactgagctaataacaggcctgctggtaatcgcaggcctttttatttggattagacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatggagagttctgcgaatagaaaaatggcggtgcaagcgggcttcagccgtgaaggggcggcagtaaaaccacgccgtagaaaagttgttaaggaactagatccaggtacagcagatgctttagtccgccgtctttgccaccttcttgataccgctgatttctttggccgacatgcaccgttatctcctcaagagaaaatcgcgaaattattcctagctagcgcggaggataaagatcaatcagacttcaactgtgccgtatctccgaaggtccgccaacaggtaccgatcttgtttcaggcaattgcaggtgtgatggaggaaaaatcaggtgcgatggttcaatctacggctgagataaatgttgagggatttgggcgaggcctactatacactggccgtgtcattttggtgcttaaaagcctacgcgcaggtgctccgttccctttcacttcagaggagaaggccatccgttacggcgttgaatgtgttgaggaggggctagcgtttttggagaagtacaaggagatcacagcgtgtcatgggcttttctcacttgagggataaggttaattcttttgtttcacgaatttattaattggcttatgaaaagacatcccggttcacagagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaa

SEQ ID NO: 42 - DNA, 1156 bp, synthetic construct
  misc_feature 1..1156: note = OTHER INFORMATION: (spacerA)-PR'-Pr_Duf269-(spacerB) expression cassette
  source 1..1156: mol_type = other DNA
tactgagctaataacaggcctgctggtaatcgcaggcctttttatttggattagacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatggaaccattggctgatggaaagatggcagttcaagccgggcctgcccgggagggggcggcggtgaagccccgccgcaaagttgtaaaggagctggatcagggtacggcggatgcttttgtccgccgtttgtgtaatctgctcgatacggcggatttcttcggacggcatgcttcgctgtctccgcaggagaagatcgccaagctgttcctggcttccgccgaggacaagaataaatcggacttcaactgtgccgtatctccgaaggtccgccagcaggttccgattttgttccaggctatcgccggagtcatggaagagaagagcggagcgatggtgcaaagcaccgctgagattaatgtggagggttttggacgggggctgctgtataccgggcgcgtgattttggtgctgaagagcctgcgggccggtgcccctttcccgttcacttcagaggagaagaccatccgctacggggtggaatgcgtcgaggaggggctggcttttttggagaaatataaggaaatgaccgcatgccacgggttgctcagccttgaaggataaggttaattcttttgtttcacgaatttattaattggcttatgaaaagacatcccggttcacagagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaa

SEQ ID NO: 43 - DNA, 1150 bp, synthetic construct
  misc_feature 1..1150: note = OTHER INFORMATION: (spacerA)-PR'-Psab_Duf269-(spacerB) expression cassette
  source 1..1150: mol_type = other DNA
tactgagctaataacaggcctgctggtaatcgcaggcctttttatttggattagacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatggatccaacggctaacgaaatcgcacccgcgaatgaacagccggctaacagacagccggcattgccgaaacgccgccccccggcccccgatcctgaaaaggctgcgcatcttgtccgccgcttgaacgacctgctcagcgcgagcgatcatttcggccggcatgcagccctgactcccgaggagaagattgcgaagctgttcctggtatcggcggaggataaagcgaaatcggatttcaactgcacggcatccgatgatgttcgccgccaagtgccgctattgttccaggccattgccggcgtgatggaagagaactgcggcttgatgattcaaagctctgcggaaatcaacgtcgagggcttcggccgcgggctgctctatagcggccgcgttattcttgtaatgaagagtctccgcgcaggctttccgtttccgttcacttctctggacaagacggtggcctacggggtagcttgcgtagaagaggggctcgcctttttggagaaatataaagaattgacctctgtgcacggactgctcagtctggagggataaggttaattcttttgtttcacgaatttattaattggcttatgaaaagacatcccggttcacagagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaa

SEQ ID NO: 44 - DNA, 1153 bp, synthetic construct
  misc_feature 1..1153: note = OTHER INFORMATION: (spacerA)-PR'-Pstel_Duf269-(spacerB) expression cassette
  source 1..1153: mol_type = other DNA
tactgagctaataacaggcctgctggtaatcgcaggcctttttatttggattagacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatggaacgaatggctgatgaattcgcgggtgtgaatggtcagactccggaacaggctcctgtttcgccgaagcgccggactaaacctctgcccgatcccgagaaatcggaacagctgatccgccggctaaatgatctgctcagcgccagcgattatttcgggcgctacgcttccctgacgccggaggagaagatctcgaagctgttcctcgcttctgcagatgacaaggccaagtccgatatcaattgcaccgtatccgatgaagtgcgttcccaggtgccggtgttgtttcaggcgattgccggcgtgctggaggagaagagcgggctgatgattcagagctcggccgagatcaatgtcgaaggctttggccgcgggctgctgtatagcggccggattatcctggtgatcaagagccttcgtgcgggctttccgttcccgttcacctcacttgcgaagacggttgcttatggagtggcatgtgtagaggaagggcttaccttcctggataaatataaagatctggcagaagtgcagagtcttctcaatctggagggataaggttaattcttttgtttcacgaatttattaattggcttatgaaaagacatcccggttcacagagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaa

SEQ ID NO: 45 - DNA, 1153 bp, synthetic construct
  misc_feature 1..1153: note = OTHER INFORMATION: (spacerA)-PR'-Pz_Duf269-(spacerB) expression cassette
  source 1..1153: mol_type = other DNA
tactgagctaataacaggcctgctggtaatcgcaggcctttttatttggattagacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatggatcaaatggctaatgaagtcgcatctgcgaacgaacagccggcggacacagctcaagtgcggccgaagcgccgttccaaaccggcgcccgatcctgaaaaggcggagcgtattgtccgccgtttaaatgatctgcttagcgcgagcgattattttggccgttatgcttcactgactcccgaggagaagattgcgaagctgttcctggcttcagcggaggataaggctaaatcggacatcaactgcacggcgtccgatgaagtccgccgccaggtgccggtactgtttcaggccatcgccggtgtgatggaggagaagagcggcttgatgattcagagctctgcggaaattaatgtggaagggttcggccgtggactgatctacagcggccgcatcattcttgtgatgaagagcctgcgcgcaggctttccatttccgttcacttctctggagaagacggttgcctatgcggttgcttgcttggatgagggccttgccttcttagataagtataaagaacagactgctgtgcacggattactcagtctggagggataaggttaattcttttgtttcacgaatttattaattggcttatgaaaagacatcccggttcacagagttcctgtgctgcggctacccatcgcctgaaatccagctggtgtcaagccatcccctctccgggggtagactacccatgagtcacaatgaacgactagagatctatgtgaggatcctaactcgagatcgagctgaagttgtgtaacgttccttcctagtggctgtattacgtgcttattaggtagtattttgtaagagttcataaggggcgcccaaatgggcgtcccttttcttgtcatccccgctgcgatccccacgactgaactactttgatattccgccaccacctgaccaagccaactaagttcaa

SEQ ID NO: 46 - protein, 78 aa, Paenibacillus zanthoxyli
MAKYTWVEKDTCIACGACGATAPDIYDYDDEGLAEVIYEGDANRGVTDIPEDLLDDLQDAADGCPTDSIKIADTPFNK

SEQ ID NO: 47 - DNA, 237 bp, synthetic construct
  misc_feature 1..237: note = OTHER INFORMATION: Reverse-Translation of SEQ ID NO: used in certain embodiments
  source 1..237: mol_type = other DNA
atggctaaatacacttgggtagagaaagatacctgcatcgcatgcggagcctgtggcgctaccgctcctgacatttacgattatgacgacgaaggactggctgaagttatctacgagggagatgccaaccgcggcgtaacggacattccggaagatttgttagacgatctgcaagatgccgctgacggatgtccgacggattccatcaagattgcggatacgcctttcaacaagtaa

SEQ ID NO: 48 - protein, 80 aa, unidentified
MSKFTWVEKDTCIACGACGATAPDIYDYDDEGLAEVIFDGDANKGIKAIPDDLFDDMQDACDGCPTDSIKVADEPFNKEG

SEQ ID NO: 49 - DNA, 243 bp, synthetic construct
  misc_feature 1..243: note = OTHER INFORMATION: Reverse-Translation of SEQ ID NO: used in certain embodiments
  source 1..243: mol_type = other DNA
atgagcaaatttacgtgggttgaaaaggatacttgcattgcgtgtggtgcttgtggggcgacggccccagacatttacgactacgacgatgaaggtttagctgaggtcatttttgacggagatgccaacaaaggtatcaaagccatcccggatgatttattcgatgatatgcaggatgcgtgcgacggttgtccgacggacagcatcaaggtagccgatgagcctttcaacaaggaaggctaa

SEQ ID NO: 50 - DNA, 627 bp, synthetic construct
  misc_feature 1..627: note = OTHER INFORMATION: czcB::PtrnQ-5' UTR:Pl_fer-Tfer
  source 1..627: mol_type = other DNA
gcctttaccgctagcgtctattttcccattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatggcgaagtttacttgggtagaaaaagatacatgtatcgcctgcggcgcatgtggggccacggctccagacatctatgattacgacgatgaggggatcgccgaggtaatcttcgagggggacgccaaccgaggagtaactgagatcccagaggacatgttcgaggacatgcaagacgcatgtgatggctgccctacggacagtattaagattgcagatgagcctttcaatgcctaaggttaattcttttgtttcacgaatttattaattggcttatgaaaagacatcccggttcacagagttctgtgtaacgttccttcctagtggctgtattacgtgcttattaggtagtattttgtaagagttcataaggggcgcccaaatgggcgtcccttttcttgtcatcggggcaggttagtgacatt

SEQ ID NO: 51 - DNA, 633 bp, synthetic construct
  misc_feature 1..633: note = OTHER INFORMATION: czcB::PtrnQ-5' UTR:Fp_fer-Tfer
  source 1..633: mol_type = other DNA
gcctttaccgctagcgtctattttcccattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatggcgaaatacacttgggttgagaaggatacgtgcatagcatgtggagcctgtggggctactgcgccagacatttacgactacgatgacgaaggactagcagaagtcatctatgagagtgatgcgaatcgaggcgtcaccgagatcccggatgatttatttgacgacctacaagacgcttgcgacggttgtcctacagacagtataaaaatcgccgatgttccatttaataaggaaggttaaggttaattcttttgtttcacgaatttattaattggcttatgaaaagacatcccggttcacagagttctgtgtaacgttccttcctagtggctgtattacgtgcttattaggtagtattttgtaagagttcataaggggcgcccaaatgggcgtcccttttcttgtcatcggggcaggttagtgacatt

SEQ ID NO: 52 - DNA, 633 bp, synthetic construct
  misc_feature 1..633: note = OTHER INFORMATION: czcB::PtrnQ-5' UTR:Sb_fer-Tfer
  source 1..633: mol_type = other DNA
gcctttaccgctagcgtctattttcccattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatgtcaaagtacacatgggtcgataaggatacttgcatcgcttgtggtgcgtgcggcgccacagctcctgacatctatgattatgatgatgagggcttagccgaggtaatttttgatggcgacgctaaccacggagtcaaagccattcctgaggacatgcacgaggatatgatagatgcctgtgacggctgccctacagacagtatcattattgcagatgagccatttaataaggaaggctaaggttaattcttttgtttcacgaatttattaattggcttatgaaaagacatcccggttcacagagttctgtgtaacgttccttcctagtggctgtattacgtgcttattaggtagtattttgtaagagttcataaggggcgcccaaatgggcgtcccttttcttgtcatcggggcaggttagtgacatt

SEQ ID NO: 53 - DNA, 633 bp, synthetic construct
  misc_feature 1..633: note = OTHER INFORMATION: czcB::PtrnQ-5' UTR:Fs_fer-Tfer
  source 1..633: mol_type = other DNA
gcctttaccgctagcgtctattttcccattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatggcaaagtacacctgggttgagaaggacacctgtattgcgtgtggggcttgcggagcaaccgcgccggacatttacgactacgatgacgagggcttagcggaagtaatttatgaaagtgatgcaaatcgtggagtcacggaaatcccggacgacctttttgatgatttgcaagacgcttgcgacggatgccctacagatagtatcaaaatagcggatgtgccgttcaataaggaaggttaaggttaattcttttgtttcacgaatttattaattggcttatgaaaagacatcccggttcacagagttctgtgtaacgttccttcctagtggctgtattacgtgcttattaggtagtattttgtaagagttcataaggggcgcccaaatgggcgtcccttttcttgtcatcggggcaggttagtgacatt

SEQ ID NO: 54 - DNA, 633 bp, synthetic construct
  misc_feature 1..633: note = OTHER INFORMATION: czcB::PtrnQ-5' UTR:WB17_fer-Tfer
  source 1..633: mol_type = other DNA
gcctttaccgctagcgtctattttcccattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatgtcaaaatacacctgggtcgataaagacacctgcattgcgtgcggtgcctgcggtgcaacggcccctgacatctacgactatgatgacgaaggactagctgaagtaatcttcgatggggatgccaaccacgggatcaaggccatacctgaagatatgcacgaggatatgatcgacgcatgcgacggatgcccaactgacagcatcatcatagctgacgagcctttcaataaagaggggtaaggttaattcttttgtttcacgaatttattaattggcttatgaaaagacatcccggttcacagagttctgtgtaacgttccttcctagtggctgtattacgtgcttattaggtagtattttgtaagagttcataaggggcgcccaaatgggcgtcccttttcttgtcatcggggcaggttagtgacatt

SEQ ID NO: 55 - DNA, 633 bp, synthetic construct
  misc_feature 1..633: note = OTHER INFORMATION: czcB::PtrnQ-5' UTR:Nm_fer-Tfer
  source 1..633: mol_type = other DNA
gcctttaccgctagcgtctattttcccattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatggcaaaatacacctgggtggaaaaggatacttgcatagcctgtggtgcttgtggtgccacggcgccggacatatatgactacgacgatgaaggtttagcggaagtgatttttaacggggattctaatcgaggtgtgatggaaataccggatgaccttcacgacgacatgcaagatgcctgcgatggctgtcctacagactcaatcaaggttgcggacgatccatttaacaaagagggataaggttaattcttttgtttcacgaatttattaattggcttatgaaaagacatcccggttcacagagttctgtgtaacgttccttcctagtggctgtattacgtgcttattaggtagtattttgtaagagttcataaggggcgcccaaatgggcgtcccttttcttgtcatcggggcaggttagtgacatt

SEQ ID NO: 56 - DNA, 633 bp, synthetic construct
  misc_feature 1..633: note = OTHER INFORMATION: czcB::PtrnQ-5' UTR:Sk_fer-Tfer
  source 1..633: mol_type = other DNA
gcctttaccgctagcgtctattttcccattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatggcgaagtacacttgggtagacaaggatacgtgtatagcttgtggagcctgcggcgcaactgctcctgatatttatgactatgatgacgaaggactagcagaggttatatttgatggcgatagcaaccacggtgtcaaagctatccctgaggacatgcatgaagatatgatcgacgcatgtgacgggtgcccgacggacagtatcatgattgcggacgagcctttcaataaagagggataaggttaattcttttgtttcacgaatttattaattggcttatgaaaagacatcccggttcacagagttctgtgtaacgttccttcctagtggctgtattacgtgcttattaggtagtattttgtaagagttcataaggggcgcccaaatgggcgtcccttttcttgtcatcggggcaggttagtgacatt

SEQ ID NO: 57 - DNA, 633 bp, synthetic construct
  misc_feature 1..633: note = OTHER INFORMATION: czcB::PtrnQ-5' UTR:Ss_fer-Tfer
  source 1..633: mol_type = other DNA
gcctttaccgctagcgtctattttcccattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatggctaagttcacctgggttgacaaggacacttgtatagcctgcggggcctgcggcgctacggccccagatatctatgattacgatgatgaaggtctagcagaagtcatcttcgacggggacggcaatcacggagttaaagcgattccagaagatatgcacgaagatatgcttgatgcgtgcgacggctgccctacagacagtataataattgctgacgagccattcaacaaggaaggataaggttaattcttttgtttcacgaatttattaattggcttatgaaaagacatcccggttcacagagttctgtgtaacgttccttcctagtggctgtattacgtgcttattaggtagtattttgtaagagttcataaggggcgcccaaatgggcgtcccttttcttgtcatcggggcaggttagtgacatt

SEQ ID NO: 58 - DNA, 176 bp, synthetic construct
  misc_feature 1..176: note = OTHER INFORMATION: PtrnQ-5' UTR downstream of Pfer
  source 1..176: mol_type = other DNA
cattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaag

SEQ ID NO: 59 - DNA, 1020 bp, synthetic construct
  misc_feature 1..1020: note = OTHER INFORMATION: Homology-Fer (Pp13) UP
  source 1..1020: mol_type = other DNA
tgccctgctcgattggagtagcacccaacaaactgctggccaaaatggcctctgatctaaaaaagccacgcgggatatctgttctacgtttacgtgatgtacccaacattttgtggaaccttccatgtggccaattgtttggtgtggggcgcaaaacagcagacaagcttctacaactaaatattcgcaccattgggcaattggccaaggcagatgagcacatgctggttagtgtatttggtgtaaatggttcttggttgaaattggcggcgaatggaattaatcattctccggtcagcactgaacgggagccaaacaaatccatcggtcacacaaccacactgcctatggatgtagtaaagttggaagaagctcaaagggtactgcttaacattagcgaccaggtagcccggcggcttcgacgacacggtatgttggcgggtggtattcaactgacgattcggaccccggatatgaagacctttacgcgttctcaagttaggagtacgcctacggaaagtgcagaggacatatataaggaagcttgtgcgttgtatcgtcgccactggggagaggataagccggtgcgtttgttagggattacgctgcagcagctcattcctaaggaggaagcggctgtacaattagatctatttgaataccaggatcagcctaaaaaagaaagcttattgaaaacgatggatgcgctgcgcgataagtttggtgaaaatgcaattttgactgcggggatgctgggagatgatccttcggtactgcttcgtaattccaagttgcgcggaacttcgctgcaaaaagacaatttacctactccggagtgaacatcggtgagggttaacgttagtattagtaatacatgttataagcattttttgcatgaaggataaaggaaaataatttgtaataatattgggtttgtattaatatgtgaatagaatgcaaaagctggtttgtttgattgtataggaggaagagaaaagtttactgagctaataacaggcc

SEQ ID NO: 60 - DNA, 1000 bp, synthetic construct
  misc_feature 1..1000: note = OTHER INFORMATION: Homology-Fer (Pp13) DN
  source 1..1000: mol_type = other DNA
atggctaagtacacttgggtagaaaaagatacatgcattgcttgtggagcttgcggcgctacggcaccagacatctatgattatgatgatgaaggtttggctgaagtaatctttgaaggggatgcaaaccaaggcatcaaggcgatcccagaagatctgtttgatgatatgcaggatgcttgcgacggctgccctacagattctatcaaagttgcagacgaaccgttcaataaagaaggctaagtgtagacttcttggacaggcatagcaccgccagtacccttcgccgtataacggtgaaagaatgctggcggtgctgtttttttatgtgctgtccattaagctcattttgaccgatatacataatagtaaacaagtaagattacccgattaggaggatccgatggaccattcgcctcatcttaaatcgtatgtaaaaaagcaccctgataataaaatggcctggtatttactcggtaaagagtatgaaagcagcggtcaggaggggaaagcaaattattgcttcaatcgtgcaggggaagtgttcgaagcctttgagcataaacagattccggcagacgtatggatggaatatcaggataaactggttcagatgtccaaggagagagaacgaagaacggctcgaacacgccaccttttaactgtgttaatggtactactactcatttgggttccttcagccaactcaccgagcatttcacctaagagcgcatccgttccatccggaacggagcatgcagcggttgatcatgtcgaaggtgcgaaagcggctactgcagatccaaaggagaaggcacagaacggtgagccttatgctggtggtgtgtttacggctcaagctcggcaaggaagcggagctggaacggcgttcacggatatgttggcaaagacgagtcaaatgccttccaagatcacggtgctgggcatggagcagagggggacatggctgatctggagagatggtatgaagccgttacta

SEQ ID NO: 61 - protein, 78 aa, synthetic construct
  REGION 1..78: note = OTHER INFORMATION: Pl_fer
MAKFTWVEKDTCIACGACGATAPDIYDYDDEGIAEVIFEGDANRGVTEIPEDMFEDMQDACDGCPTDSIKIADEPFNA

SEQ ID NO: 62 - DNA, 237 bp, synthetic construct
  misc_feature 1..237: note = OTHER INFORMATION: Pl_fer (reverse translation)
  source 1..237: mol_type = other DNA
atggcgaagtttacttgggtagaaaaagatacatgtatcgcctgcggcgcatgtggggccacggctccagacatctatgattacgacgatgaggggatcgccgaggtaatcttcgagggggacgccaaccgaggagtaactgagatcccagaggacatgttcgaggacatgcaagacgcatgtgatggctgccctacggacagtattaagattgcagatgagcctttcaatgcctaa

SEQ ID NO: 63 - protein, 80 aa, synthetic construct
  REGION 1..80: note = OTHER INFORMATION: Fp_fer
MAKYTWVEKDTCIACGACGATAPDIYDYDDEGLAEVIYESDANRGVTEIPDDLFDDLQDACDGCPTDSIKIADVPFNKEG

SEQ ID NO: 64 - DNA, 243 bp, synthetic construct
  misc_feature 1..243: note = OTHER INFORMATION: Fp_fer (reverse translation)
  source 1..243: mol_type = other DNA
atggcgaaatacacttgggttgagaaggatacgtgcatagcatgtggagcctgtggggctactgcgccagacatttacgactacgatgacgaaggactagcagaagtcatctatgagagtgatgcgaatcgaggcgtcaccgagatcccggatgatttatttgacgacctacaagacgcttgcgacggttgtcctacagacagtataaaaatcgccgatgttccatttaataaggaaggttaa

SEQ ID NO: 65 - protein, 80 aa, synthetic construct
  REGION 1..80: note = OTHER INFORMATION: Sb_fer
MSKYTWVDKDTCIACGACGATAPDIYDYDDEGLAEVIFDGDANHGVKAIPEDMHEDMIDACDGCPTDSIIIADEPFNKEG

SEQ ID NO: 66 - DNA, 243 bp, synthetic construct
  misc_feature 1..243: note = OTHER INFORMATION: Sb_fer (reverse translation)
  source 1..243: mol_type = other DNA
atgtcaaagtacacatgggtcgataaggatacttgcatcgcttgtggtgcgtgcggcgccacagctcctgacatctatgattatgatgatgagggcttagccgaggtaatttttgatggcgacgctaaccacggagtcaaagccattcctgaggacatgcacgaggatatgatagatgcctgtgacggctgccctacagacagtatcattattgcagatgagccatttaataaggaaggctaa

SEQ ID NO: 67 - protein, 80 aa, synthetic construct; identical to SEQ ID NO: 63
  REGION 1..80: note = OTHER INFORMATION: Fs_fer

SEQ ID NO: 68 - DNA, 243 bp, synthetic construct
  misc_feature 1..243: note = OTHER INFORMATION: Fs_fer (reverse translation)
  source 1..243: mol_type = other DNA
atggcaaagtacacctgggttgagaaggacacctgtattgcgtgtggggcttgcggagcaaccgcgccggacatttacgactacgatgacgagggcttagcggaagtaatttatgaaagtgatgcaaatcgtggagtcacggaaatcccggacgacctttttgatgatttgcaagacgcttgcgacggatgccctacagatagtatcaaaatagcggatgtgccgttcaataaggaaggttaa

SEQ ID NO: 69 - protein, 80 aa, synthetic construct
  REGION 1..80: note = OTHER INFORMATION: WB17_fer
MSKYTWVDKDTCIACGACGATAPDIYDYDDEGLAEVIFDGDANHGIKAIPEDMHEDMIDACDGCPTDSIIIADEPFNKEG

SEQ ID NO: 70 - DNA, 243 bp, synthetic construct
  misc_feature 1..243: note = OTHER INFORMATION: WB17_fer (reverse translation)
  source 1..243: mol_type = other DNA
atgtcaaaatacacctgggtcgataaagacacctgcattgcgtgcggtgcctgcggtgcaacggcccctgacatctacgactatgatgacgaaggactagctgaagtaatcttcgatggggatgccaaccacgggatcaaggccatacctgaagatatgcacgaggatatgatcgacgcatgcgacggatgcccaactgacagcatcatcatagctgacgagcctttcaataaagaggggtaa

SEQ ID NO: 71 - protein, 80 aa, synthetic construct
  REGION 1..80: note = OTHER INFORMATION: Nm_fer
MAKYTWVEKDTCIACGACGATAPDIYDYDDEGLAEVIFNGDSNRGVMEIPDDLHDDMQDACDGCPTDSIKVADDPFNKEG

SEQ ID NO: 72 - DNA, 243 bp, synthetic construct
  misc_feature 1..243: note = OTHER INFORMATION: Nm_fer (reverse translation)
  source 1..243: mol_type = other DNA
atggcaaaatacacctgggtggaaaaggatacttgcatagcctgtggtgcttgtggtgccacggcgccggacatatatgactacgacgatgaaggtttagcggaagtgatttttaacggggattctaatcgaggtgtgatggaaataccggatgaccttcacgacgacatgcaagatgcctgcgatggctgtcctacagactcaatcaaggttgcggacgatccatttaacaaagagggataa

SEQ ID NO: 73 - protein, 80 aa, synthetic construct
  REGION 1..80: note = OTHER INFORMATION: Sk_fer
MAKYTWVDKDTCIACGACGATAPDIYDYDDEGLAEVIFDGDSNHGVKAIPEDMHEDMIDACDGCPTDSIMIADEPFNKEG

SEQ ID NO: 74 - DNA, 243 bp, synthetic construct
  misc_feature 1..243: note = OTHER INFORMATION: Sk_fer (reverse translation)
  source 1..243: mol_type = other DNA
atggcgaagtacacttgggtagacaaggatacgtgtatagcttgtggagcctgcggcgcaactgctcctgatatttatgactatgatgacgaaggactagcagaggttatatttgatggcgatagcaaccacggtgtcaaagctatccctgaggacatgcatgaagatatgatcgacgcatgtgacgggtgcccgacggacagtatcatgattgcggacgagcctttcaataaagagggataa

SEQ ID NO: 75 - protein, 80 aa, synthetic construct
  REGION 1..80: note = OTHER INFORMATION: Ss_fer
MAKFTWVDKDTCIACGACGATAPDIYDYDDEGLAEVIFDGDGNHGVKAIPEDMHEDMLDACDGCPTDSIIIADEPFNKEG

SEQ ID NO: 76 - DNA, 243 bp, synthetic construct
  misc_feature 1..243: note = OTHER INFORMATION: Ss_fer (reverse translation)
  source 1..243: mol_type = other DNA
atggctaagttcacctgggttgacaaggacacttgtatagcctgcggggcctgcggcgctacggccccagatatctatgattacgatgatgaaggtctagcagaagtcatcttcgacggggacggcaatcacggagttaaagcgattccagaagatatgcacgaagatatgcttgatgcgtgcgacggctgccctacagacagtataataattgctgacgagccattcaacaaggaaggataa

SEQ ID NO: 77 - DNA, 187 bp, synthetic construct
  misc_feature 1..187: note = OTHER INFORMATION: PR'-5' UTR downstream of PporD
  source 1..187: mol_type = other DNA
tactgagctaataacaggccagacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaag

SEQ ID NO: 78 - DNA, 196 bp, synthetic construct
  misc_feature 1..196: note = OTHER INFORMATION: PtrnQ-5 UTR downstream of PporD
  source 1..196: mol_type = other DNA
tactgagctaataacaggcccattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaag

SEQ ID NO: 79 - DNA, 96 bp, synthetic construct
  misc_feature 1..96: note = OTHER INFORMATION: TR2-5' UTR downstream of PporD
  source 1..96: mol_type = other DNA
tactgagctaataacaggcctgctggtaatcgcaggcctttttatttggatttgctgacgttctcataacgcagtctatataaggaggtgggaaag

SEQ ID NO: 80 - DNA, 950 bp, synthetic construct
  misc_feature 1..950: note = OTHER INFORMATION: Homology-PorD (Pp14-Pp15) UP
  source 1..950: mol_type = other DNA
acgaggatatcgttcgcaatgacgttattccacagcgcttggttcgtgaccaagctagttcagtggctcagcccaacgccaaaattacggtttcgtcctgaattttggaattctaaaatcaagcaggccattgcgtcggtaatcatagaccggggacaatggccttttttttggacaaggctttgctaagcgcgccaattcatagtaaactggaacttgatgttatttagttctcaaaacaggttgaaaaggagatacaaacaatgaaaaaaggtagaatagatcttgaaaacggcggcatcgtcgaaattgactttttcccagaagaagctccaaacaccgttgctaactttgaaaagctggcaaacagtggtttctacaacggtttgagcttccaccgcgtcattccaggctttgttgcacaagggggttgtccgatcggtaacggtacaggcggtcctggctacacaattgactgtgaaacagctacgaacacaaccaagcatgccaaaggcgttctgtccatggctcatgcaggtcgcaacaccggtggcagccaattctttatttgctacgctccacagccgcatttggatggagtacatactgtatttggtaaagtaaccaaaggtatggaattcattgatgctgtaaaacaaggcgacaaaatgaaagaggttaaagtatttgacgcgtaattttaccgtcacatgccttccctattttgaatagcgttgctcgtaagccgctctgtctttggacagggcggcttttgttgtcttttgtttgacggtttgctaattttcacaatcacgtacgtattacagtgcgtaagatcacattcgaaaagtaagtgttgtgatagccttttcacaagagtgcatatttccatttattttatatgagtgttcaagctcagtaagagttggaaaagaagggggcttatcaca

SEQ ID NO: 81 - DNA, 950 bp, synthetic construct
  misc_feature 1..950: note = OTHER INFORMATION: Homology-PorD (Pp14-Pp15) DN
  source 1..950: mol_type = other DNA
ttgtctattttaacgcgcacaaatcgactcggattttttgaaatccggctggaatccattggtggactcggcgcaaatctggctggcaaaatgttggccgaaaccggggtacttggtctgggactgaatgcttcaaatttctcatcctacggttcggaaaagaagggatctccggttaaaagctttgttcgcttctgtgagccggaggtggatattcgcgatcacagtccgatcgagcaaccgcatgtggtaggcattttccatgaaacgttatacaaaacggtaaatgtaatcagcggcctgccagcagatgggattgtagtggtaaattccacacgtccggcagaagaaatacgggctgatttgggactggaatccggtacattagcagtggtggatgcgatgggcattgcactggaagagcggaccaaactaaatacgtccatgcttggtgccttgttccgtgtctgtgacttcctggacccggaagcaatgagatcggttattcgcagtacttttgaaaagaaaaatccacaattagtggaacctaatctgcgtacatttaatcgtggttttgaagaaatgcaactacatgtatatccggttactgctgacgcgcaggatcgggttttcgaacgtccgcagcctttgctagggtacgaaactcaattgctagggggcgttattgcaactcagggcaacagcatgacgaaagatgttagtggttcacgccagggttatgtacctgagtatgaagctgataaatgtactcattgtgccaagtgtgattcgatttgcccggattactgctttgtatgggaagagcaagcagacaaacgcggaagaatgatgccgtttttgcaagggattgattaccagtattgcaaagggtgtctgaagtgtgtagatatttgcccgtcgggggcattaagctcgcgtcgcgagcaatg

SEQ ID NO: 82 - DNA, 950 bp, synthetic construct
  misc_feature 1..950: note = OTHER INFORMATION: Homology-PorD (Pb48-Pb50) UP
  source 1..950: mol_type = other DNA
gaaacctacgaggatatcgtacgcaacgatgttgtaccgcagcgcttggttcgtgaactagccagtccagcggttcatccgggcgccaaaattaccgtttcgtcctgatttttgatatgtgtgagcaagcagaccattgagccggggattattataccggtacagtggtcttttttttgggacaaggctttgctaagctgagcaattcatagtaaactggaacttgatgttatttagctttcaaaacaggttgaaaaggagatacaaacaatgaaaaaaggtagaatagatcttgaaaacggcggtatcgtcgaaattgattttttcccggaagaagctcccaatacggttgctaactttgaaaagctggctaacagcggcttctacaacggtctgagcttccaccgtgtcattccgggctttgttgctcaaggcggttgcccaatcggcaatggcacaggcggccctggatacaccattgactgtgaaactgctacgaataccacaaagcatgccaaaggcgttttgtccatggctcatgcaggccgcaacacaggtggcagccaattcttcatctgctacgctccacagccgcatttggatggagtacatactgtatttggcaaagtgaccaaaggtatggaatttattgatgctgtaaaacaaggcgacaaaatgaaagaggttaaagtatttgacgcataaaatgccgttaactacttgatttccttgtatagctacatttaggccgttctgtctttcgacagggcggctttttgttgtcttttgtttgacggtttgctgtttttcacaataacgcagtaattaaagtgagtaagatcacattcgaaaagtaagcgttatgatagccttctcatgagaatgcatttttttaattttttatgagtatttaagctcagtcaaggtgcaaaagaagggggcttatcaca

SEQ ID NO: 83 - DNA, 950 bp, synthetic construct
  misc_feature 1..950: note = OTHER INFORMATION: Homology-PorD (Pb48-Pb50) DN
  source 1..950: mol_type = other DNA
ttgtccattttaacgcgtacaaatcgactcggattttttgaaatccggctggaatccattggtggactcggtgctaatctggctggcaaaatgctagccgaaaccggggtgcttggtttggggctgaatgcttcaaatttctcgtcctacggttcagaaaagaaaggaactcctgttaaaagctttgtccgcttctgtgatccagaggttgacatccgtgaccacagtccgatcgaacaaccacatgtggtaggcattttccatgaaacgttatacaaaacagtaaaagtgatcagcggacttccagctgatgggattgttgtagtcaattctacccgaccggcagaggaaatacgggcagatttgggactggtatccggtacactggcggtagtggatgcgatgggcatcgcactggaggagcgaaccaaactgaatacgtccatgctcggcgccttgttccgtgtatgtgattttttggacccggaagcgatgagatcggttattcgcagtacatttgaaaagaaaaatccacagctggtggagcctaatctgcgtacattcaaccgtggttttgacgaaatggagctacacgtatatccagttaccagtgacgcgcaggatcggatttttgaacgaccacagcctttgctgggttatgaaacacaagcattggggggcgttattgccacacaggggaacagtatgacgagggatgttagtggctcgcgtcaaggttatgtgcctgagtatgaagctgataaatgtactcaatgtgccaaatgtgattcgatttgcccggattactgttttgtgtgggaagagcaagcagataaacgcggaagaatgatgccatttttgcaagggattgattaccagtactgtaaaggttgtctgaaatgcgtagatatttgcccttcgggagcgttaagctcgcggcgcgagcaatg

SEQ ID NO: 84 - DNA, 900 bp, synthetic construct
  misc_feature 1..900: note = OTHER INFORMATION: Homology DUF269 (Pp16-Pp22) UP
  source 1..900: mol_type = other DNA
cgtgatggctggacttcttttggaattgtatctctgctgtttatttctttctgggcacttgtctttctcatctatgtagaaagcggcaaggataatgctgtaattgaaatttcattattcaagaattcaagatttacgattagtgttattgcctccagtctggtcatgatggggatgtacggaggaacgtttttgacaccgttatttttgcaaaatattcagcgtgtgtcgccaattgatacgggtattatcctcattccacaagccattgccatggcggttatgatgcctattgccgggcggctgttcgataagttcggtattgtgcctttgggacttgtcggtctgacactaaccagctttatgacgtatcatcttcatcaacttacgccagatacttcgcatgcctggctggaaacggtaatgtcattacgggggctgggaattggtatttgtatgatgccattgtccacggtcggtatgaacgctgttcatccgagcaaggtaggcaatgcatctactgcatccaatctggtacgtactgttgcttcatctatggctatcgcggtgctgacctcgatcatgcaaagtcgttcggcagctcatgcccaacaaatttcggagactgttacaccggacgcggctcatacgctacaagcgacgttgggcaactcatggatgtcatccataagtagtttaattacactggatgccacatcgagaggaattggggatacattccttatttcatccataccgctgttttgcacgattccactcatctttttgtttattcagaggaagaaagcaaagacacagccggaagcttaataaagagaagtacaaaaatatcgactggatggcttacattatttccacggacagaaagaaagggaatta

SEQ ID NO: 85 - DNA, 900 bp, synthetic construct
  misc_feature 1..900: note = OTHER INFORMATION: Homology DUF269 (Pp16-Pp22) DN
  source 1..900: mol_type = other DNA
attggtctagagactaagttttgtttttcgtactatatattcgtgctttataatagcgagatttatgaaaaagaatatgagcaggacagaccgcagctgtttatcggtctgtcctgcttttattttttcgcatattttacataggattaatgtcggaaagaggctatttttgacgaacattggtatgataggatagactgaattggaaatagatagtctactagatgggatttattacattctcagatgagcgatcggaggaattgtaaaaatggaagtgaaattagtagttgattttgatgcttatgaggatggccaaacgatggctaaatccattgaggagctggcagccaagccagagagtgctgaggttacggagctaattattggcgattggggcggagcctatgaaaattcgcctgaggattttatcccggtgctggtgcagcataaagatcaatttccgaagttgcgcaagctgtttatcggcgatatggaatatgatgagtgtgaggtttcatggataaatcaaacgaatctgtccccgttgttagaagcgtttccagcactggagtctttttatgtcaaaggaagccaggacttgagcttggagccgcttcgccatagccgccttcaggaattggttattatttgcggtggtctgccggtcagcgtccttcagcaaatatatcatgccgagctgccagagctgcgcaagctggagctgtatttgggtgtggatcaatacggttttgatggtagtctgaaagatgttttaccgtttttgaacaataatccatttcccaaacttacatacttggggctaaaggatagcgaaatacaggatgaaattgcgattgcagcagctaatgccccagtcatcgaacaattggaagtgct

SEQ ID NO: 86 - DNA, 15 bp, synthetic construct
  misc_feature 1..15: note = OTHER INFORMATION: Synthetic spacer and RBS
  source 1..15: mol_type = other DNA
aaggaggtgggaaag

SEQ ID NO: 87 - DNA, 1896 bp, synthetic construct
  misc_feature 1..1896: note = OTHER INFORMATION: czcB::PtrnQ-5' UTR-nifUS(Tv)
  source 1..1896: mol_type = other DNA
cattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatggcatatacgatcacttcacagtgcatttcatgtaagttatgcagtagtgtctgcccgacgggagccattaaagtagccgaagacggtcagcattggattgaccaagcgttgtgtaccaattgtgttgatagtgttcataccgtcccgcaatgcaaagcagggtgtccaacatgcgatggctgcgtcaaagttccgagtgattattgggaagggtggttcgcaaattacaacagagtaattgccaagctaacaaagaagcaagattactgggaacgctggtttaattgttatagtcagaagttcagtgaacagatccaaaaacatcagggtgaaatcttaggggtataaagatgtctgtgatataccttgacaataacgcaaccacgaaagtagatccagaggtcgtggaggctatcatgccatacttgacggattactacgggaacccttcaagtatgcatacgtttgggggacaattaggaaaggcggttagaacagcgagagagcaagttgccgccctattaggggctgacgagtcagagatcgtatttaccagttgcggtacagagggggacaatgcagccatcagagctgctttactagcacagccggagaagcgacacattatcacaacccaagtggaacacccggcagttctaaatgtatgtaaacaactagagactcaaggttacaccgtcacgtatttgtcagtaaacggtcacgggcaactagacttagatgagttagaagccagcctaacggggaacacagcgttggtcaccataatgtacgccaataacgaaacaggcacggtgttccctatagaggagatcggcaagcgcgtaaaggagcgaggcgctatctttcacgttgacgcagtgcaagccgtagggaagatccctctaaatatgaagacgtctactatcgatatgctaacaatatctgggcataagatacatgctcctaagggtattggcgctttatacgtcagacgcggcgttcgattccgacctctattaatcggtggtcatcaggaacgaggccgacgagcgggaaccgagaatgtacctgggattgttggactaggaaaagccgccgaactagaacttattcatattgagactgccattaagaaggagaccagacttcgcgatagattagagcaaactttgctagccaaaattccggattgcgaagtcaatggggacgttactcaacgtttaccgaataccactaatatcggcttcaaatatatagagggggaagcgatcctactttcattgaacaagtatggaatctgcgccagcagtggatctgcttgtaccagcggcagtctagagccaagccatgttcttcgcgcaatgggcttaccttacacgacacttcacggatctatacgttttagtttgtgtagatacaccacggaagcacagatagaccgagtcatagaggtgatgcctgaaattgtagagcgattgcgcgccttgtcaccgttcaagaacgatgaggctgggtggcttcaagctcaggaacaaacattggcgcatcgctaaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 88 - protein, 186 aa, synthetic construct
  REGION 1..186: note = OTHER INFORMATION: DUF269 (Pd)
MDQMANEAASANEQPADTAQVRPKRRSKPAPDPEKAERIVRRLNDLLSASDYFGRYGTLTPEEKIAKLFLASAEDKAKSDINCTASDEVRGQVPVLFQAIAGVMEEKSGLMIQSSAEINVEGFGRGLIYSGRIILVMKSLRAGFPFPFTSLEKTVAYAVACLDEGLAFLDKYKEQTAVHGLLSLEG

SEQ ID NO: 89 - DNA, 561 bp, synthetic construct
  misc_feature 1..561: note = OTHER INFORMATION: DUF269 (Pd) (reverse translation)
  source 1..561: mol_type = other DNA
atggatcaaatggctaatgaagctgcatctgcgaacgaacagccggcggacacagctcaagtgcggccgaagcgccgttccaaaccggcgcccgatcctgaaaaggcggagcgtattgtccgccgcttaaatgatctgcttagcgcgagcgattattttggccgttatggtacactgactcccgaggaaaagattgcgaagctgttcctggcttcagccgaggataaggctaaatcggacatcaactgcacggcgtccgatgaagtccgcggccaggtgccggtattgtttcaggccatcgccggtgtgatggaggagaagagcggcttaatgattcaaagctctgcggaaattaatgtggaagggttcggacgtggactgatctacagcggccgcattattcttgtgatgaagagcctccgcgcaggctttccatttccgtttacttctctggagaagaccgttgcctatgcagttgcttgcttggatgaaggccttgccttcttagataagtacaaagaacagactgctgtgcacggactgctcagtctggagggataa

SEQ ID NO: 90 - protein, 186 aa, synthetic construct
  REGION 1..186: note = OTHER INFORMATION: DUF269 (Pf)
MDQMANEAASASAQPADGAQARPKRRSKPAPDPEKAERLVRRLNDLLSASDYFGRYASMTPEEKIAKLFLVSAEDKAKSDINCTATDEVRLQVPVLFQAIAGVMEEKSGLMIQSSAEINIEGFGRGLIYSGRIILVMKSLRAGFPFPFTSLEKTVAYGVACLDEGLAFLDKYKEQTAVHGLLSLEG

SEQ ID NO: 91 - DNA, 561 bp, synthetic construct
  misc_feature 1..561: note = OTHER INFORMATION: DUF269 (Pf) (reverse translation)
  source 1..561: mol_type = other DNA
atggatcaaatggctaacgaagccgcatcagcgagcgcgcagccggctgacggagctcaggcgcggccgaagcgccgctccaaaccggctcccgatcctgaaaaggcggagcgtcttgtccgccgtctcaacgatctgctcagcgcgagcgattattttggccgttatgcttccatgactcccgaggaaaagattgcgaagctgttcctggtatcggcagaggataaggctaaatcggacattaactgcacggcgaccgatgaagtccgccttcaggtgccggtattgtttcaggccatcgctggcgtaatggaggagaagagcggcctgatgattcaaagctctgcagaaattaatatcgaaggattcggccgcggactgatctacagcggccgaattatccttgtcatgaagagtctgcgcgcaggctttcccttcccgttcacttctctggagaagacggtggcctatggggttgcttgcttggatgaaggccttgccttcctggataaatataaggaacagactgctgttcacggattgctcagtctagagggataa

SEQ ID NO: 92 - protein, 188 aa, synthetic construct
  REGION 1..188: note = OTHER INFORMATION: DUF269 (Pg)
MESSANRKIAVQAGLSREGAAAKPRRRKVVKELDPDTADAFVRRLCHLLDTADFFGRHASLSPQEKIAKLFLASPEDKDQSDFNCAVSPKVRQQVPILFQAIAGVMEEKSGAMVQSTAEINVEGFGRGLLYTGRVILVLKALRAGAPFPFTSEEKTIRYGVGCVEEGLVFLEKYKELTAGHGLFSLEG

SEQ ID NO: 93 - DNA, 567 bp, synthetic construct
  misc_feature 1..567: note = OTHER INFORMATION: DUF269 (Pg) (reverse translation)
  source 1..567: mol_type = other DNA
atggaatcgtcggctaacaggaagatagcagtccaggctgggctttcccgggagggggcggcggcgaaaccccgccgccgcaaggttgtaaaggagctggaccccgatacggcggatgcctttgtccgccgtttgtgccatctgctcgatacggctgactttttcggacggcatgcctcgctgtctccgcaggagaagattgccaagctgtttctggcatcccctgaggacaaggatcaatcggacttcaactgcgccgtatctcccaaggtccgccagcaggttccgattttgttccaggcgatcgccggagtcatggaagagaagagcggagccatggtgcagagcactgctgagattaatgtggagggttttgggcgggggctgctgtataccgggcgcgttattctggtgctgaaggccctgcgggccggtgcccctttcccgttcacttcggaggagaagaccatccgttatggggtggggtgtgtggaggaaggtctggtttttttggagaagtataaggaattgacggcaggccacggtttgttcagccttgaaggataa

SEQ ID NO: 94 - protein, 188 aa, synthetic construct
  REGION 1..188: note = OTHER INFORMATION: DUF269 (Pj)
MESSANRKMAVQAGFSREGAAVKPRRRKVVKELDPGTADALVRRLCHLLDTADFFGRHAPLSPQEKIAKLFLASAEDKDQSDFNCAVSPKVRQQVPILFQAIAGVMEEKSGAMVQSTAEINVEGFGRGLLYTGRVILVLKSLRAGAPFPFTSEEKAIRYGVECVEEGLAFLEKYKEITACHGLFSLEG

SEQ ID NO: 95 - DNA, 567 bp, synthetic construct
  misc_feature 1..567: note = OTHER INFORMATION: DUF269 (Pj) (reverse translation)
  source 1..567: mol_type = other DNA
atggagagttctgcgaatagaaaaatggcggtgcaagcgggcttcagccgtgaaggggcggcagtaaaaccacgccgtagaaaagttgttaaggaactagatccaggtacagcagatgctttagtccgccgtctttgccaccttcttgataccgctgatttctttggccgacatgcaccgttatctcctcaagagaaaatcgcgaaattattcctagctagcgcggaggataaagatcaatcagacttcaactgtgccgtatctccgaaggtccgccaacaggtaccgatcttgtttcaggcaattgcaggtgtgatggaggaaaaatcaggtgcgatggttcaatctacggctgagataaatgttgagggatttgggcgaggcctactatacactggccgtgtcattttggtgcttaaaagcctacgcgcaggtgctccgttccctttcacttcagaggagaaggccatccgttacggcgttgaatgtgttgaggaggggctagcgtttttggagaagtacaaggagatcacagcgtgtcatgggcttttctcacttgagggataa

SEQ ID NO: 96 - protein, 187 aa, synthetic construct
  REGION 1..187: note = OTHER INFORMATION: DUF269 (Pr)
MEPLADGKMAVQAGPAREGAAVKPRRKVVKELDQGTADAFVRRLCNLLDTADFFGRHASLSPQEKIAKLFLASAEDKNKSDFNCAVSPKVRQQVPILFQAIAGVMEEKSGAMVQSTAEINVEGFGRGLLYTGRVILVLKSLRAGAPFPFTSEEKTIRYGVECVEEGLAFLEKYKEMTACHGLLSLEG

SEQ ID NO: 97 - DNA, 564 bp, synthetic construct
  misc_feature 1..564: note = OTHER INFORMATION: DUF269 (Pr) (reverse translation)
  source 1..564: mol_type = other DNA
atggaaccattggctgatggaaagatggcagttcaagccgggcctgcccgggagggggcggcggtgaagccccgccgcaaagttgtaaaggagctggatcagggtacggcggatgcttttgtccgccgtttgtgtaatctgctcgatacggcggatttcttcggacggcatgcttcgctgtctccgcaggagaagatcgccaagctgttcctggcttccgccgaggacaagaataaatcggacttcaactgtgccgtatctccgaaggtccgccagcaggttccgattttgttccaggctatcgccggagtcatggaagagaagagcggagcgatggtgcaaagcaccgctgagattaatgtggagggttttggacgggggctgctgtataccgggcgcgtgattttggtgctgaagagcctgcgggccggtgcccctttcccgttcacttcagaggagaagaccatccgctacggggtggaatgcgtcgaggaggggctggcttttttggagaaatataaggaaatgaccgcatgccacgggttgctcagccttgaaggataa

SEQ ID NO: 98 - protein, 185 aa, synthetic construct
  REGION 1..185: note = OTHER INFORMATION: DUF269 (Psab)
MDPTANEIAPANEQPANRQPALPKRRPPAPDPEKAAHLVRRLNDLLSASDHFGRHAALTPEEKIAKLFLVSAEDKAKSDFNCTASDDVRRQVPLLFQAIAGVMEENCGLMIQSSAEINVEGFGRGLLYSGRVILVMKSLRAGFPFPFTSLDKTVAYGVACVEEGLAFLEKYKELTSVHGLLSLEG

SEQ ID NO: 99 - DNA, 558 bp, synthetic construct
  misc_feature 1..558: note = OTHER INFORMATION: DUF269 (Psab) (reverse translation)
  source 1..558: mol_type = other DNA
atggatccaacggctaacgaaatcgcacccgcgaatgaacagccggctaacagacagccggcattgccgaaacgccgccccccggcccccgatcctgaaaaggctgcgcatcttgtccgccgcttgaacgacctgctcagcgcgagcgatcatttcggccggcatgcagccctgactcccgaggagaagattgcgaagctgttcctggtatcggcggaggataaagcgaaatcggatttcaactgcacggcatccgatgatgttcgccgccaagtgccgctattgttccaggccattgccggcgtgatggaagagaactgcggcttgatgattcaaagctctgcggaaatcaacgtcgagggcttcggccgcgggctgctctatagcggccgcgttattcttgtaatgaagagtctccgcgcaggctttccgtttccgttcacttctctggacaagacggtggcctacggggtagcttgcgtagaagaggggctcgcctttttggagaaatataaagaattgacctctgtgcacggactgctcagtctggagggataa

SEQ ID NO: 100 - protein, 186 aa, synthetic construct
  REGION 1..186: note = OTHER INFORMATION: DUF269 (Pstel)
MERMADEFAGVNGQTPEQAPVSPKRRTKPLPDPEKSEQLIRRLNDLLSASDYFGRYASLTPEEKISKLFLASADDKAKSDINCTVSDEVRSQVPVLFQAIAGVLEEKSGLMIQSSAEINVEGFGRGLLYSGRIILVIKSLRAGFPFPFTSLAKTVAYGVACVEEGLTFLDKYKDLAEVQSLLNLEG

SEQ ID NO: 101 - DNA, 561 bp, synthetic construct
  misc_feature 1..561: note = OTHER INFORMATION: DUF269 (Pstel) (reverse translation)
  source 1..561: mol_type = other DNA
atggaacgaatggctgatgaattcgcgggtgtgaatggtcagactccggaacaggctcctgtttcgccgaagcgccggactaaacctctgcccgatcccgagaaatcggaacagctgatccgccggctaaatgatctgctcagcgccagcgattatttcgggcgctacgcttccctgacgccggaggagaagatctcgaagctgttcctcgcttctgcagatgacaaggccaagtccgatatcaattgcaccgtatccgatgaagtgcgttcccaggtgccggtgttgtttcaggcgattgccggcgtgctggaggagaagagcgggctgatgattcagagctcggccgagatcaatgtcgaaggctttggccgcgggctgctgtatagcggccggattatcctggtgatcaagagccttcgtgcgggctttccgttcccgttcacctcacttgcgaagacggttgcttatggagtggcatgtgtagaggaagggcttaccttcctggataaatataaagatctggcagaagtgcagagtcttctcaatctggagggataa

SEQ ID NO: 102 - DNA, 187 bp, synthetic construct; identical to SEQ ID NO: 77
  misc_feature 1..187: note = OTHER INFORMATION: PsufC(Pb)::PR'-5' UTR
  source 1..187: mol_type = other DNA

SEQ ID NO: 103 - DNA, 2501 bp, synthetic construct
  misc_feature 1..2501: note = OTHER INFORMATION: czcB::PtrnQ-5' UTR-nifUS(Ps)
  source 1..2501: mol_type = other DNA
cattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatgtgggattatagcgaaaaggtcaaggaacatttctataatccgaaaaacgctggagcagtggcggaggcgaacgcagtgggcgatgtggggtcattgtcatgtggcgatgcgctaagactatctttaaaggttgaccctgataccgacgtaatacttgatgcaggttttcagacatttggctgtggctctgcaatagccagcagctctgccctaacggagatgatcaaagggcttacagtagatgaggcacttaaaataagtaaccaggacatcgcggatttcctagatgggcttccaccggaaaagatgcactgtagtgtgatggggcgcgaagcattacaagcagcagttgccaattacagaggggagactcttgaagacgatcacgaagagggcgcgttagtctgcaagtgttttgcgattgatgaagtcatggtaagagaaacgatccgtgctaatcgcttgtcaagtgtagaggatgtgactaactacacgaaggcgggaggtggctgctctagttgccatgaagggattgaaagactattggtggaagagcttgcagccagaggtgaaatcttcgtaccggcgggaacaggcgccaaagcagccaagaaggctaaggcaccattagttacgttagaaacacctccggccgccccgcaagccgcgcctactgccccaagaatgaccacgttgcagagaataagacgcattgaacgcgtcctagagtcaatacgaccaactctacagcgcgatcatggcgacgtggagctattagatgtggaaggcaagaacatatacgtcaaactaacgggggcgtgtacaggatgtcaaatggcctcaatgacgttaagcggaatccaacagagattgattgaggaattaggggaattcgttaaagtcgtcccagtatcatctccagctcatagcgcgatggcggaggtgtgagatgtcaggaatataccttgataacaacgcaaccacacgcgttgatgacgaggtggttcaggcaatgctaccgttcttcaccgagcaattcggtaacccttcaagtatgcacagtttcggcaatcaagtagggatggcgcttaaaaaagcaagacaatcagtgcaacgattgttaggagccgaatatgatagcgaaattgtcttcacctcttgcggaaccgaggccgacagcaccgctattttgtcagcattgaaggcacaaccggaacgcaaaactatcataacgactgtagtagagcacccagccatccttagtttatgtgactacttagccgaggatggatataccgtccacaaattgaaagtagataaaaagggacgattagatttagacgagtacgcagcattgctagatgacgatgtagcgatagtcagcgtgatgtgggcaaataacgaaaccggtactctttttccagtggagcaaatggcacagatggctgacgacgcgggggtcatgttccacagtgacgcagtgcaagctgtgggaaaggtcccaatgaaccttaaggggtctgcaatccatatgctatctcttagtgggcacaagttgcacgcaccgaaaggggtcggggtactatatttgcgtagaggcacgcgttttcgtccattgctacgaggcggtcaccaggagagagggcgcagagctggcacggaaaatgcggcaagcatcattggcttaggggtcgctgcggaacgagcacttgcgtttatggagcacgagaacactgaagtacgacgtttacgtgataaactagaggccggtatattagccgcggtaccatacgcattcgtaacgggcgacccaggcaaccgattgcctaacacagccaacatcgcattcgaatacatcgagggggaggccattcttttgcttttgaacaaagtaggaattgccgccagcagtggcagtgcatgcacgtcaggttctttagaaccatctcacgttatgcgcgccatggacataccatataccgcagctcacgggagtgtccgcttcagtttgagcagatatacaactgaggaacaaatagattatgtaattagagaggttccacctataattgctcaacttcgaaaactaagtccatactggtcaggtaacggcccggctgaagcggttggcgattcattcgaaccagtttacgcatgaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 104 - DNA, 2398 bp, synthetic construct
  misc_feature 1..2398: note = OTHER INFORMATION: czcB::PtrnQ-5' UTR-nifUS(Kv)
  source 1..2398: mol_type = other DNA
cattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatgtggaattattcagagaaggtcaaagatcactttttccaccctcgaaacgcgagagtcgttgacaatgccaatgcagtaggggacgtagggtctctttcatgcggcgacgctcttcgcctaatgttgcgagtcgatccgcaaaccgagataatagaagaggcaggttttcagacttttggttgtggaagcgcaatagccagttcttcagcccttacggaactaattattggtcacacattgacagaggctggacaaataactaaccaacaaattgcagattatctagatggccttcctccagaaaagatgcactgttcagtcatgggtcaagaggcgttgagagcagctatagcccattttagaggcgagtcactagaggaagaacatgaggagggaaagttgatctgtaaatgtttcggcgtcgacgagggacatatccgccgcgctgtggtaaacaatggattaacgacacttgaggaggttataaactatactaaggcgggaggcggttgcactgcttgccatgagaagatagaacttgctcttgccgcaatcttggcccagcaacctccggctccattacctgtagagacggcacatgacgcccattggcagagtgtcgtggacactatcaatgaacttagacctcacatccaggctgatgggggcgatatgacccttctaaatgtcactccgagacaggtcacggtatctttgtctggctcttgcagcgggtgtatgatgaccgatatgacgcttgcttggctacaacagaaactaatggaaagaacaggaacttatatggacgtagtcgcggccccggcggccgtaaactaacccaagggggatacgatgaagccggtttacttggataataacgcaactacacgtcttgacccaatggtgttggatgcaatgatgccgtttttgacggatttctacggtaatcctagctcaattcacgatttcgggctacctgcgcaggccgcacttgagcgagcacatcagcaggtcgctgccttattaggtgctgattatccatctgagattatatttaccagttgcgcaactgaggctacaacaaccgccatagctagtgcggtagcactaatgccagagagacgcgagataataactagcgcggtggagcacccagctaccttagccgtatgcgagcagttagagagacaaggttacttgatccaccgaatagccgttgaccagcagggagcactagatatggcacagttccgtgcagctctatcacctagagtcgcgctagtgagtatcatgtgggctaataatgaaacaggagtcatgtttcctgttccggagttagcggaattggctcatgaacagggcgcgttatttcattgcgatgcagtccaggtagtcgggaagattcctatggcgctatctcaaacccgaattgatatgctaagctgctctgcgcataagtttcacggtccgaagggggtaggctgcctttggcttcgccgcggtacgcgttttcgaccgttattgcgagggggacaccaggctcatgggcgcagagccggaacggaaaacatttcaggcatagttgggatgggagccgcgagtgaattggcacaggtccatttgccggggatggcccaaattagccaattgcgagatagattggaacaacacctacaagccaccataccggcagtcacagtaatgggcgggtcacaaccacgagttccaggtacgcttaatttggcgtttgaatgcattgagggagaagctattttgcttttaatgaaccaagcaggcatagctgcttcaagtggatcagcgtgcacaagcgggtctttggagccttcacacgttatgcgagcgatgaacatcccgtacaccgcagctcacggaaccatacgattttcacttagtcgttacacgcgcgagaaagaaatagattacgtcattgcaaccctaccgagcgtaatcgaccgccttagagcattgagtccgtactggcgccaggggcagccgcatctacaagacggagcatttaccccggtttatggctaaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 105 - DNA, 2256 bp, synthetic construct
  misc_feature 1..2256: note = OTHER INFORMATION: czcB::PtrnQ-5' UTR-nifUS(Rt)
  source 1..2256: mol_type = other DNA
cattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatgcgtaagtacagaggtaagcttaagacaagtttttgcgatcataagggtgctcgcatacttcaaactgcgaagagcgaaaataaggtcgctagtggtgacgcagtggaattgatgatgggtttcgaccgcccaacagaggctataagcgcagccacacttcagagtttgagaactcgatttgcgatagcctgttcttctgagttcatcgaattcattgtgggtaaatcagcgtatgaggctattcgaaacatcaattgtgaccttacctatgtagtagatcgtccgcctagcacctcacctagtccatttgccagttgcgtggctcaatctcgtgctattcgaagccacgcctcacagctatcaaagaagagattattgcgttcacttagacgatgcagcttgaaagcctcattagggcagcgacgagtttgccgtcaggctcagcgtaaggctgcgtatagtcgatctaaaaagtacttatgtaatcgacgaggcttttcagcagcttgtcgacgcggttctcgtatgcttggtgcacgtttgaacaaagtgcatcgtagtcttaaacttccagacaatgtgccagcagccaacagcactaaccgaactttaagaagattatttttgccggagcagtgaacgacggagcctacccgaggtcactccgccaactgaggatcgcttccatgcgctctgtatacttggacaacaacgctacaacacgcgtcgaccctgaggtagtgcaggccatgctaccgttttttacggaccagtttgcaaatccgagcagctcacattcatttggagctagcgcttcagcggccataatgaccgcccgaaagagattgcaagcattaatcggagcagaatttgaacatgagatagttttcaccagtgggggtactgaatcagacaacgcagctattttgagtgccctagagataatgcctgacagaacgcagattgtaacctctgccgtggagcacccagccgtccttaccctttgtgcccatttggagcgaacgcgtggcataaaagttcaccgcattcctgttgatcgtcacggaagattggatttacgcacctatcgagcggcgttgacccctcacgtcgctctagtgagcatcatgtgggctaacaatgagactggtgcaatctttcctgtcgctgacttagccaagctagcgaaggaggtaggagcctttttccatacagatgcagtccaagcagtaggaaagttgccaatggatctaaaatcttctgcaatagatatgctatctcttagtggacacaagcttcacgggcctaagggtattggtgctctttgggtgaagcgaggtgttcgtttcgatccgatgataaagggtggtcaccaagaacgcgatagacgtgcgggcactgagaacacggcaggaatagtggggttgggcaaagctgcagaactagcttctagcttcatgaaagaagagaacggtcgcgtccgaagcttacgcgacagactagagcgcggcttattacaacgcattcctgacgccttcgtcacaggagacaaacaaaagcgacttccaaacacttctaatatagccttcgaccaggtggacggcgacgggttgctattgttgttagatcgctacgggatagcttgcagtagcgggagtgcctgcacgtctcagagccctcaacctagccatgtgttaacggccatgaatattccagacatctcttcacatggcgtaataagattttctcttagccgagataactgcgaagaggatatagaccgagtgttagacgttttgccagggatagttggtagtcttcgtgacggctcatcattcaaaccaaccccggcaaaacgccgtcgacctttagcctttatgaagccgactgaaacagatggtaatacttcatgaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 106 - DNA, 2389 bp, synthetic construct
  misc_feature 1..2389: note = OTHER INFORMATION: czcB::PR'-5' UTR-nifUS(Kv)
  source 1..2389: mol_type = other DNA
agacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatgtggaattattcagagaaggtcaaagatcactttttccaccctcgaaacgcgagagtcgttgacaatgccaatgcagtaggggacgtagggtctctttcatgcggcgacgctcttcgcctaatgttgcgagtcgatccgcaaaccgagataatagaagaggcaggttttcagacttttggttgtggaagcgcaatagccagttcttcagcccttacggaactaattattggtcacacattgacagaggctggacaaataactaaccaacaaattgcagattatctagatggccttcctccagaaaagatgcactgttcagtcatgggtcaagaggcgttgagagcagctatagcccattttagaggcgagtcactagaggaagaacatgaggagggaaagttgatctgtaaatgtttcggcgtcgacgagggacatatccgccgcgctgtggtaaacaatggattaacgacacttgaggaggttataaactatactaaggcgggaggcggttgcactgcttgccatgagaagatagaacttgctcttgccgcaatcttggcccagcaacctccggctccattacctgtagagacggcacatgacgcccattggcagagtgtcgtggacactatcaatgaacttagacctcacatccaggctgatgggggcgatatgacccttctaaatgtcactccgagacaggtcacggtatctttgtctggctcttgcagcgggtgtatgatgaccgatatgacgcttgcttggctacaacagaaactaatggaaagaacaggaacttatatggacgtagtcgcggccccggcggccgtaaactaacccaagggggatacgatgaagccggtttacttggataataacgcaactacacgtcttgacccaatggtgttggatgcaatgatgccgtttttgacggatttctacggtaatcctagctcaattcacgatttcgggctacctgcgcaggccgcacttgagcgagcacatcagcaggtcgctgccttattaggtgctgattatccatctgagattatatttaccagttgcgcaactgaggctacaacaaccgccatagctagtgcggtagcactaatgccagagagacgcgagataataactagcgcggtggagcacccagctaccttagccgtatgcgagcagttagagagacaaggttacttgatccaccgaatagccgttgaccagcagggagcactagatatggcacagttccgtgcagctctatcacctagagtcgcgctagtgagtatcatgtgggctaataatgaaacaggagtcatgtttcctgttccggagttagcggaattggctcatgaacagggcgcgttatttcattgcgatgcagtccaggtagtcgggaagattcctatggcgctatctcaaacccgaattgatatgctaagctgctctgcgcataagtttcacggtccgaagggggtaggctgcctttggcttcgccgcggtacgcgttttcgaccgttattgcgagggggacaccaggctcatgggcgcagagccggaacggaaaacatttcaggcatagttgggatgggagccgcgagtgaattggcacaggtccatttgccggggatggcccaaattagccaattgcgagatagattggaacaacacctacaagccaccataccggcagtcacagtaatgggcgggtcacaaccacgagttccaggtacgcttaatttggcgtttgaatgcattgagggagaagctattttgcttttaatgaaccaagcaggcatagctgcttcaagtggatcagcgtgcacaagcgggtctttggagccttcacacgttatgcgagcgatgaacatcccgtacaccgcagctcacggaaccatacgattttcacttagtcgttacacgcgcgagaaagaaatagattacgtcattgcaaccctaccgagcgtaatcgaccgccttagagcattgagtccgtactggcgccaggggcagccgcatctacaagacggagcatttaccccggtttatggctaaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 107 - DNA, 2483 bp, synthetic construct
  misc_feature 1..2483: note = OTHER INFORMATION: czcB::PR'-5' UTR-nifUS(Av)
  source 1..2483: mol_type = other DNA
agacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatgtgggattattctgagaaagttaaagaacatttttacaatcctaagaacgcgggagcagtggagggagccaacgccattggggacgtcggctctctttcatgtggggatgcactacgactaacacttaaagttgatcctgaaacagatgtcatattagacgcgggtttccaaacgtttggctgtggctcagcgatagcttcatcatcagctcttactgagatggtgaaggggttgaccctagacgaggcattaaagataagtaaccaggatatagccgactatttggatggattgccacctgagaagatgcattgttctgtgatgggacgcgaggccttgcaagcggcggttgctaactacagaggggagaccatcgaagacgatcacgaagagggagccctaatttgtaagtgtttcgccgttgacgaagtcatggtccgtgatacgatacgtgccaacaagttgagtaccgtcgaggacgtaacaaattacacgaaggccgggggaggttgctctgcgtgtcacgaggcaatagagagagttttgacagaagaattggcagcacgtggcgaagtctttgtcgcggctccaatcaaggcgaagaaaaaagtaaaggtccttgcccctgagcctgctcctgcaccagttgctgaagccccagcagcggcgccaaagttaagcaaccttcagcgtataagacgcatcgagactgtattagcagcaatcagacctacgcttcagcgtgataaaggggatgtggagcttattgacgttgatgggaaaaacgtttacgtgaaactaacgggcgcctgcaccgggtgccaaatggcatcaatgactttgggcggtattcagcagagacttatcgaagaattaggggaatttgtcaaagtcatccctgtttctgcagcagcacatcacgcgcagatggaggtctgaaatggcagatgtctatttagacaacaacgctacaacgcgcgttgatgacgaaatcgttcaagcgatgcttccgttctttacagaacaattcgggaatccatctagccttcacagcttcggtaatcaggttgggatggcattgaaaaaagctcgtcaatcagtgcaaaaactattgggagccgagcacgattctgaaatagtcttcactagctgcggtactgagagcgactctactgctattctaagcgccttaaaggcgcagccggagagaaagacagtgataactactgtggttgagcacccagccgtgctttcattgtgtgactacctagcatcagaagggtacacagtacataaattgcctgtcgataaaaaagggcgattggatcttgagcactacgcgagcttacttacggacgatgtagcagttgtcagcgttatgtgggctaataacgaaaccgggactttgttccctatagaggagatggcaagactagcggatgacgctgggataatgttccatacggacgcggttcaagctgtaggaaaggtcccgatagacctaaagaatagttctatccatatgctttctttatctggtcacaagttgcatgcgccgaagggcgtgggggtattatacctacgacgcggaacgagatttcgtccactactaagaggcggacatcaagagcgaggcagacgtgctggaacggagaatgccgcttctattattggacttggagtggcggcagaacgagcgctacaattcatggagcacgagaacacagaggtgaaaagattgcgcgacaaacttgaggcaggcattctagctgtggtgccgcacgcattcgtgacgggagacccagacaaccgtttgccgaatacagccaatatcgctttcgagtatatagaaggggaggctattctacttcttcttaataaggttggtattgccgctagctcaggatctgcttgtactagtggatcattagagccaagtcacgttatgcgagcgatggacataccgtacacggctgcccacggaaccgtgcgattttcattatcacgatatactacagaagaagaaatcgaccgagtcatccgtgaggtcccgccgattgtggctcagcttcgaaaattatctccgtattggagtggaaatggcccagtcgaagacccaggtaaagcctttgctccggtatatggttgaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 108 - DNA, 2492 bp, synthetic construct
  misc_feature 1..2492: note = OTHER INFORMATION: czcB::PR'-5' UTR-nifUS(Ps)
  source 1..2492: mol_type = other DNA
agacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatgtgggattatagcgaaaaggtcaaggaacatttctataatccgaaaaacgctggagcagtggcggaggcgaacgcagtgggcgatgtggggtcattgtcatgtggcgatgcgctaagactatctttaaaggttgaccctgataccgacgtaatacttgatgcaggttttcagacatttggctgtggctctgcaatagccagcagctctgccctaacggagatgatcaaagggcttacagtagatgaggcacttaaaataagtaaccaggacatcgcggatttcctagatgggcttccaccggaaaagatgcactgtagtgtgatggggcgcgaagcattacaagcagcagttgccaattacagaggggagactcttgaagacgatcacgaagagggcgcgttagtctgcaagtgttttgcgattgatgaagtcatggtaagagaaacgatccgtgctaatcgcttgtcaagtgtagaggatgtgactaactacacgaaggcgggaggtggctgctctagttgccatgaagggattgaaagactattggtggaagagcttgcagccagaggtgaaatcttcgtaccggcgggaacaggcgccaaagcagccaagaaggctaaggcaccattagttacgttagaaacacctccggccgccccgcaagccgcgcctactgccccaagaatgaccacgttgcagagaataagacgcattgaacgcgtcctagagtcaatacgaccaactctacagcgcgatcatggcgacgtggagctattagatgtggaaggcaagaacatatacgtcaaactaacgggggcgtgtacaggatgtcaaatggcctcaatgacgttaagcggaatccaacagagattgattgaggaattaggggaattcgttaaagtcgtcccagtatcatctccagctcatagcgcgatggcggaggtgtgagatgtcaggaatataccttgataacaacgcaaccacacgcgttgatgacgaggtggttcaggcaatgctaccgttcttcaccgagcaattcggtaacccttcaagtatgcacagtttcggcaatcaagtagggatggcgcttaaaaaagcaagacaatcagtgcaacgattgttaggagccgaatatgatagcgaaattgtcttcacctcttgcggaaccgaggccgacagcaccgctattttgtcagcattgaaggcacaaccggaacgcaaaactatcataacgactgtagtagagcacccagccatccttagtttatgtgactacttagccgaggatggatataccgtccacaaattgaaagtagataaaaagggacgattagatttagacgagtacgcagcattgctagatgacgatgtagcgatagtcagcgtgatgtgggcaaataacgaaaccggtactctttttccagtggagcaaatggcacagatggctgacgacgcgggggtcatgttccacagtgacgcagtgcaagctgtgggaaaggtcccaatgaaccttaaggggtctgcaatccatatgctatctcttagtgggcacaagttgcacgcaccgaaaggggtcggggtactatatttgcgtagaggcacgcgttttcgtccattgctacgaggcggtcaccaggagagagggcgcagagctggcacggaaaatgcggcaagcatcattggcttaggggtcgctgcggaacgagcacttgcgtttatggagcacgagaacactgaagtacgacgtttacgtgataaactagaggccggtatattagccgcggtaccatacgcattcgtaacgggcgacccaggcaaccgattgcctaacacagccaacatcgcattcgaatacatcgagggggaggccattcttttgcttttgaacaaagtaggaattgccgccagcagtggcagtgcatgcacgtcaggttctttagaaccatctcacgttatgcgcgccatggacataccatataccgcagctcacgggagtgtccgcttcagtttgagcagatatacaactgaggaacaaatagattatgtaattagagaggttccacctataattgctcaacttcgaaaactaagtccatactggtcaggtaacggcccggctgaagcggttggcgattcattcgaaccagtttacgcatgaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 109 - DNA, 2315 bp, synthetic construct
  misc_feature 1..2315: note = OTHER INFORMATION: czcB::PR'-5' UTR-nifUS(Fc)
  source 1..2315: mol_type = other DNA
agacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatgacggcgacggctacagccatagaactaactgaccgcgcccgcgctcaagtccgtcgactattaggttctactccgggtacggcaggttttgcactacgcattggcgttgatccgggaggttgttctggttatacgtacaatttatcactagtcccagatgctgatgccgacgacattgtctatcctcaggacggattcgatgtgttagtccatacatctatggtcgacttactacgtggaatgagagtagactatgctgagactttgactagttctggttttaccttcacgaaccctaatgcgaaaagctcatgcggctgtggatctagtttcggcgcctctgactctcctcaacgtacagaagcagatgcaaaattacgcgctcaagttgaggatgtcttgggcgagatccgcccgttcttgagaggcgatgggggagatgctgaaatagtggctgtcctagccggaggtggtgaaccagggtcagccgaggtccacctacgtttgaccggcgcatgtggaggttgtagcagcgcgaatgccactttgacgggcgttatcgagactagacttaaagaggcgctaccagaaataggacgtgttgcattggtttcttgagctgacgttctcataacgcagtctatataaggaggtgggaaaggtaatatacttggacaacaatgcaacaaccccagtcgacccggaggtcttaagtgcaatgttgccattacttcgtgaacattacgcaaaccctgctagccgacacccagcaggccaagccgtttcacgtcttgtcgaggcggcaagaagagatgtcgcagcgctagccggcgctgcaggtagagatgtcgttttcacgagcggagctaccgaggcggcgaacctagctacgcacggtgttctagccactgccgcaccaactagacgacgcattttagtaggcgcaaccgaacacccagcagtccttgctgcagcccacgctgccgccgacagacacgagggaagtgtgcagacagtacgtgtccaccctggtgggacggttgatttaagccacttgagaagcttgcttcgacctacaccagctgggggtgaggttgcacttgtagctgtcatggcagcgaatcacgaaacaggcgcacttacggatccacgtgctgtcgcagccttcgcacacggcgccggcgctttatttctttgtgacgtcacgcaagcattgggtcgcataccagttgctgtaaccgagacagggattgatctagcaatagcgagtgcgcataaagtttatggtccaaaaggggcaggcgttctattagcaggtcgagctgcgagagctcgtttggtccctttgttacacggcggtgggcaggaacgcggtcttagagccgggactctaaatacgccagctattgtagggttcggagctgcagcacgtatcgcagcagcagtacttcctgacgaagcaatccgccaacgtcagttaacagagttactatacgaccagttaataggatggcttggagcgggagcaatcgaagttaacgggccggccgaagagggaacagggcgtcctggaccggtacgattaccaaatacgttgaaccttcgtttcgtaggggcggaggcggatgctgttcaggcgtgcttacctgatgtcgcagttagtgccggaagtgcctgccatggtggcggggatgaaccaagcccagtccttttggcgatgggtagaagcgccaccgcagcaagagagagccttagattctctgtcggccgttttacaacagtagatcaaatccacgctgctgccgcaacggtcgcacgtgcagtacttcgtgtacagagtcttcgtgagatcggttctggtgaactaccggcgcctgcagatccggggggttcagctggccctggtcgccttggatctacagcagctccagttcctggtcgagccccggagttgcctgtgccgcgatctgcattgccatgaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 110 - DNA, 2324 bp, synthetic construct
  misc_feature 1..2324: note = OTHER INFORMATION: czcB::PtrnQ-5' UTR-nifUS(Fc)
  source 1..2324: mol_type = other DNA
cattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatgacggcgacggctacagccatagaactaactgaccgcgcccgcgctcaagtccgtcgactattaggttctactccgggtacggcaggttttgcactacgcattggcgttgatccgggaggttgttctggttatacgtacaatttatcactagtcccagatgctgatgccgacgacattgtctatcctcaggacggattcgatgtgttagtccatacatctatggtcgacttactacgtggaatgagagtagactatgctgagactttgactagttctggttttaccttcacgaaccctaatgcgaaaagctcatgcggctgtggatctagtttcggcgcctctgactctcctcaacgtacagaagcagatgcaaaattacgcgctcaagttgaggatgtcttgggcgagatccgcccgttcttgagaggcgatgggggagatgctgaaatagtggctgtcctagccggaggtggtgaaccagggtcagccgaggtccacctacgtttgaccggcgcatgtggaggttgtagcagcgcgaatgccactttgacgggcgttatcgagactagacttaaagaggcgctaccagaaataggacgtgttgcattggtttcttgagctgacgttctcataacgcagtctatataaggaggtgggaaaggtaatatacttggacaacaatgcaacaaccccagtcgacccggaggtcttaagtgcaatgttgccattacttcgtgaacattacgcaaaccctgctagccgacacccagcaggccaagccgtttcacgtcttgtcgaggcggcaagaagagatgtcgcagcgctagccggcgctgcaggtagagatgtcgttttcacgagcggagctaccgaggcggcgaacctagctacgcacggtgttctagccactgccgcaccaactagacgacgcattttagtaggcgcaaccgaacacccagcagtccttgctgcagcccacgctgccgccgacagacacgagggaagtgtgcagacagtacgtgtccaccctggtgggacggttgatttaagccacttgagaagcttgcttcgacctacaccagctgggggtgaggttgcacttgtagctgtcatggcagcgaatcacgaaacaggcgcacttacggatccacgtgctgtcgcagccttcgcacacggcgccggcgctttatttctttgtgacgtcacgcaagcattgggtcgcataccagttgctgtaaccgagacagggattgatctagcaatagcgagtgcgcataaagtttatggtccaaaaggggcaggcgttctattagcaggtcgagctgcgagagctcgtttggtccctttgttacacggcggtgggcaggaacgcggtcttagagccgggactctaaatacgccagctattgtagggttcggagctgcagcacgtatcgcagcagcagtacttcctgacgaagcaatccgccaacgtcagttaacagagttactatacgaccagttaataggatggcttggagcgggagcaatcgaagttaacgggccggccgaagagggaacagggcgtcctggaccggtacgattaccaaatacgttgaaccttcgtttcgtaggggcggaggcggatgctgttcaggcgtgcttacctgatgtcgcagttagtgccggaagtgcctgccatggtggcggggatgaaccaagcccagtccttttggcgatgggtagaagcgccaccgcagcaagagagagccttagattctctgtcggccgttttacaacagtagatcaaatccacgctgctgccgcaacggtcgcacgtgcagtacttcgtgtacagagtcttcgtgagatcggttctggtgaactaccggcgcctgcagatccggggggttcagctggccctggtcgccttggatctacagcagctccagttcctggtcgagccccggagttgcctgtgccgcgatctgcattgccatgaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 111 - DNA, 2157 bp, synthetic construct
  misc_feature 1..2157: note = OTHER INFORMATION: czcB::PR'-5' UTR-nifUS(Bd)
  source 1..2157: mol_type = other DNA
agacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaaggttataaacttaacagatagcgccgtaaacgccatcaaaagtgcaataagtagtagcgaacgacgagcgggtgggctacgtgtgatgatagaggcgggcggttgtaatgggtttaagtataagatggggatcgctgacgaaccaaagccagatgatacagtcattgactgtgatggattaaaggtattcgttgattctaagagcagagaacatttggccgggacgaccatagactttgttttggcacctgagtcatcaggcttcacgtttcacaacccgaacgctgcgaccaactgtagctgcggaaaatcatttggctgatggaaacattgatcaggcgataaggaaagggcaatgaaccacaccggccgaaccagaccgactgaagaactcgtcgatccccacgatgcgagagggtcccgttcgctgcgcgaacgaagagatcggacccaatcgccaagccgaagctggcgcctgttagctgatcggcacgatagcagcatgctcttggcgcggctcgacaatgctggcaagccgtacaagcttccgagcgtgagtctagaagggattcaggggcacctggtaggtaagatcggaggtttggatcgcctggtcctcgttgttgctgcaggcggggtggtgcagtatcagaaaaccgagctccaatttatttggacaataacgccacgacacgaacggacccatctgtagtccaggcaatgttgccattcttcactgagcagttcggtaatgccagcagcgcgcacgctttcgggaatgaagcggcgatcgctgtgagacaagcaagacgttcattaagtggtttgctaggaagtgcctatgatcacgagatattattcacgtctggcggaacggaggccaacaatgctgccatattatctgctcttgcaacacaggagggtagagatgaggtcgtaacaacctctgtcgagcacagcgccgtcttagcgcttaccgaacaattggccgcgcgtggtgttaagacacgcatcatcgctgttgatgcttgtgggcgtttagacatagaggcgtttagatgcgcattagggccacgcacggcaatcgcctcagtgatgtgggcgaataacgaaacgggcacgatcttcccggtaaagtgtctagcgggttggacgagagaggcgggagcgttatttcacacagacgctgttcaggcgattggcaaagtccgtcttaatcttaaggatagcacgatagacatgttgagcttatctgctcataaactacacggcccgaaaggggtgggggcattatatcttagaaagggtactaaatttcagccgttgatttgtggtggatcacaggaacgagggagacgagcgggaactgagaatatccctggcatagtagggttgggaaaggcagcagaattagctgcggaacgtcttgagccagaacgaatacgtattggagcgctacgagacagactagagcagggcattcttcgtaacggtgagtgtgtggtccttggagacatacgaaatagattggccaataccaccaatatcgccttcgatcacttggagggagaagcgatcgcacaccgattaaacagagccggaattgccgtgagcttgggctcagcttgtcgaagcggaagtatgcagccgtcacacgtcttgcgagccatgcaggtgccggcatggcgaatgcacggtgcggtccgcttcagcctatcacgagaaacgtctcttgcggaggtagacgaagccgtctgtgcggtatcagatattgtggcacgaatgagagcatctagtagagcgactgtccgtgagcaaacatcatgaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 112 - DNA, 1905 bp, synthetic construct
  misc_feature 1..1905: note = OTHER INFORMATION: czcB::PR'-5' UTR-nifUS(Rc)
  source 1..1905: mol_type = other DNA
agacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatgcgtgacatgcaggatgacgacacaaagtcaccggccccgcctcctgcggcagccgcagccgcacgtagagccgctgggcaggctgctcctgatgcgtctgccctaagagaccgatttgcaaaacttgcccaagccgacactccggaagccgccacagatgcggctgctgcggcggcagatgatgaagtaacccgcattcgcgctttaatagacgagatgcgcccgacctttcgtcgcgatggtggcgatattgaacttgtgcgagtggagggcgctaaagtcatcgtgcatctatcaggcgcttgtgcgggttgtatgcttgctgggcaaaccttgtacggggtgcaaaaacgcataacggacattcttggtcgtccgtttcgcgtgattccagacatccggcactgaccatgacccagccagcgatctatttggataacaacgctactacaagagttttccctgaggttgtcgcagcgatgttgccttatttcacggaacattttgggaatgcgtcttcagggcatggttttggggctcaagcaggcttgggattgcgtaaagcacgtttggcagtagcagccctaatcggtgcggcaagcgagcaggaaattctttttacaagcggcggaacagaggctaacacgaccgcaattcgctcagccttggctgttcaggatggcagacgcgaaattatcatctcagcggtggagcatccagcaatattgatgctagcagcggacctagaaaaaaccgaagggctacgtgtccaccgcatcccggtggaccatttgggacgcttagacctagccgcttatagagcggcactttctgatcgtgtagcagtcgccagtattatgtgggcgaataacgaaaccgggactatatttccagtaccacagcttgctgccgaggcccacgccgcaggtgcgctatttcacacagacgcggtgcaagcggttggtaagttgcctatgtcagtacaggagacagagatagacatgctatcattatcagggcataaatttcatggcccgaaaggagtcggagctttgtatttgagaaaaggggttgcgttccgaccgctattgagaggggggaaacaagaacgtggtcgtagagcaggaacagaaaatgtaccggcacttgtgggtatgggcacagcagcagaaattgcagcggcacgtatgacggccgacttaagtcgaatcgccactcttcgagattggctagaatctggcatattagcgttaggaggctgtcagcgcttaggagatcctgcgaatcgtttagccaatacttgtacattggccttcgacagaatagactctgaagcggtactaactaaattggccagagcaggcattgccatatctaccggcagcgcgtgtgcttctggagcgatggagccaagtcatgttacgcgtgccatggcggtgccgtttaccgctgcgcatggtgtagtccgctttagcttgagcgctgacacgaccgctgctgagcttagtcgcgtgctagcaatattaccagaaattttagcggagttgcgcccagtgccggtgaccgcgtgaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 113 - DNA, 1000 bp, synthetic construct
  misc_feature 1..1000: note = OTHER INFORMATION: Homology SUF(Pb51) UP
  source 1..1000: mol_type = other DNA
gtataacgaaaatcggtgaagttttgtacattttttttcttttgtttcatcgcgatataggaagggtaatgaataaacagatgtccggcaggtgatcatggcatatgatcgttacgatgcccgctggtgtctctccgtcagcatggcggtggataaagatcgtttatgaatgtcaatttaataaaaaaggaagggatttatgatggctaaaacaacaagtaactcgaaaaatacgcaaaccaattcggtggaggaacttttaaaccgtcaagtcgctaacctgaatgtgttatatgtgaaaattcataactatcactggtatgtgaaaggctccagtttttacactcttcatgttaagtttgaagagctttacaatgaaattacattgaaaatggacgagatcgccgagcgtctgctcgcattgaagggttctccttccgcaacgttaaaggaatatctggagctgtcctcgattcacgaggcaaccggcagtgaagatgctccgaaaatggtacaaacgctgatcgaggattttgcaactgtatgcgaagagtttcaggaaggtattgagcttgcagaaagcacctccgatcaacctaccgcagatttgctgatcggctataaagcggatctggaaaaacatatgtggatgctgcgttcctatctggggtaaatcagaaggaacttcagcaaaatgtcaattcggacagtttaatagcaccccatacagatgggctgctttgtaataaaccctttacatttaaaaacgctactgacgtcatgctgtcagtagcgtttttatggctaaaaaagaaatcctacaccttcaagttgagcaaaaatataagtgtataatgaatctggaggatgtatttggctatttgtgtgcaggatagaatgaacattttatgaagaaaggcgaataacgtgcgtctgttggttgcacccttaaagctatttattataaaatagcttgagaatcattgagaatcagtttaga

SEQ ID NO: 114 - DNA, 1000 bp, synthetic construct
  misc_feature 1..1000: note = OTHER INFORMATION: Homology SUF(Pb51) DN
  source 1..1000: mol_type = other DNA
atgattataaataaatttttaactattgttctgcttgaaatcagggggctcccctgtttagatatactttctgatttcgacaccacactacaaattttggagggtattccgaatatggctacaaactttaagattgaaggtctcaaagcgaccattgaaggcaaggaaattttgaaaggtattaacctcgaaatgaaaggtggagaaatccacgcgatcatgggtcctaacggtacaggtaaaagtacattggcttctgctttgatgggtcatcctaaatatgaagtcacagagggcaacgtaactttggatggagaagacgtgctggacatggaagtggatgagcgcgcacgcgcgggattgttcctggcgatgcagtatccgagcgaaattgcaggggtaacgaattccgactttttacgcagtgccattaatgcacgccgtgaagaaggacaagaaatctctctgatcaagtttatccgtcaaatggaaagcaaaatgaaggagcttgaaatgaatcctgagtttgctcaccgttatctgaacgaaggcttttccggtggtgagaaaaaacggaatgaaattctgcaaatgatgatgattgatccgaaaattgtcattcttgacgaaatcgactccggtcttgatattgatgctttgagaatcgtggctaacggtgtgaacgctatgcgttctgaggaccgtggttttctcgtgattacccactaccaacgtctgttgaactatattaagcctgattatgtgcacgttatgatgcaaggacgtatcgttaaatccggtggtcctgagctggctgaacgtctggaagcagatggctacgattggatcaaggaagagctgggcatcgttgacgaaactgtaggacaagaggcgtaagccggaagcgaggaggaaaactgatgagtacacaaacaatccttccggtagattccgagcaactgcgtgttctgtctgaacgtaatggcgagccgtcttggct

SEQ ID NO: 115 - protein, 316 aa, synthetic construct
  REGION 1..316: note = OTHER INFORMATION: nifU(Ps)
MWDYSEKVKEHFYNPKNAGAVAEANAVGDVGSLSCGDALRLSLKVDPDTDVILDAGFQTFGCGSAIASSSALTEMIKGLTVDEALKISNQDIADFLDGLPPEKMHCSVMGREALQAAVANYRGETLEDDHEEGALVCKCFAIDEVMVRETIRANRLSSVEDVTNYTKAGGGCSSCHEGIERLLVEELAARGEIFVPAGTGAKAAKKAKAPLVTLETPPAAPQAAPTAPRMTTLQRIRRIERVLESIRPTLQRDHGDVELLDVEGKNIYVKLTGACTGCQMASMTLSGIQQRLIEELGEFVKVVPVSSPAHSAMAEV

SEQ ID NO: 116 - DNA, 951 bp, synthetic construct
  misc_feature 1..951: note = OTHER INFORMATION: nifU(Ps) (reverse translation)
  source 1..951: mol_type = other DNA
atgtgggattatagcgaaaaggtcaaggaacatttctataatccgaaaaacgctggagcagtggcggaggcgaacgcagtgggcgatgtggggtcattgtcatgtggcgatgcgctaagactatctttaaaggttgaccctgataccgacgtaatacttgatgcaggttttcagacatttggctgtggctctgcaatagccagcagctctgccctaacggagatgatcaaagggcttacagtagatgaggcacttaaaataagtaaccaggacatcgcggatttcctagatgggcttccaccggaaaagatgcactgtagtgtgatggggcgcgaagcattacaagcagcagttgccaattacagaggggagactcttgaagacgatcacgaagagggcgcgttagtctgcaagtgttttgcgattgatgaagtcatggtaagagaaacgatccgtgctaatcgcttgtcaagtgtagaggatgtgactaactacacgaaggcgggaggtggctgctctagttgccatgaagggattgaaagactattggtggaagagcttgcagccagaggtgaaatcttcgtaccggcgggaacaggcgccaaagcagccaagaaggctaaggcaccattagttacgttagaaacacctccggccgccccgcaagccgcgcctactgccccaagaatgaccacgttgcagagaataagacgcattgaacgcgtcctagagtcaatacgaccaactctacagcgcgatcatggcgacgtggagctattagatgtggaaggcaagaacatatacgtcaaactaacgggggcgtgtacaggatgtcaaatggcctcaatgacgttaagcggaatccaacagagattgattgaggaattaggggaattcgttaaagtcgtcccagtatcatctccagctcatagcgcgatggcggaggtgtga

SEQ ID NO: 117 - protein, 279 aa, synthetic construct
  REGION 1..279: note = OTHER INFORMATION: nifU(Kv)
MWNYSEKVKDHFFHPRNARVVDNANAVGDVGSLSCGDALRLMLRVDPQTEIIEEAGFQTFGCGSAIASSSALTELIIGHTLTEAGQITNQQIADYLDGLPPEKMHCSVMGQEALRAAIAHFRGESLEEEHEEGKLICKCFGVDEGHIRRAVVNNGLTTLEEVINYTKAGGGCTACHEKIELALAAILAQQPPAPLPVETAHDAHWQSVVDTINELRPHIQADGGDMTLLNVTPRQVTVSLSGSCSGCMMTDMTLAWLQQKLMERTGTYMDVVAAPAAVN

SEQ ID NO: 118 - DNA, 840 bp, synthetic construct
  misc_feature 1..840: note = OTHER INFORMATION: nifU(Kv) (reverse translation)
  source 1..840: mol_type = other DNA
atgtggaattattcagagaaggtcaaagatcactttttccaccctcgaaacgcgagagtcgttgacaatgccaatgcagtaggggacgtagggtctctttcatgcggcgacgctcttcgcctaatgttgcgagtcgatccgcaaaccgagataatagaagaggcaggttttcagacttttggttgtggaagcgcaatagccagttcttcagcccttacggaactaattattggtcacacattgacagaggctggacaaataactaaccaacaaattgcagattatctagatggccttcctccagaaaagatgcactgttcagtcatgggtcaagaggcgttgagagcagctatagcccattttagaggcgagtcactagaggaagaacatgaggagggaaagttgatctgtaaatgtttcggcgtcgacgagggacatatccgccgcgctgtggtaaacaatggattaacgacacttgaggaggttataaactatactaaggcgggaggcggttgcactgcttgccatgagaagatagaacttgctcttgccgcaatcttggcccagcaacctccggctccattacctgtagagacggcacatgacgcccattggcagagtgtcgtggacactatcaatgaacttagacctcacatccaggctgatgggggcgatatgacccttctaaatgtcactccgagacaggtcacggtatctttgtctggctcttgcagcgggtgtatgatgaccgatatgacgcttgcttggctacaacagaaactaatggaaagaacaggaacttatatggacgtagtcgcggccccggcggccgtaaactaa

SEQ ID NO: 119 - protein, 213 aa, synthetic construct
  REGION 1..213: note = OTHER INFORMATION: nifU(Rt)
MRKYRGKLKTSFCDHKGARILQTAKSENKVASGDAVELMMGFDRPTEAISAATLQSLRTRFAIACSSEFIEFIVGKSAYEAIRNINCDLTYVVDRPPSTSPSPFASCVAQSRAIRSHASQLSKKRLLRSLRRCSLKASLGQRRVCRQAQRKAAYSRSKKYLCNRRGFSAACRRGSRMLGARLNKVHRSLKLPDNVPAANSTNRTLRRLFLPEQ

SEQ ID NO: 120 - DNA, 642 bp, synthetic construct
  misc_feature 1..642: note = OTHER INFORMATION: nifU(Rt) (reverse translation)
  source 1..642: mol_type = other DNA
atgcgtaagtacagaggtaagcttaagacaagtttttgcgatcataagggtgctcgcatacttcaaactgcgaagagcgaaaataaggtcgctagtggtgacgcagtggaattgatgatgggtttcgaccgcccaacagaggctataagcgcagccacacttcagagtttgagaactcgatttgcgatagcctgttcttctgagttcatcgaattcattgtgggtaaatcagcgtatgaggctattcgaaacatcaattgtgaccttacctatgtagtagatcgtccgcctagcacctcacctagtccatttgccagttgcgtggctcaatctcgtgctattcgaagccacgcctcacagctatcaaagaagagattattgcgttcacttagacgatgcagcttgaaagcctcattagggcagcgacgagtttgccgtcaggctcagcgtaaggctgcgtatagtcgatctaaaaagtacttatgtaatcgacgaggcttttcagcagcttgtcgacgcggttctcgtatgcttggtgcacgtttgaacaaagtgcatcgtagtcttaaacttccagacaatgtgccagcagccaacagcactaaccgaactttaagaagattatttttgccggagcagtga

SEQ ID NO: 121 - protein, 313 aa, synthetic construct
  REGION 1..313: note = OTHER INFORMATION: nifU(Av)
MWDYSEKVKEHFYNPKNAGAVEGANAIGDVGSLSCGDALRLTLKVDPETDVILDAGFQTFGCGSAIASSSALTEMVKGLTLDEALKISNQDIADYLDGLPPEKMHCSVMGREALQAAVANYRGETIEDDHEEGALICKCFAVDEVMVRDTIRANKLSTVEDVTNYTKAGGGCSACHEAIERVLTEELAARGEVFVAAPIKAKKKVKVLAPEPAPAPVAEAPAAAPKLSNLQRIRRIETVLAAIRPTLQRDKGDVELIDVDGKNVYVKLTGACTGCQMASMTLGGIQQRLIEELGEFVKVIPVSAAAHHAQMEV

SEQ ID NO: 122 - DNA, 942 bp, synthetic construct
  misc_feature 1..942: note = OTHER INFORMATION: nifU(Av) (reverse translation)
  source 1..942: mol_type = other DNA
atgtgggattattctgagaaagttaaagaacatttttacaatcctaagaacgcgggagcagtggagggagccaacgccattggggacgtcggctctctttcatgtggggatgcactacgactaacacttaaagttgatcctgaaacagatgtcatattagacgcgggtttccaaacgtttggctgtggctcagcgatagcttcatcatcagctcttactgagatggtgaaggggttgaccctagacgaggcattaaagataagtaaccaggatatagccgactatttggatggattgccacctgagaagatgcattgttctgtgatgggacgcgaggccttgcaagcggcggttgctaactacagaggggagaccatcgaagacgatcacgaagagggagccctaatttgtaagtgtttcgccgttgacgaagtcatggtccgtgatacgatacgtgccaacaagttgagtaccgtcgaggacgtaacaaattacacgaaggccgggggaggttgctctgcgtgtcacgaggcaatagagagagttttgacagaagaattggcagcacgtggcgaagtctttgtcgcggctccaatcaaggcgaagaaaaaagtaaaggtccttgcccctgagcctgctcctgcaccagttgctgaagccccagcagcggcgccaaagttaagcaaccttcagcgtataagacgcatcgagactgtattagcagcaatcagacctacgcttcagcgtgataaaggggatgtggagcttattgacgttgatgggaaaaacgtttacgtgaaactaacgggcgcctgcaccgggtgccaaatggcatcaatgactttgggcggtattcagcagagacttatcgaagaattaggggaatttgtcaaagtcatccctgtttctgcagcagcacatcacgcgcagatggaggtctga

SEQ ID NO: 123 - protein, 206 aa, synthetic construct
  REGION 1..206: note = OTHER INFORMATION: nifU(Fc)
MTATATAIELTDRARAQVRRLLGSTPGTAGFALRIGVDPGGCSGYTYNLSLVPDADADDIVYPQDGFDVLVHTSMVDLLRGMRVDYAETLTSSGFTFTNPNAKSSCGCGSSFGASDSPQRTEADAKLRAQVEDVLGEIRPFLRGDGGDAEIVAVLAGGGEPGSAEVHLRLTGACGGCSSANATLTGVIETRLKEALPEIGRVALVS

SEQ ID NO: 124 - DNA, 621 bp, synthetic construct
  misc_feature 1..621: note = OTHER INFORMATION: nifU(Fc) (reverse translation)
  source 1..621: mol_type = other DNA
atgacggcgacggctacagccatagaactaactgaccgcgcccgcgctcaagtccgtcgactattaggttctactccgggtacggcaggttttgcactacgcattggcgttgatccgggaggttgttctggttatacgtacaatttatcactagtcccagatgctgatgccgacgacattgtctatcctcaggacggattcgatgtgttagtccatacatctatggtcgacttactacgtggaatgagagtagactatgctgagactttgactagttctggttttaccttcacgaaccctaatgcgaaaagctcatgcggctgtggatctagtttcggcgcctctgactctcctcaacgtacagaagcagatgcaaaattacgcgctcaagttgaggatgtcttgggcgagatccgcccgttcttgagaggcgatgggggagatgctgaaatagtggctgtcctagccggaggtggtgaaccagggtcagccgaggtccacctacgtttgaccggcgcatgtggaggttgtagcagcgcgaatgccactttgacgggcgttatcgagactagacttaaagaggcgctaccagaaataggacgtgttgcattggtttcttga

SEQ ID NO: 125 - protein, 106 aa, synthetic construct
  REGION 1..106: note = OTHER INFORMATION: nifU(Bd)
VINLTDSAVNAIKSAISSSERRAGGLRVMIEAGGCNGFKYKMGIADEPKPDDTVIDCDGLKVFVDSKSREHLAGTTIDFVLAPESSGFTFHNPNAATNCSCGKSFG

SEQ ID NO: 126 - DNA, 321 bp, synthetic construct
  misc_feature 1..321: note = OTHER INFORMATION: nifU(Bd) (reverse translation)
  source 1..321: mol_type = other DNA
gttataaacttaacagatagcgccgtaaacgccatcaaaagtgcaataagtagtagcgaacgacgagcgggtgggctacgtgtgatgatagaggcgggcggttgtaatgggtttaagtataagatggggatcgctgacgaaccaaagccagatgatacagtcattgactgtgatggattaaaggtattcgttgattctaagagcagagaacatttggccgggacgaccatagactttgttttggcacctgagtcatcaggcttcacgtttcacaacccgaacgctgcgaccaactgtagctgcggaaaatcatttggctga

SEQ ID NO: 127 - protein, 136 aa, synthetic construct
  REGION 1..136: note = OTHER INFORMATION: nifU(Rc)
MRDMQDDDTKSPAPPPAAAAAARRAAGQAAPDASALRDRFAKLAQADTPEAATDAAAAAADDEVTRIRALIDEMRPTFRRDGGDIELVRVEGAKVIVHLSGACAGCMLAGQTLYGVQKRITDILGRPFRVIPDIRH

SEQ ID NO: 128 - DNA, 411 bp, synthetic construct
  misc_feature 1..411: note = OTHER INFORMATION: nifU(Rc) (reverse translation)
  source 1..411: mol_type = other DNA
atgcgtgacatgcaggatgacgacacaaagtcaccggccccgcctcctgcggcagccgcagccgcacgtagagccgctgggcaggctgctcctgatgcgtctgccctaagagaccgatttgcaaaacttgcccaagccgacactccggaagccgccacagatgcggctgctgcggcggcagatgatgaagtaacccgcattcgcgctttaatagacgagatgcgcccgacctttcgtcgcgatggtggcgatattgaacttgtgcgagtggagggcgctaaagtcatcgtgcatctatcaggcgcttgtgcgggttgtatgcttgctgggcaaaccttgtacggggtgcaaaaacgcataacggacattcttggtcgtccgtttcgcgtgattccagacatccggcactga

SEQ ID NO: 129 - protein, 116 aa, synthetic construct
  REGION 1..116: note = OTHER INFORMATION: nifU(Tv)
MAYTITSQCISCKLCSSVCPTGAIKVAEDGQHWIDQALCTNCVDSVHTVPQCKAGCPTCDGCVKVPSDYWEGWFANYNRVIAKLTKKQDYWERWFNCYSQKFSEQIQKHQGEILGV

SEQ ID NO: 130 - DNA, 351 bp, synthetic construct
  misc_feature 1..351: note = OTHER INFORMATION: nifU(Tv) (reverse translation)
  source 1..351: mol_type = other DNA
atggcatatacgatcacttcacagtgcatttcatgtaagttatgcagtagtgtctgcccgacgggagccattaaagtagccgaagacggtcagcattggattgaccaagcgttgtgtaccaattgtgttgatagtgttcataccgtcccgcaatgcaaagcagggtgtccaacatgcgatggctgcgtcaaagttccgagtgattattgggaagggtggttcgcaaattacaacagagtaattgccaagctaacaaagaagcaagattactgggaacgctggtttaattgttatagtcagaagttcagtgaacagatccaaaaacatcagggtgaaatcttaggggtataa

SEQ ID NO: 131 - protein, 402 aa, synthetic construct
  REGION 1..402: note = OTHER INFORMATION: nifS(Ps)
MSGIYLDNNATTRVDDEVVQAMLPFFTEQFGNPSSMHSFGNQVGMALKKARQSVQRLLGAEYDSEIVFTSCGTEADSTAILSALKAQPERKTIITTVVEHPAILSLCDYLAEDGYTVHKLKVDKKGRLDLDEYAALLDDDVAIVSVMWANNETGTLFPVEQMAQMADDAGVMFHSDAVQAVGKVPMNLKGSAIHMLSLSGHKLHAPKGVGVLYLRRGTRFRPLLRGGHQERGRRAGTENAASIIGLGVAAERALAFMEHENTEVRRLRDKLEAGILAAVPYAFVTGDPGNRLPNTANIAFEYIEGEAILLLLNKVGIAASSGSACTSGSLEPSHVMRAMDIPYTAAHGSVRFSLSRYTTEEQIDYVIREVPPIIAQLRKLSPYWSGNGPAEAVGDSFEPVYA

SEQ ID NO: 132 - DNA, 1209 bp, synthetic construct
  misc_feature 1..1209: note = OTHER INFORMATION: nifS(Ps) (reverse translation)
  source 1..1209: mol_type = other DNA
atgtcaggaatataccttgataacaacgcaaccacacgcgttgatgacgaggtggttcaggcaatgctaccgttcttcaccgagcaattcggtaacccttcaagtatgcacagtttcggcaatcaagtagggatggcgcttaaaaaagcaagacaatcagtgcaacgattgttaggagccgaatatgatagcgaaattgtcttcacctcttgcggaaccgaggccgacagcaccgctattttgtcagcattgaaggcacaaccggaacgcaaaactatcataacgactgtagtagagcacccagccatccttagtttatgtgactacttagccgaggatggatataccgtccacaaattgaaagtagataaaaagggacgattagatttagacgagtacgcagcattgctagatgacgatgtagcgatagtcagcgtgatgtgggcaaataacgaaaccggtactctttttccagtggagcaaatggcacagatggctgacgacgcgggggtcatgttccacagtgacgcagtgcaagctgtgggaaaggtcccaatgaaccttaaggggtctgcaatccatatgctatctcttagtgggcacaagttgcacgcaccgaaaggggtcggggtactatatttgcgtagaggcacgcgttttcgtccattgctacgaggcggtcaccaggagagagggcgcagagctggcacggaaaatgcggcaagcatcattggcttaggggtcgctgcggaacgagcacttgcgtttatggagcacgagaacactgaagtacgacgtttacgtgataaactagaggccggtatattagccgcggtaccatacgcattcgtaacgggcgacccaggcaaccgattgcctaacacagccaacatcgcattcgaatacatcgagggggaggccattcttttgcttttgaacaaagtaggaattgccgccagcagtggcagtgcatgcacgtcaggttctttagaaccatctcacgttatgcgcgccatggacataccatataccgcagctcacgggagtgtccgcttcagtttgagcagatatacaactgaggaacaaatagattatgtaattagagaggttccacctataattgctcaacttcgaaaactaagtccatactggtcaggtaacggcccggctgaagcggttggcgattcattcgaaccagtttacgcatga

SEQ ID NO: 133 - protein, 400 aa, synthetic construct
  REGION 1..400: note = OTHER INFORMATION: nifS(Kv)
MKPVYLDNNATTRLDPMVLDAMMPFLTDFYGNPSSIHDFGLPAQAALERAHQQVAALLGADYPSEIIFTSCATEATTTAIASAVALMPERREIITSAVEHPATLAVCEQLERQGYLIHRIAVDQQGALDMAQFRAALSPRVALVSIMWANNETGVMFPVPELAELAHEQGALFHCDAVQVVGKIPMALSQTRIDMLSCSAHKFHGPKGVGCLWLRRGTRFRPLLRGGHQAHGRRAGTENISGIVGMGAASELAQVHLPGMAQISQLRDRLEQHLQATIPAVTVMGGSQPRVPGTLNLAFECIEGEAILLLMNQAGIAASSGSACTSGSLEPSHVMRAMNIPYTAAHGTIRFSLSRYTREKEIDYVIATLPSVIDRLRALSPYWRQGQPHLQDGAFTPVYG

SEQ ID NO: 134 - DNA, 1203 bp, synthetic construct
  misc_feature 1..1203: note = OTHER INFORMATION: nifS(Kv) (reverse translation)
  source 1..1203: mol_type = other DNA
atgaagccggtttacttggataataacgcaactacacgtcttgacccaatggtgttggatgcaatgatgccgtttttgacggatttctacggtaatcctagctcaattcacgatttcgggctacctgcgcaggccgcacttgagcgagcacatcagcaggtcgctgccttattaggtgctgattatccatctgagattatatttaccagttgcgcaactgaggctacaacaaccgccatagctagtgcggtagcactaatgccagagagacgcgagataataactagcgcggtggagcacccagctaccttagccgtatgcgagcagttagagagacaaggttacttgatccaccgaatagccgttgaccagcagggagcactagatatggcacagttccgtgcagctctatcacctagagtcgcgctagtgagtatcatgtgggctaataatgaaacaggagtcatgtttcctgttccggagttagcggaattggctcatgaacagggcgcgttatttcattgcgatgcagtccaggtagtcgggaagattcctatggcgctatctcaaacccgaattgatatgctaagctgctctgcgcataagtttcacggtccgaagggggtaggctgcctttggcttcgccgcggtacgcgttttcgaccgttattgcgagggggacaccaggctcatgggcgcagagccggaacggaaaacatttcaggcatagttgggatgggagccgcgagtgaattggcacaggtccatttgccggggatggcccaaattagccaattgcgagatagattggaacaacacctacaagccaccataccggcagtcacagtaatgggcgggtcacaaccacgagttccaggtacgcttaatttggcgtttgaatgcattgagggagaagctattttgcttttaatgaaccaagcaggcatagctgcttcaagtggatcagcgtgcacaagcgggtctttggagccttcacacgttatgcgagcgatgaacatcccgtacaccgcagctcacggaaccatacgattttcacttagtcgttacacgcgcgagaaagaaatagattacgtcattgcaaccctaccgagcgtaatcgaccgccttagagcattgagtccgtactggcgccaggggcagccgcatctacaagacggagcatttaccccggtttatggctaa

SEQ ID NO: 135 - protein, 413 aa, synthetic construct
  REGION 1..413: note = OTHER INFORMATION: nifS(Rt)
MRIASMRSVYLDNNATTRVDPEVVQAMLPFFTDQFANPSSSHSFGASASAAIMTARKRLQALIGAEFEHEIVFTSGGTESDNAAILSALEIMPDRTQIVTSAVEHPAVLTLCAHLERTRGIKVHRIPVDRHGRLDLRTYRAALTPHVALVSIMWANNETGAIFPVADLAKLAKEVGAFFHTDAVQAVGKLPMDLKSSAIDMLSLSGHKLHGPKGIGALWVKRGVRFDPMIKGGHQERDRRAGTENTAGIVGLGKAAELASSFMKEENGRVRSLRDRLERGLLQRIPDAFVTGDKQKRLPNTSNIAFDQVDGDGLLLLLDRYGIACSSGSACTSQSPQPSHVLTAMNIPDISSHGVIRFSLSRDNCEEDIDRVLDVLPGIVGSLRDGSSFKPTPAKRRRPLAFMKPTETDGNTS

SEQ ID NO: 136 - DNA, 1242 bp, synthetic construct
  misc_feature 1..1242: note = OTHER INFORMATION: nifS(Rt) (reverse translation)
  source 1..1242: mol_type = other DNA
ctgaggatcgcttccatgcgctctgtatacttggacaacaacgctacaacacgcgtcgaccctgaggtagtgcaggccatgctaccgttttttacggaccagtttgcaaatccgagcagctcacattcatttggagctagcgcttcagcggccataatgaccgcccgaaagagattgcaagcattaatcggagcagaatttgaacatgagatagttttcaccagtgggggtactgaatcagacaacgcagctattttgagtgccctagagataatgcctgacagaacgcagattgtaacctctgccgtggagcacccagccgtccttaccctttgtgcccatttggagcgaacgcgtggcataaaagttcaccgcattcctgttgatcgtcacggaagattggatttacgcacctatcgagcggcgttgacccctcacgtcgctctagtgagcatcatgtgggctaacaatgagactggtgcaatctttcctgtcgctgacttagccaagctagcgaaggaggtaggagcctttttccatacagatgcagtccaagcagtaggaaagttgccaatggatctaaaatcttctgcaatagatatgctatctcttagtggacacaagcttcacgggcctaagggtattggtgctctttgggtgaagcgaggtgttcgtttcgatccgatgataaagggtggtcaccaagaacgcgatagacgtgcgggcactgagaacacggcaggaatagtggggttgggcaaagctgcagaactagcttctagcttcatgaaagaagagaacggtcgcgtccgaagcttacgcgacagactagagcgcggcttattacaacgcattcctgacgccttcgtcacaggagacaaacaaaagcgacttccaaacacttctaatatagccttcgaccaggtggacggcgacgggttgctattgttgttagatcgctacgggatagcttgcagtagcgggagtgcctgcacgtctcagagccctcaacctagccatgtgttaacggccatgaatattccagacatctcttcacatggcgtaataagattttctcttagccgagataactgcgaagaggatatagaccgagtgttagacgttttgccagggatagttggtagtcttcgtgacggctcatcattcaaaccaaccccggcaaaacgccgtcgacctttagcctttatgaagccgactgaaacagatggtaatacttcatga

SEQ ID NO: 137 - protein, 402 aa, synthetic construct
  REGION 1..402: note = OTHER INFORMATION: nifS(Av)
MADVYLDNNATTRVDDEIVQAMLPFFTEQFGNPSSLHSFGNQVGMALKKARQSVQKLLGAEHDSEIVFTSCGTESDSTAILSALKAQPERKTVITTVVEHPAVLSLCDYLASEGYTVHKLPVDKKGRLDLEHYASLLTDDVAVVSVMWANNETGTLFPIEEMARLADDAGIMFHTDAVQAVGKVPIDLKNSSIHMLSLSGHKLHAPKGVGVLYLRRGTRFRPLLRGGHQERGRRAGTENAASIIGLGVAAERALQFMEHENTEVKRLRDKLEAGILAVVPHAFVTGDPDNRLPNTANIAFEYIEGEAILLLLNKVGIAASSGSACTSGSLEPSHVMRAMDIPYTAAHGTVRFSLSRYTTEEEIDRVIREVPPIVAQLRKLSPYWSGNGPVEDPGKAFAPVYG

SEQ ID NO: 138 - DNA, 1209 bp, synthetic construct
  misc_feature 1..1209: note = OTHER INFORMATION: nifS(Av) (reverse translation)
  source 1..1209: mol_type = other DNA
atggcagatgtctatttagacaacaacgctacaacgcgcgttgatgacgaaatcgttcaagcgatgcttccgttctttacagaacaattcgggaatccatctagccttcacagcttcggtaatcaggttgggatggcattgaaaaaagctcgtcaatcagtgcaaaaactattgggagccgagcacgattctgaaatagtcttcactagctgcggtactgagagcgactctactgctattctaagcgccttaaaggcgcagccggagagaaagacagtgataactactgtggttgagcacccagccgtgctttcattgtgtgactacctagcatcagaagggtacacagtacataaattgcctgtcgataaaaaagggcgattggatcttgagcactacgcgagcttacttacggacgatgtagcagttgtcagcgttatgtgggctaataacgaaaccgggactttgttccctatagaggagatggcaagactagcggatgacgctgggataatgttccatacggacgcggttcaagctgtaggaaaggtcccgatagacctaaagaatagttctatccatatgctttctttatctggtcacaagttgcatgcgccgaagggcgtgggggtattatacctacgacgcggaacgagatttcgtccactactaagaggcggacatcaagagcgaggcagacgtgctggaacggagaatgccgcttctattattggacttggagtggcggcagaacgagcgctacaattcatggagcacgagaacacagaggtgaaaagattgcgcgacaaacttgaggcaggcattctagctgtggtgccgcacgcattcgtgacgggagacccagacaaccgtttgccgaatacagccaatatcgctttcgagtatatagaaggggaggctattctacttcttcttaataaggttggtattgccgctagctcaggatctgcttgtactagtggatcattagagccaagtcacgttatgcgagcgatggacataccgtacacggctgcccacggaaccgtgcgattttcattatcacgatatactacagaagaagaaatcgaccgagtcatccgtgaggtcccgccgattgtggctcagcttcgaaaattatctccgtattggagtggaaatggcccagtcgaagacccaggtaaagcctttgctccggtatatggttga

SEQ ID NO: 139 - protein, 439 aa, synthetic construct
  REGION 1..439: note = OTHER INFORMATION: nifS(Fc)
VIYLDNNATTPVDPEVLSAMLPLLREHYANPASRHPAGQAVSRLVEAARRDVAALAGAAGRDVVFTSGATEAANLATHGVLATAAPTRRRILVGATEHPAVLAAAHAAADRHEGSVQTVRVHPGGTVDLSHLRSLLRPTPAGGEVALVAVMAANHETGALTDPRAVAAFAHGAGALFLCDVTQALGRIPVAVTETGIDLAIASAHKVYGPKGAGVLLAGRAARARLVPLLHGGGQERGLRAGTLNTPAIVGFGAAARIAAAVLPDEAIRQRQLTELLYDQLIGWLGAGAIEVNGPAEEGTGRPGPVRLPNTLNLRFVGAEADAVQACLPDVAVSAGSACHGGGDEPSPVLLAMGRSATAARESLRFSVGRFTTVDQIHAAAATVARAVLRVQSLREIGSGELPAPADPGGSAGPGRLGSTAAPVPGRAPELPVPRSALP

SEQ ID NO: 140 - DNA, 1320 bp, synthetic construct
  misc_feature 1..1320: note = OTHER INFORMATION: nifS(Fc) (reverse translation)
  source 1..1320: mol_type = other DNA
gtaatatacttggacaacaatgcaacaaccccagtcgacccggaggtcttaagtgcaatgttgccattacttcgtgaacattacgcaaaccctgctagccgacacccagcaggccaagccgtttcacgtcttgtcgaggcggcaagaagagatgtcgcagcgctagccggcgctgcaggtagagatgtcgttttcacgagcggagctaccgaggcggcgaacctagctacgcacggtgttctagccactgccgcaccaactagacgacgcattttagtaggcgcaaccgaacacccagcagtccttgctgcagcccacgctgccgccgacagacacgagggaagtgtgcagacagtacgtgtccaccctggtgggacggttgatttaagccacttgagaagcttgcttcgacctacaccagctgggggtgaggttgcacttgtagctgtcatggcagcgaatcacgaaacaggcgcacttacggatccacgtgctgtcgcagccttcgcacacggcgccggcgctttatttctttgtgacgtcacgcaagcattgggtcgcataccagttgctgtaaccgagacagggattgatctagcaatagcgagtgcgcataaagtttatggtccaaaaggggcaggcgttctattagcaggtcgagctgcgagagctcgtttggtccctttgttacacggcggtgggcaggaacgcggtcttagagccgggactctaaatacgccagctattgtagggttcggagctgcagcacgtatcgcagcagcagtacttcctgacgaagcaatccgccaacgtcagttaacagagttactatacgaccagttaataggatggcttggagcgggagcaatcgaagttaacgggccggccgaagagggaacagggcgtcctggaccggtacgattaccaaatacgttgaaccttcgtttcgtaggggcggaggcggatgctgttcaggcgtgcttacctgatgtcgcagttagtgccggaagtgcctgccatggtggcggggatgaaccaagcccagtccttttggcgatgggtagaagcgccaccgcagcaagagagagccttagattctctgtcggccgttttacaacagtagatcaaatccacgctgctgccgcaacggtcgcacgtgcagtacttcgtgtacagagtcttcgtgagatcggttctggtgaactaccggcgcctgcagatccggggggttcagctggccctggtcgccttggatctacagcagctccagttcctggtcgagccccggagttgcctgtgccgcgatctgcattgccatga

SEQ ID NO: 141 - protein, 384 aa, synthetic construct
  REGION 1..384: note = OTHER INFORMATION: nifS(Bd)
MDNNATTRTDPSVVQAMLPFFTEQFGNASSAHAFGNEAAIAVRQARRSLSGLLGSAYDHEILFTSGGTEANNAAILSALATQEGRDEVVTTSVEHSAVLALTEQLAARGVKTRIIAVDACGRLDIEAFRCALGPRTAIASVMWANNETGTIFPVKCLAGWTREAGALFHTDAVQAIGKVRLNLKDSTIDMLSLSAHKLHGPKGVGALYLRKGTKFQPLICGGSQERGRRAGTENIPGIVGLGKAAELAAERLEPERIRIGALRDRLEQGILRNGECVVLGDIRNRLANTTNIAFDHLEGEAIAHRLNRAGIAVSLGSACRSGSMQPSHVLRAMQVPAWRMHGAVRFSLSRETSLAEVDEAVCAVSDIVARMRASSRATVREQTS

SEQ ID NO: 142 - DNA, 1155 bp, synthetic construct
  misc_feature 1..1155: note = OTHER INFORMATION: nifS(Bd) (reverse translation)
  source 1..1155: mol_type = other DNA
ttggacaataacgccacgacacgaacggacccatctgtagtccaggcaatgttgccattcttcactgagcagttcggtaatgccagcagcgcgcacgctttcgggaatgaagcggcgatcgctgtgagacaagcaagacgttcattaagtggtttgctaggaagtgcctatgatcacgagatattattcacgtctggcggaacggaggccaacaatgctgccatattatctgctcttgcaacacaggagggtagagatgaggtcgtaacaacctctgtcgagcacagcgccgtcttagcgcttaccgaacaattggccgcgcgtggtgttaagacacgcatcatcgctgttgatgcttgtgggcgtttagacatagaggcgtttagatgcgcattagggccacgcacggcaatcgcctcagtgatgtgggcgaataacgaaacgggcacgatcttcccggtaaagtgtctagcgggttggacgagagaggcgggagcgttatttcacacagacgctgttcaggcgattggcaaagtccgtcttaatcttaaggatagcacgatagacatgttgagcttatctgctcataaactacacggcccgaaaggggtgggggcattatatcttagaaagggtactaaatttcagccgttgatttgtggtggatcacaggaacgagggagacgagcgggaactgagaatatccctggcatagtagggttgggaaaggcagcagaattagctgcggaacgtcttgagccagaacgaatacgtattggagcgctacgagacagactagagcagggcattcttcgtaacggtgagtgtgtggtccttggagacatacgaaatagattggccaataccaccaatatcgccttcgatcacttggagggagaagcgatcgcacaccgattaaacagagccggaattgccgtgagcttgggctcagcttgtcgaagcggaagtatgcagccgtcacacgtcttgcgagccatgcaggtgccggcatggcgaatgcacggtgcggtccgcttcagcctatcacgagaaacgtctcttgcggaggtagacgaagccgtctgtgcggtatcagatattgtggcacgaatgagagcatctagtagagcgactgtccgtgagcaaacatcatga

SEQ ID NO: 143 - protein, 386 aa, synthetic construct
  REGION 1..386: note = OTHER INFORMATION: nifS(Rc)
MTQPAIYLDNNATTRVFPEVVAAMLPYFTEHFGNASSGHGFGAQAGLGLRKARLAVAALIGAASEQEILFTSGGTEANTTAIRSALAVQDGRREIIISAVEHPAILMLAADLEKTEGLRVHRIPVDHLGRLDLAAYRAALSDRVAVASIMWANNETGTIFPVPQLAAEAHAAGALFHTDAVQAVGKLPMSVQETEIDMLSLSGHKFHGPKGVGALYLRKGVAFRPLLRGGKQERGRRAGTENVPALVGMGTAAEIAAARMTADLSRIATLRDWLESGILALGGCQRLGDPANRLANTCTLAFDRIDSEAVLTKLARAGIAISTGSACASGAMEPSHVTRAMAVPFTAAHGVVRFSLSADTTAAELSRVLAILPEILAELRPVPVTA

SEQ ID NO: 144 - DNA, 1161 bp, synthetic construct
  misc_feature 1..1161: note = OTHER INFORMATION: nifS(Rc) (reverse translation)
  source 1..1161: mol_type = other DNA
atgacccagccagcgatctatttggataacaacgctactacaagagttttccctgaggttgtcgcagcgatgttgccttatttcacggaacattttgggaatgcgtcttcagggcatggttttggggctcaagcaggcttgggattgcgtaaagcacgtttggcagtagcagccctaatcggtgcggcaagcgagcaggaaattctttttacaagcggcggaacagaggctaacacgaccgcaattcgctcagccttggctgttcaggatggcagacgcgaaattatcatctcagcggtggagcatccagcaatattgatgctagcagcggacctagaaaaaaccgaagggctacgtgtccaccgcatcccggtggaccatttgggacgcttagacctagccgcttatagagcggcactttctgatcgtgtagcagtcgccagtattatgtgggcgaataacgaaaccgggactatatttccagtaccacagcttgctgccgaggcccacgccgcaggtgcgctatttcacacagacgcggtgcaagcggttggtaagttgcctatgtcagtacaggagacagagatagacatgctatcattatcagggcataaatttcatggcccgaaaggagtcggagctttgtatttgagaaaaggggttgcgttccgaccgctattgagaggggggaaacaagaacgtggtcgtagagcaggaacagaaaatgtaccggcacttgtgggtatgggcacagcagcagaaattgcagcggcacgtatgacggccgacttaagtcgaatcgccactcttcgagattggctagaatctggcatattagcgttaggaggctgtcagcgcttaggagatcctgcgaatcgtttagccaatacttgtacattggccttcgacagaatagactctgaagcggtactaactaaattggccagagcaggcattgccatatctaccggcagcgcgtgtgcttctggagcgatggagccaagtcatgttacgcgtgccatggcggtgccgtttaccgctgcgcatggtgtagtccgctttagcttgagcgctgacacgaccgctgctgagcttagtcgcgtgctagcaatattaccagaaattttagcggagttgcgcccagtgccggtgaccgcgtga

SEQ ID NO: 145 - protein, 400 aa, synthetic construct
  REGION 1..400: note = OTHER INFORMATION: nifS(Tv)
MSVIYLDNNATTKVDPEVVEAIMPYLTDYYGNPSSMHTFGGQLGKAVRTAREQVAALLGADESEIVFTSCGTEGDNAAIRAALLAQPEKRHIITTQVEHPAVLNVCKQLETQGYTVTYLSVNGHGQLDLDELEASLTGNTALVTIMYANNETGTVFPIEEIGKRVKERGAIFHVDAVQAVGKIPLNMKTSTIDMLTISGHKIHAPKGIGALYVRRGVRFRPLLIGGHQERGRRAGTENVPGIVGLGKAAELELIHIETAIKKETRLRDRLEQTLLAKIPDCEVNGDVTQRLPNTTNIGFKYIEGEAILLSLNKYGICASSGSACTSGSLEPSHVLRAMGLPYTTLHGSIRFSLCRYTTEAQIDRVIEVMPEIVERLRALSPFKNDEAGWLQAQEQTLAHR

SEQ ID NO: 146 - DNA, 1203 bp, synthetic construct
  misc_feature 1..1203: note = OTHER INFORMATION: nifS(Tv) (reverse translation)
  source 1..1203: mol_type = other DNA
atgtctgtgatataccttgacaataacgcaaccacgaaagtagatccagaggtcgtggaggctatcatgccatacttgacggattactacgggaacccttcaagtatgcatacgtttgggggacaattaggaaaggcggttagaacagcgagagagcaagttgccgccctattaggggctgacgagtcagagatcgtatttaccagttgcggtacagagggggacaatgcagccatcagagctgctttactagcacagccggagaagcgacacattatcacaacccaagtggaacacccggcagttctaaatgtatgtaaacaactagagactcaaggttacaccgtcacgtatttgtcagtaaacggtcacgggcaactagacttagatgagttagaagccagcctaacggggaacacagcgttggtcaccataatgtacgccaataacgaaacaggcacggtgttccctatagaggagatcggcaagcgcgtaaaggagcgaggcgctatctttcacgttgacgcagtgcaagccgtagggaagatccctctaaatatgaagacgtctactatcgatatgctaacaatatctgggcataagatacatgctcctaagggtattggcgctttatacgtcagacgcggcgttcgattccgacctctattaatcggtggtcatcaggaacgaggccgacgagcgggaaccgagaatgtacctgggattgttggactaggaaaagccgccgaactagaacttattcatattgagactgccattaagaaggagaccagacttcgcgatagattagagcaaactttgctagccaaaattccggattgcgaagtcaatggggacgttactcaacgtttaccgaataccactaatatcggcttcaaatatatagagggggaagcgatcctactttcattgaacaagtatggaatctgcgccagcagtggatctgcttgtaccagcggcagtctagagccaagccatgttcttcgcgcaatgggcttaccttacacgacacttcacggatctatacgttttagtttgtgtagatacaccacggaagcacagatagaccgagtcatagaggtgatgcctgaaattgtagagcgattgcgcgccttgtcaccgttcaagaacgatgaggctgggtggcttcaagctcaggaacaaacattggcgcatcgctaa

SEQ ID NO: 147 - DNA, 96 bp, synthetic construct; identical to SEQ ID NO: 79
  misc_feature 1..96: note = OTHER INFORMATION: PfldA(Ppe)::TR2-5' UTR
  source 1..96: mol_type = other DNA

SEQ ID NO: 148 - DNA, 856 bp, synthetic construct
  misc_feature 1..856: note = OTHER INFORMATION: czcB::PtrnQ-5' UTR-nifF(Ss)
  source 1..856: mol_type = other DNA
cattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatgaagttcaccatagtgtttgcctctgctacagggcacaccgaagacattgcggaacgcctagatcagttgttaccagactcagaattaaaagagctttctgacttacaggatattaaggatatagagggatgcgaagcattaatttgctgcactccaacctggaatacagggagcgatgtcaagagatctggcacggcatgggatgagcacatagagcagataccgaccttgaactgttctggtaaaccagtagccatagtggggcttggagacagtgccgcctttagtaagttcttttgcgacgccatggaagaactatacacagcatttcaaaaagcgggtggtaagagcataggccacgtcagcggagaggattatatattcgacgattcaaagagcatgattaacggcatgttttgcgggttgccaattgacgaggacaatgaaagtgagaagacccaggaccgcctagagagctggtgtaaaactattttagaagaatctaatcagtaaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 149 - DNA, 883 bp, synthetic construct
  misc_feature 1..883: note = OTHER INFORMATION: czcB::PtrnQ-5' UTR-nifF(Ps)
  source 1..883: mol_type = other DNA
cattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatggcaagaattggcctattcttcggatctaacacaggtaagacccgtaaggttgcaaagatgatcaagaagagattcgatgacgacacaatggcggacgcactaaatgtcaatcgagcaagtgctgaggatgtggcacagtatcaatatcttataatcggcacccctactctaggcgacggggaactaccgggactatctgctgactgcgaaaatgagagctgggaggagttcttacctaagctagaaggcgtggatttttcaggtaagactgtcgccctatttggccttggggatcaggtagggtatccaaaggaatttttgaacgcaatgggaatattgtatgatttcgtagttgagcgaggggcgacagtagttggcgcctggcctacggaaggttatgagttcgaggagtctgaggccgtgcgagatggccaatttgttggcttagccttagaccttgacaatcaatcaggtttaacggaagagagattaaatgtctggttgaaaactatagctccagcttttgaacttccactataaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 150 - DNA, 784 bp, synthetic construct
  misc_feature 1..784: note = OTHER INFORMATION: czcB::PtrnQ-5' UTR-fldA(Bs)
  source 1..784: mol_type = other DNA
agacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatggctaaattacttgttgcgtacgctagtatgacaggaaataccgaagagattgccgagttgatcgtcgagggtattaaacaaagtgggcatgaggcggatttaaaatcagtgacggattgtaacgcggcagatgtgttggactacgatggattcctaataggtgtatatacttggggtgatggtgagttacctgacgaattcttagacttctatgaggagcttgatgagcttgatttgacgggcaagagagccgcggtgttcgggtctggggacacttcttatgagcagtattgtggcgcagtagatttagcagccgagaaacttcaagaacgaggagcagaggtgtacccagaaatgctaaaaatcgaatacagcccattggagcaagaaaaggatacctgtagagattttggaaaaaagtttatcgcggctggattgcaagttagttaaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 151 - DNA, 871 bp, synthetic construct
  misc_feature 1..871: note = OTHER INFORMATION: czcB::PtrnQ-5' UTR-nifF(Km)
  source 1..871: mol_type = other DNA
cattttccccaaaaatacaggggttcaaaccatcgtatgtcagattgccaattaagatgctttgtctatttaaaaaacggcctctcgaaatagagggttgttatttgaaaggaattatcgtataattagttgtgctgacgttctcataacgcagtctatataaggaggtgggaaagatggcgaatatcgggatatttttcggcactgataccgggaaaacacgcaaaatcgctaagatgatccacaaacaactaggcgaactagccgatgctcctgtcaatataaaccgaacgacactagacgattttatggcttatcctgtgttgttattaggaactccaactctaggagatgggcaactaccggggcttgaggcaggatgcgaaggtgagtcttggagtgaatttataagtggattggatgacgcaagtcttagcggcaaaactgtagcgttgttcgggttaggagaccagaagggctatccagataatttcgtatctggaatgcgacctctttatgatgccctaagagctcgtggagcccagattattggctcttggccaaatgaaggctatgagttttctgccagtgctgctcttgagggtgagcgtttcattggtttagtccttgaccaagacaaccagtttgatcaaactgaagcgcgattggcaagctggttggaggaaataaaacgtgtagtgctttaaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 152 - DNA, 880 bp, synthetic construct
  misc_feature 1..880: note = OTHER INFORMATION: czcB::PR'-5' UTR-nifF(Rc)
  source 1..880: mol_type = other DNA
agacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatggcgaaaattggattgttctttgggtctgacactggaactacacgtaaaattgctaaacagataaaggatatgtttgatgacgaagtgatggccaagccactaaacgtcaatcgagccgatgttgcggatttcatggcttatgactttttgatacttggaactccaaccttaggtgatggtcagctaccaggattgtctgcaaacgcagcatctgagtcttgggaagagtttcttccgcgcattgccgaccaggatttctcagggaaaaccatagccttgttcggtttgggtgatcaagtcacttatcctttagaatttgttaatgcgttgttcttcttgcatgaattttttagtgatcgtggcgcgaaattggttggtcgctggccagcgaaggggtatggctttgaggacagccttgccgtagttgaaggggaatttcttggtcttgctcttgaccaagataatcaagctgcgttgacaccagagcgattaaagggctggctatctttgatcgctgctgatttcggtcttgtgttgcctgcttaaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 153 - DNA, 784 bp, synthetic construct; identical to SEQ ID NO: 150
  misc_feature 1..784: note = OTHER INFORMATION: czcB::PR'-5' UTR-fldA(Pt)
  source 1..784: mol_type = other DNA

SEQ ID NO: 154 - DNA, 790 bp, synthetic construct
  misc_feature 1..790: note = OTHER INFORMATION: czcB::PR'-5' UTR-fldA(Cf)
  source 1..790: mol_type = other DNA
agacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatgtctacgatcttgatagtttatgcatctttgaccgggaatacggaggatatagctggtcttatcgcagaaggtattcgcacggaaggatctgaagctaagctaatacttgctgatgagtgccgcgcggaagatttgtacctatatgacgcattccttctaggggcgtacacgtggggcgatggagagttaccggatgagtttttggactttacagatgagttggaggagcttgcgttggaaggacgtcgcgcggcagtgtttggatctggggatatgagctaccgaatttattgtggggccgtggatgtgttagaagagaagttacgtcagcgaggcgctacaatagtacaggacagtctaaaaatagagtacggaccgtcacgtgaggaacaagaacagtgccgcgagtatggcagaaagttcgttcgccatgcacgacctagtagtcagtcataaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 155 - DNA, 784 bp, synthetic construct
  misc_feature 1..784: note = OTHER INFORMATION: czcB::PR'-5' UTR-fldA(Bm)
  source 1..784: mol_type = other DNA
agacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatggctaatatagtattagtgtatgcgagcatgacggggaatactgaggaaatagcaaatgctgtcgccgaaggaattcaggaagcaggcgagtcagttgtcgtaaagcaagtgttggacgctacggccgccgagcttgcagaatatgaggctatcttgttgggggcttatacatggggcgatggtgaactaccggatgaatttttagacttttacgatgagctagaccaactagatctaaccggcaagaaggcagcggtcttcgggagctgtgatagctcttatgagcattacggggctgctgtagacatcttaacggctaagttgcgtgaaagagggtgcgagatagtgtctgaaggtttgaaaattgaacttgctccgtctgatgaagaacgtgaattatgccgagcgtttggccgtcaatttgtaagcgccttacaacctgcaagataaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 156 - DNA, 874 bp, synthetic construct
  misc_feature 1..874: note = OTHER INFORMATION: czcB::PR'-5' UTR-nifF(Ac)
  source 1..874: mol_type = other DNA
agacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatggccaagataggactattctttggaagtaataccggaaagactagaaaggtagccaaatctattaaaaagcgatttgacgatgaaacaatgtcagacgcagttaatgtcaacagagtatctgctgaggactttgcccaataccagtttttgatcttgggcactcctactttaggagagggggaattgcctggactatctagcgattgtgagaatgagagttgggaggaattccttccgaaaattgagggacttgatttctctgggaaaactgtggcactttttgggcttggcgaccaagtcggttatcctgagaatttcctagatgcgatgggcgagcttcacagcttcttcaccgaaagaggggccaaagtcgtcggggcatggagtactgacggttatgaattcgagggctcaacagcggtcgtcgatggcaagtttgtagggcttgctttggaccttgacaaccagtctggtaagacggatgaacgagtcgctgcatggttagcccaaatagcaccggagttcgggcttagtttataaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 157 - DNA, 847 bp, synthetic construct
  misc_feature 1..847: note = OTHER INFORMATION: czcB::PR'-5' UTR-nifF(Ss)
  source 1..847: mol_type = other DNA
agacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatgaagttcaccatagtgtttgcctctgctacagggcacaccgaagacattgcggaacgcctagatcagttgttaccagactcagaattaaaagagctttctgacttacaggatattaaggatatagagggatgcgaagcattaatttgctgcactccaacctggaatacagggagcgatgtcaagagatctggcacggcatgggatgagcacatagagcagataccgaccttgaactgttctggtaaaccagtagccatagtggggcttggagacagtgccgcctttagtaagttcttttgcgacgccatggaagaactatacacagcatttcaaaaagcgggtggtaagagcataggccacgtcagcggagaggattatatattcgacgattcaaagagcatgattaacggcatgttttgcgggttgccaattgacgaggacaatgaaagtgagaagacccaggaccgcctagagagctggtgtaaaactattttagaagaatctaatcagtaaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 158 - DNA, 874 bp, synthetic construct
  misc_feature 1..874: note = OTHER INFORMATION: czcB::PR'-5' UTR-nifF(Ps)
  source 1..874: mol_type = other DNA
agacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatggcaagaattggcctattcttcggatctaacacaggtaagacccgtaaggttgcaaagatgatcaagaagagattcgatgacgacacaatggcggacgcactaaatgtcaatcgagcaagtgctgaggatgtggcacagtatcaatatcttataatcggcacccctactctaggcgacggggaactaccgggactatctgctgactgcgaaaatgagagctgggaggagttcttacctaagctagaaggcgtggatttttcaggtaagactgtcgccctatttggccttggggatcaggtagggtatccaaaggaatttttgaacgcaatgggaatattgtatgatttcgtagttgagcgaggggcgacagtagttggcgcctggcctacggaaggttatgagttcgaggagtctgaggccgtgcgagatggccaatttgttggcttagccttagaccttgacaatcaatcaggtttaacggaagagagattaaatgtctggttgaaaactatagctccagcttttgaacttccactataaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 159 - DNA, 784 bp, synthetic construct
  misc_feature 1..784: note = OTHER INFORMATION: czcB::PR'-5' UTR-fldA(Bs)
  source 1..784: mol_type = other DNA
agacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatggctaaggtccttgttgcctacgcttctttgacggggaatacggaggaaattgcggaactaattgtcgaagggatcaatcaggcaggtggggaagcggtgcttaaatcagtaacggattgtaacgctgacgagataaaagaatatgaaggggttttactaggcgcatatacgtggggggacggagaactacctgatgagtttttggatttttacgaggaaatggatgagttaaacttatcaggattcaaagcagcggctttcgggtctggcgatacaggctatgaaatatattgcggagccgtcgatactattgaacaaaagttgaaggaacttggtgcggaaatagtacaagagagtcttaaaattgagtacggaccgtcagccacagaaaaggatgcctgtcgacagtttgggcgtcaatttattgaagcgtgtgcggcggtttcataaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 160 - DNA, 868 bp, synthetic construct
  misc_feature 1..868: note = OTHER INFORMATION: czcB::PR'-5' UTR-nifF(Mc)
  source 1..868: mol_type = other DNA
agacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatgggtaagattggcatattttttgggtctgatacagggaatacaagacgcgtcgccaagcaattagcaaaaaaacttggtgaggatgccgatgctccggttgacgttaaaaaagctgatattgacgatgttcttaaatacgacgctcttatacttggtactccaactttaggtgacggagagttgccggggttagatagtggcgcaagtgaagagagctggttggagtttctaccaaaatttaagggcaaagatatgagtggaaagaccgtagcgcttttcggcttgggcgatcaacaaggctatggacatgagtttgtggacgccttgcgagaaatatacgaccaggtagtcgagtgtggcgcacgcgtggttggcgcatggccgacagatacctatgaattcgaaaaatctaaagccgtcgtggatgggaaattcgtcggcttagtgatcgaccatgaaaaccaatcagagatgacggacgagcgtttggacgagtggcttgaaatagtgcgcccagctcttgtcggtgcctaaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 161 - DNA, 778 bp, synthetic construct
  misc_feature 1..778: note = OTHER INFORMATION: czcB::PR'-5' UTR-fldA(Ta)
  source 1..778: mol_type = other DNA
agacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatggcgaaggcaattatggtatttactagcatgactggaaacacggaagaaatggctgaggctattgcggagggagtacgccaacaaggcatcgaactagaggtaaaggaggcacttgacgcaaccgccgcagagttggaacaatatgatggaatcttactaggtgcgtacacatggggagatggggagttaccagacgactttttggatttctatgatgaattagatgaagttgatttgaccggcaaaaaagcagcggtatttggttcatgcgactcttcatacgagaaatacggggccgccgtagacatattgattgagaagttgcaagaacgaggagcggaagtggtcttggaggggttgaaggtcgaacttaccccgaccgacgaagataaagaattatgcgagaaatttggtagagattttgcaaaacaacttcagctttaaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 162 - DNA, 862 bp, synthetic construct
  misc_feature 1..862: note = OTHER INFORMATION: czcB::PR'-5' UTR-nifF(Km)
  source 1..862: mol_type = other DNA
agacaacattttgaatgcggtcacacgttagcagcatgattgccacggatggcaacatattaacggcatgacattgacttattgaataaaattgggtaaatttgactcaacgatgggttaattcgctgacgttctcataacgcagtctatataaggaggtgggaaagatggcgaatatcgggatatttttcggcactgataccgggaaaacacgcaaaatcgctaagatgatccacaaacaactaggcgaactagccgatgctcctgtcaatataaaccgaacgacactagacgattttatggcttatcctgtgttgttattaggaactccaactctaggagatgggcaactaccggggcttgaggcaggatgcgaaggtgagtcttggagtgaatttataagtggattggatgacgcaagtcttagcggcaaaactgtagcgttgttcgggttaggagaccagaagggctatccagataatttcgtatctggaatgcgacctctttatgatgccctaagagctcgtggagcccagattattggctcttggccaaatgaaggctatgagttttctgccagtgctgctcttgagggtgagcgtttcattggtttagtccttgaccaagacaaccagtttgatcaaactgaagcgcgattggcaagctggttggaggaaataaaacgtgtagtgctttaaggttaattcttttgtttcacgaatttattaattggcttatgaaaagacatcccggttcacagatgtgtaacgttccttcctagtggctgtattacgtgcttattaggtagtattttgtaagagttcataaggggcgcccaaatgggcgtcccttttcttgtcat

SEQ ID NO: 163 - protein, 171 aa, synthetic construct
  REGION 1..171: note = OTHER INFORMATION: nifF(Ss)
MKFTIVFASATGHTEDIAERLDQLLPDSELKELSDLQDIKDIEGCEALICCTPTWNTGSDVKRSGTAWDEHIEQIPTLNCSGKPVAIVGLGDSAAFSKFFCDAMEELYTAFQKAGGKSIGHVSGEDYIFDDSKSMINGMFCGLPIDEDNESEKTQDRLESWCKTILEESNQ

SEQ ID NO: 164 - DNA, 516 bp, synthetic construct
  misc_feature 1..516: note = OTHER INFORMATION: nifF(Ss)
  source 1..516: mol_type = other DNA
atgaagttcaccatagtgtttgcctctgctacagggcacaccgaagacattgcggaacgcctagatcagttgttaccagactcagaattaaaagagctttctgacttacaggatattaaggatatagagggatgcgaagcattaatttgctgcactccaacctggaatacagggagcgatgtcaagagatctggcacggcatgggatgagcacatagagcagataccgaccttgaactgttctggtaaaccagtagccatagtggggcttggagacagtgccgcctttagtaagttcttttgcgacgccatggaagaactatacacagcatttcaaaaagcgggtggtaagagcataggccacgtcagcggagaggattatatattcgacgattcaaagagcatgattaacggcatgttttgcgggttgccaattgacgaggacaatgaaagtgagaagacccaggaccgcctagagagctggtgtaaaactattttagaagaatctaatcagtaa

SEQ ID NO: 165 - protein, 180 aa, synthetic construct
  REGION 1..180: note = OTHER INFORMATION: nifF(Ps)
MARIGLFFGSNTGKTRKVAKMIKKRFDDDTMADALNVNRASAEDVAQYQYLIIGTPTLGDGELPGLSADCENESWEEFLPKLEGVDFSGKTVALFGLGDQVGYPKEFLNAMGILYDFVVERGATVVGAWPTEGYEFEESEAVRDGQFVGLALDLDNQSGLTEERLNVWLKTIAPAFELPL

SEQ ID NO: 166 - DNA, 543 bp, synthetic construct
  misc_feature 1..543: note = OTHER INFORMATION: nifF(Ps)
  source 1..543: mol_type = other DNA
atggcaagaattggcctattcttcggatctaacacaggtaagacccgtaaggttgcaaagatgatcaagaagagattcgatgacgacacaatggcggacgcactaaatgtcaatcgagcaagtgctgaggatgtggcacagtatcaatatcttataatcggcacccctactctaggcgacggggaactaccgggactatctgctgactgcgaaaatgagagctgggaggagttcttacctaagctagaaggcgtggatttttcaggtaagactgtcgccctatttggccttggggatcaggtagggtatccaaaggaatttttgaacgcaatgggaatattgtatgatttcgtagttgagcgaggggcgacagtagttggcgcctggcctacggaaggttatgagttcgaggagtctgaggccgtgcgagatggccaatttgttggcttagccttagaccttgacaatcaatcaggtttaacggaagagagattaaatgtctggttgaaaactatagctccagcttttgaacttccactataa

SEQ ID NO: 167 - protein, 150 aa, synthetic construct
  REGION 1..150: note = OTHER INFORMATION: fldA(Bs)
MAKVLVAYASLTGNTEEIAELIVEGINQAGGEAVLKSVTDCNADEIKEYEGVLLGAYTWGDGELPDEFLDFYEEMDELNLSGFKAAAFGSGDTGYEIYCGAVDTIEQKLKELGAEIVQESLKIEYGPSATEKDACRQFGRQFIEACAAVS

SEQ ID NO: 168 - DNA, 453 bp, synthetic construct
  misc_feature 1..453: note = OTHER INFORMATION: fldA(Bs)
  source 1..453: mol_type = other DNA
atggctaaggtccttgttgcctacgcttctttgacggggaatacggaggaaattgcggaactaattgtcgaagggatcaatcaggcaggtggggaagcggtgcttaaatcagtaacggattgtaacgctgacgagataaaagaatatgaaggggttttactaggcgcatatacgtggggggacggagaactacctgatgagtttttggatttttacgaggaaatggatgagttaaacttatcaggattcaaagcagcggctttcgggtctggcgatacaggctatgaaatatattgcggagccgtcgatactattgaacaaaagttgaaggaacttggtgcggaaatagtacaagagagtcttaaaattgagtacggaccgtcagccacagaaaaggatgcctgtcgacagtttgggcgtcaatttattgaagcgtgtgcggcggtttcataa

SEQ ID NO: 169 - protein, 176 aa, synthetic construct
  REGION 1..176: note = OTHER INFORMATION: nifF(Km)
MANIGIFFGTDTGKTRKIAKMIHKQLGELADAPVNINRTTLDDFMAYPVLLLGTPTLGDGQLPGLEAGCEGESWSEFISGLDDASLSGKTVALFGLGDQKGYPDNFVSGMRPLYDALRARGAQIIGSWPNEGYEFSASAALEGERFIGLVLDQDNQFDQTEARLASWLEEIKRVVL

SEQ ID NO: 170 - DNA, 531 bp, synthetic construct
  misc_feature 1..531: note = OTHER INFORMATION: nifF(Km)
  source 1..531: mol_type = other DNA
atggcgaatatcgggatatttttcggcactgataccgggaaaacacgcaaaatcgctaagatgatccacaaacaactaggcgaactagccgatgctcctgtcaatataaaccgaacgacactagacgattttatggcttatcctgtgttgttattaggaactccaactctaggagatgggcaactaccggggcttgaggcaggatgcgaaggtgagtcttggagtgaatttataagtggattggatgacgcaagtcttagcggcaaaactgtagcgttgttcgggttaggagaccagaagggctatccagataatttcgtatctggaatgcgacctctttatgatgccctaagagctcgtggagcccagattattggctcttggccaaatgaaggctatgagttttctgccagtgctgctcttgagggtgagcgtttcattggtttagtccttgaccaagacaaccagtttgatcaaactgaagcgcgattggcaagctggttggaggaaataaaacgtgtagtgctttaa

SEQ ID NO: 171 - protein, 182 aa, synthetic construct
  REGION 1..182: note = OTHER INFORMATION: nifF(Rc)
MAKIGLFFGSDTGTTRKIAKQIKDMFDDEVMAKPLNVNRADVADFMAYDFLILGTPTLGDGQLPGLSANAASESWEEFLPRIADQDFSGKTIALFGLGDQVTYPLEFVNALFFLHEFFSDRGAKLVGRWPAKGYGFEDSLAVVEGEFLGLALDQDNQAALTPERLKGWLSLIAADFGLVLPA

SEQ ID NO: 172 - DNA, 549 bp, synthetic construct
  misc_feature 1..549: note = OTHER INFORMATION: nifF(Rc)
  source 1..549: mol_type = other DNA
atggcgaaaattggattgttctttgggtctgacactggaactacacgtaaaattgctaaacagataaaggatatgtttgatgacgaagtgatggccaagccactaaacgtcaatcgagccgatgttgcggatttcatggcttatgactttttgatacttggaactccaaccttaggtgatggtcagctaccaggattgtctgcaaacgcagcatctgagtcttgggaagagtttcttccgcgcattgccgaccaggatttctcagggaaaaccatagccttgttcggtttgggtgatcaagtcacttatcctttagaatttgttaatgcgttgttcttcttgcatgaattttttagtgatcgtggcgcgaaattggttggtcgctggccagcgaaggggtatggctttgaggacagccttgccgtagttgaaggggaatttcttggtcttgctcttgaccaagataatcaagctgcgttgacaccagagcgattaaagggctggctatctttgatcgctgctgatttcggtcttgtgttgcctgcttaa

SEQ ID NO: 173 - protein, 150 aa, synthetic construct
  REGION 1..150: note = OTHER INFORMATION: fldA(Pt)
MAKLLVAYASMTGNTEEIAELIVEGIKQSGHEADLKSVTDCNAADVLDYDGFLIGVYTWGDGELPDEFLDFYEELDELDLTGKRAAVFGSGDTSYEQYCGAVDLAAEKLQERGAEVYPEMLKIEYSPLEQEKDTCRDFGKKFIAAGLQVS

SEQ ID NO: 174 - DNA, 453 bp, synthetic construct
  misc_feature 1..453: note = OTHER INFORMATION: fldA(Pt)
  source 1..453: mol_type = other DNA
atggctaaattacttgttgcgtacgctagtatgacaggaaataccgaagagattgccgagttgatcgtcgagggtattaaacaaagtgggcatgaggcggatttaaaatcagtgacggattgtaacgcggcagatgtgttggactacgatggattcctaataggtgtatatacttggggtgatggtgagttacctgacgaattcttagacttctatgaggagcttgatgagcttgatttgacgggcaagagagccgcggtgttcgggtctggggacacttcttatgagcagtattgtggcgcagtagatttagcagccgagaaacttcaagaacgaggagcagaggtgtacccagaaatgctaaaaatcgaatacagcccattggagcaagaaaaggatacctgtagagattttggaaaaaagtttatcgcggctggattgcaagttagttaa

SEQ ID NO: 175 - protein, 152 aa, synthetic construct
  REGION 1..152: note = OTHER INFORMATION: fldA(Cf)
MSTILIVYASLTGNTEDIAGLIAEGIRTEGSEAKLILADECRAEDLYLYDAFLLGAYTWGDGELPDEFLDFTDELEELALEGRRAAVFGSGDMSYRIYCGAVDVLEEKLRQRGATIVQDSLKIEYGPSREEQEQCREYGRKFVRHARPSSQS

SEQ ID NO: 176 - DNA, 459 bp, synthetic construct
  misc_feature 1..459: note = OTHER INFORMATION: fldA(Cf)
  source 1..459: mol_type = other DNA
atgtctacgatcttgatagtttatgcatctttgaccgggaatacggaggatatagctggtcttatcgcagaaggtattcgcacggaaggatctgaagctaagctaatacttgctgatgagtgccgcgcggaagatttgtacctatatgacgcattccttctaggggcgtacacgtggggcgatggagagttaccggatgagtttttggactttacagatgagttggaggagcttgcgttggaaggacgtcgcgcggcagtgtttggatctggggatatgagctaccgaatttattgtggggccgtggatgtgttagaagagaagttacgtcagcgaggcgctacaatagtacaggacagtctaaaaatagagtacggaccgtcacgtgaggaacaagaacagtgccgcgagtatggcagaaagttcgttcgccatgcacgacctagtagtcagtcataa

SEQ ID NO: 177 - protein, 150 aa, synthetic construct
  REGION 1..150: note = OTHER INFORMATION: fldA(Bm)
MANIVLVYASMTGNTEEIANAVAEGIQEAGESVVVKQVLDATAAELAEYEAILLGAYTWGDGELPDEFLDFYDELDQLDLTGKKAAVFGSCDSSYEHYGAAVDILTAKLRERGCEIVSEGLKIELAPSDEERELCRAFGRQFVSALQPAR

SEQ ID NO: 178 - DNA, 453 bp, synthetic construct
  misc_feature 1..453: note = OTHER INFORMATION: fldA(Bm)
  source 1..453: mol_type = other DNA
atggctaatatagtattagtgtatgcgagcatgacggggaatactgaggaaatagcaaatgctgtcgccgaaggaattcaggaagcaggcgagtcagttgtcgtaaagcaagtgttggacgctacggccgccgagcttgcagaatatgaggctatcttgttgggggcttatacatggggcgatggtgaactaccggatgaatttttagacttttacgatgagctagaccaactagatctaaccggcaagaaggcagcggtcttcgggagctgtgatagctcttatgagcattacggggctgctgtagacatcttaacggctaagttgcgtgaaagagggtgcgagatagtgtctgaaggtttgaaaattgaacttgctccgtctgatgaagaacgtgaattatgccgagcgtttggccgtcaatttgtaagcgccttacaacctgcaagataa

SEQ ID NO: 179 - protein, 180 aa, synthetic construct
  REGION 1..180: note = OTHER INFORMATION: nifF(Ac)
MAKIGLFFGSNTGKTRKVAKSIKKRFDDETMSDAVNVNRVSAEDFAQYQFLILGTPTLGEGELPGLSSDCENESWEEFLPKIEGLDFSGKTVALFGLGDQVGYPENFLDAMGELHSFFTERGAKVVGAWSTDGYEFEGSTAVVDGKFVGLALDLDNQSGKTDERVAAWLAQIAPEFGLSL

SEQ ID NO: 180 - DNA, 543 bp, synthetic construct
  misc_feature 1..543: note = OTHER INFORMATION: nifF(Ac)
  source 1..543: mol_type = other DNA
atggccaagataggactattctttggaagtaataccggaaagactagaaaggtagccaaatctattaaaaagcgatttgacgatgaaacaatgtcagacgcagttaatgtcaacagagtatctgctgaggactttgcccaataccagtttttgatcttgggcactcctactttaggagagggggaattgcctggactatctagcgattgtgagaatgagagttgggaggaattccttccgaaaattgagggacttgatttctctgggaaaactgtggcactttttgggcttggcgaccaagtcggttatcctgagaatttcctagatgcgatgggcgagcttcacagcttcttcaccgaaagaggggccaaagtcgtcggggcatggagtactgacggttatgaattcgagggctcaacagcggtcgtcgatggcaagtttgtagggcttgctttggaccttgacaaccagtctggtaagacggatgaacgagtcgctgcatggttagcccaaatagcaccggagttcgggcttagtttataa

SEQ ID NO: 181 - protein, 178 aa, synthetic construct
  REGION 1..178: note = OTHER INFORMATION: nifF(Mc)
MGKIGIFFGSDTGNTRRVAKQLAKKLGEDADAPVDVKKADIDDVLKYDALILGTPTLGDGELPGLDSGASEESWLEFLPKFKGKDMSGKTVALFGLGDQQGYGHEFVDALREIYDQVVECGARVVGAWPTDTYEFEKSKAVVDGKFVGLVIDHENQSEMTDERLDEWLEIVRPALVGA

SEQ ID NO: 182 - DNA, 537 bp, synthetic construct
  misc_feature 1..537: note = OTHER INFORMATION: nifF(Mc)
  source 1..537: mol_type = other DNA
atgggtaagattggcatattttttgggtctgatacagggaatacaagacgcgtcgccaagcaattagcaaaaaaacttggtgaggatgccgatgctccggttgacgttaaaaaagctgatattgacgatgttcttaaatacgacgctcttatacttggtactccaactttaggtgacggagagttgccggggttagatagtggcgcaagtgaagagagctggttggagtttctaccaaaatttaagggcaaagatatgagtggaaagaccgtagcgcttttcggcttgggcgatcaacaaggctatggacatgagtttgtggacgccttgcgagaaatatacgaccaggtagtcgagtgtggcgcacgcgtggttggcgcatggccgacagatacctatgaattcgaaaaatctaaagccgtcgtggatgggaaattcgtcggcttagtgatcgaccatgaaaaccaatcagagatgacggacgagcgtttggacgagtggcttgaaatagtgcgcccagctcttgtcggtgcctaa

SEQ ID NO: 183 - protein, 148 aa, synthetic construct
  REGION 1..148: note = OTHER INFORMATION: fldA(Ta)
MAKAIMVFTSMTGNTEEMAEAIAEGVRQQGIELEVKEALDATAAELEQYDGILLGAYTWGDGELPDDFLDFYDELDEVDLTGKKAAVFGSCDSSYEKYGAAVDILIEKLQERGAEVVLEGLKVELTPTDEDKELCEKFGRDFAKQLQL

SEQ ID NO: 184 - DNA, 447 bp, synthetic construct
  misc_feature 1..447: note = OTHER INFORMATION: fldA(Ta)
  source 1..447: mol_type = other DNA
atggcgaaggcaattatggtatttactagcatgactggaaacacggaagaaatggctgaggctattgcggagggagtacgccaacaaggcatcgaactagaggtaaaggaggcacttgacgcaaccgccgcagagttggaacaatatgatggaatcttactaggtgcgtacacatggggagatggggagttaccagacgactttttggatttctatgatgaattagatgaagttgatttgaccggcaaaaaagcagcggtatttggttcatgcgactcttcatacgagaaatacggggccgccgtagacatattgattgagaagttgcaagaacgaggagcggaagtggtcttggaggggttgaaggtcgaacttaccccgaccgacgaagataaagaattatgcgagaaatttggtagagattttgcaaaacaacttcagctttaa

SEQ ID NO: 185 - DNA, 1000 bp, synthetic construct
  misc_feature 1..1000: note = OTHER INFORMATION: Homology-fld (Pp34) UP
  source 1..1000: mol_type = other DNA
cgtaccggacggtcagccaggccggacaattgaacgttttcctttgcccactgcaccatacctttggcggcatccacatgaactacactggctccggcatacgctgcggcagtagtggctcctcctgtgtaagcaaacaggttcaatacggaaatgggtctacctgcgcctttgattttatccatcatccagctccagttggctgcctgctctggaaaaagaccagtatgtttgaaactggtcggtttaatgtggaatttaagcggtccatagctaatcgtccagcgctcagggataggctttttcatatcccattggccgcccccggaagagctgcgatgataatgcccatgaacattgcgccattcggctgtttcttttgccaacggccatatgatttgcggatcgggacgacgcaggacaatattgccccaacgttccagcttttcaccgccgcctgtatccattacttcgtagtctttccactcttgtgctacatacatgtacaatccatccttcagcagtcaattcaggatttaggcagcaagctgcgtaaaatccccacatggagtctattgtacaacaaaccttagcaacaatcgaatgattccatgaataagtaaggagtcagacagagcgaaaataatttttttcgatgtacttttaattagagcgctttcatattaaaatgaaatcaatcacacttgtacgtacaagtgtttaattttcgttttatttatgaaaaacggattaggttgtacgtacataatatttttacattgaagctttgcaggagcgaagagcttccctattggaaggctaccttaccatcttaaagactttaacagtgaacagccgcattgtaatgcggctgttttgcgtatatattttcaaaatgcacatcatgcttgaaatttttatttgacaatgagaatcattatcaaatattatttgagtaatagggagtgaaagcatcccatatttcaaataaaggtgaggg

SEQ ID NO: 186 - DNA, 1000 bp, synthetic construct
  misc_feature 1..1000: note = OTHER INFORMATION: Homology-fld (Pp34) DN
  source 1..1000: mol_type = other DNA
atgaacttgggtaaaatcatgattgcttatgctagcatgaccggtaatacagaggaaatcgcggaattgatcgccgagggcgttcgtcaggctggacatgaggcggagttgaaggcttcgtatgattgtagcgcgcaagaactgctcgcatatgacggattcctgttaggtgtatatacatggggagacggtgagcttcctgatgaatttttagatttttacgaggaactagatgagcttgatctgtctggtaaaaagactgctgtatttggcagtggagatacatcttacacacaattttgtggcgcagtggatctggtagaggaaaaagtcaaagaacgcggcgcatccgtcattcaggaaagtctcaagattgaattcaatccattggatgacgagaaggagaattgccgagcttacggaagacaattcgcacaggtcggtattggagtgtcctaagaagagagtgagggataggtatgcgcaacggcgatagcttaagcggctttcctcttctgcacgactatcagcttgaggagatgtggagaaccccgcagccaatccgtccattacagagaaaacgagggcgtgagggctggaattggcggcaacgggttcaatatgcgatagggcatgcgttgaacgaatactttggcatggaagtagaggtacggcgagaagtccctgtccaatatgtgctggagaaatggtggcctaagcagtcgaacagttttgattcattgttccattactgggatgtcaaaagtaaagtgtcaggcgagttgtcaaaaatatgtgccttaaataatgacttatggatgcctgtaatgttgtatgaacaattttgcacagtcgtgcctgagctggaggtcgatatttcgctgattattcaggcagcttggcagctctcagaacaagcggactcattgctgattcaaaaatatatggtggactacaaccccaacgttattcgtacatttcaacatctaacaaatgtatt

(19) United States
(12) Patent Application Publication
(10) Pub. No.: US 2026/0317693 A1
(43) Pub. Date: Oct. 15, 2026

(54) INCREASED NITROGEN FIXATION USING BACTERIA WITH MODIFIED ELECTRON TRANSPORT GENES

(71) Applicants: Bayer CropScience LLC, St. Louis, MO (US); Ginkgo Bioworks, Inc., Boston, MA (US)

(72) Inventors: Gregory Benjamin FOLEY, Somerville, MA (US); Guillaume BARBIER, Waterloo (AU); Eammon Patrick RILEY, Weymouth, MA (US); Timothy HARDEN, Somerville, MA (US)

(21) Appl. No.: 19/382,716

(22) Filed: Nov. 7, 2025

Related U.S. Application Data
(60) Provisional application No. 63/718,158, filed on Nov. 8, 2024.

(51) Int. Cl. C12N 1/20; A01N 63/25; A01P 21/00; C12N 9/02; C12N 15/52; C12R 1/01

(52) U.S. Cl. CPC C12N 1/20; A01N 63/25; A01P 21/00; C12N 9/0008; C12N 9/0095; C12N 15/52; C12Y 102/07001; C12Y 118/06001; C12N 2830/002; C12N 2830/34; C12R 2001/01

(57) ABSTRACT

Paenibacillus bacterial strains are engineered to express or overexpress (i) one or more proteins (pyruvate ferredoxin oxidoreductase, flavodoxin, and/or ferredoxin) involved in the electron transport chain, (ii) the DUF protein and/or (iii) gene products of the suf operon. This enables a greater rate/level of nitrogen fixation by the diazotrophic bacteria. The engineered bacteria are introduced to agricultural plants, or seeds for growing such, forming a symbiotic relationship that results in increased nitrogen availability to the plant and improved plant agronomic traits.

### CROSS-REFERENCE TO RELATED APPLICATIONS

This application claims the benefit of U.S. Provisional Patent Application No. 63/718,158, filed Nov. 8, 2024, the entire disclosure of which is incorporated herein by reference.

### STATEMENT REGARDING SEQUENCE LISTING

A sequence listing contained in the file named “BCS249001US01.xml” created on Nov. 6, 2025, which is 430, 199 bytes, is incorporated herein by reference in its entirety.

### FIELD OF THE DISCLOSURE

The present disclosure relates to Paenibacillus microbes engineered to increase nitrogen fixation and their association with an agricultural plant to provide increased nitrogen to the plant and improved agronomic traits for the plant. Compositions including Paenibacillus microbes and/or plants associated with the microbes and methods of preparing and using the compositions are also disclosed.

### BACKGROUND OF THE DISCLOSURE

Plant growth requires nitrogen. Although nitrogen makes up approximately 80% of the atmosphere in the form of dinitrogen gas (N₂), plants cannot utilize N₂ directly. Plants are able to obtain reduced nitrogen in the form of ammonia (NH₃), ammonium (NH₄⁺) and nitrates (NO₃⁻) from the soil by absorption through their roots. Nitrogen-based (N-based) fertilizers have been developed to address the fact that availability of reduced nitrogen is often the limiting factor for plant growth, but inefficient use of the N-based fertilizers by plants has contributed to contamination of soils and water resources and poses a hazard to human health. Additionally, manufacturing N-based fertilizer is more energy-intensive than producing either phosphorus-based or potassium-based fertilizers and consumes a significant amount of fossil fuels and is a significant source of greenhouse gases.

Some microorganisms are able to convert atmospheric N₂ to ammonia (NH₃) or ammonium (NH₄⁺), which can be used by plants. Microorganisms that can perform this biological nitrogen fixation are called diazotrophs. Diazotrophic microorganisms are able to express a nitrogenase enzyme complex that reduces N₂ to NH₃ or NH₄⁺.

Paenibacillus is a rhizobacteria that has been isolated from the rhizosphere, rhizoplane and plant tissue of many important agricultural crops across the world. The genus includes species that produce industrially relevant enzymes and antimicrobials, solubilize phosphates, inhibit the growth of plant pathogens, and express enzymes that may be useful for bioremediation or production of chemicals. Some Paenibacillus species can directly influence plant growth because they are able to produce the phytohormone indole-3-acetic acid (IAA). As plant-associated diazotrophic rhizobacteria, Paenibacillus species can also promote the growth of plants through nitrogen fixation.

Nitrogen-fixing microorganisms hold promise as a more environmentally friendly alternative to chemical fertilizers and could offer a route to enhance plant growth, improve sustainability of agriculture, and reduce damage to the environment and human health. The use of diazotrophic bacteria as biofertilizers has been limited due to environmental factors such as soil pH, salinity, moisture content, and temperature. With the advent of technologies that allow the sequencing and study of genomes, it is now possible to exploit newly available knowledge, of the genomes of diazotrophs to engineer them for more efficient nitrogen fixation.

### SUMMARY OF THE DISCLOSURE

The present disclosure provides a Paenibacillus microbe genetically modified to increase nitrogen fixation compared to a non-genetically modified Paenibacillus microbe. Nitrogen fixation is increased in Paenibacillus by strategies including: expressing or overexpressing one or more electron transport chain component and/or protein associated with nitrogen fixation or its regulation. The expression or overexpression includes integrating a genetic construct including an expression cassette into the genome of a Paenibacillus microbe. The genetically modified Paenibacillus microbe is associated with agricultural plants, or the seeds for growing such agricultural plants, forming a commensal relationship that results in increased nitrogen availability to the plant and improved plant agronomic traits.

In particular embodiments, the genetically modified Paenibacillus microbe or genetic construct includes at least one native or heterologous gene encoding an electron transport chain component. In particular embodiments, at least one native or heterologous gene encoding an electron transport chain component includes: pyruvate ferredoxin-oxidoreductase porCDAB E.C. 1.2.7.1; flavodoxin fldA; sufA, sufB, sufC, sufD, sufE, sufS, and/or sufU genes of a suf operon for iron-sulfur cluster biosynthesis; ferredoxin fer, or combinations thereof. In particular embodiments, the genetically modified Paenibacillus microbe or genetic construct further includes at least one heterologous gene associated with nitrogen fixation or its regulation. In particular embodiments, the gene of a nif operon includes DUF269, encoding a protein involved in protection of nitrogenase from oxidative damage.

In particular embodiments, the genetically modified Paenibacillus microbe or genetic construct includes porCDAB E.C. 1.2.7.1 from Paenibacillus zanthoxyli (Pz) or Paenibacillus forsythiae (Pf). In particular embodiments, the genetically modified Paenibacillus microbe or genetic construct includes a sufCDSUB operon including sufB, sufC, sufD, sufS, and sufU genes from Pz. In particular embodiments, the genetically modified Paenibacillus microbe or genetic construct includes a sufCBSUB operon including sufB, sufC, sufS, and sufU genes from Pz or Pf. In particular embodiments, the genetically modified Paenibacillus microbe or genetic construct includes an fldA gene from Pz or Pf. In particular embodiments, the genetically modified Paenibacillus microbe or genetic construct includes a fer gene from Pz or Pf. In particular embodiments, the genetically modified Paenibacillus microbe or genetic construct includes an operon including DUF269, genes from Pz, Pf, Paenibacillus durus (Pd), Paenbacillus graminis (Pg), Paenibacillus jilunlii (Pj), Paenibacillus riograndensis (Pr), Paenibacillus sabinae (Psab), or Paenibacillus stellifer (Pstel). In particular embodiments, the genetically modified Paenibacillus microbe or genetic construct includes an operon including flavodoxin genes from Pz. In particular embodiments, the genetically modified Paenibacillus microbe or genetic construct includes an operon including ferredoxin and flavodoxin genes from Pf. In particular embodiments, the genetically modified Paenibacillus microbe or genetic construct includes a DUF269 gene from Pz, Pf, Pd, Pg, Pj, Pr, Psab, or Pstel. In particular embodiments, the at least one native or heterologous gene encoding an electron transport chain component and/or the at least one native or heterologous gene associated with nitrogen fixation or regulation is operably linked to a heterologous constitutive or inducible promoter. In particular embodiments, the heterologous constitutive promoter is selected from: P43, PR′, PSigX, PtmQ, PclpE, PywbO, PypuA, PywrK, PsigV, PydaH, PyjoB, PyqeZ, PyacL, PyceG, PyrhK, PradA, PywaC, PmurB, PfabHA, PhtrB, PaprE, PycbR, PhtrA, PyrhH, PpbpE, PybfP, PywnJ, PytpA, PoatA, PmreBH, PyeaA, PybfO, PmurF, PyuaF, PypbG, PbcrC, PmetA, PylxX, PybgB, Pyusl, PcsbB, PyjbC, PclpC, PythP, PhtpG, Pyxil, PtilS, PspoOM, PdivlB, PypuD, PcssR, PyxzE, PdnaJ, PybfQ, PxpaC, PyngC, PyvlA, PpssA, PyoaF, PminC, PdltE, ProdA, PpspA, PclpP, PdivIC, PydjO, PydbS, PysdB, PyoaG, Pddl, PfosB, Pabh, Pspa, PyceE, PyknW, or PmreB. In particular embodiments, the heterologous constitutive promoter is derived from the Bacillus or Paenibacillus genus. In particular embodiments, the heterologous constitutive promoter is derived from Bacillus subtilis. In particular embodiments, the heterologous constitutive promoter is selected from: PsigX, PtrnQ, and PmreB. In particular embodiments, the at least one native or heterologous gene encoding an electron transport chain component and/or the at least one heterologous gene associated with nitrogen fixation or regulation is operably linked to a native or heterologous inducible promoter. In particular embodiments, the heterologous inducible promoter is heat shock promoter PgroES or other inducible promoter.

In particular embodiments, the at least one heterologous gene encoding an electron transport chain component and/or the at least one heterologous gene of a nif operon associated with nitrogen fixation is part of an expression cassette integrated at a thymine adenine (TA) dinucleotide site in the Paenibacillus microbe genome.

In particular embodiments, the at least one heterologous gene encoding an electron transport chain component and/or the at least one heterologous gene of a nif operon associated with nitrogen fixation is part of an expression cassette integrated at a czcB locus in the Paenibacillus microbe genome.

In particular embodiments, the at least one heterologous gene encoding an electron transport chain component and/or the at least one heterologous gene of a nif operon associated with nitrogen fixation is part of an expression cassette integrated at a PROKKA_03723 locus in the Paenibacillus microbe genome.

Particular embodiments provide for a genetic construct including, in 5′ to 3′ order: (a) a heterologous promoter; and (b) at least one heterologous gene encoding an electron transport chain component and/or the at least one heterologous gene associated with nitrogen fixation or regulation, wherein the genetic construct includes a sequence having at least 90% sequence identity to: SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 28; SEQ ID NO: 29; SEQ ID NO: 30; SEQ ID NO: 31; SEQ ID NO: 32; SEQ ID NO: 33; SEQ ID NO: 38; SEQ ID NO: 39; SEQ ID NO: 40; SEQ ID NO: 41; or SEQ ID NO: 42; SEQ ID NO: 43, SEQ ID NO: 44, SEQ ID NO: 45; SEQ ID NO: 47; or SEQ ID NO: 49 or includes a sequence as set forth in: SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 28; SEQ ID NO: 29; SEQ ID NO: 30; SEQ ID NO: 31; SEQ ID NO: 32; SEQ ID NO: 33; SEQ ID NO: 38; SEQ ID NO: 39; SEQ ID NO: 40; SEQ ID NO: 41; or SEQ ID NO: 42; SEQ ID NO: 43, SEQ ID NO: 44, SEQ ID NO: 45; SEQ ID NO: 47; or SEQ ID NO: 49. In particular embodiments, the heterologous promoter is a constitutive promoter. In particular embodiments, the heterologous promoter is an inducible promoter. In particular embodiments, the genetic construct includes, in 5′ to 3′ order: a heterologous constitutive promoter; and an fldA gene encoding a flavodoxin, wherein the genetic construct includes a sequence having at least 90% sequence identity to SEQ ID NO: 4 or includes a sequence as set forth in SEQ ID NO: 4.

In particular embodiments, nitrogenase activity of a genetically modified Paenibacillus microbe is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to nitrogenase activity of a control Paenibacillus microbe as measured by an acetylene reduction assay, an ¹⁵N₂ fixing assay, an ¹⁵N dilution assay, and/or an ammonia biosensor assay. In particular embodiments, the nitrogenase activity of the genetically modified Paenibacillus microbe is increased by up to 10%, 20%, 30%, 40% or 50%, as compared to nitrogenase activity of a wild-type control Paenibacillus microbe of the same species as measured by an acetylene reduction assay. In particular embodiments, ammonium secretion from a genetically modified Paenibacillus microbe is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to ammonium secretion of a control Paenibacillus microbe as measured by a biosensor indicator assay.

In particular embodiments, ammonium production of the genetically modified Paenibacillus microbe is increased by up to 10×, 20×, or 30×, as compared to nitrogenase activity of a wild-type control Paenibacillus microbe of the same species as measured by an ammonia excretion assay.

In particular embodiments, a control Paenibacillus microbe includes a non-genetically modified Paenibacillus microbe, for example the parental strain of the engineered microbe. In particular embodiments, a control Paenibacillus microbe includes a Paenibacillus microbe with increased nitrogen fixation as compared to a non-genetically modified Paenibacillus microbe.

The disclosure also provides a formulation including a genetically modified Paenibacillus microbe and a carrier. In particular embodiments, the formulation includes a buffer, a tackifier, a plant growth regulator, a stabilizer, a surfactant, an adherent, a desiccant, a fungicide, a nematicide, a rodenticide, an insecticide, an herbicide, a virucide, a nutrient, or any combination thereof. In particular embodiments, the formulation includes a seed coating.

The disclosure also provides a composition including a plant or plant part and a genetically modified Paenibacillus microbe disclosed herein. In particular embodiments, the genetically modified Paenibacillus microbe is included in a formulation. In particular embodiments, the plant part is a seed and the formulation includes a seed coating. In particular embodiments, the composition further includes a medium that promotes plant growth.

Particular embodiments provide for a method for preparing a composition, including contacting the surface of a plant or plant part with a formulation including a genetically modified Paenibacillus microbe disclosed herein to produce an inoculated plant or plant part including the genetically modified Paenibacillus microbe, wherein the genetically modified Paenibacillus microbe is present in the formulation in an amount capable of improving an agronomic trait of a plant grown from the inoculated plant or plant part.

Particular embodiments provide for a method of inoculating a plant or plant part, including contacting a plant or plant part with a formulation including a genetically modified Paenibacillus microbe disclosed herein. In particular embodiments, the method further includes growing the inoculated plant or plant part. Particular embodiments provide for a plant or part thereof grown from the inoculated plant or plant part.

Particular embodiments provide for a method of improving an agronomic trait in a plant, including growing a plant from a plant or plant part that has been contacted with a formulation including a genetically modified Paenibacillus microbe disclosed herein. In particular embodiments, chlorophyll content of a genetically-modified Paenibacillus microbe-associated plant is increased 3 to 20% as compared to chlorophyll content of a reference plant. In particular embodiments, shoot fresh weight of a genetically modified Paenibacillus microbe-associated plant is increased 3 to 20% as compared to shoot fresh weight of a reference plant. In particular embodiments, shoot dry weight of a genetically modified Paenibacillus microbe-associated plant is increased 3 to 20% as compared to shoot dry weight of a reference plant.

Particular embodiments provide for a method of reducing nitrogen fertilizer application, including: inoculating a plant or plant part with a formulation including a genetically-modified Paenibacillus microbe disclosed herein, and growing a plant from the inoculated plant or plant part. In particular embodiments, the application of nitrogen to the plant is reduced as compared to application of nitrogen to a corresponding plant grown from a plant or plant part that has not been inoculated with the formulation. In particular embodiments, the reduction in the application of nitrogen (N) is measured as N replacement, and wherein the N replacement is at least 5 ppm of N, at least 6 ppm of N, at least 7 ppm of N, at least 8 ppm of N, at least 9 ppm of N, at least 10 ppm of N, at least 11 ppm of N, at least 12 ppm of N, at least 13 ppm of N, at least 14 ppm of N, at least 15 ppm of N, at least 16 ppm of N, at least 17 ppm of N, at least 18 ppm of N, at least 19 ppm of N, at least 20 ppm of N, at least 21 ppm of N, at least 22 ppm of N, at least 23 ppm of N, at least 24 ppm of N, at least 25 ppm of N or greater.

In particular embodiments, the genetically modified Paenibacillus microbe is P. abyssi, P. aestuarii, P. agarexedens, P. agaridevorans, P. alba, P. algeriensis, P. alginolyticus, P. algorifonticola, P. alkaliterrae, P. alvei, P. amylolyticus, P. anaericanus, P. antarcticus, P. antibioticophila, P. apiarius, P. assamensis, P. azoreducens, P. barcinonensis, P. barengoltzii, P. beijingensis, P. borealis, P. bovis, P. brasilensis, P. brassicae, P. camelliae, P. camerounensis, P. campinasensis, P. castaneae, P. catalpa, P. cathormii, P. cavernae, P. cellulositrophicus, P. cellulosilyticus, P. chartarius, P. chibensis, P. chinensis, P. chinjuensis, P. chitinolyticus, P. chondroitinus, P. chungangensis, P. cineris, P. contaminans, P. cookii, P. cucumis, P. curdlanotyticus, P. daejeonensis, P. dakarensis, P. darwinianus, P. dauci, P. dendritiformis, P. dongdonensis, P. donghaensis, P. doosanensis, P. durus, P. edaphicus, P. ehimensis, P. elgii, P. endophyticus, P. enshidis, P. etheri, P. faecis, P. favisporus, P. ferrarius, P. filicis, P. fonticola, P. forsythiae, P. frigoriresistens, P. gansuensis, P. gelatinilyticus, P. ginsengarvi, P. ginsengihumi, P. ginsengiterrae, P. glacialis, P. glucanolyticus, P. glycanilyticus, P. gorillae, P. graminis, P. granivorans, P. guangzhouensis, P. harenae, P. hemerocallicola, P. herberti, P. hodogayensis, P. hongkongensis, P. hordei, P. humi, P. humicus, P. hunanensis, P. ihumii, P. illinoisensis, P. insulae, P. jamilae, P. jilunlii, P. kobensis, P. koleovorans, P. konsidensis, P. koreensis, P. kribbensis, P. kyungheensis, P. lactis, P. larvae, P. lautus, P. lemnae, P. lentimorbus, P. lentus, P. lupini, P. macerans, P. macquariensis, P. marchantiophytorum, P. marinisediminis, P. marinum, P. massiliensis, P. medicaginis, P. mendelii, P. montaniterrae, P. motobuensis, P. mucilaginosus, P. nanensis, P. naphthalenovorans, P. nasutitermitis, P. nematophilus, P. nicotianae, P. oceanisediminis, P. odorifer, P. oenotherae, P. pabuli, P. panacisoli, P. panaciterrae, P. pasadenensis, P. pectinilyticus, P. peoriae, P. periandrae, P. phoenicis, P. phyllosphaerae, P. physcomitrellae, P. pinesoli, P. pini, P. pinihumi, P. pocheonensis, P. polymyxa, P. popilliae, P. populi, P. profundus, P. prosopidis, P. provencensis, P. pueri, P. puldeungensis, P. purispatii, P. qingshengii, P. quercus, P. radicis, P. relictisesami, P. residui, P. rhizoryzae, P. rhizosphaerae, P. rigui, P. riograndensis, P. ripae, P. sabinae, P. sacheonensis, P. sanguinis, P. sediminis, P. selenii, P. selenitireducens, P. senegalensis, P. septentrionalis, P. sepulcri, P. shenyangensis, P. shirakamiensis, P. siamensis, P. soli, P. sonchi, P. sophorae, P. sputi, P. stellifer, P. susongensis, P. swuensis, P. taichungensis, P. taihuensis, P. taiwanensis, P. taohuashanense, P. tarimensis, P. telluris, P. terrae, P. terreus, P. terrigena, P. tezpurensis, P. thailandensis, P. thermoaerophilus, P. thermophilus, P. thiaminolyticus, P. tianmuensis, P. tibetensis, P. timonensis, P. tundrae, P. turicensis, P. tylopili, P. typhae, P. tyraminigenes, P. uliginis, P. urinalis, P. validus, P. vini, P. vortex, P. vulneris, P. wenxiniae, P. wooponensis, P. woosongensis, P. wulumuqiensis, P. wynnii, P. xanthinilyticus, P. xinjiangensis, P. xylanexedens, P. xylaniclasticus, P. xylanisolvens, P. xylanilyticus, P. yonginensis, P. yunnanensis, P. zanthoxyli, or P. zeae. In particular embodiments, the genetically modified Paenibacillus microbe is P. brasilensis.

### BRIEF DESCRIPTION OF THE DRAWINGS

FIG. 1. Simplified electron production and transport to nitrogenase. Adapted from Gu and Milton. Chemistry 2020; 2:21.

FIGS. 2A-2G. Final constructs and integration sites for genetically-modified Paenibacillus strains.

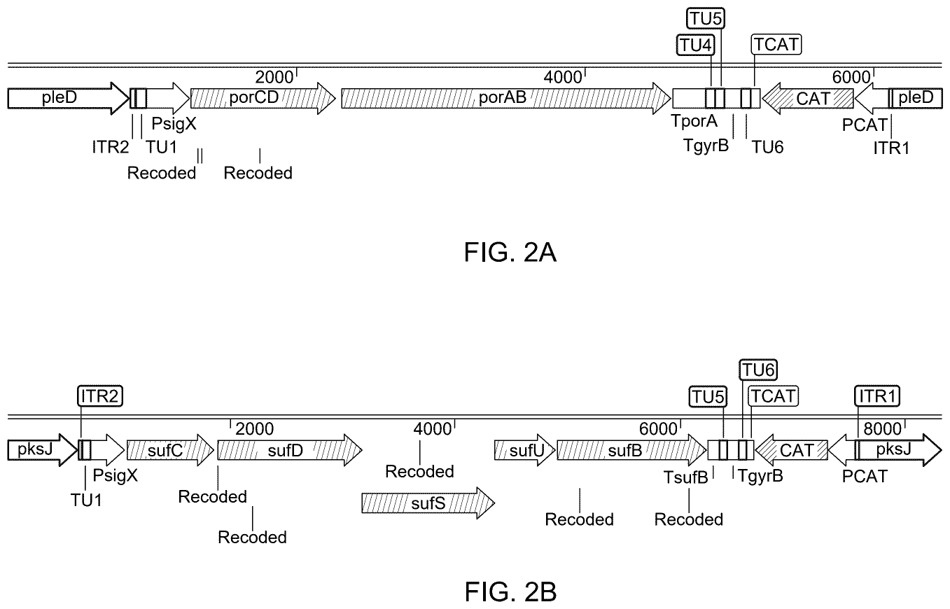

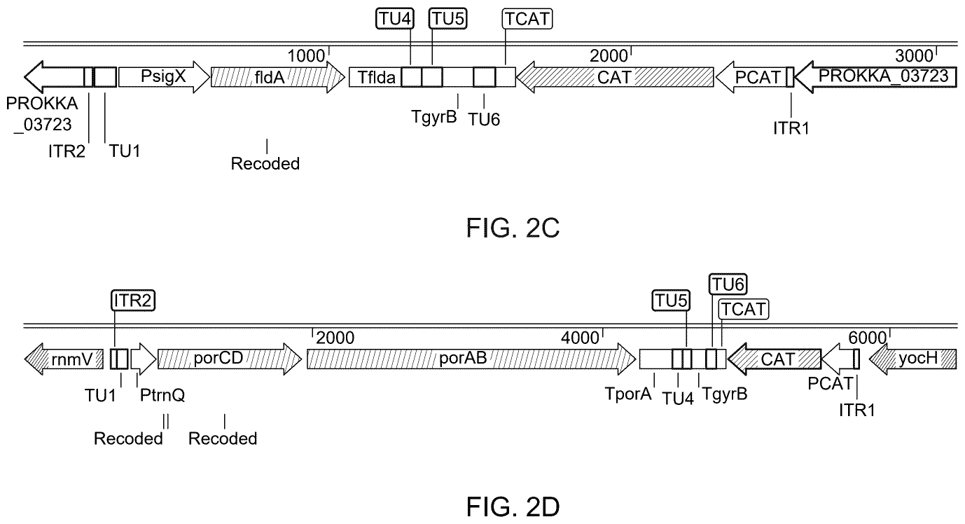

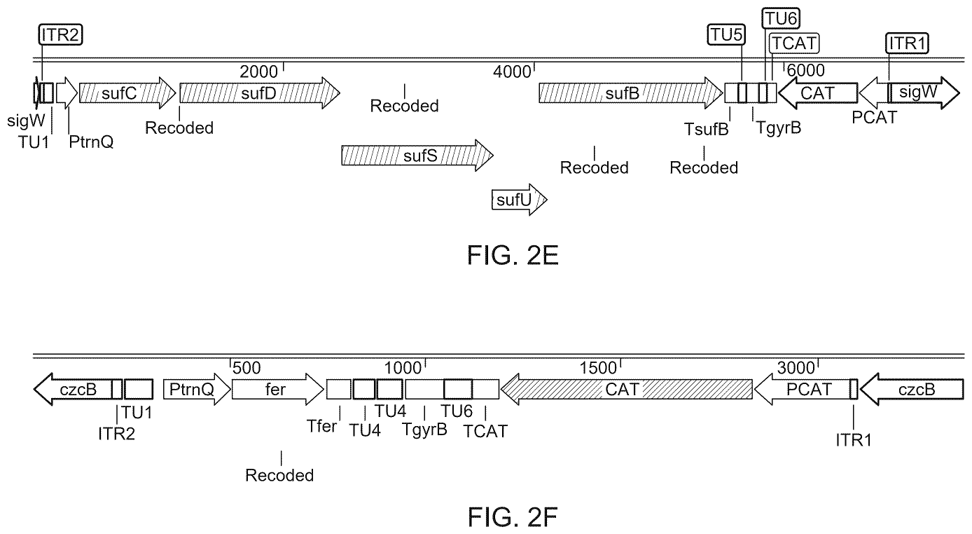

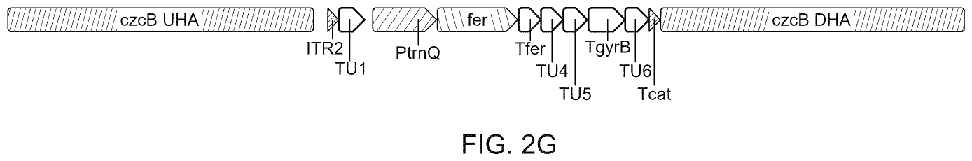

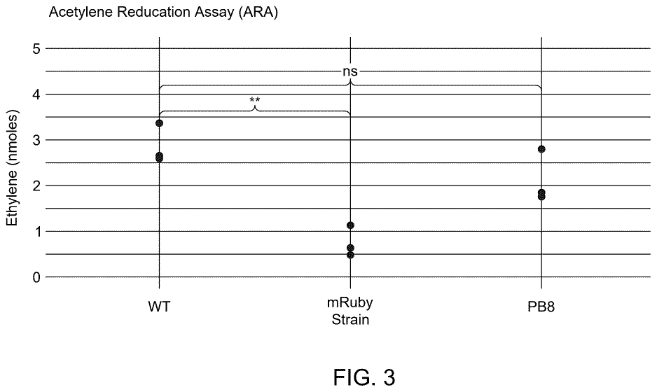

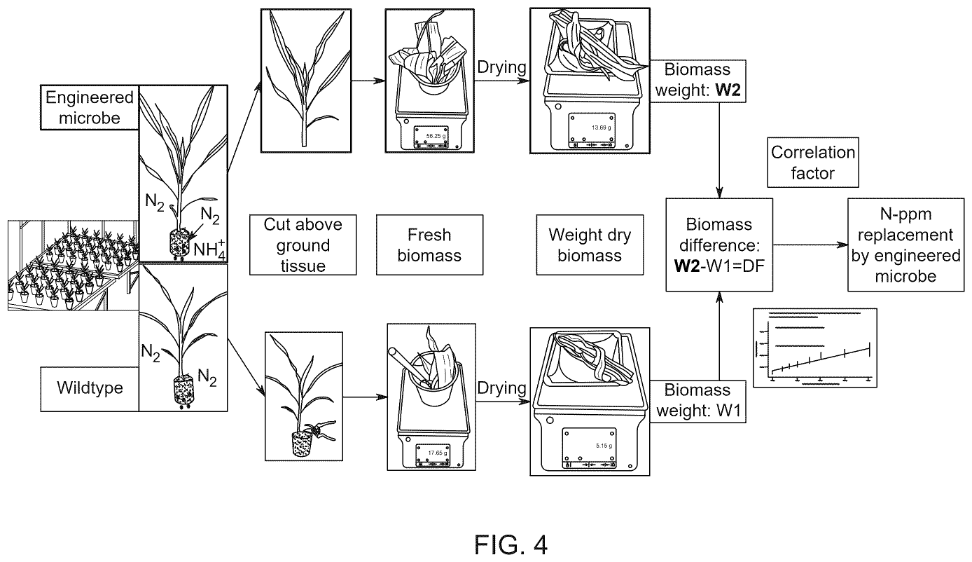

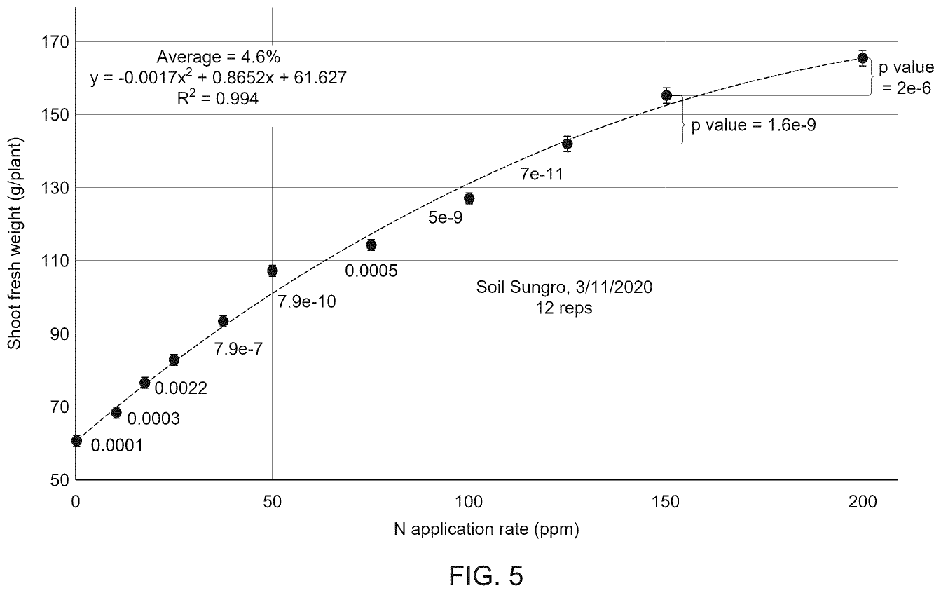

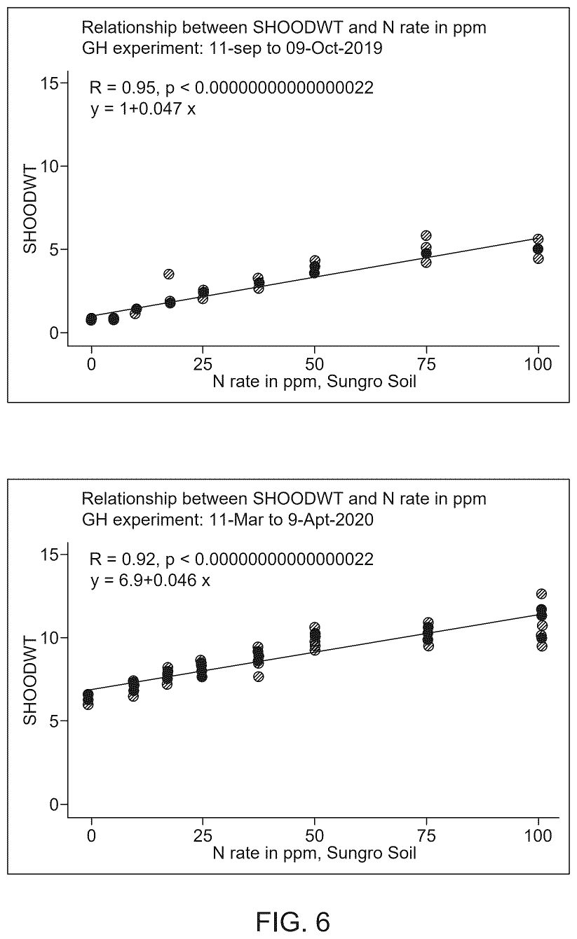

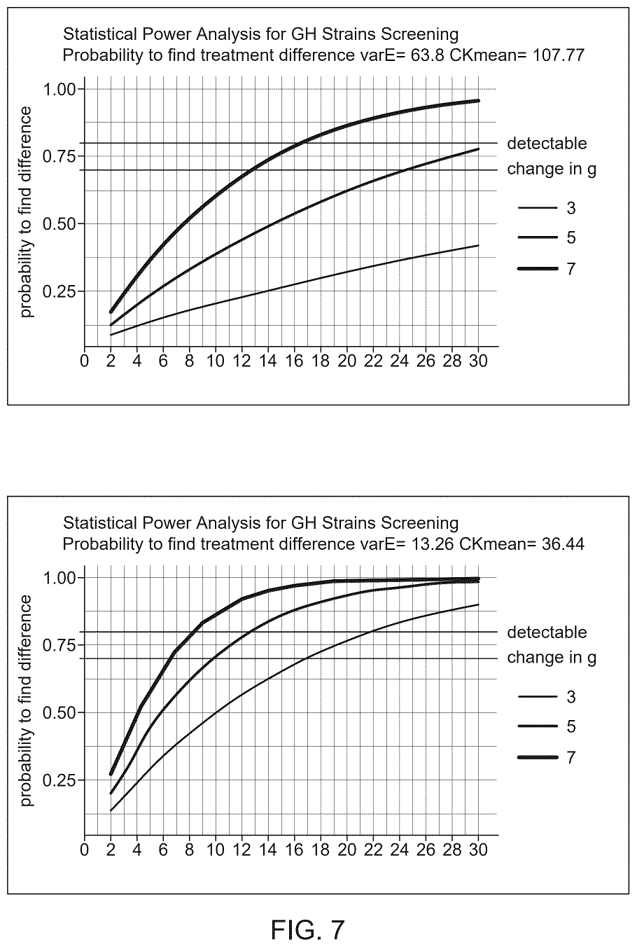

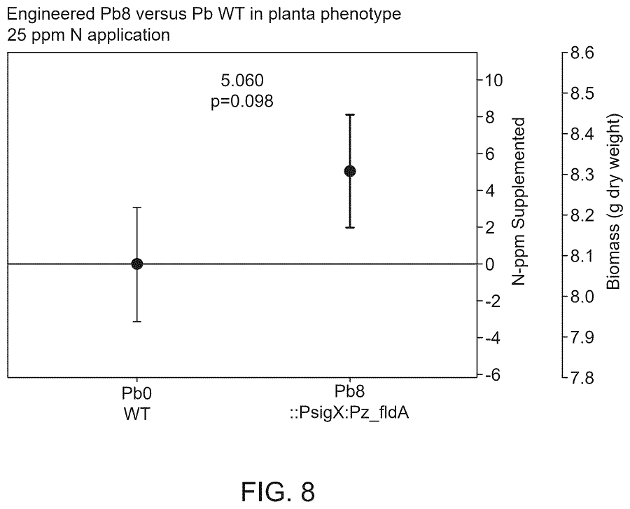

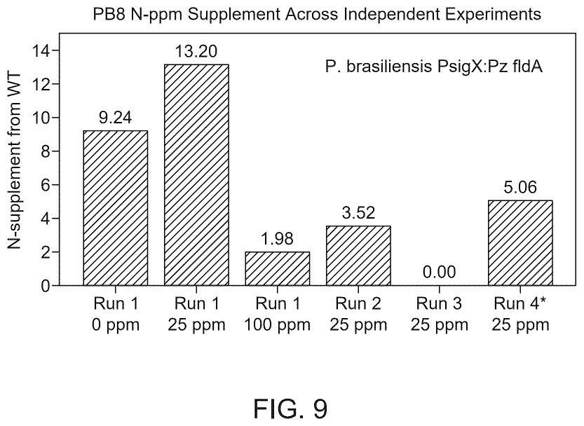

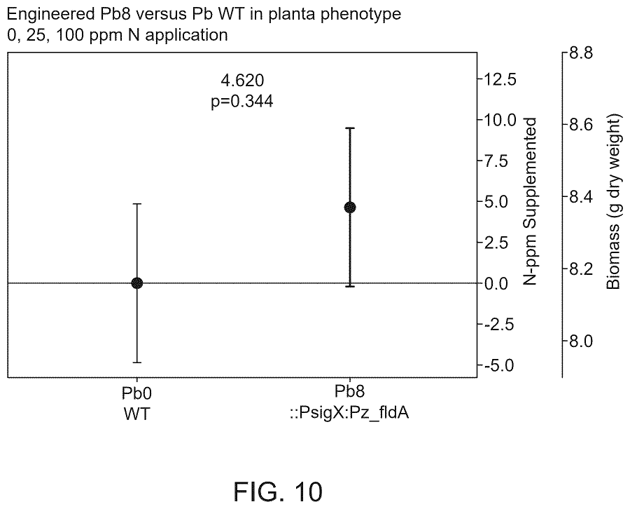

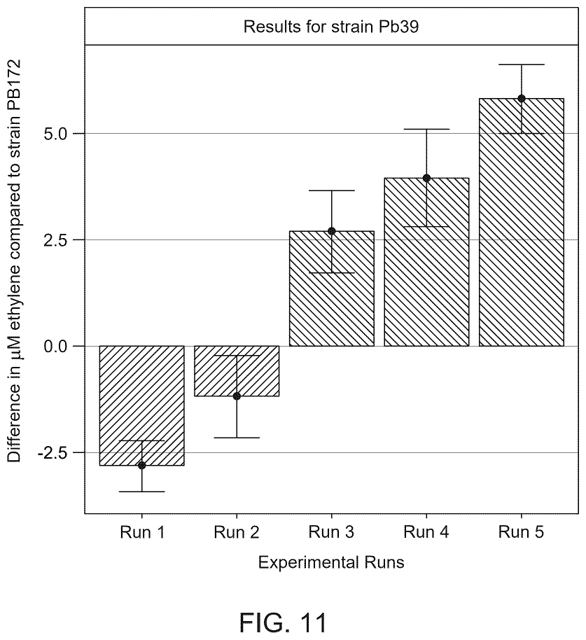

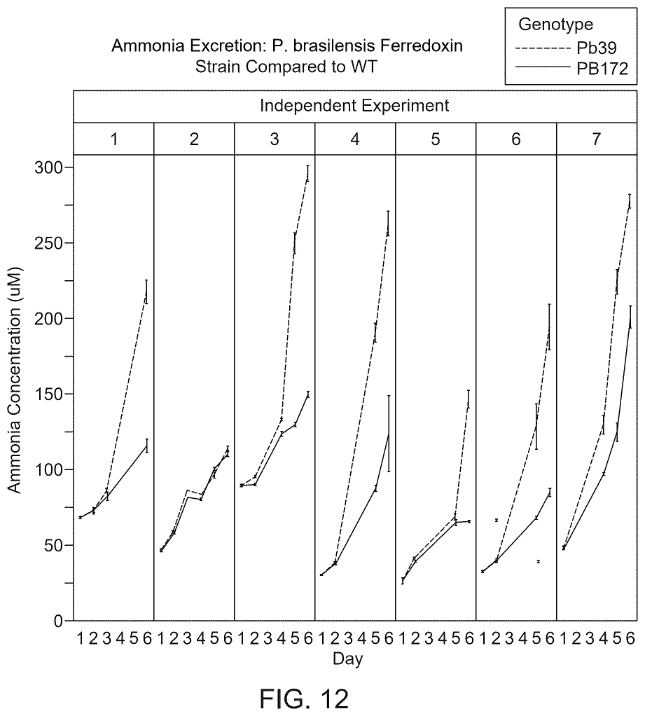

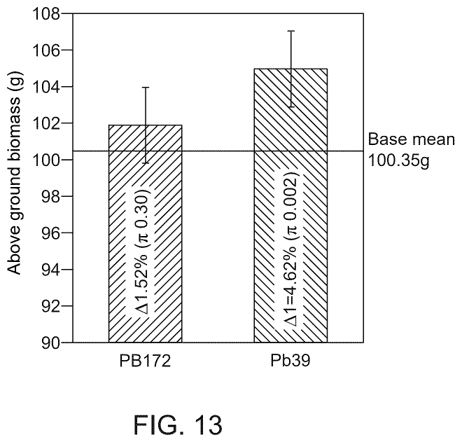

(FIG. 2A) Pb6. PsigX: 311 bp sequence for sigma factor X promoter of Paenibacillus polymyxa; porCD: 1017 bp sequence encoding subunit C and subunit D of pyruvate:flavodoxin oxidoreductase from Paenibacillus zanthoxyli; porAB: 2304 bp sequence encoding subunit A and subunit B of pyruvate:flavodoxin oxidoreductase from Paenibacillus zanthoxyli; pleD: construct insertion site-recipient organism's disrupted pleD gene which is described as a diguanylate cyclase involved in response regulation.

(FIG. 2B) Pb16. PsigX: 311 bp sequence for sigma factor X promoter of Paenibacillus polymyxa; sufC: 801 bp gene encoding subunit C of Paenibacillus forsythiae sulfur formation operon; sufD: 1302 bp gene encoding subunit D of Paenibacillus forsythiae sulfur formation operon; sufS: 1224 bp gene encoding subunit S of Paenibacillus forsythiae sulfur formation operon; sufU: 435 bp gene encoding subunit U of Paenibacillus forsythiae sulfur formation operon; sufB: 1398 bp gene encoding subunit B of Paenibacillus forsythiae sulfur formation operon; pksJ: construct insertion site—recipient organism's disrupted gene for polyketide synthase gene J involved in intermediate steps of the synthesis of the antibiotic polyketide bacillaene, which is involved in secondary metabolism.

(FIG. 2C) Pb8. PsigX: 311 bp sequence for sigma factor X promoter of B. subtilis; fidA: 453 bp flavodoxin gene from Paenibacillus zanthoxyli; PROKKA_03723: construct insertion site—recipient organism's disrupted gene which is annotated as a hypothetical protein.

(FIG. 2D) Pb10. PtrnQ: 182 bp sequence of a tmnQ tRNA promoter of B. subtilis; porCD: 1017 bp sequence encoding subunit C and subunit D of pyruvate:flavodoxin oxidoreductase from Paenibacillus zanthoxyli; porAB: 2304 bp sequence encoding subunit A and subunit B of pyruvate:flavodoxin oxidoreductase from Paenibacillus zanthoxyli; ycoH and mmV: construct insertion site—recipient organism's intergenic region is between ycoH, described as a precursor of the cell wall-binding protein and rnmV, encoding ribonuclease M5 which processes the 5s rRNA.

(FIG. 2E) Pb11. PtrnQ: 182 bp sequence of a trnQ tRNA promoter of B. subtilis; sufC: 801 bp gene encoding subunit C of Paenibacillus forsythiae sulfur formation operon; sufD: 1302 bp gene encoding subunit D of Paenibacillus forsythiae sulfur formation operon; sufS: 1224 bp gene encoding subunit S of Paenibacillus forsythiae sulfur formation operon; sufU: 435 bp gene encoding subunit U of Paenibacillus forsythiae sulfur formation operon; sufB: 1398 bp gene encoding subunit B of Paenibacillus forsythiae sulfur formation operon; sigW_6: construct insertion site-recipient organism's disrupted locus, PROKKA_04708 in the coding sequence denoted sigW_6, one of seven genes annotated as RNA polymerase sigma factor SigW, which controls the expression of genes involved in the response to cell envelope stress such as antimicrobial peptides, alkaline pH, transport processes and detoxification.

(FIG. 2F) Pb 12. PtrnQ: 182 bp sequence of a trnQ tRNA promoter of B. subtilis; fer: 237 bp sequence encoding ferredoxin from Paenibacillus zanthoxyli which is involved in accepting electrons from pyruvate and donating electrons to the metallo-cluster of nitrogenase; czcB: construct insertion site—recipient organism's disrupted gene, czcB, which is involved in resistance to cobalt, zinc, and cadmium.

(FIG. 2G) Pb39, Pp1, Pp2, Pp3 and Pp4. FIG. 2G outlines the construct used to generate Pb39, Pp1, Pp2, Pp3, and Pp4. The construct used to generate Pb39 is shown. Pp1 is similar to the construct used to generate Pb39 with some differences in the spacer 5′ of the promoter. For Pp2, Pp3, and Pp4 different promoters and ferredoxin (fer) genes are used. Ferredoxin is involved in accepting electrons from pyruvate and donating electrons to the metallo-cluster of nitrogenase. In Pb39 and Pp1 the ferredoxin corresponds to Paenibacillus zanthoxyli JH29 ferredoxin. In Pp2, Pp3 and Pp4 the ferredoxin corresponds to Paenibacillus (multispecies) ferredoxin. The promoters used in Pp2, Pp3, and Pp4 are, respectively, PtrnQ, PR′, and P43. In Pb39, Pp1 and Pp2 the promoter is the trnQ promoter of B. subtilis. In Pp3 the promoter is the R′ promoter of Bacteriophage lambda. In Pp4 the promoter is the P43 promoter of B. subtilis. To the 3′ of the ferredoxin gene are Tfer, the ferredoxin (fer) transcriptional terminator from P. zanthoxyli JH29 and TgyrB, the DNA gyrase B (gyrB) transcriptional terminator from Paenibacillus polymyxa. czcB UHA and czcB DHA define the 5′ (upstream) and the 3′ (i.e. downstream) of the construct insertion site respectively. The construct insertion site is the recipient organism's disrupted gene, czcB, which is involved in resistance to cobalt, zinc, and cadmium.

FIG. 3. Acetylene reduction assay (ARA) that measures nmoles of ethylene produced by engineered strain Pb8 (n=3), in comparison to wt and mRuby expressing strains. P-value annotations are as follows: not significant (ns), p-value<0.1 (*), p-value<0.01 (**) and p-value <0.001 (***) based on a t-test.

FIG. 4. Depiction of an in planta assay to screen and characterize engineered microbes in the greenhouse.

FIG. 5. Nitrogen (N) dose response of corn shoot fresh weight. Arrow shows actual testing regime at 25 N application rate.

FIG. 6. Dose response experiments. Regression slope is consistent from 2 different dose response experiments 0.047~0.046 g dry weight per 1 ppm N. Conversion of 1 g dry weight into ppm N is 22.

FIG. 7. Power curves generated to detect difference of 3, 5, and 7 grams in large plants (top graph) and smaller plants (bottom graph) with a statistical significance level of 0.05. Based on this analysis, 8 reps will detect with 80% power a 10% difference in larger plants (10 g), and a 5 g difference in smaller plants.

FIG. 8. Significant N supplement effect of engineered strain Pb8 (Pb PsigX:Pz_fldA) on corn biomass 28 days after planting (in-furrow inoculation) under 25 ppm N application condition, p=0.098. The graph indicates that corn plants contacted with the non-genetically engineered wild-type PB172 (control) (and thus receiving no supplemental nitrogen from the bacteria) has a biomass of 8.1 g dry weight, while corn plants contacted with the genetically engineered Pb8 (and thus supplemented with 5 ppm nitrogen from the Pb8 microbe) has a biomass of 8.3 g dry weight.

FIG. 9. Four independent greenhouse experiments show N supplemented by Pb8, when compared to wt Paenibacillus. Least squares mean difference is shown for each experiment by N level.

FIG. 10. N supplement effect of engineered strain Pb8 (Pb PsigX:Pz_fldA) on corn biomass 28 days after planting (in-furrow inoculation) under a range of N application conditions, p=0.344. Mixed model and mean effect aggregated across N levels. The graph is similar to that of FIG. 9 except that the N supplement effect of Pb8 is aggregated across 0, 25, and 100 ppm N application.

FIG. 11. Acetylene reduction assay (ARA) that measures nmoles of ethylene produced by engineered strain Pb1, in comparison to wild-type parent strain PB172. Results of five independent experimental runs are shown in which n=3 for each strain. Bars show the difference in ethylene produced between the wild type strain PB172 (which, for each round is set at 0, shown by the dashed line) and the strain Pb39 [overexpressing the fer gene from Paenibacillus zanthoxyli (Pz_fer) encoding ferredoxin], with positive values showing strain Pb1 producing more ethylene than wild-type strain PB172 and negative values showing strain Pb1 producing less ethylene than wild-type strain PB172. Error bars show standard deviation.

FIG. 12. Ammonia excretion (in μM) measured using the ammonia excretion assay. Results for strain Pb1 (dashed lines) and PB172 (continuous lines) are shown for 7 independent experiments over six days of growth. Error bars show standard deviation (n=4).

FIG. 13. Total above ground dry mass in corn treated with wild-type microbe, PB172 and edited microbe Pb39 [overexpressing the fer gene from Paenibacillus zanthoxyli (Pz_fer) encoding ferredoxin].

### DETAILED DESCRIPTION

The present disclosure describes Paenibacillus bacterial strains that are engineered to increase nitrogen fixation. The Paenibacillus strains are genetically modified to express or overexpress electron transport chain components and/or certain proteins involved in nitrogen fixation and/or its regulation, resulting in increased nitrogen fixation. Without being bound by any one hypothesis, the present disclosure suggests that electrons can be limiting in nitrogen fixation, and reducing power (the ability to gain electrons) may be obtained by expressing an alternate source of electrons. When introduced to agricultural plants, the engineered bacteria form a close association with the host plants, which results in increased nitrogen availability to the plant and improved agronomic traits of the plants such as increased biomass production. In particular, nitrogen fertilizer application on agricultural plants can be decreased by introducing the engineered microbes of the disclosure to the agricultural plants. In some embodiments, a microbe genetically modified to increase nitrogen fixation is advantageous because it promotes plant growth even at low N application. The N replacement is the decrease in the amount of nitrogen applied for a given yield of a plant when the plant is associated with a genetically-modified Paenibacillus microbe disclosed herein as compared to when the plant is associated with a wild-type (wt) non-genetically-modified Paenibacillus microbe.

Reducing atmospheric nitrogen (N₂) to ammonia (NH₃) requires the activity of a nitrogenase enzyme. This enzyme includes two proteins, a molybdenum-iron (MoFe) protein and an iron (Fe) protein. The MoFe protein is a heterotetramer (encoded by nifD and nifK genes) that includes two metalloclusters: FeMo-co, a [Mo-7 Fe-9S—C-homocitrate] cluster that serves as the active site of substrate binding and reduction; and a [8Fe-7S] cluster (P-cluster) that shuttles electrons to FeMo-co. The Fe protein (encoded by nifH gene) is a homodimer that is an obligate electron donor to the MoFe protein. The two subunits of the Fe protein are bridged by a [4Fe-4S] cluster. In addition to the structural nifD, nifK, and nifH genes, several genes are required for the biosynthesis of the metalloclusters, and along with other gene products, are needed to produce a functional nitrogenase. Studies have shown that nifE, nifN, nifX, nifB, nifQ, nifV, nifY, and nifH contribute to the synthesis and insertion of FeMo-co into nitrogenase, while nifU, nifS, and nifZ contribute to synthesis of metalloclusters. nifM is required for proper folding of nitrogenase Fe protein.

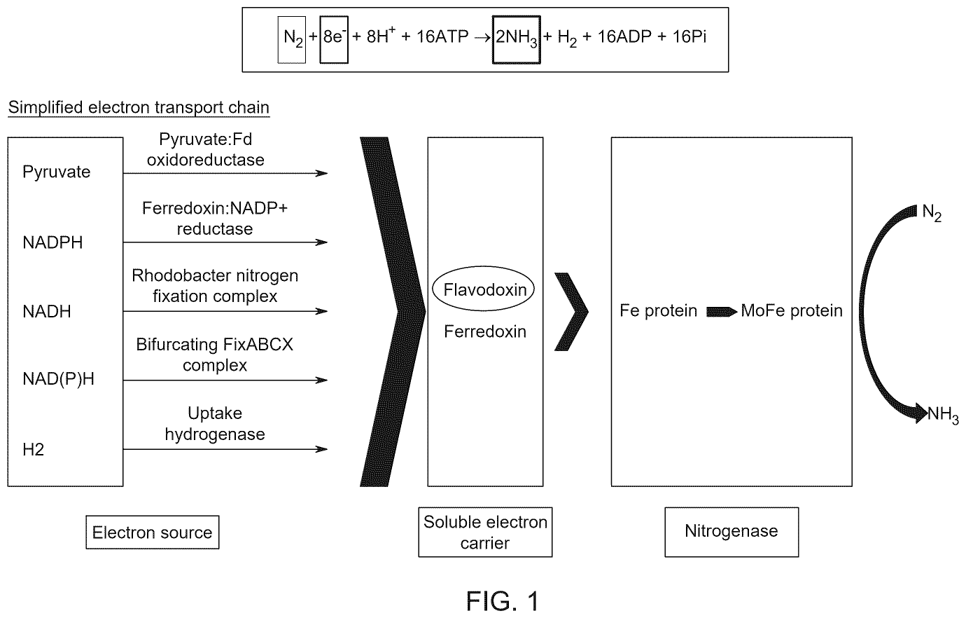

Nitrogen fixation is an energy-intensive endeavor, requiring a supply of magnesium adenosine triphosphate (MgATP). Reversible inhibition of nitrogenase activity can occur post-translationally once levels of extracellular ammonium (NH4⁺) increases. The nitrogenase reduction of molecular atmospheric dinitrogen to two molecules of ammonia is extremely electron intensive, requiring at least 8 electrons per reaction (FIG. 1). By comparison, most redox enzyme catalyzed reactions usually involve not more than 2 electrons. There are few sources of electrons available, such as pyruvate, nucleotides (NAD(P)H) and dihydrogen (FIG. 1).

There are more than 20 species of Paenibacillus that fix nitrogen. The nif cluster of Paenibacillus typically includes 9 genes within 10.5-12 kb.

The following aspects and options related to the current disclosure are now described in additional detail as follows: (i) Genetic modifications of electron transport chain components; (ii) Genetic modifications of genes involved in nitrogen fixation and/or regulation; (iii) Assays; (iv) Compositions and formulations; (v) Methods of use; (vi) Examples; (vii) Variants; and (viii) Closing Paragraphs.

### (i) GENETIC MODIFICATIONS WITH ELECTRON TRANSPORT CHAIN COMPONENTS

An electron transport chain (ETC) includes membrane-bound proteins that transfer electrons through the membrane through a series of reduction (gain of electrons)/oxidation (loss of electrons) (redox) reactions. In particular embodiments, the energy, difference between an electron donor and an electron acceptor can be harnessed to create an ion gradient across the membrane. Movement of the ion gradient back across the membrane can be coupled to ATP synthesis, thus converting electrical potential into chemical energy for the cell. In bacteria, molecules associated with the ETC are found at the plasma membrane.

Reduced coenzymes such as NADH and FADH₂ (from the citric acid cycle) are oxidized to NAD⁺ and FAD, respectively. The electrons from the oxidation are transferred through a series of electron carriers in the ETC. The step-by-step transfer of electrons allows incremental capture of the free energy from NADH and FADH₂. The electrons are transferred to a final acceptor, such as oxygen, nitrate, or fumarate.

The present disclosure provides a genetically-modified Paenibacillus microbe including at least one heterologous gene associated with an ETC. In particular embodiments, the Paenibacillus microbe is Paenibacillus brasilensis or Paenibacillus peoriae or Paenibacillus borealis. In particular embodiments, a gene associated with an ETC includes a gene encoding an ETC component. In particular embodiments, an ETC component includes any molecule associated with the function of an ETC. In particular embodiments, an ETC component includes: enzymes (e.g., dehydrogenase, pyruvate oxidoreductase, terminal oxidase, terminal reductase), electron carrying co-factors (e.g., quinones, flavines, cytochromes, heme, iron-sulfur clusters, copper ions), ion pumps, structural proteins, and ATP synthase.

In particular embodiments, an ETC component includes a pyruvate flavodoxin oxidoreductase enzyme EC 1.2.7.1. Pyruvate flavodoxin oxidoreductase catalyzes the following reaction:

pyruvate+CoA+2 oxidized ferredoxin=acetyl-CoA+CO₂+2 reduced ferredoxin+2H+

Pyruvate flavodoxin oxidoreductase (also known as pyruvate ferredoxin oxidoreductase or pyruvate synthase) is a member of the 2-oxoacid oxidoreductases, a family of enzymes that oxidatively decarboxylate different 2-oxoacids to form their coenzyme A (CoA) derivatives. It has an Fe—S protein as an acceptor. The porCDAB (WP_025689130.1, WP_025689129.1) operon from Paenibacillus encodes the A, B, C and D subunits of the pyruvate flavodoxin oxidoreductase protein. PorCDAB oxidizes pyruvate molecules that are produced as part of central carbon metabolism, thus freeing electrons that could potentially be utilized by the nitrogenase enzyme. PorCDAB can catalyze the indicated reaction on oxidized ferredoxin or oxidized flavodoxin substrates. Without being bound by any one hypothesis, overexpressing porCDAB may shift electron flux from the central carbon metabolism pathway towards the nitrogen fixation pathway. In particular embodiments, a genetically-modified Paenibacillus microbe or genetic construct of the disclosure includes a heterologous porCDAB operon encoding a pyruvate flavodoxin oxidoreductase E.C. 1.2.7.1. In particular embodiments, the porCDAB E.C. 1.2.7.1 may be from Paenibacillus zanthoxyli (Pz) or Paenibacillus forsythiae (Pf).

In particular embodiments, a genetically-modified Paenibacillus microbe or genetic construct of the disclosure includes a native porCDAB operon or heterologous Pz porCDAB operon which may be operably linked to a heterologous promoter, such as the medium strength constitutive PsigX promoter from Bacillus subtilis (B. subtilis). In particular embodiments, the genetic construct includes, in 5′ to 3′ order: a heterologous PsigX promoter; and a Pz porCDAB operon encoding a pyruvate flavodoxin oxidoreductase E.C. 1.2.7.1, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 2 or includes a sequence as set forth in SEQ ID NO: 2.

In particular embodiments, a genetically-modified Paenibacillus microbe or genetic construct of the disclosure includes a native porCDAB operon or heterologous Pz porCDAB operon which may be operably linked to a heterologous promoter, such as the strong constitutive PtrnQ promoter from B. subtilis. In particular embodiments, the genetic construct includes, in 5′ to 3′ order: a heterologous PtrnQ promoter; and a Pz porCDAB operon encoding a pyruvate flavodoxin oxidoreductase E.C. 1.2.7.1, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 6 or includes a sequence as set forth in SEQ ID NO: 6.

In particular embodiments, a genetically-modified Paenibacillus microbe or genetic construct of the disclosure includes a native porCDAB operon or heterologous Pz porCDAB operon which may be operably linked to a heterologous promoter, such as the constitutive PmreB promoter from B. subtilis. In particular embodiments, the genetic construct includes, in 5′ to 3′ order: a heterologous PmreB promoter; and a Pz porCDAB operon encoding a pyruvate flavodoxin oxidoreductase E.C. 1.2.7.1, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 16or includes a sequence as set forth in SEQ ID NO: 16.

In particular embodiments, a genetically-modified Paenibacillus microbe or genetic construct of the disclosure includes native porCDAB operon or a heterologous Pf porCDAB operon which may be operably linked to a heterologous promoter, such as the constitutive PmreB promoter from B. subtilis. In particular embodiments, the genetic construct includes, in 5′ to 3′ order: a heterologous PmreB promoter; and a Pf porCDAB operon encoding a pyruvate flavodoxin oxidoreductase E.C. 1.2.7.1, wherein the genetic construct includes a sequence having at least 90% sequence identity to SEQ ID NO: 11 or includes a sequence as set forth in SEQ ID NO: 11.

In particular embodiments, a genetically-modified Paenibacillus microbe or genetic construct of the disclosure includes a native porCDAB operon or heterologous Pf porCDAB operon which may be operably linked to a heterologous promoter, such as the medium strength constitutive PsigX promoter from B. subtilis. In particular embodiments, the genetic construct includes, in 5′ to 3′ order: a heterologous PsigX promoter; and a Pf porCDAB operon encoding a pyruvate flavodoxin oxidoreductase E.C. 1.2.7.1, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 13 or includes a sequence as set forth in SEQ ID NO: 13.

In particular embodiments, an ETC component includes a flavodoxin. In particular embodiments, a flavodoxin includes an electron transfer protein that includes flavin mononucleotide. Flavodoxin is involved in transporting electrons from pyruvate to the [4Fe-4S] metallo-cluster of the iron nitrogenase homodimer. Pyruvate is oxidized by pyruvate flavodoxin oxidoreductase (PorCDAB). PorCDAB then reduces flavodoxin, which in turn reduces the [4Fe-4S] cluster (Poudel et al. J Bacteriol. 2018; 200(10):e00757-17). Without being bound by any one hypothesis, overexpression of flavodoxin may increase the supply of electrons to nitrogenase and thereby increase the capacity of the cells to reduce nitrogen. In particular embodiments, a genetically-modified Paenibacillus microbe or genetic construct of the disclosure includes a heterologous fldA gene encoding a flavodoxin. In particular embodiments, the fldA gene may be from Pz or Pf.

In particular embodiments, a genetically-modified Paenibacillus microbe or genetic construct of the disclosure includes a native fldA gene or heterologous Pz fldA (WP_02569146) gene, either of which may be operably linked to a heterologous promoter, such as the medium strength constitutive PsigX promoter from B. subtilis. In particular embodiments, the genetic construct includes, in 5′ to 3′ order: a heterologous PsigX promoter; and a heterologous Pz fldA gene encoding a flavodoxin, wherein the genetic construct includes a sequence having at least 90% sequence identity to SEQ ID NO: 4 or includes a sequence as set forth in SEQ ID NO: 4.

In particular embodiments, a genetically-modified Paenibacillus microbe or genetic construct of the disclosure includes a native fldA gene or heterologous Pz fldA gene, either of which may be operably linked to a heterologous promoter, including a promoter, such as the strong constitutive PtrnQ promoter from B. subtilis. In particular embodiments, the genetic construct includes, in 5′ to 3′ order: a heterologous PtrnQ promoter; and a heterologous Pz fldA gene encoding a flavodoxin, wherein the genetic construct includes a sequence having at least 90% sequence identity to SEQ ID NO: 9 or includes a sequence as set forth in SEQ ID NO: 9.

In particular embodiments, a genetically-modified Paenibacillus microbe or genetic construct of the disclosure includes a native fldA gene or heterologous Pz fldA gene, either of which may be operably linked to a heterologous promoter, such as the constitutive PmreB promoter from B. subtilis. In particular embodiments, the genetic construct includes, in 5′ to 3′ order: a heterologous PmreB promoter; and a heterologous Pz fldA gene encoding a flavodoxin, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 14 or includes a sequence as set forth in SEQ ID NO: 14.

In particular embodiments, a genetically-modified Paenibacillus microbe or genetic construct of the disclosure includes a native fldA gene or heterologous Pf fldA gene, either of which may be operably linked to a heterologous promoter, such as the medium strength constitutive PsigX promoter from B. subtilis. In particular embodiments, the genetic construct includes, in 5′ to 3′ order: a heterologous PsigX promoter; and a heterologous Pf fldA gene encoding a flavodoxin, wherein the genetic construct includes a sequence having at least 90% sequence identity to SEQ ID NO: 12 or includes a sequence as set forth in SEQ ID NO: 12.

In particular embodiments, a genetically-modified Paenibacillus microbe or genetic construct of the disclosure includes a native fldA gene or heterologous Pf fldA gene, either of which may be operably linked to a promoter, such as the inducible PgroES promoter from B. subtilis. In particular embodiments, the genetic construct includes, in 5′ to 3′ order: a heterologous PgroES promoter; and a heterologous Pf fldA gene encoding a flavodoxin, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 17 or includes a sequence as set forth in SEQ ID NO: 17.

In particular embodiments, an ETC component includes a ferredoxin. In particular embodiments, a ferredoxin includes a small protein that has iron (Fe) and sulfur(S) atoms formed as Fe—S clusters. Fe—S clusters can accept or discharge electrons. Similar to flavodoxin encoded by the fldA gene, ferredoxin encoded by the fer gene is involved in electron transport from pyruvate to the [4Fe-4S] cluster of the iron nitrogenase homodimer. Like flavodoxin, ferredoxin serves as the electron acceptor from pyruvate and the electron donor to nitrogenase. Under anaerobic conditions both ferredoxin and flavodoxin can perform this function, but under aerobic conditions only flavodoxin can be active because ferredoxin must be spatially or temporally separated from oxygen (Poudel et al. J Bacteriol. 2018; 200(10):e00757-17). Without being bound by any one hypothesis, overexpression of ferredoxin may increase the supply of electrons to nitrogenase and thereby increase the capacity of the cells to reduce nitrogen, especially if the cells exist in an anaerobic or microaerobic environment in the rhizosphere. In particular embodiments, a genetically modified Paenibacillus microbe or genetic construct of the disclosure includes a heterologous fer gene encoding a ferredoxin. In particular embodiments, the fer gene (WP_036737310) may be from Pz or Pf. In other embodiments, the fer gene may be from Pm fer, Ss_fer, Nm_fer, Pl_fer, Fp_fer, Sb_fer, Fs_fer, WB17_fer, or Sk_fer. In yet other embodiments, the fer gene may encode a protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to a ferredoxin protein selected from the following group: Pz_fer, Pm_fer, Ss_fer, Nm_fer, Pl_fer, Fp_fer, Sb_fer, Fs_fer, WB17_fer, and Sk_fer, amino acid sequences of which are provided in Tables 12 and 16.

In particular embodiments, a genetically-modified Paenibacillus microbe or genetic construct of the disclosure includes a native fer gene or heterologous Pz fer gene, either of which may be operably linked to a heterologous promoter, such as the inducible PgroES promoter from B. subtilis. In particular embodiments, the genetic construct includes, in 5′ to 3′ order: a heterologous PgroES promoter; and a heterologous Pz fer gene encoding a ferredoxin, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 5 or includes a sequence as set forth in SEQ ID NO: 5.

In particular embodiments, a genetically-modified Paenibacillus microbe or genetic construct of the disclosure includes a native fer gene or heterologous Pz fer gene, either of which may be operably linked to a heterologous promoter, such as the strong constitutive PtrnQ promoter from B. subtilis. An exemplary amino acid sequence of a protein encoded by an exemplary Paenibacillus zanthoxyli fer gene is included as SEQ ID NO: 46. A skilled artisan would also understand that an amino-acid sequence can also be converted to DNA sequence using a codon table, and optionally codon-optimized for the host microbe. An example DNA sequence derived through such a method with respect to SEQ ID NO: 46 is provided as SEQ ID NO: 47. In particular embodiments, the Paenibacillus microbe is Paenibacillus brasilensis or Paenibacillus peoriae or Paenibacillus borealis. In particular embodiments, the genetic construct includes, in 5′ to 3′ order: a heterologous PtrnQ promoter; and a heterologous Pz fer gene encoding a ferredoxin, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 29 or SEQ ID NO: 30 or includes a sequence as set forth in SEQ ID NO: 29 or SEQ ID NO: 30.

In particular embodiments, a genetically-modified Paenibacillus microbe or genetic construct of the disclosure includes a native fer gene or heterologous Paenibacillus spp. fer gene, either of which may be operably linked to a heterologous promoter, such as the strong constitutive PtrnQ promoter from B. subtilis. An exemplary amino acid sequence of a protein encoded by an exemplary Paenibacillus spp. fer gene is included as SEQ ID NO: 48. In particular embodiments the gene encoding such a protein may be used instead of or in addition to the Paenibacillus spp. fer gene. A skilled artisan would also understand that such amino-acid sequence could also be converted to DNA sequence using a codon table. An exemplary DNA sequence derived through such a method with respect to SEQ ID NO: 48 is provided as SEQ ID NO: 49. In particular embodiments, the genetic construct includes, in 5′ to 3′ order: a heterologous PtrnQ promoter; and a heterologous gene encoding a Paenibacillus spp. ferredoxin protein, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 31 or includes a sequence as set forth in SEQ ID NO: 31.

In particular embodiments, a genetically-modified Paenibacillus microbe or genetic construct of the disclosure includes a native fer gene or heterologous gene encoding a Paenibacillus spp. ferredoxin protein, either of which gene may be operably linked to a heterologous promoter, such as the medium strength constitutive PR′ promoter from Bacteriophage lambda. In particular embodiments, the genetic construct includes, in 5′ to 3′ order: a heterologous PR′ promoter; and a heterologous gene encoding a Paenibacillus spp. ferredoxin protein, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 32 or includes a sequence as set forth in SEQ ID NO: 32.

In particular embodiments, a genetically-modified Paenibacillus microbe or genetic construct of the disclosure includes a native fer gene or heterologous gene encoding a Paenibacillus spp. ferredoxin protein, either of which gene may be operably linked to a heterologous promoter, such as the medium strength constitutive P43 promoter from B. subtilis. In particular embodiments, the genetic construct includes, in 5′ to 3′ order: a heterologous P43 promoter; and a heterologous gene encoding a Paenibacillus spp. ferredoxin protein, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 33 or includes a sequence as set forth in SEQ ID NO: 33.

In particular embodiments, a genetically-modified Paenibacillus microbe or genetic construct of the disclosure includes a native fer gene or heterologous Pz fer gene, either of which may be operably linked to a heterologous promoter, such as the constitutive PmreB promoter from B. subtilis. In particular embodiments, the genetic construct includes, in 5′ to 3′ order: a heterologous PmreB promoter; and a heterologous Pz fer gene encoding a ferredoxin, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 18 or includes a sequence as set forth in SEQ ID NO: 18.

### (ii) GENETIC MODIFICATIONS WITH GENES INVOLVED IN NITROGEN FIXATION AND/OR REGULATION

A Paenibacillus microbe of the present disclosure can be genetically modified to increase the amount of nitrogen available to a plant associated with the microbe. In particular embodiments, expressing or overexpressing at least one heterologous gene associated with nitrogen fixation or its regulation increases iron-sulfur cluster assembly of the modified Paenibacillus microbe. In particular embodiments, expressing or overexpressing at least one heterologous gene associated with nitrogen fixation or its regulation increases oxygen protection of the modified Paenibacillus microbe.

In particular embodiments, the genetic modification includes expressing or overexpressing gene products encoded by a suf operon. The SUF system is involved with biogenesis of the electron accepting iron (Fe)-sulfur(S) cluster of the nitrogenase enzyme (Perard and Choudens (2018) Journal of Biological Inorganic Chemistry 23:581-596). In particular embodiments, Fe—S cluster biogenesis includes: a cysteine desulfurase enzyme that uses l-cysteine as a source of sulfur to provide sulfide; iron (ferric or ferrous); a scaffold protein on which the iron and sulfide combine; and chaperones and/or carriers that mediate trafficking and targeting of Fe—S in apo forms of metalloproteins to the mature Fe—S proteins. Without being bound by any one hypothesis, the present disclosure suggests that overexpressing the suf operon may increase availability of the four iron-four sulfur [4Fe-4S] metallo-cluster cofactor to the nitrogenase enzyme, thus increasing the capacity of the cells to accept electrons and therefore to fix nitrogen.

In particular embodiments, a suf operon can include two genes. In particular embodiments, a suf operon can include sufB and sufC. In particular embodiments, a suf operon can include more than 6 genes. In particular embodiments, a suf operon can include sufA, sufB, sufC, sufD, sufS, sufE, and sufU. In particular embodiments, a suf operon can include sufB, sufC, sufD, sufS, and sufU. In particular embodiments, a suf operon can include sufB, sufC, sufS, and sufU, sufB encodes a scaffold protein that helps to assemble an Fe—S cluster, sufSE encodes a cysteine desulfurase, with SufE and SufS forming a SufS4E4 complex, sufC encodes a protein that binds to SufB in a SufB2C2 complex. SufD is a paralog of SufB and appears to play a role in iron acquisition, sufU may encode a scaffold protein for an Fe—S cluster, sufA encodes an A-type carrier protein for an Fe—S cluster. In particular embodiments, the genetically-modified Paenibacillus microbe or genetic construct may include a heterologous sufCDSUB operon including sufB, sufC, sufD, sufS, and sufU genes from Pz. In particular embodiments, the genetically-modified Paenibacillus microbe or genetic construct may include a heterologous sufCBSUB operon including sufB, sufC, sufS, and sufU genes from Pf.

In particular embodiments, a genetically-modified Paenibacillus microbe or genetic construct of the disclosure includes a native sufCDSUB operon including sufB, sufC, sufD, sufS, and sufU genes or a heterologous Pz sufCDSUB operon including sufB, sufC, sufD, sufS, and sufU genes which may be operably linked to a heterologous promoter, such as the medium strength constitutive PsigX promoter from B. subtilis. In particular embodiments, the genetic construct includes, in 5′ to 3′ order: a heterologous PsigX promoter; and a heterologous Pz sufCDSUB operon including sufB, sufC, sufD, sufS, and sufU genes, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 3 or includes a sequence as set forth in SEQ ID NO: 3.

In particular embodiments, a genetically-modified Paenibacillus microbe or genetic construct of the disclosure includes a native sufCDSUB operon including sufB, sufC, sufD, sufS, and sufU genes or a heterologous Pz sufCDSUB operon including sufB, sufC, sufD, sufS, and sufU genes which may be operably linked to a heterologous promoter, such as the strong constitutive PtrnQ promoter from B. subtilis. In particular embodiments, the genetic construct includes, in 5′ to 3′ order: a heterologous PtrnQ promoter; and a heterologous Pz sufCDSUB operon including sufB, sufC, sufD, sufS, and sufU genes, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 7 or includes a sequence as set forth in SEQ ID NO: 7.

In particular embodiments, a genetically-modified Paenibacillus microbe or genetic construct of the disclosure includes a native sufCDSUB operon including sufB, sufC, sufD, sufS, and sufU genes or a heterologous Pf sufCBSUB operon including sufB, sufC, sufS, and sufU genes which may be operably linked to a heterologous promoter, such as the medium strength constitutive PsigX promoter from B. subtilis. In particular embodiments, the genetic construct includes, in 5′ to 3′ order: a heterologous PsigX promoter; and a heterologous Pf sufCBSUB operon including sufB, sufC, sufS, and sufU genes, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 10 or includes a sequence as set forth in SEQ ID NO: 10.

In particular embodiments, a genetically-modified Paenibacillus microbe or genetic construct of the disclosure includes a native sufCDSUB operon including sufB, sufC, sufD, sufS, and sufU genes or a heterologous Pf sufCBSUB operon including sufB, sufC, sufS, and sufU genes which may be operably linked to a heterologous promoter, such as the constitutive PmreB promoter from B. subtilis. In particular embodiments, the genetic construct includes, in 5′ to 3′ order: a heterologous PmreB promoter; and a heterologous Pf sufCBSUB operon including sufB, sufC, sufS, and sufU genes, wherein the genetic construct includes a sequence having at least 90% sequence identity to SEQ ID NO: 15 or includes a sequence as set forth in SEQ ID NO: 15.

In particular embodiments, the genetic modification includes expressing or overexpressing gene products encoded by the DUF269 gene, also known as orf1. DUF269 is a conserved domain of the Orf1 protein encoded by the orf1 gene, and the term DUF269 is used throughout the application to refer to the orf1 gene or Orf1 protein, as applicable. DUF269 (WP_025697219.1) encodes a protein involved in protection of nitrogenase from oxidative damage and has been implicated in nitrogen fixation (Li et al. Int. J. Mol. Sci. 2019; 20 (5): 1145). In particular embodiments, the genetically-modified Paenibacillus microbe or genetic construct includes a heterologous DUF269 gene operably linked to a heterologous promoter. In particular embodiments, the genetically-modified Paenibacillus microbe has an increase in oxygen protection. In particular embodiments, the DUF269 gene may be from Pz, Pf, Paenibacillus durus (Pd), Paenbacillus graminis (Pg), Paenibacillus jilunlii (Pj), Paenibacillus riograndensis (Pr), Paenibacillus sabinae (Psab), or Paenibacillus stellifer (Pstel).

In particular embodiments, the genetically-modified Paenibacillus microbe or genetic construct includes a Pz DUF269 (WP_156934351.1) gene, which may be operably linked to a heterologous promoter, such as the medium strength constitutive PsigX promoter from B. subtilis. In particular embodiments, the genetic construct includes, in 5′ to 3′ order: a heterologous PsigX promoter; and a heterologous Pz DUF269 gene, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 19 or includes a sequence as set forth in SEQ ID NO: 19.

In particular embodiments, the genetically modified Paenibacillus microbe or genetic construct includes a Pz DUF269 gene, which may be operably linked to a heterologous promoter, such as the constitutive PmreB promoter from B. subtilis. In particular embodiments, the genetic construct includes, in 5′ to 3′ order: a heterologous PmreB promoter; and a heterologous Pz DUF269 gene, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 20 or includes a sequence as set forth in SEQ ID NO: 20.

In particular embodiments, a genetically modified Paenibacillus microbe or genetic construct includes a Pd DUF269 (WP_025698314.1) gene, which may be operably linked to a heterologous promoter, such as the medium strength constitutive PR′ promoter from Bacteriophage lambda. In particular embodiments, the genetic construct includes, in 5′ to 3′ order: a heterologous PR′ promoter; and a heterologous Pd DUF269 gene, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 38 or includes a sequence as set forth in SEQ ID NO: 38.

In particular embodiments, a genetically modified Paenibacillus microbe or genetic construct includes a Pf DUF269 (WP_025697219.1) gene, which may be operably linked to a heterologous promoter, such as the medium strength constitutive PR′ promoter from Bacteriophage lambda. In particular embodiments, the genetic construct includes, in 5′ to 3′ order: a heterologous PR′ promoter; and a heterologous Pf DUF269 gene, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 39 or includes a sequence as set forth in SEQ ID NO: 39.

In particular embodiments, a genetically modified Paenibacillus microbe or genetic construct includes a Pg DUF269 (WP_340457365.1) gene, which may be operably linked to a heterologous promoter, such as the medium strength constitutive PR′ promoter from Bacteriophage lambda. In particular embodiments, the genetic construct includes, in 5′ to 3′ order: a heterologous PR′ promoter; and a heterologous Pg DUF269 gene, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 40 or includes a sequence as set forth in SEQ ID NO: 40.

In particular embodiments, a genetically modified Paenibacillus microbe or genetic construct includes a Pj DUF269 (WP_062523379.1) gene, which may be operably linked to a heterologous promoter, such as the medium strength constitutive PR′ promoter from Bacteriophage lambda. In particular embodiments, the genetic construct includes, in 5′ to 3′ order: a heterologous PR′ promoter; and a heterologous Pj DUF269 gene, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 41 or includes a sequence as set forth in SEQ ID NO: 41.

In particular embodiments, a genetically modified Paenibacillus microbe or genetic construct includes a Pr DUF269 (WP_020426658.1) gene, which may be operably linked to a heterologous promoter, such as the medium strength constitutive PR′ promoter from Bacteriophage lambda. In particular embodiments, the genetic construct includes, in 5′ to 3′ order: a heterologous PR′ promoter; and a heterologous Pr DUF269 gene, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 42 or includes a sequence as set forth in SEQ ID NO: 42.

In particular embodiments, a genetically modified Paenibacillus microbe or genetic construct includes a Psab DUF269 (WP_025336123.1) gene, which may be operably linked to a heterologous promoter, such as the medium strength constitutive PR′ promoter from Bacteriophage lambda. In particular embodiments, the genetic construct includes, in 5′ to 3′ order: a heterologous PR′ promoter; and a heterologous Psab DUF269 gene, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 43 or includes a sequence as set forth in SEQ ID NO: 43.

In particular embodiments, a genetically modified Paenibacillus microbe or genetic construct includes a Pstel DUF269 (WP_038698114.1) gene, which may be operably linked to a heterologous promoter, such as the medium strength constitutive PR′ promoter from Bacteriophage lambda. In particular embodiments, the genetic construct includes, in 5′ to 3′ order: a heterologous PR′ promoter; and a heterologous Pstel DUF269 gene, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 44 or includes a sequence as set forth in SEQ ID NO: 44.

In particular embodiments, a genetically modified Paenibacillus microbe or genetic construct includes a Pz DUF269 gene, which may be operably linked to a heterologous promoter, such as the medium strength constitutive PR′ promoter from Bacteriophage lambda. In particular embodiments, the genetic construct includes, in 5′ to 3′ order: a heterologous PR′ promoter; and a heterologous Pz DUF269 gene, wherein the genetic construct includes a sequence having at least 90%, at least 91%, at least 92%, at least 93%, at least 94%, at least 95%, at least 96%, at least 97%, at least 98%, at least 99% sequence identity to SEQ ID NO: 45 or includes a sequence as set forth in SEQ ID NO: 45.

As used herein, the term “genetically modified” or “genetically engineered” refers to (a) the addition of extra genetic material in the form of DNA or RNA into the total genetic material in a cell; or (b) modification of the genome of a cell such that the genome contains insertions, deletions, mutations, and/or rearrangements of the genomic DNA after introduction of extra genetic material as compared to a cell that is not genetically modified. For clarity the term “genetically modified” or “genetically engineered” also includes the removal of DNA from a genome without the insertion of extra genetic material. The term “genetically modified” or “genetically engineered” includes artificial manipulation of a cell to alter the genotype of that cell to modulate physiology or function of that cell, such as expressing a heterologous gene product, deleting endogenous genes, and/or altering regulation or expression of endogenous genes. The extra genetic material can be derived from the same organism as the genome it is inserted into or it can be derived from a different genome or be synthetic. The terms “genetically modified microbe” and “modified microbe” are used interchangeably. The term “genetically modified” or “genetically engineered” also refers to multiple genetic modifications, e.g., 2, 3, 4, 5, 6, 7, 8, 9, 10 or more genetic modifications, for example, a microbe which has a heterologous gene introduced for expression of an electron transport chain component, and another heterologous gene introduced for expression of a gene product associated with nitrogen fixation or nitrogen regulation.

The term “microbe” refers to an individual microorganism or a class of microorganisms. In particular embodiments, a microbe can refer to a single microbial cell (e.g., a Paenibacillus microbe includes a single Paenibacillus cell). In particular embodiments, a microbe can refer to a microbe of a taxa, a class, a genus, a species, etc. (e.g., a Paenibacillus microbe includes a microorganism belonging to the Paenibacillus genus).

The term “heterologous” refers to a molecule (e.g., nucleic acid, gene, RNA, protein) that originates outside a microbe and is introduced into a microbe by genetic engineering. In particular embodiments, a heterologous molecule can include sequences that are not native to a microbe to which the heterologous molecule is introduced; the heterologous molecule is synthesized outside the microbe and introduced into the microbe. For example, the disclosure includes a genetically modified Paenibacillus microbe including at least one heterologous gene encoding an electron transport chain component. The at least one heterologous gene encoding an electron transport chain component has a sequence that is not found naturally in (not native to) the Paenibacillus microbe. In particular embodiments, a heterologous molecule can include sequences that are native to a microbe to which the heterologous molecule is introduced, but the heterologous molecule is synthesized outside the microbe and introduced into the microbe. For example, the disclosure includes a genetically modified Pb microbe including at least one heterologous gene encoding Pb fer. The fer gene is native to Pb but is synthesized and introduced into Pb as part of an expression cassette such that the expression of the introduced Pb fer is driven by a heterologous promoter.

The term “endogenous” refers to a molecule (e.g., nucleic acid, gene, RNA, protein) that is naturally occurring or naturally produced in a given microbe. For example, genes or proteins found naturally in a Paenibacillus microbe are genes or proteins that are endogenous to the Paenibacillus microbe. The term “native” can be used interchangeably with “endogenous”. In particular embodiments, the term “endogenous” may refer to a wild-type version of a molecule in a given microbe.

In particular embodiments, the term “gene” refers to a nucleic acid sequence (used interchangeably with polynucleotide or nucleotide sequence) that encodes, e.g., a protein associated with the electron transport chain, nitrogen fixation, or regulation of nitrogen fixation, as described herein. This definition includes various sequence polymorphisms, mutations, and/or sequence variants wherein such alterations do not substantially affect the function of the encoded protein. The nucleic acid sequences can include both the full-length nucleic acid sequences as well as non-full-length sequences derived from a full-length protein coding sequence. The sequences can also include degenerate codons of the native sequence or sequences that may be introduced to provide codon preference in a specific microbe. In particular embodiments, the term “gene” may include not only coding sequences but also regulatory regions such as promoters, enhancers, 5′ UTR, 3′UTR, termination regions, and non-coding regions. Gene sequences encoding a molecule can be DNA or RNA that directs the expression of the molecule. These nucleic acid sequences may be a DNA strand sequence that is transcribed into RNA or an RNA sequence that is translated into protein. An essential gene is an endogenous (e.g., endogenous to a microbe) or heterologous gene (e.g., a selectable marker or gene of interest) that produces a polypeptide (e.g., an essential protein) that is necessary for the growth and/or viability of a microbe.

“Encoding” refers to the property of specific sequences of nucleotides in a gene, such as a complementary DNA (cDNA), or a messenger RNA (mRNA), to serve as templates for synthesis of other macromolecules such as a defined sequence of amino acids or a functional polynucleotide (e.g., siRNA). In particular embodiments, a gene encodes or codes for a protein if the gene is transcribed into mRNA and translation of the mRNA produces the protein in a cell or other biological system. A “gene sequence encoding a protein” includes all nucleotide sequences that are degenerate versions of each other and that code for the same amino acid sequence or amino acid sequences of substantially similar form and function.

A “gene deletion” or “gene knockout” refers to a combination of genetic techniques that can render a specific gene inoperable or inactive. In particular embodiments, a gene deletion reduces or eliminates expression of a polypeptide encoded by the gene. In particular embodiments, the expression of the gene is substantially reduced or eliminated. Substantially reduced means that the expression of a gene is reduced by at least 80%, at least 90%, at least 95%, or at least 98% when compared to an endogenous level of expression of the gene. Expression of a gene can be determined by a suitable technique (e.g., by measuring transcript or expressed protein levels). Any suitable technique can be used to generate a gene deletion in a microbe. In particular embodiments, a gene deletion in Paenibacillus is mediated by a gene editing system such as Clustered Regularly Interspaced Short Palindromic Repeats/CRISPR-associated (CRISPR/Cas), transcription activator-like effector nucleases (TALENs), zinc-finger nucleases (ZFNs), and meganucleases. In particular embodiments, a gene is deleted by disabling an endogenous promoter, operon or regulatory element that is essential for transcription or translation of the gene. In particular embodiments, a gene is deleted by introducing one or more mutations that disable the function of a protein encoded by the gene. In particular embodiments, a gene is partially or completely removed from the genome of a microbe. In particular embodiments, an endogenous gene is deleted by replacing the gene with a different gene or a selectable marker (e.g., antibiotic selectable marker, auxotrophic selectable marker).

The terms “peptide,” “oligopeptide,” “polypeptide,” “polyprotein,” and “protein” are used interchangeably herein and refer to a polymeric form of amino acids of any length, which can include coded and non-coded amino acids, chemically or biochemically modified or derivatized amino acids, and polypeptides having modified peptide backbones.

The term “recombinant” refers to a particular DNA or RNA sequence that is the product of various combinations of cloning, restriction, and/or ligation steps resulting in a construct having a structural coding or non-coding sequence distinguishable from homologous sequences found in natural systems. Generally, DNA sequences encoding the structural coding sequence can be assembled from cDNA fragments and short oligonucleotide linkers, or from a series of oligonucleotides, to provide a synthetic gene which is capable of being expressed in a recombinant transcriptional unit. Such sequences can be provided in the form of an open reading frame uninterrupted by internal non-translated sequences, or introns. Genomic DNA comprising the relevant sequences could also be used. Sequences of non-translated DNA may be present 5′ or 3′ from the open reading frame, where such sequences do not interfere with manipulation or expression of the coding regions. In particular embodiments, the term “recombinant” polynucleotide or nucleic acid refers to one which is not naturally occurring or is made by the artificial combination of two otherwise separated segments of sequence. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions.

A “genetic construct” includes a recombinant nucleic acid, generally recombinant DNA, which has been generated for the purpose of the expression of a specific nucleotide sequence(s) or is to be used in the construction of other recombinant nucleotide sequences. In particular embodiments, the term genetic construct includes plasmids and vectors. In particular embodiments, a genetic construct can be circular or linear. Genetic constructs can include, for example, an origin of replication, a multicloning site, and/or a selectable marker. In particular embodiments, a genetic construct includes nucleic acid to enable deletion of a gene in an Paenibacillus microbe. In particular embodiments, a genetic construct includes an expression construct. An expression construct typically includes an expression cassette. In particular embodiments, an expression construct of the disclosure includes: (a) a heterologous promoter; and (b) at least one heterologous gene selected from: pyruvate ferredoxin-oxidoreductase porCDAB E.C. 1.2.7.1; flavodoxin fldA; sufB, sufC, sufD, sufS, and/or sufU genes of a sufCDSUB operon; sufB, sufC, sufS, and/or sufU genes of a sufCBSUB operon; ferredoxin fer, DUF269 or a combination thereof. In particular embodiments, an expression construct of the disclosure can further include a selectable marker.

Similarly, a “recombinant polypeptide” refers to a polypeptide or polyprotein which is not naturally occurring or is made by the artificial combination of two otherwise separated segments of amino acid sequences. This artificial combination may be accomplished by standard techniques of recombinant DNA technology, i.e., a recombinant polypeptide may be encoded by a recombinant polynucleotide. Thus, a recombinant polypeptide is an amino acid sequence encoded by all or a portion of a recombinant polynucleotide.

A genetic construct of the disclosure can include a gene encoding a selectable marker and/or counter-selectable marker. In particular embodiments, cells expressing a selectable marker can grow in the presence of a selective agent or under a selective growth condition. Examples of selectable markers include antibiotic resistance markers (e.g., chloramphenicol resistance, erythromycin resistance, ampicillin resistance, carbenicillin resistance, kanamycin resistance, spectinomycin resistance, streptomycin resistance, tetracycline resistance, bleomycin resistance, and polymyxin B resistance), markers that complement an essential gene (e.g., diaminopimelic acid auxotrophy (dapD), thymidine auxotrophy (thyA), proline auxotrophy (proBA), glycine auxotrophy (glyA), carbon source auxotrophy (TpiA)), chemical resistance (e.g., tellurite resistance, Fabl for triclosan resistance, bialaphos herbicide resistance, mercury resistance, arsenic resistance), and visual markers (e.g., green fluorescent protein (GFP), luciferase, β-galactosidase (lacZ)). In particular embodiments, a genetic construct of the disclosure includes a chloramphenicol acetyl transferase resistance gene (CAT) operably linked to a chloramphenicol responsive promoter (PCAT) and terminator (TCAT) from the Staphylococcus plasmid pC194 (Horinouchi and Weisblum. J Bacteriol. 1982; 150 (2): 815-825). In particular embodiments, cells may be positively selected that have lost expression of a counter-selectable marker (i.e. cells expressing a counter-selectable marker are selected against). Examples of genes encoding counter-selectable markers include: sacB (gene encoding levansucrase that converts sucrose to levans, which is harmful to bacteria); rpsL (strA) (encodes the ribosomal subunit protein (S12) target of streptomycin); tetAR (confers sensitivity to lipophilic compounds such as fusaric and quinalic acids); pheS (encodes the a subunits of Phe-tRNA synthetase, which renders bacteria sensitive to p-chlorophenylalanine, a phenylalanine analog); thyA (encodes thymidilate synthetase, which confers sensitivity to trimethoprim and related compounds); lacY (encodes lactose permease, which renders bacteria sensitive to t-o-nitrophenyl-β-D-galactopyranoside); gata-1 (encodes a zinc finger DNA-binding protein which inhibits the initiation of bacterial replication); and ccdB (encodes a cell-killing protein which is a potent poison of bacterial gyrase).

The term “expression cassette” includes a polynucleotide construct that is generated recombinantly or synthetically and includes regulatory sequences operably linked to a selected polynucleotide to facilitate expression of the selected polynucleotide in a microbe. For example, the regulatory sequences can facilitate transcription of the selected polynucleotide in a microbe, or transcription and translation of the selected polynucleotide in a microbe. In particular embodiments, the expression cassette includes an operon, a cluster of genes under the control of a common promoter. Therefore, genes within an operon are expressed together. In particular embodiments, the expression cassette is introduced as part of a genetic construct into a Paenibacillus microbe, and the expression cassette is subsequently integrated into the genome of the Paenibacillus microbe. A heterologous expression cassette can be integrated into the genome of the Paenibacillus microbe by any method known to one of skill in the art, including by gene editing systems such as CRISPR/Cas, site-specific recombination, and transposon-mediated gene transposition.

Site-specific recombination uses site-specific recombinases, or phase integrases, that catalyze recombination between two specific sequences of DNA. Site-specific recombinases that can be used include tyrosine recombinases Flp and Cre that catalyze reversible recombination; and serine integrases that catalyze unidirectional DNA recombination between distinct attP and attB sites to generate new attL and attR sites.

Transposon-mediated gene transposition takes advantage of a discrete nucleic acid segment's (transposon) ability to move from one location in a genome to another location by a ‘cut and paste’ mechanism. The mobilization of the transposon is enabled by a transposase, which binds to terminal inverted repeats (TIR) flanking the transposon. Thus, a heterologous nucleic acid from a donor plasmid can be integrated in a genome by including TIRs (transposase binding sites) at the ends of the heterologous nucleic acid and expressing a transposase to excise the heterologous nucleic acid from the donor plasmid and integrate the heterologous nucleic acid at a genomic site.

In particular embodiments, transposon-mediated integration of a heterologous expression cassette in the genome of a Paenibacillus microbe includes: (1) a plasmid that is temperature sensitive for replication and expresses a transposase and a first selectable marker; and (2) a donor plasmid that is temperature sensitive for replication and includes a heterologous expression cassette to be integrated and a second selectable marker. In particular embodiments, the transposase plasmid is a temperature sensitive tnpA plasmid constitutively expressing an erythromycin resistance marker ermC from the pE194 plasmid of Staphylococcus aureus and the Himar1 mariner transposase gene from Haematobia irritans, and the donor plasmid includes a Himar1 transposon cassette including a heterologous expression cassette to be integrated, the PCAT chloramphenicol responsive promoter from the Staphylococcus plasmid pC194, the CAT chloramphenicol acetyl transferase resistance gene from pC194, and the TCAT terminator from pC194. The transposon cassette is flanked by inverted mariner terminal repeat sequences from Haematobia irritans. Growth of transformed Paenibacillus at a permissive temperature of 30° C. and growth on appropriate antibiotic allows selection for cells that have been transformed with the plasmids, while growth at a subsequent non-permissive temperature of 37° C. selects for cells that have chromosomally integrated the transposon cassette and against cells that retained replicating plasmids. The mariner transposon inserts into a wide variety of host genomes in pseudo-random fashion at thymine-adenine (TA) dinucleotide DNA sites.

The term “overexpression” refers to a greater expression level of a gene encoding a given polypeptide in a genetically modified microbe as compared to expression in a wild-type microbe at any developmental or temporal stage for the gene. In particular embodiments, overexpression can occur when the gene is under the control of a strong promoter (e.g., the PsigX promoter). Overexpression may also occur under the control of an inducible promoter. In particular embodiments, overexpression may occur in a microbe where endogenous expression of a given polypeptide normally occurs, but such normal expression is at a lower level. In particular embodiments, overexpression may also occur in a microbe lacking expression of a given polypeptide. Overexpression thus results in a greater than normal production or “overproduction” of a given polypeptide in a microbe. Increased activity of a protein can result from overexpression or the modification of a peptide or a polypeptide such that it causes the peptide or polypeptide to have a higher activity. For example, in the case where a peptide is an enzyme the enzyme can have an increased catalytic turnover rate.

In particular embodiments, a genetically modified microbe includes a nucleic acid (e.g., a gene) where expression of the gene is regulated by a promoter and/or regulatory elements. A promoter and/or regulatory elements are often introduced at a suitable location relative to a gene of interest. For example, a promoter (e.g., an inducible promoter) is often placed 5′ of a transcription start site of a gene of interest. In particular embodiments, a nucleic acid includes a promoter and/or regulatory elements necessary to drive the expression of a gene (e.g., a heterologous gene or an endogenous gene). A promoter can be an endogenous promoter, a heterologous promoter, or a combination thereof. In particular embodiments, a promoter is a constitutive promoter. In particular embodiments, a heterologous constitutive promoter includes: P43, PR′, PsigX, PtrnQ, PclpE, PywbO, PypuA, PywrK, PsigV, PydaH, PyjoB, PyqeZ, PyacL, PyceG, PyrhK, PradA, PywaC, PmurB, PfabHA, PhtrB, PaprE, PycbR, PhtrA, PyrhH, PpbpE, PybfP, PywnJ, PytpA, PoatA, PmreBH, PyeaA, PybfO, PmurF, PyuaF, PypbG, PbcrC, PmetA, PylxX, PybgB, Pyusl, PcsbB, PyjbC, PclpC, PythP, PhtpG, Pyxil, PtilS, PspoOM, PdivlB, PypuD, PcssR, PyxzE, PdnaJ, PybfQ, PxpaC, PyngC, PyvIA, PpssA, PyoaF, PminC, PdltE, ProdA, PpspA, PclpP, PdivlC, PydjO, PydbS, PysdB, PyoaG, Pddl, PfosB, Pabh, Pspa, PyceE, PyknW, or PmreB (Song 2016 PLoS ONE 11(7): e0158447). In particular embodiments, the heterologous constitutive promoter is derived from Paenibacillus genus. In particular embodiments, the heterologous constitutive promoter is derived from Paenibacillus genus or Bacillus subtilis.

In particular embodiments, a microbe is genetically engineered to include a gene under the control of an inducible promoter. An inducible promoter is often a nucleic acid sequence that directs the conditional expression of a gene. An inducible promoter can be an endogenous promoter, a heterologous promoter, or a combination thereof. An inducible promoter can include an operon system. In particular embodiments, an inducible promoter requires the presence of a certain compound, nutrient, amino acid, sugar, peptide, protein or condition (e.g., light, oxygen, heat, cold) to induce gene activity (e.g., transcription). In particular embodiments, an inducible promoter includes one or more repressor elements. In particular embodiments, an inducible promoter including a repressor element requires the absence of a certain compound, nutrient, amino acid, sugar, peptide, protein or condition to induce gene activity (e.g., transcription). Any suitable inducible promoter, system, or operon can be used to regulate the expression of a gene. Non-limiting examples of inducible promoters include temperature inducible promoters (e.g., heat inducible PgroES promoter, heat inducible phage lambda pL promoter, heat inducible phage lambda pR promoter, cold inducible cspA promoter), lactose regulated systems (e.g., lactose operon systems), sugar regulated systems, metal regulated systems, steroid regulated systems, alcohol regulated systems, IPTG inducible systems (e.g., pLac promoter), arabinose regulated systems (e.g., arabinose operon systems, pBad promoter), synthetic amino acid regulated systems (e.g., see Rovner et al. (2015) Nature 518 (7537): 89-93), fructose repressors, a tac promoter/operator (pTac), tryptophan promoters (e.g., Ptrp, induced by tryptophan depletion or by addition of β-indoleacrylic acid), alkaline phosphatase promoters (e.g., PhoA promoter induced by phosphate limitation), recA promoters (e.g., recA promoter induced by UV light), proU promoters (e.g., osmotically inducible proU promoter), cst promoters (e.g., cst promoter inducible by carbon starvation), tetA promoters (e.g., tetracycline inducible tetA promoter), cadA and cadR promoters (e.g., PcadA and PcadR induced by cadmium), nar promoters (e.g., nar promoter induced by oxygen), or combinations thereof.

In particular embodiments, expression of a gene can be controlled in additional ways known to one of skill in the art including modifying: gene copy number, number of copies of transcription factors binding the promoter operably linked to the gene; transcription factor binding to the gene promoter; RNA polymerase binding affinity for the gene promoter; ribosome binding affinity for the RBS; mRNA decay rate; and protein decay rate (Brewster et al. (2012) PLOS Comput Biol 8(12): e1002811). In particular embodiments, a promoter such as T7 can be regulated using a system with a temperature sensitive intein inserted in the protein sequence of T7 RNA polymerase (Korvin and Yadav (2018) Molecular Systems Design and Engineering 3 (3): 550-559). The polymerase is only active and able to drive gene expression when the intein is spliced out at the appropriate temperature.

The term “operably linked” refers to polynucleotide sequences or amino acid sequences placed into a functional relationship with one another. For instance, a promoter or enhancer is operably linked to a coding sequence if it regulates, or contributes to the modulation of, the transcription of the coding or non-coding sequence. In particular embodiments, regulatory sequences operably linked to a coding sequence are typically contiguous to the coding sequence. However, enhancers can function when separated from a promoter by up to several kilobases or more. Accordingly, some polynucleotide elements may be operably linked but not contiguous. In particular embodiments, a heterologous promoter or heterologous regulatory elements include promoters and regulatory elements that are not normally associated with a particular nucleic acid in nature.

A termination region may be provided by the naturally occurring or endogenous transcriptional termination region of the polynucleotide sequence encoding a protein of the disclosure. Alternatively, the termination region may be derived from a different source. For the most part, the source of the termination region is generally not considered to be critical to the expression of a recombinant protein and a wide variety of termination regions can be employed without adversely affecting expression.

In particular embodiments, a genetic construct of the disclosure can be propagated in vitro in a host cell suitable for replication of the genetic construct. Host cells can include bacterial cells, mammalian cells, yeast cells, insect cells, or plant cells. In particular embodiments, the host cell is a bacterium, e.g., E. coli. The selection of an appropriate host is deemed to be within the scope of those skilled in the art. A recombinant host cell includes a host cell into which has been introduced a genetic construct.

In particular embodiments, a genetic construct is introduced into a microbe using a suitable technique. In particular embodiments, a microbe is transformed with a genetic construct by a suitable technique. Non-limiting examples of suitable techniques for introducing a nucleic acid into a microbe include conjugation, electroporation, transduction (e.g., injection of a nucleic acid by a bacteriophage), microinjection, by inducing competence (e.g., by addition of alkali cations, cesium, lithium, polyethylene glycol or by osmotic shock), or combinations thereof.

As would be appreciated by one of ordinary skill in the art, the genetic engineering strategies described herein to increase atmospheric nitrogen fixation in a microorganism may also be applied to one or more of the following microorganisms: Proteobacteria (e.g., Pseudomonas, Enterobacter, Slenotrophomonas, Burkholderia, Rhizobium, Herbaspirillum, Pantoea, Serratia, Rahnella, Azospirillum, Azorhizobium, Azotobacter, Duganella, Delftia, Bradyrhizobiun, Sinorhizobium and Halomonas), Firmicutes (e.g., Bacillus, Paenibacillus, Lactobacillus, Mycoplasma, and Acetobacterium), and Actinobacteria (such as Streptomyces, Rhodacoccus, Microbacterium, and Curtobacterium). In some embodiments, a microorganism from one or more of the following taxa may be genetically modified as described herein: Achromobacter, Acidithiobacillus, Acidovorax, Acinetobacter, Actinoplanes, Adlercreutzia, Aerococcus, Aeromonas, Afipia, Agromyces, Ancylobacter, Arthrobacter, Atopostipes, Azospirillum, Bacillus, Bdellovibrio, Beijerinckia, Bosea, Bradyrhizobium, Brevibacillus, Brevundimonas, Burkholderia, Candidatus, Caulobacter, Cellulomonas, Cellvibrio, Chryseobacterium, Citrobacter, Clostridium, Coraliomargarita, Corynebacterium, Cupriavidus, Curtobacterium, Curvibacter, Deinococcus, Delftia, Desemzia, Devosia, Dokdonella, Dyella, Enhydrobacter, Enterobacter, Enterococcus, Erwinia, Escherichia, Escherichia/Shigella, Exiguobacterium, Ferroglobus, Filimonas, Finegoldia, Flavisolibacter, Flavobacterium, Frigoribacterium, Gluconacetobacter, Hafnia, Halobaculum, Halomonas, Halosimplex, Herbaspirillum, Hymenobacter, Klebsiella, Kocuria, Kosakonia, Lactobacillus, Leclercia, Lentzea, Luteibacter, Luteimonas, Massilia, Mesorhizobium, Methylobacterium, Microbacterium, Micrococcus, Microvirga, Mycobacterium, Neisseria, Nocardia, Oceanibaculum, Ochrobactrum, Okibacterium, Oligotropha, Oxalobacter, Paenibacillus, Pantoea, Pelomonas, Perlucidibaca, Plantibacter, Polynucleobacter, Propionibacterium, Propionibacterium, Pseudoclavibacter, Pseudomonas, Pseudonocardia, Pseudoxanthomonas, Psychrobacter, Rahnella, Ralstonia, Rheinheimera, Rhizobium, Rhodococcus, Rhodopseudomonas, Roseateles, Ruminococcus, Sebaldella, Sediminibacterium, Serratia, Shigella, Shinella, Sinorhizobium, Sinosporangium, Sphingobacterium, Sphingomonas, Sphingopyxis, Sphingosinicella, Staphylococcus, Stenotrophomonas, Streptococcus, Streptomyces, Stygiolobus, Sulfurisphaera, Tatumella, Tepidimonas, Thermomonas, Thiobacillus, Variovorax, Xanthomonas, and Zimmermannella.

In particular embodiments, a genetically modified Paenibacillus microbe of the present disclosure includes P. abyssi, P. aestuarii, P. agarexedens, P. agaridevorans, P. alba, P. algeriensis, P. alginolyticus, P. algorifonticola, P. alkaliterrae, P. alvei, P. amylolyticus, P. anaericanus, P. antarcticus, P. antibioticophila, P. apiarius, P. assamensis, P. azoreducens, P. barcinonensis, P. barengoltzii, P. beijingensis, P. borealis, P. bovis, P. brasilensis, P. brassicae, P. camelliae, P. camerounensis, P. campinasensis, P. castaneae, P. catalpa, P. cathormii, P. cavernae, P. cellulositrophicus, P. cellulosilyticus, P. chartarius, P. chibensis, P. chinensis, P. chinjuensis, P. chitinolyticus, P. chondroitinus, P. chungangensis, P. cineris, P. contaminans, P. cookii, P. cucumis, P. curdlanotyticus, P. daejeonensis, P. dakarensis, P. darwinianus, P. dauci, P. dendritiformis, P. dongdonensis, P. donghaensis, P. doosanensis, P. durus, P. edaphicus, P. ehimensis, P. elgii, P. endophyticus, P. enshidis, P. etheri, P. faecis, P. favisporus, P. ferrarius, P. filicis, P. fonticola, P. forsythiae, P. frigoriresistens, P. gansuensis, P. gelatinilyticus, P. ginsengarvi, P. ginsengihumi, P. ginsengiterrae, P. glacialis, P. glucanolyticus, P. glycanilyticus, P. gorillae, P. graminis, P. granivorans, P. guangzhouensis, P. harenae, P. hemerocallicola, P. herberti, P. hodogayensis, P. hongkongensis, P. hordei, P. humi, P. humicus, P. hunanensis, P. ihumii, P. illinoisensis, P. insulae, P. jamilae, P. jilunlii, P. kobensis, P. koleovorans, P. konsidensis, P. koreensis, P. kribbensis, P. kyungheensis, P. lactis, P. larvae, P. lautus, P. lemnae, P. lentimorbus, P. lentus, P. lupini, P. macerans, P. macquariensis, P. marchantiophytorum, P. marinisediminis, P. marinum, P. massiliensis, P. medicaginis, P. mendelii, P. montaniterrae, P. motobuensis, P. mucilaginosus, P. nanensis, P. naphthalenovorans, P. nasutitermitis, P. nematophilus, P. nicotianae, P. oceanisediminis, P. odorifer, P. oenotherae, P. pabuli, P. panacisoli, P. panaciterrae, P. pasadenensis, P. pectinilyticus, P. peoriae, P. periandrae, P. phoenicis, P. phyllosphaerae, P. physcomitrellae, P. pinesoli, P. pini, P. pinihumi, P. pocheonensis, P. polymyxa, P. popilliae, P. populi, P. profundus, P. prosopidis, P. provencensis, P. pueri, P. puldeungensis, P. purispatii, P. qingshengii, P. quercus, P. radicis, P. relictisesami, P. residui, P. rhizoryzae, P. rhizosphaerae, P. rigui, P. riograndensis, P. ripae, P. sabinae, P. sacheonensis, P. sanguinis, P. sediminis, P. selenii, P. selenitireducens, P. senegalensis, P. septentrionalis, P. sepulcri, P. shenyangensis, P. shirakamiensis, P. siamensis, P. soli, P. sonchi, P. sophorae, P. sputi, P. stellifer, P. susongensis, P. swuensis, P. taichungensis, P. taihuensis, P. taiwanensis, P. taohuashanense, P. tarimensis, P. telluris, P. terrae, P. terreus, P. terrigena, P. tezpurensis, P. thailandensis, P. thermoaerophilus, P. thermophilus, P. thiaminolyticus, P. tianmuensis, P. tibetensis, P. timonensis, P. tundrae, P. turicensis, P. tylopili, P. typhae, P. tyraminigenes, P. uliginis, P. urinalis, P. validus, P. vini, P. vortex, P. vulneris, P. wenxiniae, P. wooponensis, P. woosongensis, P. wulumuqiensis, P. wynnii, P. xanthinilyticus, P. xinjiangensis, P. xylanexedens, P. xylaniclasticus, P. xylanisolvens, P. xylanilyticus, P. yonginensis, P. yunnanensis, P. zanthoxyli, or P. zeae.

### (iii) ASSAYS

In vitro assays can be performed to assess whether a genetically modified Paenibacillus microbe of the disclosure has increased nitrogen fixation as compared to a control Paenibacillus microbe. In particular embodiments, a control Paenibacillus microbe includes a non-genetically-modified Paenibacillus microbe. In particular embodiments, a non-genetically-modified Paenibacillus microbe includes a parent Paenibacillus microbe that is used as the starting microbe for the genetic modifications of a test microbe. In particular embodiments, a parent Paenibacillus microbe is considered to be a wild-type Paenibacillus microbe. For example, a control Paenibacillus microbe includes parent Pb strain PB172 used in the Examples. In particular embodiments, a control Paenibacillus microbe includes a parent Paenibacillus microbe that is used as the starting microbe for the genetic modifications of a test microbe but includes an integration of an unrelated genetic construct at the same location in the genome as the genetically modified test microbe. An unrelated genetic construct can include: a heterologous gene unrelated to electron transport chain, nitrogen fixation, and regulation of nitrogen fixation; only includes a selectable marker expression cassette; and/or serves to disrupt the same location in the genome as the genetically modified test microbe but does not express the same heterologous genes as the genetically modified test microbe. In particular embodiments, a control Paenibacillus microbe includes a Paenibacillus microbe with increased nitrogen fixation as compared to a non-genetically modified Paenibacillus microbe. In particular embodiments, a negative control Paenibacillus microbe includes a Paenibacillus microbe that is the same as a non-genetically-modified Paenibacillus microbe but has a deletion of a nif gene that renders the microbe unable to fix nitrogen. In particular embodiments, a negative control Paenibacillus microbe includes a Paenibacillus microbe that is the same as a non-genetically-modified Paenibacillus microbe but expresses a heterologous gene unrelated to electron transport chain, nitrogen fixation, and regulation of nitrogen fixation. An example of a heterologous gene unrelated to electron transport chain, nitrogen fixation, and regulation of nitrogen fixation includes a detectable marker such as a fluorescent protein (e.g., mRuby, green fluorescent protein).

In particular embodiments, nitrogenase activity can be measured in a genetically modified Paenibacillus microbe of the disclosure by an acetylene reduction assay (ARA) (Stewart et al. (1967) Science 158(3800):536-536; David et al. (1980) Appl Environ Microbiol 39(5):1078-1080). An ARA can provide a highly sensitive and inexpensive method to quantify nitrogenase enzyme activity in N₂ fixing samples indirectly by measuring the ability of the samples to reduce acetylene gas (C₂H₂) to ethylene gas (C₂H₄). The gases can be quantified by gas chromatography. In particular embodiments, nitrogenase activity of a genetically modified Paenibacillus microbe is measured as an inhibitory concentration 90 (IC90), which is the concentration of ammonium that decreases the potential ethylene production by 90% in an ARA. In particular embodiments, nitrogenase activity of a genetically modified Paenibacillus microbe is expressed as IC90. In particular embodiments, the IC90 of a genetically modified Paenibacillus microbe of the disclosure is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to the IC90 of an modified Paenibacillus microbe comprising an expression cassette with mRuby as measured by ARA (see further description herein).

In particular embodiments, nitrogenase activity can be measured indirectly in a genetically modified Paenibacillus microbe of the disclosure by an ¹⁵N dilution assay. An ¹⁵N dilution assay measures incorporation of fixed nitrogen into amino acids. Cells are initially grown on rich media containing a ¹⁵N enriched nitrogen source. As the cells grow in this media, they will fix and incorporate ¹⁵N in their biomass. After reaching a target biomass, cells are transferred to a nitrogen free media under regular atmosphere. As the cells are growing, they will incorporate atmospheric ¹⁴N into their biomass, thus diluting the initial pool of ¹⁵N. Arginine is used as a marker and the ratio of ¹⁴NArg/¹⁵NArg is reported. In particular embodiments, nitrogenase activity of a genetically modified Paenibacillus microbe is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to nitrogenase activity of an unmodified Paenibacillus microbe as measured by an ¹⁵N dilution assay.

In particular embodiments, nitrogenase activity can be measured directly in a genetically modified Paenibacillus microbe of the disclosure by an ¹⁵N₂ fixing assay. In particular embodiments, an ¹⁵N₂ fixing assay includes adding ¹⁵⁻¹⁵N₂ gas directly as a bubble to water. Rates of N₂ fixation can then be calculated from the incorporation of ¹⁵⁻¹⁵N₂ gas into biomass (Montoya et al. (1996) Appl. Environ. Microbiol. 62:986-993). In particular embodiments, an ¹⁵N₂ fixing assay is performed when the rates of nitrogen fixation are high and high sensitivity is not necessary. In particular embodiments, nitrogenase activity of a genetically modified Paenibacillus microbe is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to nitrogenase activity of an unmodified Paenibacillus microbe as measured by an ¹⁵N₂ fixing assay.

In particular embodiments, nitrogenase activity can be measured in a genetically modified Paenibacillus microbe of the disclosure by an ammonia biosensor assay. This assay uses an “indicator” strain that constitutively expresses a detectable marker (e.g., a green fluorescent protein (GFP) and is modified to be NH₃ auxotrophic). The ammonia produced by a candidate genetically modified Paenibacillus microbe diffuses through the membrane into the broth and is assimilated by the indicator strain. As the indicator strain grows, the level of the detectable marker (e.g., GFP fluorescence) increases proportionally. The amount or level of the detectable marker of the “indicator strain” allows measurement of ammonia produced by the candidate genetically modified Paenibacillus microbe and thus reflects how well the candidate genetically modified Paenibacillus microbe can fix nitrogen.

In particular embodiments, nitrogenase activity is measured indirectly by determining nitrogen content use elemental analysis. For example, nitrogen content can be measured in a genetically modified Paenibacillus microbe of the disclosure by a carbon, hydrogen, and nitrogen analyzer (CHN analyzer), which includes flash combustion of a sample to cause an instantaneous oxidization into simple compounds which are then detected with thermal conductivity detection or infrared spectroscopy. In particular embodiments, the CHN analyzer can be used when rates of nitrogen fixation are very high. In particular embodiments, nitrogenase activity of a genetically modified Paenibacillus microbe is increased 1.1×, 1.2×, 1.3×, 1.4×, 1.5×, 1.6×, 1.7×, 1.8×, 1.9×, 2.0×, 2.1×, 2.2×, 2.3×, 2.4×, 2.5×, 2.6×, 2.7×, 2.8×, 2.9×, 3.0×, 3.5×, 4×, 4.5×, 5×, 6×, 7×, 8×, 9×, 10×, 15×, 20×, 25×, 30×, 35×, 40×, 50×, or more as compared to nitrogenase activity of an unmodified Paenibacillus microbe, or another genetically modified Paenibacillus, as measured by a CHN analyzer.

Measurements of plant agronomic characteristics can be measured to assess the effects of association of a genetically modified Paenibacillus microbe of the disclosure with an agricultural plant. Nitrogen (N) is an essential element for plant growth. N fertilizer application significantly enhances plant growth, biomass accumulation, and grain yield in plants. In some embodiments, final grain yield is highly correlated to early plant growth including biomass accumulation. Reductions in these metrics are known to correlate to applied nitrogen conditions in a dose dependent manner under greenhouse and field conditions. In particular embodiments, early plant responses to N application applied in a greenhouse translates to plant growth and yield responses to N fertilizer application responses in the field.

In some embodiments, in planta assays can be used to screen and characterize engineered microbes in the greenhouse. Plant seedlings inoculated with a genetically engineered microbe of the disclosure can be grown with fertilizer solution with different amounts of N. For example, inoculated plants can be fertilized using solution containing 0 ppm N, 5 ppm N, 10 ppm N, 15 ppm N, 20 ppm N, 25 ppm N, 100 ppm and grown in appropriate containers for a period of time (e.g., 2 weeks, 4 weeks, 6 weeks, 8 weeks, etc.). To estimate the amount of N supplemented by a genetically engineered microbe of the disclosure when it is applied to a plant, plant characteristics including plant height, leaf number, leaf greenness, plant biomass (e.g., shoot fresh weight and/or shoot dry weight) can be measured and compared to a control. In particular embodiments, a control includes a plant or a population of plants of the same genus and species that has not been inoculated with the genetically engineered microbe. In particular embodiments, a control includes a plant or a population of plants of the same genus and species that has been inoculated with a corresponding microbe (i.e. microbe of the same genus and species) that has not been genetically engineered to increase nitrogen fixation. In particular embodiments, a control includes a wild-type plant or a population of wild-type plants of the same genus and species. Plant characteristics can be evaluated weekly or at a particular time after planting. In particular embodiments, the plant is corn.

In particular embodiments, growth stage can be assessed as leaf number, which is recorded weekly for each plant by counting fully expanded leaves. In particular embodiments, plant height can be measured weekly from soil surface to the tallest extended leaf tip (manually straightened up).

In particular embodiments, leaf greenness is an indicator of plant N and can be measured by a Soil Plant Analysis Development (SPAD, Minolta Camera Co., Osaka, Japan) chlorophyll meter. SPAD measurements provide a quick and non-destructive method that enables users to measure chlorophyll content in the field. In particular embodiments, SPAD is useful to determine in situ nitrogen (N) status (Arregui et al. (2006) Eur. J. Agron. 24:140-148; Ziadi et al. (2008) Agron. J. 100:1264-1273; Yuan et al. (2016) Field Crops Res. 185:12-20). The SPAD meter measures the difference between the transmittance of a red (650 nm) and an infrared (940 nm) light through the leaf, generating a three-digit SPAD value (Uddling et al. (2007) Photosynth. Res. 91:37-46). An exemplary protocol includes the following. One day before harvest, leaf greenness is measured on the upmost fully expanded leaf by using a SPAD meter. Four readings are taken in the middle portion of the leaf and the average of the 4 readings for each plant is recorded.

In particular embodiments, shoot fresh weight can be measured as follows. On day 28 after planting, all plants are watered early in the morning to make sure the soil in every pot is not dried to have uniform plant water content across the experiment. Plants are watered again early afternoon if the harvest is not completed in the morning. A plant is cut from the soil surface and shoot fresh weight measured immediately and placed into a paper bag with a plant tag on the bag for drying. All plants are cut uniformly from soil surface across the entire experiment.

In particular embodiments, shoot dry weight can be measured as follows. Bags with plants are placed in the oven in the growth chamber room with 105° C. temperature for 15 min to stop all biological activities. Then the plant samples are dried with oven temperature at 75° C. until constant weight (weigh 10 bags from different positions in the oven with less than 1% weight decrease over 24 hours). Shoot dry weight (0% moisture) will be taken immediately (within 30 seconds after the bag is removed from the oven).

In some embodiments, an in planta assay in a greenhouse can include the following. Inoculated corn seedlings are grown under constant supply of nutrient solution with 25 ppm nitrogen (N) (plus 0 ppm and 100 ppm for characterization assays) in four inch pots for four weeks. Plant growth is evaluated weekly by measuring plant height and leaf number. Leaf greenness is measured by a SPAD meter. Plant biomass (shoot fresh weight and dry weight) is evaluated on day 28 after planting. To estimate the amount of N supplemented by the strains applied, plant biomass is compared between engineered microbe treated plants and non-engineered microbe treated (wild-type) plants and supplied N was inferred from a standard curve with chemical fertilizer.

### (iv) COMPOSITIONS AND FORMULATIONS

The genetically modified Paenibacillus microbe described herein is intended to be useful in the improvement of agricultural plants, and as such, may be formulated with other compositions as part of an agriculturally compatible carrier. The carrier composition including the genetically modified Paenibacillus microbe may be prepared for agricultural application as a liquid, a solid, or a gas formulation. In particular embodiments, the carrier composition includes a vehicle to associate the genetically modified Paenibacillus microbe with an agricultural plant part. Suitable formulations that may be prepared include wettable powders, granules, gels, agar strips or pellets, thickeners, biopolymers, microencapsulated particles; aqueous flowables; aqueous suspensions; and water-in-oil emulsions.

In particular embodiments, a formulation can include: a buffer, a tackifier, a microbial stabilizer, a fungicide, an herbicide, a nematicide, an insecticide, a virucide, a plant growth regulator, a rodenticide, a desiccant, a nutrient, or combinations thereof.

The carrier can be a solid carrier or liquid carrier, and in various forms including microspheres, powders, and emulsions. In particular embodiments, the agricultural carrier may be soil or a plant growth medium. The carrier may be any one or more of a number of carriers that confer a variety of properties, such as increased stability, wettability, or dispersibility. Wetting agents such as natural or synthetic surfactants, which can be nonionic or ionic surfactants, or a combination thereof, can be included in a composition of the disclosure.

Solid compositions can be prepared by dispersing the genetically modified Paenibacillus microbe of the disclosure in and on an appropriately divided solid carrier, including: loam, sand, kaolin clay, talc, pyrophyllite, bentonite, montmorillonite, diatomaceous earth, fuller's earth, acid white soil, pasteurized soil, vermiculite, and pearlite; and inorganic salts such as ammonium sulfate, ammonium phosphate, ammonium nitrate, urea, ammonium chloride, and calcium carbonate. Also, organic fine powders such as wheat flour, wheat bran, and rice bran may be used. Mixtures of any of the aforementioned ingredients are also included as carriers, such as pesta (flour and kaolin clay), or agar or flour-based pellets in loam, sand, or clay. When such formulations are used as wettable powders, biologically compatible dispersing agents such as non-ionic, anionic, amphoteric, or cationic dispersing and emulsifying agents can be used.

In particular embodiments, a genetically modified Paenibacillus microbe of the present disclosure can be mixed or suspended in water or in aqueous solutions. Suitable liquid diluents or carriers include water, aqueous solutions, petroleum distillates, vegetable oils such as soybean oil and cottonseed oil, glycerol, ethylene glycol, polyethylene glycol, propylene glycol, polypropylene glycol, or other liquid carriers. In particular embodiments, a liquid carrier can include a pH ranging from 5-9. In particular embodiments, a liquid carrier has a pH of 7. Water-in-oil emulsions can also be used to formulate a composition that includes the genetically modified Paenibacillus microbe (see, for example, U.S. Pat. No. 7,485,451). Formulations may include food sources for the cultured genetically modified Paenibacillus microbe, such as barley, rice, or other biological materials such as seed, plant parts, sugar cane bagasse, hulls or stalks from grain processing, ground plant material or wood from building site refuse, sawdust or small fibers from recycling of paper, fabric, or wood. Other suitable formulations will be known to those skilled in the art.

In particular embodiments, the formulation can include a tackifier, sticker, or adherent. Such agents are useful for combining the genetically modified Paenibacillus microbe disclosed herein with carriers that can contain other compounds (e.g., control agents that are not biologic) to yield a coating composition. Such compositions help create coatings around the plant or plant part to maintain contact between the genetically modified Paenibacillus microbe and other agents with the plant or plant part. In particular embodiments, adherents (stickers, or tackifiers) include: alginate, gums, starches, lecithins, formononetin, polyvinyl alcohol, alkali formononetinate, hesperetin, polyvinyl acetate, cephalins, gum arabic, xanthan gum, carragennan, polyglutamic acid (PGA), other biopolymers, mineral oil, polyethylene glycol (PEG), polyvinyl pyrrolidone (PVP), arabino-galactan, methyl cellulose, PEG 400, chitosan, polyacrylamide, polyacrylate, polyacrylonitrile, glycerol, triethylene glycol, vinyl acetate, gellan gum, polystyrene, polyvinyl, carboxymethyl cellulose, gum ghatti, and polyoxyethylene-polyoxybutylene block copolymers. Other examples of adherent compositions that can be used in the synthetic preparation include those described in EP0818135, CA1229497, WO2013/090628, EP0192342, WO2008/103422, and CA1041788.

In particular embodiments, the formulation may include an anti-caking agent.

In particular embodiments, the formulation can include a surfactant, wetting agent, emulsifier, stabilizer, or anti-foaming agent. Examples of surfactants include: nitrogen-surfactant blends such as Prefer 28 (Cenex), Surf-N (US), Inhance (Brandt), P-28 (Wilfarm) and Patrol (Helena); esterified seed oils such as Sun-It II (AmCy), MSO (UAP), Scoil (Agsco), Hasten (Wilfarm) and Mes-100 (Drexel); and organo-silicone surfactants such as Silwet L77 (UAP), Silikin (Terra), Dyne-Amic (Helena), Kinetic (Helena), Sylgard 309 (Wilbur-Ellis) and Century (Precision); polysorbate 20; polysorbate 80; Tween 20; Tween 80; Scattics; Alktest TW20; Canarcel; Peogabsorb 80; Triton X-100; Conco NI; Dowfax 9N; Igebapl CO; Makon; Neutronyx 600; Nonipol NO; Plytergent B; Renex 600; Solar NO; Sterox; Serfonic N; T-DET-N; Tergitol NP; Triton N; IGEPAL CA-630; Nonident P-40; and Pluronic. In particular embodiments, the surfactant is present at a concentration of between 0.01% v/v to 10% v/v. In particular embodiments, the surfactant is present at a concentration of between 0.1% v/v to 1% v/v. An example of an anti-foaming agent would be Antifoam-C.

In particular embodiments, the formulation includes a microbial stabilizer. Such an agent can include a desiccant. A “desiccant” can include any compound or mixture of compounds that can be classified as a desiccant regardless of whether the compound or compounds are used in such concentrations that they in fact have a desiccating effect on the liquid inoculant. Such desiccants are ideally compatible with the genetically modified Paenibacillus microbe and should promote the ability of the genetically modified Paenibacillus microbe to survive application on the seeds and to survive desiccation. Examples of suitable desiccants include one or more of trehalose, sucrose, glycerol, and methylene glycol. Other suitable desiccants include other non-reducing sugars and sugar alcohols (e.g., mannitol or sorbitol). The amount of desiccant introduced into the formulation can range, for example, from 5% to 50% by weight/volume (w/v), between 10% to 40% w/v, between 15% and 35% w/v, or between 20% and 30% w/v.

In particular embodiments, it is advantageous for the formulation to include agents such as a fungicide, an herbicide, a nematicide, an insecticide, a plant growth regulator, a rodenticide, a virucide, a nutrient, or any combination thereof. Such agents are ideally compatible with the agricultural plant part or plant onto which the formulation is applied. In particular embodiments, an agent that is compatible with an agricultural plant part or plant is not deleterious to the growth or health of the plant part or plant. In particular embodiments, the agent is ideally one which does not cause safety concerns for human, animal or industrial use (e.g., no safety issues, or the compound is sufficiently labile that the commodity plant product derived from the plant contains negligible amounts of the compound). In particular embodiments, an herbicide includes: imazethapyr, 2,2-dichloropropionic acid, glyphosate, 2,4-dichlorophenoxyacetic acid (2,4-D), and derivatives thereof. In particular embodiments, formulations of the disclosure include a pesticide. In particular embodiments, a pesticide includes: O, S-dimethyl acetylphos-phoramidothioate (acephate), carbamate, carbaryl, chrlopyrifos-methyl, dicrotophos, indoxacarb, 2-(dimethoxyphosphinothioylthio) (malathion), methomyl, methoxyfenozide, methyl parathion, pyrethrins, synthetic pyrethroids (such as bifenthrin, cypermethrin and the like), pyrethroids, protenophos, phorate, spinosyn, dimethyl N,N′-[thiobis[(methylimino)carbonyloxy]]-bis[ethanimidothioate](thiodicarb), and derivatives thereof.

Nutrient additives to the formulation may include fertilizer compositions such as nitrogen, phosphorous, or potassium.

In particular embodiments, the formulation is suited for coating of a genetically modified Paenibacillus microbe onto a plant or plant part. The genetically modified Paenibacillus microbe described in the present disclosure can confer many fitness benefits to a host plant. The ability to confer such benefits by coating the genetically modified Paenibacillus microbe on the surface of plant parts has many potential advantages, particularly when used in a commercial (agricultural) scale. In particular embodiments, the present disclosure provides a composition including a plant or plant part and a genetically modified Paenibacillus microbe described herein. In particular embodiments, the plant part is a seed and the genetically modified Paenibacillus microbe is part of a seed coating.

The genetically modified Paenibacillus microbe described herein can be combined with one or more of the agents described above to yield a formulation suitable for combining with an agricultural plant part or plant. A genetically modified Paenibacillus microbe of the disclosure can be obtained from growth in culture, for example, using a synthetic growth medium. In addition, a genetically modified Paenibacillus microbe disclosed herein can be cultured on solid media, for example on petri dishes, scraped off and suspended into the preparation. A genetically modified Paenibacillus microbe at different growth phases can be used. For example, a genetically modified Paenibacillus microbe at lag phase, early-log phase, mid-log phase, late-log phase, stationary phase, early death phase, or death phase can be used.

The formulations can include a genetically modified Paenibacillus microbe disclosed herein that is between 1% and 90% wet weight, between 3% and 75% wet weight, between 5% and 60% wet weight, or between 10% and 50% in wet weight of the formulation. In particular embodiments, a formulation includes a genetically modified Paenibacillus microbe disclosed herein that is 1% wet weight, 2% wet weight, 3% wet weight, 4% wet weight, 5% wet weight, 6% wet weight, 7% wet weight, 8% wet weight, 9% wet weight, 10% wet weight, 11% wet weight, 12% wet weight, 13% wet weight, 14% wet weight, 15% wet weight, 16% wet weight, 17% wet weight, 18% wet weight, 19% wet weight, 20% wet weight, 21% wet weight, 22% wet weight, 23% wet weight, 24% wet weight, 25% wet weight, 26% wet weight, 27% wet weight, 28% wet weight, 29% wet weight, 30% wet weight, 31% wet weight, 32% wet weight, 33% wet weight, 34% wet weight, 35% wet weight, 36% wet weight, 37% wet weight, 38% wet weight, 39% wet weight, 40% wet weight, 41% wet weight, 42% wet weight, 43% wet weight, 44% wet weight, 45% wet weight, 46% wet weight, 47% wet weight, 48% wet weight, 49% wet weight, 50% wet weight, 51% wet weight, 52% wet weight, 53% wet weight, 54% wet weight, 55% wet weight, 56% wet weight, 57% wet weight, 58% wet weight, 59% wet weight, 60% wet weight, 61% wet weight, 62% wet weight, 63% wet weight, 64% wet weight, 65% wet weight, 66% wet weight, 67% wet weight, 68% wet weight, 69% wet weight, 70% wet weight, 71% wet weight, 72% wet weight, 73% wet weight, 74% wet weight, 75% wet weight, 76% wet weight, 77% wet weight, 78% wet weight, 79% wet weight, 80% wet weight, 81% wet weight, 82% wet weight, 83% wet weight, 84% wet weight, 85% wet weight, 86% wet weight, 87% wet weight, 88% wet weight, 89% wet weight, 90% wet weight, or more of the formulation.

The concentration of the genetically modified Paenibacillus microbe in a carrier may depend upon the carrier. However, any concentration that will achieve plant-enhancing characteristics is desired. In particular embodiments, the formulation includes at least 10² CFU genetically modified Paenibacillus microbe per mL of liquid formulation, between 10² and 10³ CFU per mL, at least 10³ CFU per mL, between 10³ and 10⁴ CFU per mL, at least 10⁴ CFU per mL, between 10⁴ and 10⁵ CFU per mL, at least 10⁵ CFU per mL, between 10⁵ and 10⁶ CFU per mL, between 10⁵ and 10⁹ CFU per mL, at least 10⁶ CFU per mL, between 10⁶ and 10⁷ CFU per mL, at least 10⁷ CFU per mL, between 10⁷ and 10⁸ CFU per mL, at least 10⁸ CFU per mL, between 10⁸ and 10⁹ CFU per mL, or greater than 10⁹ CFU genetically modified Paenibacillus microbe per mL of liquid formulation.

In particular embodiments, the formulation includes at least 10² CFU genetically modified Paenibacillus microbe per gram of non-liquid formulation, between 10² and 10³ CFU per gram, at least 10³ CFU per gram, between 10³ and 10⁴ CFU per gram, at least 10⁴ CFU per gram, between 10⁴ and 10⁵ CFU per gram, at least 10⁵ CFU per gram, between 10⁵ and 10⁶ CFU per gram, between 10⁵ and 10⁹ CFU per gram, at least 10⁶ CFU per gram, between 10⁶ and 10⁷ CFU per gram, at least 10⁷ CFU per gram, between 10⁷ and 10⁸ CFU per gram, at least 10⁸ CFU per gram, between 10⁸ and 10⁹ CFU per gram, or greater than 10⁹ CFU genetically modified Paenibacillus microbe per gram of non-liquid formulation.

In particular embodiments, the formulation is applied to a plant or plant part in an amount of at least 10² CFU genetically modified Paenibacillus microbe per plant or plant part, between 10² and 10³ CFU per plant or plant part, at least 10³ CFU per plant or plant part, between 10³ and 10⁴ CFU per plant or plant part, at least 10⁴ CFU per plant or plant part, between 10⁴ and 10⁵ CFU per plant or plant part, at least 10⁵ CFU per plant or plant part, between 10⁵ and 10⁶ CFU per plant or plant part, between 10⁵ and 10⁹ CFU per plant or plant part, at least 10⁶ CFU per plant or plant part, between 10⁶ and 10⁷ CFU per plant or plant part, at least 10⁷ CFU per plant or plant part, between 10⁷ and 10⁸ CFU per plant or plant part, or greater than 10⁸ CFU per plant or plant part.

In particular embodiments, the formulation is applied to a plant seed in an amount of at least 10² CFU genetically modified Paenibacillus microbe per seed, between 10² and 10³ CFU per seed, at least 10³ CFU per seed, between 10³ and 10⁴ CFU per seed, at least 10⁴ CFU per seed, between 10⁴ and 10⁵ CFU per seed, at least 10⁵ CFU per seed, between 10⁵ and 10⁶ CFU per seed, between 10⁵ and 10⁹ CFU per seed, at least 10⁶ CFU per seed, between 10⁶ and 10⁷ CFU per seed, at least 10⁷ CFU per seed, between 10⁷ and 10⁸ CFU per seed, or greater than 10⁸ CFU per seed.

Particular embodiments provide for a composition including a plant or plant part associated with a genetically modified Paenibacillus microbe of the disclosure. The composition can include a formulation of the genetically modified Paenibacillus microbe as described herein. In particular embodiments, the plant part includes a seed and the formulation includes a seed coating. Seed coatings can include: polymers, guar, film coating layers, binders, active ingredients (e.g., herbicides, plant growth regulators, crop dessicants, fungicides, bactericides, bacteriostats, insecticides, insect repellants, adjuvants, surfactants, fertilizers), filler, and nutrients. Seed coatings are described in, e.g., WO2012/118795; WO2010/111309; and U.S. Pat. No. 8,685,886. In particular embodiments, the composition can further include a medium that promotes plant growth. Media to promote plant growth typically allows ample drainage, permits air around plant roots, allows enough water for the plants, provides nutrients, and supports the plant. In particular embodiments, media that promotes plant growth include: soil, nitrogen, peat, peat-like material, bark, coconut coir, wood residues, bagasse, rice hulls, sand, perlite, pumice, vermiculite, calcined clays, hydrogel, expanded polystyrene, and urea formaldehydes. In particular embodiments, media that promotes plant growth include soil-less hydroponic growing media. In particular embodiments, hydroponic growing media include: rockwool, grow rock (lightweight expanded clay aggregate), coconut fiber, coconut chips, perlite, vermiculite, oasis cubes, floral foam, growstone (e.g., recycled glass), river rock, pine shavings, composted and aged pine bark, polyurethane foam insulation, water-absorbing crystals, sand, and rice hulls.

Particular embodiments provide for a commodity plant product, as well as methods for producing a commodity plant product, that is derived from a plant of the present disclosure. A commodity plant product includes any composition or product that includes material derived from a plant, seed, plant cell, or other plant part of the present disclosure. Commodity plant products may be sold to consumers and can be viable or nonviable. Nonviable commodity products include: nonviable seeds and grains; processed seeds, seed parts, and plant parts; dehydrated plant tissue, frozen plant tissue, and processed plant tissue; seeds and plant parts processed for animal feed for terrestrial and/or aquatic animal consumption; oil, meal, flour, flakes, bran, fiber, paper, tea, coffee, silage, crushed or whole grain, and any other food for human or animal consumption; biomasses and fuel products; and raw material in industry. Industrial uses of oils derived from the agricultural plants described herein include ingredients for paints, plastics, fibers, detergents, cosmetics, lubricants, and biodiesel fuel. Commodity plant products also include industrial compounds, such as a wide variety of resins used in the formulation of adhesives, films, plastics, paints, coatings and foams.

### (v) METHODS OF USE

The compositions and formulations including a genetically modified Paenibacillus microbe of the present disclosure may be used in a method to associate or contact the genetically modified Paenibacillus microbe with a plant or plant part. In particular embodiments, the genetically modified Paenibacillus microbe is associated or contacted with a plant or plant part by inoculation. Inoculation includes introducing a genetically modified Paenibacillus microbe onto or into a plant (for example, seed injection) or plant part by any method of association. Methods of association or contacting can include: seed treatment, root wash, seedling soak, drench, foliar application, soil inoculation, in-furrow application, side dress application, soil pre-treatment, wound inoculation, drip tape irrigation, vector-mediation via a pollinator, injection, osmopriming, hydroponics, aquaponics, and aeroponics. In particular embodiments, inoculation includes contacting a plant or plant part with a formulation or composition including a genetically modified Paenibacillus microbe described herein. Contacting or associating includes bringing a plant or plant part together with a formulation or composition including a genetically modified Paenibacillus microbe described herein such that the plant or plant part is touching or in physical contact with the formulation or composition to allow nitrogen fixed by the Paenibacillus microbe to be used by the plant or plant part to improve an agronomic trait of a plant grown from the inoculated plant or plant part.

Particular embodiments provide for a method for preparing a composition described herein, including associating or contacting the surface of a plant or plant part with a genetically modified Paenibacillus microbe of the present disclosure to produce a composition including an inoculated plant or plant part and a genetically modified Paenibacillus microbe of the present disclosure. In particular embodiments, the genetically modified Paenibacillus microbe is present in a formulation. In particular embodiments, the genetically modified Paenibacillus microbe is in an amount capable of improving an agronomic trait of the plant grown from the inoculated plant or plant part.

Any number of single carrier compositions and single methods of association or contacting, as well as combinations of carrier compositions and methods of association or contacting, are intended to be within the scope of the present disclosure. In some embodiments the Paenibacillus is associated with a seed using any method known in the art, for example, enrobing, co-drying, coating, infusing, injecting, and the like, prior to planting the seed in soil. In particular embodiments, application of the genetically modified Paenibacillus microbe to the plant may be achieved as a powder for surface deposition onto plant leaves, as a spray to the whole plant or selected plant part, as part of a drip to the soil or the roots, or as a coating onto the plant part prior to planting. In particular embodiments, a plant part may first become associated or contacted with a genetically modified Paenibacillus microbe by virtue of seed treatment with a solid (dry) formulation including a genetically modified Paenibacillus microbe, and upon germination and leaf emergence, the plant may be then subjected to a foliar spray of a liquid formulation including a genetically modified Paenibacillus microbe. In particular embodiments, a plant may become associated or contacted with a genetically modified Paenibacillus microbe by virtue of inoculation of the growth medium (soil or hydroponic) with a liquid or solid formulation including a genetically modified Paenibacillus microbe, and be subjected to repeated (two, three, four, five, or more subsequent) inoculations with a liquid or solid formulation including a genetically modified Paenibacillus microbe. In particular embodiments, a plant seed is associated or contacted with a formulation including a genetically modified Paenibacillus microbe.

In particular embodiments, the genetically modified Paenibacillus microbe disclosed herein can move from one plant tissue type to another, such as from the seed exterior into the tissues of a plant. In particular embodiments, the genetically modified Paenibacillus microbe disclosed herein is coated onto the seed of a plant and, upon germination of the seed into a germinated state, localizes to tissue of the plant. In particular embodiments, a Paenibacillus can colonize one or more tissues of a plant. Such tissues include for example, the root, adventitious root, seminal root, root hair, shoot, leaf, flower, bud, tassel, meristem, pollen, pistil, ovaries, stamen, fruit, stolon, rhizome, nodule, tuber, trichome, guard cells, hydathode, petal, sepal, glume, rachis, vascular cambium, phloem, and xylem. In particular embodiments, the genetically modified Paenibacillus microbe localizes to the root and/or the root hair of the plant. In particular embodiments, the genetically modified Paenibacillus microbe localizes to the photosynthetic tissues, for example, leaves and shoots of the plant. In particular embodiments, the genetically modified Paenibacillus microbe localizes to the vascular tissues of the plant, for example, in the xylem and phloem. In particular embodiments, the genetically modified Paenibacillus microbe localizes to the reproductive tissues (flower, pollen, pistil, ovaries, stamen, fruit) of the plant. In particular embodiments, the genetically modified Paenibacillus microbe localizes to the root, shoots, leaves and reproductive tissues of the plant. In particular embodiments, the genetically modified Paenibacillus microbe colonizes the plant such that it is present on the surface of the plant (i.e., its presence is detectably present on the plant exterior, or the episphere of the plant). In particular embodiments, the genetically modified Paenibacillus microbe localizes to substantially all, or all, tissues of the plant. In particular embodiments, the genetically modified Paenibacillus microbe does not localize to the root of a plant. In particular embodiments, the genetically modified Paenibacillus microbe does not localize to the photosynthetic tissues of the plant.

In particular embodiments, the genetically modified Paenibacillus microbe replicates within the host plant and colonizes the plant. In particular embodiments, the genetically modified Paenibacillus microbe can colonize, leaves, stems, fruit and/or seed tissue of the plant. Successful colonization can be confirmed by detecting the presence of the bacterial population anywhere within or on the plant. The genetically modified Paenibacillus can also colonize the soil in the vicinity of the plant roots or the surface of the plant roots, for example the rhizosphere or rhizoplane. The Paenibacillus can be detected after it is associated with plant tissue in the roots and/or shoots of the plants that germinate from the seeds. The titer of the Paenibacillus can be determined using any method known in the art. Detecting the presence of the genetically modified Paenibacillus microbe inside the plant can be accomplished by measuring the viability of the genetically modified Paenibacillus microbe after surface sterilization of the seed or the plant: genetically modified Paenibacillus microbe colonization can result in internal localization of the genetically modified Paenibacillus microbe, rendering it resistant to conditions of surface sterilization. Internal localization can be localization in the interstitial fluid, interstitial space between plant cells, intercellularly and the like. The presence and quantity of the genetically modified Paenibacillus microbe can also be established using any means known in the art, for example, immunofluorescence microscopy using microbe-specific antibodies, or fluorescence in situ hybridization (see, for example, Amann et al. (2001) Current Opinion in Biotechnology 12:231-236). Alternatively, specific nucleic acid probes recognizing conserved sequences from a genetically modified Paenibacillus microbe can be employed to amplify a region, for example by quantitative PCR, and correlated to CFUs (Colony Forming Units) by means of a standard curve. In particular embodiments, a CFU refers to a unit used to estimate the concentration of a microbe in a test sample. The number of visible colonies (CFUs) present on an agar plate can be multiplied by the dilution factor to obtain a CFU per volume.

In particular embodiments, the genetically modified Paenibacillus microbe is disposed, for example, on the surface of an agricultural plant part, in an amount effective to be detectable in the mature agricultural plant. In particular embodiments, the genetically modified Paenibacillus microbe is disposed in an amount effective to be detectable in an amount of at least 100 CFU, between 100 and 200 CFU, at least 200 CFU, between 200 and 300 CFU, at least 300 CFU, between 300 and 400 CFU, at least 500 CFU, between 500 and 1,000 CFU, at least 1,000 CFU, between 1,000 and 3,000 CFU, at least 3,000 CFU, between 3,000 and 10,000 CFU, at least 10,000 CFU, between 10,000 and 30,000 CFU, at least 30,000 CFU, between 30,000 and 100,000 CFU, at least 100,000 CFU, at least 1 million CFU, at least 1.5 million CFU, at least 10 million CFU, at least 100 million CFU, at least 1 billion CFU, or more per gram of dry tissue, or per gram of wet tissue, or per gram of dry soil in the case the genetically modified Paenibacillus colonized the rhizosphere, in the mature agricultural plant.

In particular embodiments, the genetically modified Paenibacillus microbe colonizes particular plant parts or tissue types of the plant. In particular embodiments, the genetically modified Paenibacillus microbe is disposed on the seed or seedling in an amount effective to be detectable within a target tissue of the mature agricultural plant selected from a fruit, a seed, a leaf, or a root, or portion thereof. For example, the genetically modified Paenibacillus microbe can be detected in an amount of at least 100 CFU, between 100 and 200 CFU, at least 200 CFU, between 200 and 300 CFU, at least 300 CFU, between 300 and 500 CFU, at least 500 CFU, between 500 and 1,000 CFU, at least 1,000 CFU, between 1,000 and 3,000 CFU, at least 3,000 CFU, between 3,000 and 10,000 CFU, at least 10,000 CFU, between 10,000 CFU and 30,000 CFU, at least 30,000 CFU, between 30,000 and 100,000 CFU, between at least 100,000 CFU and 1 million CFU, between at least 1 million CFU and 10 million CFU, between at least 10 million CFU and 1 billion CFU per gram in the target tissue (dry or wet) of the mature agricultural plant.

The compositions and formulations of the present disclosure may be used to improve any characteristic of any agricultural plant. In particular embodiments, the present disclosure includes the use of a genetically modified Paenibacillus microbe disclosed herein to confer a beneficial agronomic trait upon a plant part or plant with which the genetically modified Paenibacillus microbe is associated.

In particular embodiments, the agronomic trait includes increased nitrogen fixation, reduced nitrogen usage, increased nitrogen content, increased plant yield, increased plant biomass, increased shoot biomass, increased shoot length, increased dry shoot weight, increased fresh shoot weight, increased seedling shoot length, increased dry seedling weight, increased fresh seedling weight, increased leaf surface area, increased root biomass, increased root length, increased root surface area, increased germination rate, increased emergence rate, increased photosynthetic capability, increased chlorophyll content, increased vigor, increased seed yield, increased dry weight of mature seeds, increased fresh weight of mature seeds, increased number of mature seeds per plant, increased number of pods per plant, increased length of pods per plant, increased plant height, increased pathogen resistance, increased pest resistance, earlier or increased flowering, increased protein content, increased carbohydrate content, and/or increased antioxidant content relative to a reference agricultural plant grown under the same conditions. In particular embodiments, the reference agricultural plant includes an uninoculated plant. In particular embodiments, at least two agronomic traits are improved in the agricultural plant. “Yield” or “plant yield” refers to increased plant growth, increased crop growth, increased biomass, and/or increased plant product production, and is dependent to some extent on temperature, plant size, organ size, planting density, light, water and nutrient availability, and how the plant copes with various stresses, such as through temperature acclimation and water or nutrient use efficiency. In specific embodiments plant yield refers to the bushels per acre of a desired crop, such as corn, or soybeans. Yield can also be enhanced by the genetically engineered Paenibacillus microbe in other non-row crops such as fruit, vegetables, for example, grapes, pome fruit and citrus.

In particular embodiments, the genetically modified Paenibacillus microbe may provide an improved benefit, e.g., increased plant yield, to a plant associated with the microbe that is of at least 3%, between 3% and 5%, between 3% and 20%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, or at least 300% or more, when compared with a reference agricultural plant grown under the same conditions. In particular embodiments, the reference agricultural plant includes an uninoculated plant. In other embodiments, the reference plant is inoculated with the parental strain or wild-type strain from which the genetically engineered Paenibacillus microbe is derived.

Particular embodiments provide plants, and fields of plants, that are associated with a genetically modified Paenibacillus microbe, such that the overall fitness, productivity or health of the plant or a portion thereof, is maintained, increased and/or improved over a period of time. Improvement in overall plant health can be assessed using numerous physiological parameters including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, nitrogen content or any combination thereof. Improved plant health, or improved field health, can also be demonstrated through improved resistance or response to a given stress, either biotic or abiotic stress, or a combination of one or more abiotic stresses.

Particular embodiments provide a method of reducing nitrogen fertilizer application, including growing a plant from a plant part that has been contacted with a formulation including a genetically modified Paenibacillus microbe described herein, wherein application of nitrogen to the plant is reduced as compared to application of nitrogen to a referenced agricultural plant. In particular embodiments, the referenced agricultural plant is a corresponding plant grown from a plant part that has not been contacted with the formulation. “Fertilizer” refers to any organic material or inorganic material of natural or synthetic origin which is added to soil to provide nutrients, including all three elements of nitrogen, phosphorus, and potassium, necessary to sustain plant growth. In particular embodiments, a fertilizer includes nitrogen.

In particular embodiments, the reduction in the application of nitrogen is measured as N replacement per application, and wherein the N replacement is at least 5 ppm of N, at least 6 ppm of N, at least 7 ppm of N, at least 8 ppm of N, at least 9 ppm of N, at least 10 ppm of N, at least 11 ppm of N, at least 12 ppm of N, at least 13 ppm of N, at least 14 ppm of N, at least 15 ppm of N, at least 16 ppm of N, at least 17 ppm of N, at least 18 ppm of N, at least 19 ppm of N, at least 20 ppm of N, at least 21 ppm of N, at least 22 ppm of N, at least 23 ppm of N, at least 24 ppm of N, at least 25 ppm of N or greater. In some examples, in a green house setting, the genetically engineered Paenibacillus microbe will perform better in some measurable quality as compared to a parental or wild-type strain, in that the genetically engineered Paenibacillus microbe will perform at least as well at the parental or wild-type strain does when the parental or wild-type strain is grown in the presence of 10 ppm more nitrogen than the genetically engineered Paenibacillus microbe.

Particular embodiments methods of improving an agronomic trait in a plant are described, including growing a plant from a plant or plant part that has been contacted with a formulation including a genetically modified Paenibacillus microbe described herein, wherein an agronomic trait is improved in the plant as compared to the corresponding agronomic trait in a referenced agricultural plant. In particular embodiments, the referenced agricultural plant is a corresponding plant grown from a plant part that has not been contacted with the formulation. In particular embodiments, the improved trait can be an increase in overall biomass of the plant or plant part, including its fruit or seed (for example, kernel or bean). In particular embodiments, a genetically modified Paenibacillus microbe is disposed on the surface or within a plant or plant part in an amount effective to increase the biomass of the plant or plant grown from the plant part. The increased biomass is useful in the production of commodity products derived from the plant. The increase in biomass can occur in a plant part (e.g., the root tissue, shoots, leaves, etc.), or can be an increase in overall biomass. In particular embodiments, an increase in overall biomass of a plant associated with a genetically modified Paenibacillus microbe can include an increase of at least 3%, between 3% and 5%, between 3% and 20%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, as compared to overall biomass from a reference uninoculated agricultural plant grown under the same conditions, or a plant inoculated with plant with a wild-type or parental strain. In particular embodiments, the reference agricultural plant includes an uninoculated plant. In particular embodiments, such increase in overall biomass can be under relatively stress-free conditions. In particular embodiments, the increase in biomass can be in plants grown under any number of abiotic or biotic stresses, including drought stress, nitrogen stress, salt stress, heat stress, cold stress, low nutrient stress, nematode stress, insect herbivory stress, fungal pathogen stress, bacterial pathogen stress, and viral pathogen stress.

In particular embodiments, a genetically modified Paenibacillus microbe disclosed herein is disposed in an amount effective to increase chlorophyll content (e.g., as measured by a SPAD meter). In particular embodiments, an increase in chlorophyll content of a plant associated with a genetically modified Paenibacillus microbe can include an increase of at least 3%, between 3% and 5%, between 3% and 20%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, as compared to chlorophyll content from a reference agricultural plant grown under the same conditions. In particular embodiments, the reference agricultural plant includes an uninoculated plant. In particular embodiments, the reference agricultural plant includes a plant inoculated with a wild-type or parental strain of Paenibacillus.

In particular embodiments, a genetically modified Paenibacillus microbe disclosed herein is disposed in an amount effective to increase shoot fresh weight. In particular embodiments, an increase in shoot fresh weight of a plant associated with a genetically modified Paenibacillus microbe can include an increase of at least 3%, between 3% and 5%, between 3% and 20%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, as compared to shoot fresh weight from a reference agricultural plant grown under the same conditions. In particular embodiments, the reference agricultural plant includes an uninoculated plant. In particular embodiments, the reference agricultural plant includes a plant inoculated with a wild-type or parental strain of Paenibacillus.

In particular embodiments, a genetically modified Paenibacillus microbe disclosed herein is disposed in an amount effective to increase shoot dry weight. In particular embodiments, an increase in shoot dry weight of a plant associated with a genetically modified Paenibacillus microbe can include an increase of at least 3%, between 3% and 5%, between 3% and 20%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, as compared to shoot dry weight from a reference agricultural plant grown under the same conditions. In particular embodiments, the reference agricultural plant includes an uninoculated plant. In particular embodiments, the reference agricultural plant includes a plant inoculated with a wild-type or parental strain of Paenibacillus.

In particular embodiments, a genetically modified Paenibacillus microbe disclosed herein is disposed in an amount effective to increase root biomass of a plant associated with the genetically modified Paenibacillus microbe by at least 3%, between 3% and 5%, between 3% and 20%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, as compared to root biomass of a reference agricultural plant grown under the same conditions. In particular embodiments, the reference agricultural plant includes an uninoculated plant. In particular embodiments, the reference agricultural plant includes a plant inoculated with a wild-type or parental strain of Paenibacillus.

In particular embodiments, a genetically modified Paenibacillus microbe is disposed on the surface or within a plant or plant part in an amount effective to increase the biomass of the fruit or cob from the resulting plant at least 3%, between 3% and 5%, between 3% and 20%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, as compared to biomass of the fruit or cob of a reference agricultural plant grown under the same conditions. In particular embodiments, the reference agricultural plant includes an uninoculated plant. In particular embodiments, the reference agricultural plant includes a plant inoculated with a wild-type or parental strain of Paenibacillus.

In particular embodiments, a genetically modified Paenibacillus microbe is disposed on the surface or within a plant or plant part in an amount effective to increase the photosynthetic capability of the resulting plant by at least 3%, between 3% and 5%, between 3% and 20%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, or at least 100%, as compared to photosynthetic capability of a reference agricultural plant grown under the same conditions. In particular embodiments, photosynthetic capability can be expressed as a rate at which leaves are able to fix carbon during photosynthesis. Photosynthetic capability is typically measured as the amount of carbon dioxide that is fixed per metre squared per second, for example as μmol/m²/sec. Assays to measure photosynthetic capability include leaf gas exchange, thermal imagery, hyperspectral reflectance, chlorophyll fluorescence, normalized difference vegetation index (NDVI) and infrared thermography. In particular embodiments, photosynthetic capability can be indirectly measured by measuring plant yield, plant biomass, shoot biomass, shoot length, dry shoot weight, fresh shoot weight, seedling shoot length, dry seedling weight, fresh seedling weight, leaf surface area, number of stomata per leaf, root biomass, root length, root surface area, germination rate, emergence rate, chlorophyll content, vigor, seed yield, dry weight of mature seeds, fresh weight of mature seeds, number of mature seeds per plant, number of pods per plant, length of pods per plant, plant height, or a combination thereof.

Particular embodiments provide for genetically modified Paenibacillus microbe-associated plants with increased resistance to an abiotic stress. Exemplary abiotic stresses include: drought, heat, cold, salt stress, high metal content, low nitrogen and low nutrient.

Particular embodiments provide for genetically modified Paenibacillus microbe-associated plants with increased resistance to biotic stress. Exemplary biotic stresses include: insect infestation, nematode infestation, complex infection, fungal infection, bacterial infection, oomycete infection, protozoal infection, viral infection, and herbivore grazing, or a combination thereof.

Other plant traits can be improved when a plant or plant part is associated with a genetically modified Paenibacillus microbe disclosed herein. In particular embodiments, the genetically modified Paenibacillus microbe-associated plant can have an increase in plant growth hormones such as auxin as compared to a reference agricultural plant grown under the same conditions. In particular embodiments, the genetically modified Paenibacillus microbe-associated plant can have an altered hormone status or altered levels of hormone production as compared with a reference agricultural plant. An alteration in hormone status may affect many physiological parameters, including flowering time, water efficiency, apical dominance and/or lateral shoot branching, increase in root hair, and alteration in fruit ripening.

In particular embodiments, the genetically modified Paenibacillus microbe-associated plant can have an improved nutritional content of the plant or plant part as compared to a reference agricultural plant or plant part from a reference agricultural plant grown under the same conditions. Examples of such nutrients include: amino acid; protein; oil (including oleic acid, linoleic acid, alpha-linoleic acid, saturated fatty acids, palmitic acid, stearic acid and trans fats); carbohydrates (including sugars such as sucrose, glucose and fructose, starch, or dietary fiber); Vitamin A; thiamine (vitamin B1); riboflavin (vitamin B2); nitrogen content, niacin (vitamin B3); pantothenic acid (vitamin B5); vitamin B6; folate (vitamin B9); choline; vitamin C; vitamin E; vitamin K; calcium; iron; magnesium; manganese; phosphorus; potassium; sodium; and zinc.

In particular embodiments, the genetically modified Paenibacillus microbe-associated plant can have a reduced content of a harmful or undesirable substance as compared with a reference agricultural plant. Such compounds include those which are harmful when ingested in large quantities or are bitter tasting (for example, oxalic acid, amygdalin, certain alkaloids such as solanine, caffeine, nicotine, quinine and morphine, tannins, cyanide). As such, in particular embodiments, the genetically modified Paenibacillus microbe-associated plant or part thereof contains less of the undesirable substance as compared with a reference agricultural plant. In particular embodiments, the improved trait can include improved taste of the genetically modified Paenibacillus microbe-associated plant or part thereof, including the fruit or seed.

The association between a genetically modified Paenibacillus microbe disclosed herein and a plant can be detected using methods known in the art. For example, the biochemical, metabolomics, proteomic, genomic, epigenomic and/or transcriptomic profiles of a genetically modified Paenibacillus microbe-associated plant can be compared with the corresponding profile of a reference agricultural plant grown under the same conditions. In particular embodiments, the reference agricultural plant includes an uninoculated plant. Transcriptome analysis of a genetically modified Paenibacillus microbe disclosed herein and reference agricultural plants can also be performed to detect changes in expression of at least one transcript, or a set or network of genes upon microbe association. Similarly, epigenetic changes can be detected using methylated DNA immunoprecipitation followed by high-throughput sequencing.

Metabolomic differences between the plants can be detected using methods known in the art. Metabolites, proteins, or other compounds can be detected using any suitable method, including: gel electrophoresis; liquid chromatography; gas phase chromatography; mass spectrometry; nuclear magnetic resonance (NMR); immunoassays (e.g., enzyme-linked immunosorbent assays (ELISAs)); chemical assays; spectroscopy; optical imaging techniques (such as magnetic resonance spectroscopy (MRS); magnetic resonance imaging (MRI); CAT scans; ultra sound; and mass spectrometry-based tissue imaging or X-ray detection methods (e.g., energy dispersive x-ray fluorescence detection)). In particular embodiments, commercial systems for chromatography and NMR analysis are utilized. Such metabolomic methods can be used to detect differences in levels or content of, for example, hormones, nutrients, secondary metabolites, root exudates, phloem sap, xylem sap, and heavy metals. Such methods are also useful for detecting alterations of metabolites in the genetically modified Paenibacillus microbe.

The term “plant” is used in its broadest sense. It includes any species of grass (e.g., turf grass), sedge, rush, ornamental or decorative, crop or cereal, fodder or forage, fruit or vegetable, fruit plant or vegetable plant, flowers, and trees. In particular embodiments, a plant includes: wheat, soybean, maize, barley, millet, rice, turfgrass, cotton, canola, rapeseed, alfalfa, tomato, sugarbeet, oats, rye, sorghum, almond, walnut, apple, cannabis, peanut, strawberry, lettuce, orange, potato, banana, sugarcane, cassava, mango, guava, palm, onions, olives, peppers, tea, yams, cacao, sunflower, asparagus, carrot, coconut, lemon, lime, watermelon, cabbage, cucumber, and grape. A plant part is any part of a plant, tissue of a plant, or cell of a plant. In particular embodiments, a plant or plant part includes: a whole plant, a seedling, cotyledon, meristematic tissue, ground tissue, vascular tissue, dermal tissue, seed, pod, tiller, sprig, leaf, stomata, root, shoot, stem, flower, fruit, pistil, ovaries, pollen, stamen, phloem, xylem, stolon, plug, bulb, tuber, corm, keikis, bud, and blade. In particular embodiments, a plant part includes a root, a stem, or a leaf. “Leaf” and “leaves” refer to a usually flat, green structure of a plant where photosynthesis and transpiration take place and attached to a stem or branch. “Stem” refers to a main ascending axis of a plant. “Seed” refers to a ripened ovule, including the embryo and a casing. In particular embodiments, compositions of the present disclosure include a plant and/or a plant part described herein.

Particular embodiments provide for a plant to which a genetically modified Paenibacillus microbe-associated plant described herein is compared to assess agronomic trait improvement or reduction in nitrogen application for the genetically modified Paenibacillus microbe-associated plant. In particular embodiments, a reference agricultural plant includes a plant of the same genus and species as the genetically modified Paenibacillus microbe-associated plant and grown under the same conditions but that has not been associated with a genetically modified Paenibacillus microbe (i.e., an uninoculated plant). In particular embodiments, a reference agricultural plant includes an uninoculated plant of the same genus and species as the genetically modified Paenibacillus microbe-associated plant and grown under the same conditions and that is a commercially grown plant. In particular embodiments, a commercially grown plant is a plant that is or has been grown by farmers and sold as a commodity. In particular embodiments, a reference agricultural plant includes a plant of the same genus and species as the genetically modified Paenibacillus microbe-associated plant and grown under the same conditions and that is associated or contacted (i.e., inoculated) with a different genetically modified Paenibacillus microbe. In particular embodiments, a different genetically modified Paenibacillus microbe includes different genetic modifications in its genome as compared to the genetically modified Paenibacillus microbe associated with the test plant.

### (vi) EXAMPLES

### Example 1. Exemplary Gram Positive Enhanced Nitrogen Fixation Strains

Microbial nitrogen fixation is catalyzed by the enzyme nitrogenase. The reaction is high energy and electron-demanding, requiring 16 to 32 adenosine triphosphate (ATP) molecules and 8 electrons to complete the reduction of one atmospheric dinitrogen into two molecules of ammonia (see FIG. 1). The electrons necessary for the nitrogenase reaction are produced by diverse biological reactions. Once generated, electrons are carried by soluble electron protein carriers to the acceptor site of the nitrogenase enzyme. Inside the nitrogenase enzyme, three redox centers allow the electron to be transported from the acceptor site to the catalytic center where the reduction of dinitrogen to ammonia takes place. Exemplary strains Paenibacillus brasilensis (Pb) and Paenibacillus peoriae (Pp) were engineered to overexpress genes involved in the generation or the transport of electrons.

Pb strain PB172 was obtained from the German Collection of Microorganisms and Cell Cultures (DSMZ) with the deposition number DSM 14914. The Paenibacillus peoriae strain described in the examples has been deposited on Nov. 22, 2021, with the Agricultural Research Service Culture Collection located at the National Center for Agricultural Utilization Research, Agricultural Research Service, U.S. Department of Agriculture, 1815 North University Street, Peoria, Illinois 61604, U.S.A., and has been assigned the following depository designation: NRRL B-68085.

PB172 was engineered to increase electron availability using several approaches as described in Tables 1 and 2.

TABLE 1
List of Paenibacillus strain IDs, genotype, purpose
of genetic modification, and SEQ ID NOs for cassettes.

| | | | | SEQ |
|---|---|---|---|---|
| | | Enzyme | | ID |
| | | Commission | | NO: |
| | Expression cassette | Number/ | Purpose | for |
| Strain | integrated in Pb | protein | of genetic | cas- |
| ID | strain | class Gene | modification | sette |
| Pb6 | PsigX:Pz_porCA- | EC 1.2.7.1 | Increase flux of | 2 |
| | TporA | | electrons to nitrogen | |
| | | | fixation reaction | |
| Pb8 | PsigX:Pz_fldA- | protein | Increase flux of | 4 |
| | TfldA | electron | electrons to nitrogen | |
| | | carrier | fixation reaction | |
| | | (Flavodoxin) | | |
| Pb10 | PtrnQ:Pz_porCA- | E.C. 1.2.7.1 | Increase flux of | 6 |
| | TporA | | electrons to nitrogen | |
| | | | fixation reaction | |
| Pb11 | PtrnQ:Pz_sufCDSUB- | Fe—S | Increase availability | 7 |
| | TsufB | cluster | of the [4Fe—4S] | |
| | | assembly | metallo-cluster to | |
| | | | nitrogenase | |
| Pb12 | PtrnQ:Pz_fer-Tfer | Protein | Increase flux of | 8 |
| | | electron | electrons to nitrogen | |
| | | carrier | fixation reaction | |
| | | (ferredoxin) | | |
| Pb16 | PsigX:Pf_sufCDSUB- | Fe—S | Increase availability | 10 |
| | TsufB | cluster | of the [4Fe—4S] | |
| | | assembly | metallo-cluster to | |
| | | | nitrogenase | |

TABLE 2
Components of genetic constructs and source of each component.

| | | | | GenBank |
|---|---|---|---|---|
| | | | GenBank | accession # of |
| | | | accession # of | introduced |
| Name | Description | Source | source genome | component |
| Himar1 | Inverted terminal | Haematobia | GCA_003123925.1. | U11645.1 |
| ITR 1 | repeat | irritans | EMBO J. | |
| | | | 1996 Oct. 1; | |
| | | | 15(19): 5470-9. | |
| | | | A purified | |
| | | | mariner | |
| | | | transposase is | |
| | | | sufficient to | |
| | | | mediate | |
| | | | transposition in | |
| | | | vitro | |
| | | | D J Lampe 1, | |
| | | | M E Churchill, | |
| | | | H M Robertson | |
| Himar1 | Inverted terminal | Haematobia | GCA_003123925.1. | U11645.1 |
| ITR 2 | repeat | irritans | EMBO J. | |
| | | | 1996 Oct. 1; | |
| | | | 15(19): 5470-9. | |
| | | | A purified | |
| | | | mariner | |
| | | | transposase is | |
| | | | sufficient to | |
| | | | mediate | |
| | | | transposition in | |
| | | | vitro | |
| | | | D J Lampe 1, | |
| | | | M E Churchill, | |
| | | | H M Robertson | |
| CAT | Chloramphenicol | pC194 plasmid | NC_002013.1 | NG_047564.1 |
| | acetyl transferase | isolated from | | |
| | | Staphylococcus | | |
| | | aureus | | |
| PsigX | Medium-strength | Bacillus | e.g., | nt 2527576- |
| | constitutive | subtilis | CP053102.1 | 2527882 of |
| | promoter | | | CP053102.1 |
| PtrnQ | Very strong | Bacillus | e.g., | Locus tag |
| | constitutive | subtilis | CP053102.1 | HIR77_19330 |
| | promoter | | | (nt 3646432- |
| | | | | 3646619 of |
| | | | | CP053102.1) |
| PmreB | constitutive | Bacillus | e.g., | nt 1495909- |
| | promoter | subtilis | CP054177.1 | 1496122 of |
| | | | | CP054177.1 |
| PgroES | Inducible heat | Bacillus | e.g., | nt 649628- |
| | shock promoter | subtilis | CP053102.1 | 649914 of |
| | | | | CP053102.1 |
| porCDAB | Pyruvate | Paenibacillus | GCF_000520715.1 | WP_025689130.1, |
| | flavodoxin | zanthoxyli | | WP_025689129.1 |
| | oxidoreductase | JH29 | | |
| sufCDSUB | Sulfur formation | Paenibacillus | GCF_000520735.1 | WP_02570271.1 |
| | operon | forsythiae | | WP_02570271.1 |
| | | T98 | | WP_02570271.1 |
| | | | | WP_02569685.1 |
| | | | | WP_02569685.1 |
| fldA | Flavodoxin | Paenibacillus | GCF_000520715.1 | WP_02569146 |
| | | zanthoxyli JH29 | | |
| sufCDSUB | Sulfur formation | Paenibacillus | GCF_000520715.1 | WP_025689975.1, |
| | operon | zanthoxyli JH29 | | WP_025689976.1, |
| | | | | WP_025689977.1, |
| | | | | WP_025690804.1, |
| | | | | WP_025690805.1 |
| Pz_fer | Ferredoxin | Paenibacillus | GCF_000520715.1 | WP_036737311.1 |
| | | zanthoxyli JH29 | | |

The taxonomy of the subject microorganisms was validated after selection on chloramphenicol medium when integrants and integration sites were verified through whole genome Illumina sequencing as belonging to Pb.

The morphological features of the subject microorganisms were expected to be similar to the recipient organism. All eight subject microorganisms were resistant to 5 μg/ml chloramphenicol through the expression of the CAT gene. In addition, all strains were expected to be improved for nitrogen fixation as described in Table 1.

The final constructs as inserted into the genome of the recipient organism PB172 to generate the various strains described in Table 1 are depicted in FIGS. 2A-2F.

The exemplary strains described in Table 1 were made using the following general technique.

Integration of the exogenous genes was performed using pseudo-random, transposon-mediated integration resulting in strains expressing the gene or operon of interest and a selectable marker. However, one or ordinary skill in the art can envision using alternative methods of gene editing to arrive at substantially similar strains.

Briefly, the subject organisms were constructed in a three-step process.

Step 1: Recipient Pb strain PB172 was transformed with the temperature sensitive tnpA plasmid constitutively expressing an erythromycin resistance marker ermC from the pE194 plasmid of Staphylococcus aureus and the Himar1 mariner transposase gene from Haematobia irritans. Cells were plated on erythromycin medium at the permissive temperature of 30° C. to select for episomal plasmid transformants. The transformation rate achieved with this method was approximately 10,000 colonies per reaction.

Step 2: The presence of the tnpA plasmid and its sequence was verified by inoculating liquid cultures from individual colonies and subjecting them to whole cell sequencing, then the cells were transformed with a donor plasmid containing the fragment that was integrated into the genome of the recipient organism (fragments as illustrated in FIGS. 2A-2F).

In addition, the donor plasmid contained a temperature sensitive origin of replication, the Himar1 transposon cassette comprising the PCAT chloramphenicol responsive promoter from the Staphylococcus plasmid pC194, the CAT chloramphenicol acetyl transferase resistance gene from pC194, and the TCAT terminator from pC194. The transposon cassette was flanked by inverted mariner terminal repeat sequences from Haematobia irritans. Cells were initially plated on chloramphenicol medium at the permissive temperature of 30° C. to select for episomal plasmid transformants. At this stage, the subject organisms carried two plasmids, comprising the transposase plasmid and the transposon plasmid.

Step 3: Lastly, four colonies from each subject organism were struck out on chloramphenicol medium at the non-permissive temperature of 37° C. to select for cells that had chromosomally integrated the transposon cassette and against cells that retained replicating plasmids. Single colonies from each were sequenced to verify insertion of the transposon and eviction of the plasmid.

The mariner transposon is reported to insert into a wide variety of host genomes in pseudo-random fashion at thymine-adenine (TA) DNA sites. The mariner inverted tandem repeat sequences were confirmed to be intact and the desired transposon cassette containing the CAT marker and the genes of interest were confirmed to be inserted without any deviation from the synthesized sequence. Following is a detailed description of the process to create and verify the Pb8 strain.

Having confirmed that the integrated sequence was correct and that the plasmids had been evicted, the site of integration of the transposon cassette was determined. The Mariner transposase integrates pseudo-randomly at any site in the genome between T and A bases. By analyzing the aligned reads that mapped to the edge of the transposon cassette and spanned the inverted terminal repeat (ITR) sequences, but without alignment to the plasmid backbone, a consensus sequence of the part of the reads that extended beyond the ITRs was obtained. These upstream and downstream overhanging consensus sequences ranged from 10 and 11 bases long, respectively. The consensus sequence that extended beyond the upstream ITR2 site ended with 3′ T and in all cases the consensus sequence that extended beyond the downstream ITR1 site began with 5′ A as would be expected based on the reported preference of the Himar1 transposon to integrate between T and A bases.

These consensus sequences were concatenated to form a prediction of what the flanking genome sequence looked like prior to being interrupted by the transposon cassette. The concatenated sequence was 21 bases and exactly matched one genomic region in the Pb genome. This matching site was the putative integration site of the transposon. To verify the integration site, in silico constructs were created by inserting each transposon cassette into its corresponding putative integration site flanked by its native genomic locus. The Illumina reads were aligned to this new in silico construct to verify that this was indeed the site of integration. In all cases, reads spanned the transposon cassette and the ITR sites and parts of the flanking genomic region without any mismatches to the in silico construct, thus confirming that the constructs were indeed integrated at the putative locus.

The following includes details for the Pb8 strain, illustrating that the transposon cassette was integrated into the genome. Alignment of the sequencing reads to the genomic sequence of Pb showed integration of the PsigX fldA CAT transposon cassette into the genome. The sequencing sample, s23533075, was assembled to transposon cassette, m2875002 PsigX Pz fldA. 16,814 reads out of 60,677,211 reads mapped to the transposon cassette (the remaining reads mapped to the genome). Downstream of ITR1 had the following 10 bp sequence: ATTTGTTAAT (SEQ ID NO: 21). Upstream of ITR2 had the following 11 bp sequence: ACGTTCCTTGT (SEQ ID NO: 22). The concatenated 21 bp sequence was: ACGTTCCTTGTATTTGTTAAT (SEQ ID NO: 23). The concatenated sequence matched one site in the Pb genome at PROKKA_03723, which is annotated as a gene encoding a hypothetical protein. The Pb8 strain was tested on different types of media and disrupting this gene did not cause any obvious growth defects relative to the wild-type Pb strain.

An in silico construct was created by inserting the sequence of the transposon cassette into the putative integration site within PROKKA_03723 and the sequencing reads were aligned to this new in silico construct. This was done to verify that the transposon cassette was in fact integrated within PROKKA_03723 by looking at 101 bp Illumina reads that align to both the transposon cassette and to the Pb genome. If reads span the transposon cassette and the putative flanking region of the genome, then it can be assumed that the construct was integrated in that site. For Pb8, the same reads align to both the genomic region and the transposon cassette of the in silico model of the integration event.

200 bp upstream of integration at 03723:
(SEQ ID NO: 24)
ttaatctaccttaacacccaagttccgaaaaaatccccgcatat
tctgcatataagagtcctgagtagcggaagcttccttcagtaga
gtttgcatactgttcataaatgcggaagccgaagtcgtatcagc
tttgtcaaaccgattctttaactgtgtgttatttaacagctctt
ccatctgtcccgtacgttccttgt
200 bp downstream of integration at 03723:
(SEQ ID NO: 25)
atttgttaatccaatcagggttttgagggaccttttgcgcaaag
cttaacagttggctcagtccatttcctccacccttaatgtcatt
catgaatgccgcatattgttgaggatccgcctgcttcatcactt
ccgacagtttgacgtaatcctcaggagcacctaacgggcgatct
gttggagtcgtgtagctaatatct
400 bp of wt PROKKA_03723 locus flanking
integration at 03723 (concatenation of SEQ
ID NOs: 24 and 25):
(SEQ ID NO: 26)
ttaatctaccttaacacccaagttccgaaaaaatccccgcatat
tctgcatataagagtcctgagtagcggaagcttccttcagtaga
gtttgcatactgttcataaatgcggaagccgaagtcgtatcagc
tttgtcaaaccgattctttaactgtgtgttatttaacagctctt
ccatctgtcccgtacgttccttgtatttgttaatccaatcaggg
ttttgagggaccttttgcgcaaagcttaacagttggctcagtcc
atttcctccacccttaatgtcattcatgaatgccgcatattgtt
gaggatccgcctgcttcatcacttccgacagtttgacgtaatcc
tcaggagcacctaacgggcgatctgttggagtcgtgtagctaat
atct

The wild-type PROKKA_03723 sequence is provided in SEQ ID NO: 27. The PROKKA_03723 locus with the integration of the transposon cassette of Pb8 is included in SEQ ID NO: 28.

Applicants observed that Pb8 lost its ability to sporulate and/or exhibited abnormal growth and, for this reason, conducted whole genome sequencing to determine the changes to the strain. The Pb8 strain used in the examples below contain the following unintended changes in addition to the engineered changes described above: tuaD (disruptive inframe deletion), transposase IS116/IS110/IS902 family protein (frameshift variant) and farnesyl diphosphate synthase (frameshift variant). These unintended changes may have affected the sporulation and growth phenotype of Pb8 and its derivatives.

Pb6: This strain illustrated the overexpression of an exemplary enzyme from E.C. 1.2.7.1. E.C. 1.2.7.1 is a class of pyruvate oxidoreductase that was engineered with the constitutive, medium strength PsigX promoter from B. subtilis to overexpress the porCDAB operon from Paenibacillus zanthoxyli which encodes the A, B, C and D subunits of the pyruvate flavodoxin oxidoreductase protein. PorCDAB oxidizes pyruvate molecules that are produced as part of central carbon metabolism, thus freeing electrons that could potentially be utilized by the nitrogenase enzyme. Without being bound by any one hypothesis, overexpressing porCDAB may shift electron flux from the central carbon metabolism pathway towards the nitrogen fixation pathway.

Pb8: This strain illustrated the expression of an exemplary Paenibacillus zanthoxyli flavodoxin enzyme driven by a medium strength constitutive promoter. Since nitrogenase is inhibited by oxygen, Paenibacillus may rely mainly on pyruvate as its main electron source once in the soil (fermentative or pseudo-fermentative metabolism). RNA-seq performed under N-free condition and oxygen limitation in the closely related species Paenibacillus zanthoxyli supported this hypothesis and allowed identification of a target soluble electron carrier gene, fldA, to overexpress in Pb (FIG. 1). The gene fldA encodes a flavodoxin, a soluble cytoplasmic protein electron carrier containing a flavodoxin mononucleotide (FMN) redox center. fldA expression may be a regulatory point of the nitrogenase activity; thus constitutive expression of fldA could alleviate a rate limiting step. Therefore, this strain was engineered with the constitutive, medium strength PsigX promoter from B. subtilis to overexpress the fldA gene from Paenibacillus zanthoxyli. Flavodoxin is involved in transporting electrons from pyruvate to the [4Fe-4S] metallo-cluster of the iron nitrogenase homodimer. Pyruvate is oxidized by pyruvate flavodoxin oxidoreductase (PorABCD). PorABCD then reduces flavodoxin, which in turn reduces the [4Fe-4S] cluster (Poudel et al. J Bacteriol. 2018; 200(10):e00757-17). Without being bound by any one hypothesis, overexpression of flavodoxin may increase the supply of electrons to nitrogenase and thereby increase the capacity of the cells to reduce nitrogen.

Pb10: This strain was engineered with the very strong constitutive tRNA promoter PtrnQ from B. subtilis to overexpress the porCDAB operon from Paenibacillus zanthoxyli like Pb6 described above. As with PB6, the porCDAB operon expressed in this strain contains the A, B, C and D subunits of the pyruvate flavodoxin oxidoreductase protein, which oxidizes pyruvate molecules that are produced as part of central carbon metabolism, thus freeing electrons that could potentially be utilized by the nitrogenase enzyme. As with Pb6, overexpressing porCDAB may shift electron flux from the central carbon metabolism pathway towards the nitrogen fixation pathway. The difference between Pb6 and Pb10 is in the strength of the constitutive promoter. Multiple strains were made with different promoter strengths to test the effect of expression level. Pb6 uses the medium strength PsigX promoter, while Pb10 uses the very strong PtrnQ promoter. Without being bound by any one hypothesis, the stronger PtrnQ promoter may have a larger beneficial impact on the nitrogen fixation capacity of the cells.

Applicants observed that Pb10 lost its ability to sporulate and exhibited abnormal growth and, for this reason, conducted whole genome sequencing to determine the changes to the strain. The Pb10 strain used in the examples below contain the following unintended changes in addition to the engineered changes described above: tuaD (disruptive inframe deletion) and transposase IS116/IS110/IS902 family protein (frameshift variant). These unintended changes may have affected the sporulation and growth phenotype of Pb10 and its derivatives.

Pb11: This strain was engineered with the very strong constitutive tRNA promoter PtrnQ from B. subtilis to overexpress the sufCDSUB operon of Paenibacillus zanthoxyli. As with strain Pb16, the suf operon of Paenibacillus zanthoxyli is involved in the formation of the electron accepting, iron-sulfur cluster of the nitrogenase enzyme. Without being bound by any one hypothesis, overexpressing the suf operon may increase availability of the [4Fe-4S] metallo-cluster cofactor to the nitrogenase enzyme, thus increasing the capacity of the cells to fix nitrogen.

Applicants observed that Pb11 lost its ability to sporulate and exhibited abnormal growth and, for this reason, conducted whole genome sequencing to determine the changes to the strain. The Pb11 strain used in the examples below contain the following unintended change in addition to the engineered changes described above: tuaD (disruptive inframe deletion). These unintended changes may have affected the sporulation and growth phenotype of Pb11 and its derivatives.

There are two differences between Pb11 and Pb16. In Pb11 the suf operon was cloned from Paenibacillus zanthoxyli, whereas in Pb16, the suf operon was cloned from Paenibacillus forsythiae. In both cases the suf operon originated in a strain with high nitrogenase activity, but the two different operons may perform differently from each other when heterologously expressed in Pb. The second difference is in the promoter strength. In Pb11 the suf operon is being expressed from the very strong PtrnQ promoter, whereas in Pb16 the operon is being expressed from the medium strength PsigX promoter. As with the porCDAB strains Pb6 and Pb10 described above, multiple strains were made with different promoter strengths to test the effect of expression level, and the stronger PtrnQ promoter may have a larger beneficial impact on the nitrogen fixation capacity of the cells.

Pb12: This strain was engineered with the very strong constitutive tRNA promoter PtrnQ from B. subtilis to overexpress the fer gene from Paenibacillus zanthoxyli encoding ferredoxin. As with the flavodoxin gene in Pb8 described above, ferredoxin is involved in electron transport from pyruvate to the [4Fe-4S] cluster of the iron nitrogenase homodimer. Like flavodoxin, ferredoxin serves as the electron acceptor from pyruvate and the electron donor to nitrogenase. Under anaerobic conditions both ferredoxin and flavodoxin can perform this function but under aerobic conditions only flavodoxin can be active because ferredoxin must be spatially or temporally separated from oxygen (Poudel et al. J Bacteriol. 2018; 200(10):e00757-17). Without being bound by any one hypothesis, overexpression of ferredoxin may increase the supply of electrons to nitrogenase and thereby increase the capacity of the cells to reduce nitrogen especially if the cells exist in an anaerobic or microaerobic environment in the rhizosphere.

Applicants observed that Pb12 lost its ability to sporulate and exhibited abnormal growth and, for this reason, conducted whole genome sequencing to determine the changes to the strain. The Pb12 strain used in the examples below contain the following unintended changes in addition to the engineered changes described above: tuaD (disruptive inframe deletion), traX (frameshift variant), and transposase IS116/IS110/IS902 family protein (frameshift variant). These unintended changes may have affected the sporulation and growth phenotype of Pb12 and its derivatives.

Pb16: This strain was engineered with the constitutive, medium strength PsigX promoter from B. subtilis to overexpress the sufCDSUB operon from Paenibacillus forsythiae. The suf operon is involved in the formation of the electron accepting, iron-sulfur cluster of the nitrogenase enzyme. Without being bound by any one hypothesis, overexpressing the suf operon may increase availability of the four iron-four sulfur [4Fe-4S] metallo-cluster cofactor to the nitrogenase enzyme, thus increasing the capacity of the cells to accept electrons and therefore to fix nitrogen.

Additional Pb strains were constructed and insertion of the expression cassettes were verified by sequencing. Additional strains, along with Pb strains of Table 1, are included in Table 3.

TABLE 3
Additional genetically modified Pb strains of the disclosure.

| | | | SEQ ID |
|---|---|---|---|
| Strain | Expression cassette integrated | Purpose of genetic | NO: for |
| Code | in Pb strain | modification | cassette |
| Pb1 | Pb PtrnQ mRuby | Fluorescent reporter; | 1 |
| | | constitutive promoter | |
| Pb6 | Pb PsigX | Increase oxidoreductase | 2 |
| | Pz_porCA | activity; constitutive | |
| | | promoter | |
| Pb7 | Pb PsigX | Increase iron-sulfur | 3 |
| | Pz_sufCDSUB | cluster assembly; | |
| | | constitutive promoter | |
| Pb8 | Pb PsigX | Increase protein | 4 |
| | Pz_fldA | electron carrier activity | |
| | | (flavodoxin); constitutive | |
| | | promoter | |
| Pb9 | Pb PgroES | Increase protein | 5 |
| | Pz_fer | electron carrier activity | |
| | | (ferredoxin); constitutive | |
| | | promoter | |
| Pb10 | Pb PtrnQ | Increase oxidoreductase | 6 |
| | Pz_porCA | activity; constitutive | |
| | | promoter | |
| Pb11 | Pb PtrnQ | Increase iron-sulfur | 7 |
| | Pz_sufCDSUB | cluster assembly; | |
| | | constitutive promoter | |
| Pb12 | Pb PtrnQ | Increase protein | 8 |
| | Pz_fer | electron carrier activity | |
| | | (ferredoxin); constitutive | |
| | | promoter | |
| Pb13 | Pb PtrnQ | Increase protein | 9 |
| | Pz_fldA | electron carrier activity | |
| | | (flavodoxin); constitutive | |
| | | promoter | |
| Pb16 | PsigX | Increase iron-sulfur | 10 |
| | Pf_sufCBSUB | cluster assembly; | |
| | | constitutive promoter | |
| Pb21 | Pb PmreB | Increase oxidoreductase | 11 |
| | Pf_porCA | activity; constitutive | |
| | | promoter | |
| Pb22 | Pb PsigX | Increase protein | 12 |
| | Pf_fldA | electron carrier activity | |
| | | (flavodoxin); constitutive | |
| | | promoter | |
| Pb26 | Pb PsigX | Increase oxidoreductase | 13 |
| | Pf_porCA | activity; constitutive | |
| | | promoter | |
| Pb28 | Pb PmreB | Increase protein | 14 |
| | Pz_fldA | electron carrier activity | |
| | | (flavodoxin); constitutive | |
| | | promoter | |
| Pb29 | Pb PmreB | Increase iron-sulfur | 15 |
| | Pf_sufCBSUB | cluster assembly; | |
| | | constitutive promoter | |
| Pb30 | Pb PmreB | Increase oxidoreductase | 16 |
| | Pz_porCA | activity; constitutive | |
| | | promoter | |
| Pb31 | Pb PgroES | Increase protein | 17 |
| | Pf_fldA | electron carrier activity | |
| | | (flavodoxin); constitutive | |
| | | promoter | |
| Pb33 | PmreB Pz | Increase protein | 18 |
| | fer | electron carrier activity | |
| | | (ferredoxin); constitutive | |
| | | promoter | |
| Pb34 | PsigX Pz | Increase oxygen | 19 |
| | DUF269 | protection; constitutive | |
| | | promoter | |
| Pb38 | PmreB Pz | Increased oxygen | 20 |
| | DUF269 | protection; constitutive | |
| | | promoter | |

Pb strains that were targeted to increase protein electron carrier activity included Pb8, Pb9, Pb12, Pb13, Pb22, Pb28, Pb31 and Pb33. Pb strains that were targeted to increase iron-sulfur cluster assembly included Pb7, Pb11, Pb16, and Pb29. Pb strains that were targeted to increase oxygen protection included Pb34, and Pb38. Pb strains that were targeted to increase oxidoreductase activity include Pb6, Pb10, Pb21, Pb26, and Pb30.

### Example 2. Nitrogenase Activity of Engineered Pb Strains as Measured by Acetylene Reduction Assay (ARA)

The in vitro ARA was used to determine nitrogenase activity for Pb strains. Pb strains were struck out on TSA plates supplemented with 5 g/L ammonium chloride and 5 μg/ml chloramphenicol (when applicable) and grown for 3 days at 37° C. Colonies were picked into 96-well, deep well plates filled with 1 ml of the same medium in liquid form and incubated for 24 hours at 37° C. with shaking at 999 RPM and 1 mm orbit to produce biomass for inoculum. Cultures were centrifuged and washed twice in phosphate buffered saline (PB solution to remove residual ammonium before resuspending in 1 ml PBS solution. Five μl of this resuspension was diluted into 1 ml Seldin nitrogen-free medium for Paenibacillus and cells were incubated for 22 hours in an anaerobic chamber at 30° C. without shaking to acclimate the cells to the nitrogen fixation mode.

Cells were resuspended by pipetting and 500 μl of each culture was diluted into 5 ml of the same medium in a 10 ml gas chromatograph vial. The vials were capped with silicone septums and crimped inside the anaerobic chamber to maintain the anoxic environment before acetylene was added according to the acetylene reduction assay (ARA) protocol. Vials were then incubated at 30° C. with shaking at 220 RPM and 25 mm orbit for 24 hours before analysis on the gas chromatograph.

ARA for Pb was analyzed by a fixed effect linear regression model to determine whether significant differences between means were present in the data. Removal of Run as a random effect was determined by Likelihood Ratio Test of nested models. Approximate chi-squared statistic and p-values were included in the main text. Ethylene production in nanomoles was used as the outcome, with Strain as the predictor. A Wald test was used to assess the effect of strain on ethylene production and p-values were reported in the main text. Retrospective power analysis was performed by calculating detectable effect size of mean differences at a significance level of 0.05 and power of 80%. The maximum standard deviation of the Pb strains were used to generate mean estimates. Dose response for Pb ARA fit using an n-parameter logistic model, where n=2, 3, 4, or 5 did not converge because ammonium dose levels provided insufficient information to constrain a model.

Results. The engineered strain Pb8 was subjected to an in vitro ARA to measure the effects of the genetic modification (n=3). The Pb8 was compared to a wt and a mRuby expressing strain (Pb1) as a heterogeneous protein expression control (FIG. 3). The Pb8 can also be compared to a strain with a knockout of PROKKA_03723, the gene in which the Pb8 expression cassette was integrated.

The mRuby control (Pb1) carries an insertion for a fluorophore in locus PROKKA 04492. Pb1 showed a significant decrease when compared to wt (FIG. 3; p-value=0.006). The mRuby strain has no engineering for the nitrogen fixation genes, so the expectation was there would not be a significant difference between these two strains. The mRuby control strain also includes the following unintended changes: tuaD (disruptive inframe deletion) and transposase IS116/IS110/IS902 family protein (frameshift variant). These unintended changes may have affected the sporulation and growth phenotype of the mRuby control strain and its derivatives.

There was a significant improvement over the mRuby control (2.6 fold improvement, p-value=0.03), or a decrease compared to the wt (0.75 fold, p-value=0.16) (Table 4).

TABLE 4
Summary table of Pb8 performance in ARA.

| | | | Fold | | Fold |
|---|---|---|---|---|---|
| | ARA | | improve- | | improve- |
| | (nmoles | P-value | ment | P-value | ment |
| Strain | C₂H₄/OD) | (vs wt) | vs wt | (vs mRuby) | vs mRuby |
| Wt | 2.8 | — | 1 | 0.006 | 3.5 |
| Pb8 | 2.1 | 0.16 | 0.75 | 0.03 | 2.6 |
| mRuby | 0.8 | 0.06 | 0.3 | — | — |

Pb8 may have an improved ARA activity and was further tested in greenhouse in planta assays.

### Example 3. In Planta Performance in the Greenhouse

In planta phenotype determination experimental design and statistical data analysis. A high level in planta experimental plan is shown below in Table 5 and FIG. 4. The greenhouse experiments were designed to measure the effect of the genetically modified Pb microbes on plant phenotype.

TABLE 5
High level specifications of greenhouse experiments
for in planta phenotype determination.

| | |
|---|---|
| Number of independent | 3 independent experiments per strain |
| experiments per strain | on corn |
| Number of replicates per | 16 corn plants |
| experiment | |
| Soil type, volume per pot | Sungro, 800 ml/pot |
| Inoculation | Germinated corn seed drench |
| | 10⁹ cfu/seed |
| Initial Nitrogen | 25 ppm (allowing detection of 4-5 ppm |
| application | differences) |
| (sensitivity estimate) | |
| Greenhouse conditions | Photoperiod: 15 h |
| | Lightening: >225 w/m²* |
| | Humidity*: day: 50-55%; night: 70-75% |
| | Temperature: heating SP 60 F, cooling SP 80 F |
| | Soil moisture: 60% |
| | *may vary based on seasonal conditions |
| Phenotype determination | Engineered versus wild-type of corn shoot dry |
| | weight difference |
| | Extrapolated N-replacement |
| Controls | Mock: corn inoculated with buffer only |
| | (no bacteria) |
| | Wild-type (wt)*: corn inoculated with |
| | wild-type bacteria |
| | *used instead of nif- strain. The wt strain |
| | is the base strain used in the engineering |
| | iterations and thus a better benchmark |
| | of actual improvement (engineered |
| | versus parental strain). |
| Experiment statistical | Randomized complete block design (RCBD), |
| analysis | block as random factor, strain as fixed |
| | factor, linear mixed model, analysis of |
| | variance (ANOVA), Fisher's Least |
| | Significant Difference (LSD) test is used |
| | to compare engineered strains and wild-type |
| Inter-experimental | Combining multiple independent RCBD |
| statistical | experiments, adding experiment ID as another |
| analysis method | random factor to run the mixed model, and |
| | LSD test is used to compare strains and |
| | wild-type differences across experiments. |
| Power simulation | Pilot experimental data were collected and |
| | used to simulate power (probability to detect |
| | treatment difference) at given variation |
| | level, number of replicates, and expected |
| | effect size. |

A power analysis was conducted to estimate the number of replicates and was set at 16 (further described below). Given 16 replicates, the screening experiment has 80% probability to find 6-10% plant weight difference (corresponding to 4 N-ppm supplemented) between engineered and wt strains.

Plant inoculation. Corn seeds were germinated for two days on moist filter paper prior to inoculation, to the point that the radicle emerged 1-3 cm. Uniform seedlings were selected for inoculation. Microbe strains of interest were streaked out on solid media from cryogenic stocks three days before plant inoculation, and grown overnight at 30° C. Two days before plant inoculation, a lawn of cells was scraped with a sterile loop and inoculated into 5 ml of media in a 14 ml culture tube and grown overnight. Pb strains were grown in rich LB media. One day before plant inoculation, 100 μl to 5 ml of the starter culture was added to 50-100 ml of appropriate media in a 250 ml baffled flask and grown overnight in a shaker at 250 rpm to reaching OD₆₀₀=1 by the following morning. On days of inoculation, cultures were centrifuged at 4,000×g for 10 min, media was decanted, and pellets were resuspended in PBS buffer. PBS alone was used as a negative control in all subsequent steps and was referred to as “mock”. Cultures were normalized to OD₆₀₀=1 (10⁹ CFU/ml), and two 200 μl samples were taken for serial dilution in PBS and plating on respective solid media to determine CFU/ml of inoculum.

The germinated seedlings were incubated with the corresponding inoculum treatments for 30 min at room temperature with mild shaking. Inoculated seedlings were planted into a 3-cm deep hole in the center of corresponding pots for each microbial treatment in the greenhouse. Then 1 ml of corresponding inoculum was drenched around each seedling. Extra pots were prepared for each microbial treatment in order to replace the pot(s) with abnormal seedling 4 days after planting. An automated irrigation system was used to deliver nutrient solutions with 25 ppm N at 60% soil moisture setting. Every pot and plant was closely monitored every day and abnormal events were recorded. In some earlier experiments, 0 ppm N and 100 ppm N were used.

Nutrient solution preparation. The nutrient stock solutions contained the following chemicals (per liter, they were 100× solution) of each nutrient delivered in the specified form as follows.

25 ppm Nitrogen nutrient solution stock solutions (100×)—Solution A: 12.38 g KH₂PO₄, 1.10 g NH₄H₂PO₄, 23.44 g MgSO₄, 5.15 g KNO₃, 41.74 g K₂SO₄. Solution B: 10.70 g Ca(NO₃)₂, 4.71 g CaCl₂), 1.51 g FeEDTA, 0.38 g MnEDTA, 0.24 g Na₂B₈O₁₃ 4H₂O per liter solution.

0 ppm Nitrogen nutrient solution stock solutions: (100×)—Solution A: 13.64 g KH₂PO₄, 23.44 g MgSO₄, 45.63 g K₂SO₄. Solution B: 6.71 g CaCl₂), 1.51 g FeEDTA, 0.38 g MnEDTA, 0.24 g Na₂B₈O₁₃ 4H₂O per liter solution.

100 ppm Nitrogen nutrient solution stock solution (100×)—Solution A: 11.63 g NH₄H₂PO₄, 23.44 g MgSO₄, 5.15 g KNO₃, 49.78 g K₂SO₄. Solution B: 50.13 g Ca (NO₃)₂, 1.51 g FeEDTA, 0.38 g MnEDTA, 0.24 g Na₂B₈O₁₃ 4H₂O per liter solution.

Before fertigation each time, the final solution volume was calculated based on the solution volume (ml) needed for each pot and the number of pots to be fertigated. Then 1 part of A solution and 1 part of B solution were mixed to 100 parts of the final solution.

Data collection. Growth stage (GWTPBTG): Leaf number was recorded weekly for each plant by counting fully expanded leaves. Plant height (PLTHT_tip): Plant height was measured weekly from soil surface to the tallest extended leaf tip (manually straightened up). Leaf greenness (CHLSPAD): One day before harvest, leaf greenness was measured on the upmost fully expanded leaf by using a SPAD meter. Four readings were taken in the middle portion of the leaf and the average of the 4 readings for each plant was recorded.

Shoot fresh weight (SHOOFWT): On day 28 after planting, all plants were watered early in the morning to make sure the soil in every pot was not dried to have uniform plant water content across the experiment. Plants were watered again early afternoon if the harvest was not completed in the morning. A plant was cut from the soil surface and shoot fresh weight measured immediately and placed into a paper bag with a plant tag on the bag for drying. All plants were cut uniformly from soil surface across the entire experiment.

Shoot dry weight (SHOODWT): Bags with plants were placed in the oven in the growth chamber room with 105° C. temperature for 15 min to stop all biological activities. Then the plant samples were dried with oven temperature at 75° C. until constant weight (weigh 10 bags from different positions in the oven with less than 1% weight decrease over 24 hours). Shoot dry weight (0% moisture) was taken immediately (within 30 seconds after the bag is removed from oven).

Greenhouse experiments were combined. For example, experiments can be 1-factor (strains only) or 2-factor (strains+N rates) RBCD with multiple experiments. The N rate was 25 ppm or was a combination of a number of N rates (e.g., 0, 25, 100 ppm N).

In planta greenhouse assay N-response. The response of corn fresh weight to various concentrations of nitrogen measured in parts per million (ppm) was evaluated. The goal of this experiment was to establish the screening conditions for routine screening. A concentration of supplemented N was selected that allowed for detection of microbial N supplementation, which was calculated from the observed dry biomass difference between plants treated with wild-type and engineered strains. In particular, an N-dose was selected where stark differences in physiological parameters between nitrogen doses would be observed. Moreover, the experiment allowed determination of the sensitivity of N-response at the lower N concentrations. This work showed that a linear relationship between applied N and plant mass was observed within the range of 0 to 100 ppm of added N and that nitrogen dependent responses at the lower end of the dose response could be detected (FIG. 6). This experiment was repeated twice in two different soil conditions (Turface and Sungro) and a consistent slope was calculated for both datasets (FIG. 7; 0.047 and 0.046 gram increase per 1 ppm increase in N), despite the difference in absolute means of biomass between the two experiments, leading to a different intercept in each regression line. Since plant growth and biomass accumulation responses to the fixed N microbiologically are the same as fertilizer N application, the value of these slopes allows for estimation of the approximate increase in N that would be required to achieve an equivalent increase in biomass. Based on the data, a correlation of 22 ppm N per 1 gram increase of plant biomass was observed. The 25 ppm N allowed for 1) the plants to exhibit clear nitrogen deprivation and 2) even modest nitrogen supplementation by the strain to be detected. Results showed that plants fertilized with 25 ppm N showed distinct nitrogen limitation phenotypes, and the assay allowed for reliable detection of 4-5 ppm differences. Therefore, 25 ppm N was selected as the basic N-condition for screening so as not to skew selection of strains based on physiological stress of the plants and to detect significant strain effects even if the nitrogen fixed by the strain was modest.

In planta power analysis. A power analysis was run to assess the ability to detect differences in the mean biomass in grams between engineered strains and wt. FIG. 8 shows a power analysis for plants of large and smaller sizes. An initial power analysis was done using variance and mean fresh weight from model results of an experiment that produced the larger plants under treatment with the wt strain (M=107 g/plant, varE=63.8). Experimental protocols were expected to have plant biomass most similar to these larger plants. To detect a 10% increase in biomass (10.7 g) in plants of this size with 80% power at a significance level of 0.05, 8 replicates would be needed per level of N. For experiments that produce smaller plants (M=36.44 g/plant), a second power analysis was run using a smaller variance as seen in the results of earlier experiments (varE=13.26). Under these conditions, a 5 g increase in biomass would be detectable with 80% power at a significance level of 0.05, given the same number of replicates (8). An increase of 5 grams in fresh weight is equivalent to an increase of 4.6 ppm of applied N by a dose-response curve using fresh weight in grams as outcome.

These power calculations were done using least-squared means and residual variance as estimated by mixed model from a series of independent experiments. Mixed models for each experiment included applied N and strain as fixed effects, and replicate or block as random effect with fresh weight in grams as outcome. Fresh weight was used as the indicator for biomass in place of dry weight, as dry weight was not collected for some early experiments.

The above power analysis described the number of replicates that were determined for future experiments. Given 16 replicates, the screening experiment had 80% probability to find 6-10% plant fresh weight difference between engineered strains and wt. Some experiments included a higher number of replicates than recommended. This is due in part to an unexpected surplus of greenhouse resources which allowed for an increase in replicates that was greater than the experimental plan originally designed.

Results. The Pb8 strain was tested 3 times independently (16 replicates each time) and found to have a consistently positive in planta phenotype. Since variation occurs between experiments (seasonal change, culture conditions), an inter-experimental statistical analysis was used to bolster confidence in the results.

The engineered Pb8 was first tested in the GH in planta assay at a fixed N-application rate of 25 ppm. The results shown in FIG. 8 and Table 6 are based on an inter-experiment analysis run on 3 independent GH experiments. The overall dry weight of control plants varied across experiments, and combining multiple experiments (inter-experimental analysis) accounted for experimental variation due to microbial and seasonal difference and allowed increased power to detect the mean difference between plants treated with wt and engineered strains. As a result, this inter-experiment analysis detected a statistically significant increase of dry biomass in plants treated with an engineered strain when compared to those treated with wt microbe with 25 ppm N treated plants.

TABLE 6
Summary of Pb8 25 ppm N-application GH experiment.

| | | Biomass | Biomass | | | |
|---|---|---|---|---|---|---|
| | | (g dry | difference | N-ppm | Std Err | p- |
| Strain | Genotype | weight) | (g) | supplemented | (g) | value |
| Pb0 | wt | 8.08 | — | — | — | — |
| Pb8 | ::PsigX:Pz_fldA | 8.31 | 0.23 | 5.06 | 0.14 | 0.098 |

An inter-experiment analysis of Pb8 across three independent experiments at a fixed N-application rate of 25 ppm showed an improvement of biomass by plants treated with the engineered Pb8 strain above the wt strain (FIG. 8). The average N-ppm supplemented by the Pb8 across the 3 independent experiments was estimated to be 5.06 ppm N (corresponding to a dry weight difference of 0.23 g), with a p-value of 0.098. The positive phenotype observed with Pb8 was confirmed in a fourth independent experiment (FIG. 9).

FIG. 9 and Table 7 indicate Pb8 performance by N-supplement estimated from mean dry weight comparisons with wild-type. The results of 3 independent experiments were defined by run number and separated by nitrogen rate where applicable. Run 3 plants treated with the engineered Pb8 strain showed no change from wt treated plants. This result may be due to overperformance of the wt strain in Run 3. A small increase in fresh weight was seen in Run 3 plants. The Pb8 strain was engineered to improve the electron flux to the nitrogenase via over-expression of the fldA gene. Thus, it was not expected that there would be a positive GH phenotype sustained through an increasing concentration of exogenous nitrogen (0, 25 and 100 ppm).

TABLE 7
Three independent experiments show improvement in dry weight
of plants treated with Pb8 in comparison to wild-type plants.

| | | | N-ppm of | Weight | | |
|---|---|---|---|---|---|---|
| | | | the | difference | P value | WT plant |
| | | | difference | from wt in | from | size |
| | Applied | | from wt | grams dry | mixed | (g dry |
| Run | Nitrogen | | (supplemented) | weight | model | weight) |
| Run 1 | 0 | ppm | 0.44 | 0.02 | 0.83 | 2.08 |
| Run 1 | 25 | ppm | 4.84 | 0.22 | 0.10 | 3.37 |
| Run 1 | 100 | ppm | 3.52 | 0.16 | 0.30 | 6.17 |
| Run 2 | 25 | ppm | 9.68 | 0.44 | 0.09 | 12.66 |
| Run 3 | 25 | ppm | 3.08 | 0.14 | 0.28 | 7.25 |

The effect of microbial treatment was assessed by a mixed model and mean effect was aggregated across nitrogen levels to improve statistical significance. A single effect was estimated with standard error that accounted for additional variation due to multiple nitrogen levels. Based on the result of the statistical analysis performed on the data and presented in FIG. 10 and Table 8, the engineered Pb8 strain led to a non-significant increase in dry weight biomass of 0.21 g. This increase corresponds to 4.62 N-ppm supplemented, but this effect was not found to be statistically significant through a wide range of N-application (p-value=0.344).

This result indicates that the in planta gain is not sustained through different N application rates. In this engineering strategy, the fldA gene is overexpressed to favor electron flow to the nitrogenase enzyme, and the nitrogen fixation genes are not deregulated. Thus, the increased NH₃ concentration most likely led to a repression of the genes involved in nitrogen fixation, decreasing the nitrogenase total activity in the cell and reducing NH₃ production.

TABLE 8
Four independent experiments show change in dry weight
of plants treated with Pb8 in comparison to wt plants.

| | | | | Weight | P value | wt plant |
|---|---|---|---|---|---|---|
| | | | N-ppm | difference | from | size |
| | Applied | | increase | from wt | mixed | (g dry |
| Run | Nitrogen | | over wt | in grams | model | weight) |
| Run 1 | 0 | ppm | 9.24 | 0.42 | 0.077 | 8.50 |
| Run 1 | 25 | ppm | 13.2 | 0.60 | 0.01 | 9.89 |
| Run1 | 100 | ppm | 1.98 | 0.09 | 0.74 | 12.33 |
| Run 2 | 25 | ppm | 3.52 | 0.16 | 0.44 | 5.74 |
| Run 3 | 25 | ppm | 0 | 0.00 | 0.998 | 7.8 |
| Run 4* | 25 | ppm | 5.06 | 0.23 | 0.27 | 5.45 |

*Run 4 data had not been included in the inter-experiment analysis.

This analysis showed that a significant increase in dry biomass was observed only at a N application rate of 25 ppm (FIG. 8 and Table 6). The mean dry weight in plants treated was 0.23 grams higher, a 2.8% increase, when compared with wt corresponding to 5 N-ppm supplementation; these results are statistically significant at the 0.1 probability level. These results were expected since improving the electron flow to the nitrogenase enzyme does not alleviate the repression of the N-fixation genes by the exogenous applied N. This study suggested that improving FldA expression may lead to an increased electron flow to the nitrogenase, improving the overall N fixation process. A subsequent strategy will be to introduce N-fixation gene deregulation modification to this strain.

A summary of engineered Pb microbe Pb8 performance compared to wt in in vitro assays and in planta are presented in Table 9.

TABLE 9
In vitro and in vivo engineered strain summary.

| | | Corn root | | In | In | | |
|---|---|---|---|---|---|---|---|
| | | Colonization | | vitro: | vitro: | | |
| | | wt strains | | ARA | ARA | | In planta |
| | | (3 weeks | In vitro: | gain | gain | In vitro: | gain |
| | | after | NH₃ gain | over | over | ¹⁵N over | (N-ppm |
| Strain | Genotype | planting) | over wt | wt | mRuby | wt | supplemented) |
| Pb8 | ::PsigX:Pz_fldA | 10\hat{ }5 cfu/g | To be | No | Gain | To be | 5 ppm |
| | | fcw | determined | gain | | determined | |
| | | inoc. at | | | | | |
| | | 10\hat{ }5 cfu/g | | | | | |
| | | fcw | | | | | |

### Example 4. In Planta Performance in the Field

Experimental approach. Microbial strains were tested in field studies with N limited conditions. The entire field area received a non-optimal rate of 75 lb N/acre before planting. N was provided as 32% or 28% liquid urea ammonium nitrate (UAN). Six replicates of a Randomized Completed Block Design were established at each location (U of IL and Agldea_MO). Forced pair-wise comparisons between each engineered microbial strain and its adjacent WT control were evaluated. This design managed field spatial variability which naturally exists due to the differences in soil N across fields, where the response to N typically changes over short distances. In each of the 6 replicates, one additional plot received a supplemental side-dress N application of 75 lbs N/acre at the V6 growth stage. The crop response to the N fixing microbial treatments was also compared to the crop response to this side-dress N treatment. This trial design typically identifies efficacious N fixing microbial strains and provides an estimate of N replacement value.

Field trials were conducted to assess the efficacy of the genetically engineered Pb microbes to enhance plant yield in the field. As shown in Table 10, the indicated genetically engineered Pb microbes provided increased yield in field trials compared to wild-type controls. “NA” indicates that the strain was tested at only one location due to limited inoculum.

TABLE 10
Yield from field trials.

| | | Yield (bushels/acre | | | |
|---|---|---|---|---|---|
| Strain | | improvement over wt) | | | |
| name | Genotype | U of II | p-value | Agldea | p-value |
| Pb08 | PsigX:Pz_fldA | −7.9 | 0.92 | 3.85 | 0.71 |
| Pb10 | PtrnQ:porCA | NA | 0.06 | 7.8 | 0.37 |
| Pb11 | PtrnQ:sufCDSUB | 7 | 0.21 | NA | 0.31 |
| Pb12 | PtrnQ:fer | 11.4 | 0.23 | 3.6 | 0.67 |

### Example 5. Construction of Additional Engineered Strains

Additional engineering was conducted to obtain strains without the unintended changes described above, e.g., changes in genes such as tuaD, traX, farnesyl diphosphate synthase, and transposase IS116/IS110/IS902 family protein. PB172 was engineered to increase electron availability as described in Tables 11 and 12, resulting in strain Pb39. NRRL B-68085 was similarly engineered, resulting in strains Pp1-Pp4. Regarding the exact DNA sequences used for the expression of a protein, e.g., ferredoxin, in Pb39 and Pp1-Pp4, it is well known that, instead of cloning the DNA sequence encoding such protein from the genome of an organism, one can alternatively design a DNA sequence that, when transcribed into RNA and subsequently translated, will result in the same amino-acid sequence of said protein. This is readily achieved through the use of codon tables that are available to those skilled in the art. As previously discussed, it is further known that specific codons can be selected in this process to provide codon preference in a specific microbe. Examples of such designs of DNA sequences derived from original amino acid sequences are SEQ ID NO: 47 (derived from SEQ ID NO: 46) and SEQ ID NO: 49 (derived from SEQ ID NO: 48).

TABLE 11
List of Paenibacillus strain IDs, genotype, purpose
of genetic modification, and SEQ ID NOs for cassettes.

| | | Enzyme | | |
|---|---|---|---|---|
| | | Commission | Purpose | SEQ ID |
| Strain | Expression cassette | Number/protein | of genetic | NO: for |
| ID | integrated in Pb strain | class Gene | modification | cassette |
| Pb39 | czcB::PtrnQ:Pz_fer-Tfer | Protein electron | Increase flux of | 29 |
| | | carrier (ferredoxin) | electrons to nitrogen | |
| | | | fixation reaction | |
| Pp1 | czcB::PtrnQ:Pz_fer-Tfer | Protein electron | Increase flux of | 30 |
| | | carrier (ferredoxin) | electrons to nitrogen | |
| | | | fixation reaction | |
| Pp2 | czcB::PtrnQ:Pm_fer-Tfer | Protein electron | Increase flux of | 31 |
| | | carrier (ferredoxin) | electrons to nitrogen | |
| | | | fixation reaction | |
| Pp3 | czcB::PR′:Pm_fer-Tfer | Protein electron | Increase flux of | 32 |
| | | carrier (ferredoxin) | electrons to nitrogen | |
| | | | fixation reaction | |
| Pp4 | czcB::P43:Pm_fer-Tfer | Protein electron | Increase flux of | 33 |
| | | carrier (ferredoxin) | electrons to nitrogen | |
| | | | fixation reaction | |

TABLE 12
Components of genetic constructs and component source.

| | | | GenBank | GenBank |
|---|---|---|---|---|
| | | | accession | accession # |
| | | | # of source | of introduced |
| Name | Description | Source | genome | component |
| PtrnQ | Strong | Bacillus subtilis | CP053102.1 | Locus tag |
| | constitutive | | | HIR77_19330 (nt |
| | promoter | | | 3646432-3646619 |
| | | | | of CP053102.1) |
| PR′ | Medium- | Bacteriophage | NC_001416.1 | Locus tag |
| | strength | lambda | | lambdap72 Gene |
| | constitutive | | | ID: 2703521 (nt |
| | promoter | | | 44479-44603 of |
| | | | | NC_001416.1) |
| P43 | Medium- | Bacillus subtilis | CP136402.1 | Locus tag |
| | strength | | | RX583_13720 |
| | constitutive | | | WOA97549.1 (nt |
| | promoter | | | 2612735-2612840) |
| Pz_fer | Ferredoxin | Paenibacillus | GCF_000520715.1 or | Amino-acid (AA) |
| | | zanthoxyli JH29 | NZ_ASSD01000464.1 | sequence source is |
| | | | | WP_03673731.1. |
| | | | | The amino-acid |
| | | | | sequence is |
| | | | | provided as SEQ ID |
| | | | | NO: 46 |
| | | | | An exemplary |
| | | | | genomic source for |
| | | | | DNA encoding the |
| | | | | protein of SEQ ID |
| | | | | NO: 46 can be |
| | | | | NZ_ASSD01000464.1 |
| | | | | (Locus tag |
| | | | | L691_RS29285). |
| | | | | Alternatively, DNA |
| | | | | encoding the |
| | | | | protein can be |
| | | | | designed through |
| | | | | reverse translation |
| | | | | using a codon table |
| | | | | to obtain a DNA |
| | | | | sequence. For |
| | | | | example, SEQ ID |
| | | | | NO: 47. |
| Pm_fer | Ferredoxin | Paenibacillus | NZ_DOOE01000013.1 | Amino-acid (AA) |
| | | (multispecies) | | sequence source is |
| | | | | WP_024629252.1. |
| | | | | The amino-acid |
| | | | | sequence is |
| | | | | provided as SEQ ID |
| | | | | NO: 48 |
| | | | | An exemplary |
| | | | | genomic source for |
| | | | | DNA encoding the |
| | | | | protein of SEQ ID |
| | | | | NO: 48 can be |
| | | | | NZ_DOOE0100001 |
| | | | | 3.1 (nt 127584- |
| | | | | 127826) |
| | | | | (+). Alternatively, |
| | | | | DNA encoding the |
| | | | | protein can be |
| | | | | designed through |
| | | | | reverse translation |
| | | | | using a codon table |
| | | | | to obtain a DNA |
| | | | | sequence. For |
| | | | | example, SEQ ID |
| | | | | NO: 49. |
| Tfer | Transcriptional | Paenibacillus | GCF_000520715.1 | WP_03673731 |
| | terminator | zanthoxyli JH29 | | |
| TgyrB | Transcriptional | Paenibacillus | CP000154.2 | Locus tag |
| | terminator | polymyxa | | ADM67911.1 (nt |
| | | | | 6761-6861) |
| TcatA | Transcriptional | Bacillus subtilis | CP052842.1 | Locus tag |
| | terminator | | | AHIR78_01790 (nt |
| | | | | 330583-330603) |

The taxonomy of the subject microorganisms was validated after selection on chloramphenicol medium when integrants and integration sites were verified through whole genome Illumina sequencing as belonging to Pb or Pp respectively.

The morphological features of the subject microorganisms were similar to the recipient organism. In addition, all strains were expected to be improved for nitrogen fixation as described in Table 11.

The general form of the final constructs as inserted into the genome of the recipient organism PB172 or NRRL B-68085 respectively to generate the various strains described in Table 11 is depicted in FIG. 2G. The cassettes (i.e. the combinations of promoter regions+ferredoxin gene+terminator regions) for each strain are defined by SEQ ID NOs: 29-33 for each respective strain, as described in Table 11. The upstream and downstream homology arms for each strain are defined as SEQ ID NOs: 34-37, as described in Table 13.

TABLE 13
Sequences of homology pairs used for targeting integrations.

| | SEQ ID NO: | SEQ ID NO: |
|---|---|---|
| Applicable | for upstream | for downstream |
| strains | homology arm | homology arm |
| Pb27, Pb28, Pb29, | 84 | 85 |
| Pb30, Pb31, Pb32, | | |
| Pb33, Pb34 | | |
| Pb39 | 34 | 35 |
| Pb48, Pb49, Pb50 | 82 | 83 |
| Pb51 | 113 | 114 |
| Pp1, Pp2, Pp3, Pp4, | 36 | 37 |
| Pp5, Pp6, Pp7, Pp8, | | |
| Pp9, Pp10, Pp11, | | |
| Pp12, Pp23, Pp24, | | |
| Pp25, Pp26, Pp27, | | |
| Pp28, Pp29, Pp30, | | |
| Pp31, Pp32, Pp33, | | |
| Pp35, Pp36, Pp37, | | |
| Pp38, Pp39, Pp40, | | |
| Pp41, Pp42, Pp43, | | |
| Pp44, Pp45, Pp46, | | |
| Pp47, Pp48, Pp49 | | |
| Pp13 | 59 | 60 |
| Pp14, Pp15 | 80 | 81 |
| Pp34 | 185 | 186 |

The exemplary strains described in Table 11 were made using the following general technique.

Integration of the exogenous genes was performed using targeted integration resulting in strains expressing the gene or operon of interest. The homology sequences used for targeting are shown in Table 13. However, one or ordinary skill in the art can envision using alternative methods of gene editing to arrive at substantially similar strains.

Briefly, the subject organisms were constructed in the following process using a multi-plasmid system.

A non-replicative integration vector containing an antibiotic marker for selection in Paenibacillus spp., a restriction site for counterselection in Paenibacillus spp., an antibiotic marker and origin of replication for selection and cloning in Escherichia coli, and ~1 kb homology arms to direct site-specific, homologous recombination into the Paenibacillus spp. genome, is constructed using standard cloning techniques. Homologous recombination is generally considered to be high fidelity, and to yield a negligible rate of off-target integration events. Nucleic acid sequences encoding cargoes of interest can be inserted between the upstream and downstream homology arms to direct their insertion at the desired locus. In the case of deletions, the 3′ end of the upstream homology arm and the 5′ end of the downstream homology arm immediately flank the sequence to be deleted, typically the entire coding sequence of the gene to be deleted, from the start codon to the stop codon, inclusive. The integration vector is prepared from a methylation-deficient strain of E. coli and transformed into a Paenibacillus strain harboring a temperature-sensitive helper plasmid via electroporation. The temperature-sensitive helper plasmid provides an essential replication factor that allows the integration vector to replicate, only in its presence. Furthermore, it contains a unique antibiotic resistance cassette that allows for its maintenance and for screening.

To select for integrants, transformants are streaked on media selecting for the integration vector and grown at a restrictive temperature that blocks the replication of the temperature-sensitive helper plasmid. At this point, the entire integration plasmid sequence, including the antibiotic resistance marker and endonuclease recognition site, has been introduced at the desired locus via a single crossover (X-over) event by homologous recombination. The loss of the helper plasmid and the integration of the non-replicative plasmid at the desired locus are confirmed by plating on selective media and a series of PCRs to detect the upstream and downstream integrative vector-genome junctions (to ensure integration occurred at the intended locus), as well as to probe for the helper plasmid to ensure it has been lost. At this stage, correct transformants should be sensitive to the antibiotic marker encoded on the helper plasmid and resistant to the antibiotic marker encoded in the integrative vector backbone.

Next, the single crossover strains are transformed with a temperature-sensitive variant of the helper plasmid that additionally encodes an endonuclease. The endonuclease induces a lethal double strand break (at the recognition sequence that has been introduced into the genome during the previous step), which must be resolved by one of two possible homologous recombination events, between either the upstream (reverting to wild-type) or downstream (inserting the desired, markerless edit to the genome) homology arms. These two recombination events, in theory, occur with equal probability and can be easily distinguished by PCR. The second crossover event results in the excision of the integration vector backbone, rendering the resulting strain sensitive to antibiotic treatment. Transformants are subsequently grown on plates lacking antibiotics at a restrictive temperature to prevent replication of the endonuclease plasmid, to give rise to the final, edited strain. Therefore, the final strain is devoid of all resistance markers. The loss of the plasmid encoding the endonuclease, excision of the non-replicative plasmid backbone, and the presence of the desired edit are all confirmed by plating on selective media and by a PCR spanning the edited region or a region of the plasmid encoding the endonuclease. Whole-genome sequencing is performed to verify the sequence and location of the integrated construct, as well as to ensure no deleterious single-nucleotide polymorphisms (SNPs) have arisen in the genetic background during editing. Whole-genome sequencing analysis is aided by the use of automated software, which identifies the insertion location and any SNPs in the integrated construct, and ensures no off-target integrations have occurred.

Following a round of engineering, the edited strains can be transformed with the temperature-sensitive helper plasmid to facilitate further rounds of editing.

Pb39: PB172 was engineered with the strong constitutive tRNA promoter PtrnQ from B. subtilis to overexpress the fer gene from Paenibacillus zanthoxyli (Pz_fer) encoding ferredoxin. Since nitrogenase is inhibited by oxygen, Paenibacillus may rely mainly on pyruvate as its main electron source once in the soil (fermentative or pseudo-fermentative metabolism). Ferredoxin, along with flavodoxin, is involved in electron transport from pyruvate to the [4Fe-4S] cluster of the iron nitrogenase homodimer (FIG. 1). Ferredoxin serves as the electron acceptor from pyruvate and the electron donor to nitrogenase. Under anaerobic conditions both ferredoxin and flavodoxin can perform this function but under aerobic conditions only flavodoxin can be active because ferredoxin must be spatially or temporally separated from oxygen (Poudel et al. J Bacteriol. 2018; 200(10):e00757-17). fer expression may be a regulatory point of the nitrogenase activity; thus constitutive expression of fer could alleviate a rate limiting step. Without being bound by any one hypothesis, overexpression of ferredoxin may increase the supply of electrons to nitrogenase and thereby increase the capacity of the cells to reduce nitrogen especially if the cells exist in an anaerobic or microaerobic environment in the rhizosphere. The gene Pz_fer, along with its promoter and terminator region (together defined by SEQ ID NO: 29) was inserted in the czcB locus, i.e. in between SEQ ID NO: 34 and SEQ ID NO: 35.

Pp1: The NRRL B-68085 strain was engineered with the strong constitutive tRNA promoter PtrnQ from B. subtilis to overexpress the fer gene from Paenibacillus zanthoxyli (Pz_fer) encoding ferredoxin. As described above in greater detail, constitutive expression of ferredoxin could alleviate the rate limiting step in nitrogenase activity by possibly increasing the supply of electrons to nitrogenase and thereby increase the capacity of the cells to reduce nitrogen under different environments. The gene Pz_fer, along with its promoter and terminator region (together defined by SEQ ID NO: 30) was inserted in the czcB locus, i.e. in between SEQ ID NO: 36 and SEQ ID NO: 37.

Pp2: The NRRL B-68085 strain was engineered with the strong constitutive tRNA promoter PtrnQ from B. subtilis to overexpress the fer gene from Paenibacillus spp. (Pm_fer) encoding ferredoxin. As described above in greater detail, constitutive expression of ferredoxin could alleviate the rate limiting step in nitrogenase activity by possibly increasing the supply of electrons to nitrogenase and thereby increase the capacity of the cells to reduce nitrogen under different environments. The gene Pm_fer, along with its promoter and terminator region (together defined by SEQ ID NO: 31) was inserted in the czcB locus, i.e. in between SEQ ID NO: 36 and SEQ ID NO: 37.

Pp3: The NRRL B-68085 strain was engineered with the medium-strength constitutive tRNA promoter PR′ from Bacteriophage lambda to overexpress the fer gene from Paenibacillus spp. (Pm_fer) encoding ferredoxin. As described above in greater detail, constitutive expression of ferredoxin could alleviate the rate limiting step in nitrogenase activity by possibly increasing the supply of electrons to nitrogenase and thereby increase the capacity of the cells to reduce nitrogen under different environments. The gene Pm_fer, along with its promoter and terminator region (together defined by SEQ ID NO: 32) was inserted in the czcB locus, i.e. in between SEQ ID NO: 36 and SEQ ID NO: 37.

Pp4: The NRRL B-68085 strain was engineered with the medium-strength constitutive tRNA promoter P43 from B. subtilis to overexpress the fer gene from Paenibacillus spp. (Pm_fer) encoding ferredoxin. As described above in greater detail, constitutive expression of ferredoxin could alleviate the rate limiting step in nitrogenase activity by possibly increasing the supply of electrons to nitrogenase and thereby increase the capacity of the cells to reduce nitrogen under different environments. The gene Pm_fer, along with its promoter and terminator region (together defined by SEQ ID NO: 33) was inserted in the czcB locus, i.e. in between SEQ ID NO: 36 and SEQ ID NO: 37.

Additional Pb strains were designed to be constructed using the methods described in this example. Additional details on said strains are included in Table 14.

TABLE 14
Additional designs for genetically modified
Pb strains of the disclosure.

| | Expression cassette | | SEQ ID |
|---|---|---|---|
| Strain | integrated | Purpose of genetic | NO: for |
| Code | in Pb strain | modification | cassette |
| Pb40 | PR′-DUF269(Pd) | Increased oxygen | 38 |
| | | protection; constitutive | |
| | | promoter | |
| Pb41 | PR′-DUF269(Pf) | Increased oxygen | 39 |
| | | protection; constitutive | |
| | | promoter | |
| Pb42 | PR′-DUF269(Pg) | Increased oxygen | 40 |
| | | protection; constitutive | |
| | | promoter | |
| Pb43 | PR′-DUF269(Pj) | Increased oxygen | 41 |
| | | protection; constitutive | |
| | | promoter | |
| Pb44 | PR′-DUF269(Pr) | Increased oxygen | 42 |
| | | protection; constitutive | |
| | | promoter | |
| Pb45 | PR′-DUF269(Psab) | Increased oxygen | 43 |
| | | protection; constitutive | |
| | | promoter | |
| Pb46 | PR′-DUF269(Pstel) | Increased oxygen | 44 |
| | | protection; constitutive | |
| | | promoter | |
| Pb47 | PR′-DUF269(Pz) | Increased oxygen | 45 |
| | | protection; constitutive | |
| | | promoter | |

### Example 6. Nitrogenase Activity of Engineered Pb Strains as Measured by Acetylene Reduction Assay (ARA)

The in vitro acetylene reduction assay (ARA) was used to determine nitrogenase activity for Pb strains. Pb strains were struck out on TSA plates and grown for 3 days at 30° C. Colonies were picked into 15 mL culture tubes filled with 3 ml of the same medium in liquid form and incubated overnight at 30° C. with shaking at 225 RPM for seed cultures 1. The optical density at 600 nm (OD600) was measured for the initial seed cultures, and then 25 mL of TSB in a 125 mL baffled Erlynmyer flask was inoculated at an OD600 of 0.08 and incubated until mid log phase (approximately 4-5 hours) at 30° C. with shaking at 225 RPM for second seed cultures. The second seed cultures were centrifuged and washed twice in phosphate buffered saline (PBS) solution to remove residual nitrogenous compounds before resuspending in PBS solution at an OD600 of 3.0. Glass 10 ml gas chromatograph vials were filled with 4.5 mL fresh Seldin media and inoculated with 500 μL of the washed and normalized seed cultures for a starting OD600 of 0.3. Vials were transferred into an anaerobic chamber with a 97% nitrogen/3% hydrogen atmosphere and then capped with silicone septums and crimped before 200 μL acetylene was added according to the ARA protocol. Vials were then incubated at 30° C. with shaking at 220 RPM and 25 mm orbit for 18 hours before analysis on the gas chromatograph.

Results: The engineered strain Pb39 [overexpressing the fer gene from Paenibacillus zanthoxyli (Pz_fer) encoding ferredoxin] and its wild-type parent PB172 were subjected to multiple in vitro ARA rounds to measure the effects of the genetic modification (n=3). The Pb39 strain was compared to the wild-type parent PB172 (FIG. 11). ARA for the strains was analyzed using a linear model with a two sided post-hoc dunnett test at 0.95 confidence intervals to determine significant improvement in Ethylene production of edited strain compared to wild-type (n=6).

In the majority of the experimental runs, there was a significant improvement in Pb39 over wild-type strain PB172 (FIG. 11).

FIG. 11 shows a summary of nitrogenase activity as measured by ARA in engineered Pb1 microbe disclosed herein. In 2 out of the 5 experimental runs, strain Pb39 produced significantly less ethylene than its wild-type parent strain PB172, indicating worse performance. In three out of the five experimental runs, strain Pb39 produced significantly more ethylene than its wild-type parent strain PB172, indicating better performance. Based on the observed results, it is concluded that the genetic editing performed in Pb39 results in higher nitrogenase activity and therefore superior nitrogen fixation performance. Variability in results between different experimental rounds can be attributed to inter-run variability due to sampling constraints. Moreover, it should be noted that the magnitude of the best positive result is roughly twice that of the worst negative result.

### Example 7. Efficacy of Engineering in as Measured Via the OPP Ammonia Excretion Assay

The ammonia excretion assay was used to determine the concentration of ammonia that Pb strains accumulate in the culture media. Pb strains were struck out on TSA plates and grown for 3 days at 30° C. Colonies were picked into 15 mL culture tubes filled with 3 ml of the same medium in liquid form and incubated overnight at 30° C. with shaking at 225 RPM for seed cultures 1. The optical density at 600 nm (OD600) was measured for the initial seed cultures, and then 25 mL of TSB in a 125 mL baffled Erlenmeyer flask was inoculated at an OD600 of 0.08 and incubated until mid log phase (approximately 4-5 hours) at 30° C. with shaking at 225 RPM for second seed cultures. The second seed cultures were centrifuged and washed twice in phosphate buffered saline (PBS) solution to remove residual nitrogenous compounds before resuspending in PBS solution at an OD600 of 5.0. A 96 well plate (2 mL, square wells, v-bottom, polypropylene) was filled with 1 mL fresh Seldin per well and wells inoculated with 20 μL of washed seed culture for a starting OD600 of 0.1 at a minimum of six replicates per strain. The plates were sealed with gas-permeable membranes and transferred into an anaerobic chamber with a 97% nitrogen/3% hydrogen atmosphere and incubated statically at 30° C. for 1 to 6 days. Cultures were sterile filtered assayed for ammonia/ammonium concentration via a modified Berthelot reaction using o-phenylphenol (OPP) in place of phenol. Ammonia concentration in micromolar was calculated using an ammonia analytical standard curve on each plate and using linear regression analysis.

Results: The engineered strain Pb39 [overexpressing the fer gene from Paenibacillus zanthoxyli (Pz_fer) encoding ferredoxin] and its wild-type parent PB172 were subjected to multiple ammonia excretion assay experimental runs, each run lasting 6 days, to measure the effects of the genetic modification (n=3). The results of ammonia excreted are shown in FIG. 12. FIG. 12 shows the ammonia measured using the ammonia excretion assay in 7 independent experiments for both strain Pb39 and its wild-type parent strain PB172. As shown in FIG. 12, it was observed that the edited strain Pb39 (dashed lines) had superior ammonia excretion compared to the wild-type parent strain PB172 (continuous lines) after six days of growth in seven independent experiments, showing that the genetic editing performed in Pb39 results in higher ammonia excretion and therefore higher expected nitrogen fixation performance.

### Example 8. Nitrogen-Stressed Greenhouse Experiments

PB172 (wild-type parent Paenibacillus brasilensis) and Pb39 [overexpressing the fer gene from Paenibacillus zanthoxyli (Pz_fer) encoding ferredoxin] were tested in a greenhouse trial. A fermentation broth of each strain was prepared as follows: The Paenibacillus brasilensis wild-type and edited strains were each cultured in shake flasks containing a soluble complex seed medium, such as TSB. Briefly, a seed culture was created by inoculating 50 ml of the medium in a 250 mL baffled flask with 400 uL of an archived glycerol stock of each strain. This seed culture was incubated 16-20 hours on a rotary shaker at 200 rpm at 30° C. For spore production, 5 mL of the seed culture was used to inoculate 250 ml of a soy-based medium in a 1000 mL flask, and incubated on a rotary shaker at 200 rpm at 30° C. for 5 days to facilitate sporulation. A sample of the culture was streaked on TSA and incubated for 3 days at 30° C. to check for purity and contamination. Spore enumeration was performed by plating serial dilutions of heat-treated sporulation culture onto TSA.

Corn seeds were treated first with a fungicide seed treatment. The microbial fermentation broths described above were concentrated by centrifugation and then applied as an overtreatment to reach a target CFU of 1×10⁶ per seed. Seeds were gently spun until dry and stored at 4° C. until planting. The trial entry list was composed of the two microbial entries (wild-type PB172 and Pb39, overexpressing the fer gene from Paenibacillus zanthoxyli (Pz_fer) encoding ferredoxin) and three repeats of the base fungicide treatment. There were twenty replicants blocked across the greenhouse to reduce variability. The entry list within each replicant was randomized and mapped before planting.

Seeds were planted the day after treating into 1.5 gallon pots containing Berger BM2 germination mix (Hummert International). Irrigation was applied daily through a drip irrigation system to bring the soil to field capacity. Weekly all macro and micro nutrients, except nitrogen, were applied with the same irrigation system. Nitrogen, in the form of ammonium nitrate and potassium nitrate dissolved into water, was applied to reach 80 lbs N per plant at a commercial seeding density, which is a reduced rate of N compared to standard practices. 40% of total nitrogen was applied at V3 and the remaining 60% of N was applied at V7. Plants were grown 49 days and then were destructively harvested. At harvest, the plants were initiating tasseling and were either classified as V17 or VT. At harvest the plant shoot was cut at 1 inch above the soil surface to avoid aerial roots. All above ground biomass was bagged and then dried at 80° C. for at least 14 days. After drying the material was weighed and recorded as grams.

Total above ground dry mass in corn treated with wild-type microbe, PB172, did not show a significant increase as compared to the base seed treatment (1.52% increase, p=0.30) Biomass of corn treated with the edited microbe, Pb39 [overexpressing the fer gene from Paenibacillus zanthoxyli (Pz_fer) encoding ferredoxin], was significantly greater than that of corn with a base seed treatment (4.62% increase, p=0.002). Biomass of corn treated with edited microbe was also significantly greater than biomass of corn treated with wild-type microbe (3.05% increase, p<0.01). Data is summarized in FIG. 13.

### Example 9. Additional Methods for Assaying the Performance of Strains Engineered for Improved Nitrogen Fixation

28-day nitrogen-stress greenhouse assay: In this assay, plants are grown under suboptimal nitrogen conditions and are thus stressed. By growing plants together with microbial nitrogen fixers and assaying the improvement in plant health, one can determine the performance of the microbial nitrogen fixer. The assay is run as follows:

A fermentation broth of each strain is prepared as follows: Wild-type and engineered strains are each cultured in shake flasks containing a soluble complex seed medium, such as TSB. Briefly, a seed culture is created by inoculating 50 mL of the medium in a 250 mL baffled flask with 400 uL of an archived glycerol stock of each strain. This seed culture is incubated 16-20 hours on a rotary shaker at 200 rpm at 30° C. For spore production, 5 mL of the seed culture is used to inoculate 250 mL of a soy-based medium in a 1000 mL flask, and incubated on a rotary shaker at 200 rpm at 30° C. for 5 days to facilitate sporulation. A sample of the spore culture is streaked on TSA and incubated for 3 days at 30° C. to check for purity and contamination. Spore enumeration is performed by plating serial dilutions of heat-treated sporulation culture onto TSA agar media.

Corn seeds are treated first with a base fungicide seed treatment. The microbial fermentation broths described above are concentrated by centrifugation and then applied as an overtreatment to reach a target CFU of 1×10⁶ per seed. Seeds are gently spun until dry and stored at 4° C. until planting. The trial entry list is composed of wild-type and engineered strains and three repeats of the base fungicide treatment. There are thirty replicates blocked across the greenhouse to reduce variability. The entry list within each replicate is randomized and mapped before planting.

Seeds are planted after being treated into 1.9 L pots containing a custom Berger BM2 germination mix (Hummert International) without added nutrients. Irrigation is applied daily through a drip irrigation system to bring the soil to field capacity. Weekly, all macro- and micro-nutrients, except nitrogen, are applied with the same irrigation system. Nitrogen is applied in the form of ammonium nitrate dissolved into water at a concentration of 500 ppm of N. 50% of the total nitrogen is applied at 7 days and another 50% at 14 days after planting. Plants are grown for 28 days, scanned with the PlantEye (Phenospex), and then destructively harvested. At harvest, the plants are usually either classified as growth stage V5 or V6. At harvest, the plant shoot is cut at the soil surface. All above-ground biomass is bagged and then dried at 80° C. for at least 14 days. After drying, the material is weighed and recorded in grams.

In planta acetylene reduction assay: The in planta acetylene reduction assay (in planta ARA) is used to determine nitrogenase activity for nitrogen-fixing strains grown in association with corn seedling roots. This assay is an adaptation of the in vitro Acetylene Reduction Assay, described in Examples 2 and 6, assaying the activity of nitrogenase when strains are grown in the presence of a seedling.

Nitrogen-fixing strains are struck out on TSA plates and grown for 3 days at 30° C. Individual colonies are then grown in 10 mL TSB medium in 50 mL culture tubes and incubated overnight at 30° C. with shaking. Next day, 1 mL seed culture is inoculated into 50 mL TSB medium in a 250 mL baffled flask and grown until mid-log phase. Optical density (OD600) is measured, then cells are collected by centrifugation (8000 g for 10 min) and washed in equal volume (50 mL) of PB (Butterfield's Phosphate Buffer). Washing is repeated 1×, and cells are resuspended to final OD600=1.

Experiments are conducted in and are grown in a controlled environment (16 h L/8 h D at 28 C/25 C and 65% RH). The corn seeds used are treated first with a fungicide seed treatment. Individual seeds are then planted in a 50 ml conical tube in 30 ml of sand:vermiculite (1:1) that has been autoclaved and rehydrated in N-free Hoagland's media. Then, the microbial fermentations are diluted 1:10 (final OD=0.1) in PB and 1 ml per seed is applied as drench. Corn seedlings are grown in 50 ml conical tubes with caps on until corn seedlings grow beyond the confines of the tube (~4 days post planting) at which point caps are removed and corn are allowed to continue growth in a controlled environment chamber through day 7. Seedlings are treated with 1 ml dl water on subsequent days through day 6. On day 7, seedlings are enclosed in the 50 ml conical tube, using an airtight rubber septum, and 5% acetylene is injected into each sample. After 5 days, 2 ml of the headspace is sampled and delivered to a GCMS vial. Acetylene and ethylene are quantified by GC-FID/MS.

Growth-based ammonia excretion assay: A wild-type E. coli K12 MG1655 is used as a biosensor for secreted ammonia in supernatants of wild-type and engineered diazotrophic strains. This assay assumes that E. coli growth is limited by bioavailable secreted fixed nitrogen in the supernatant. Thus, E. coli biomass is proportional to ammonia concentration in the supernatant. This assay allows for quantitative ranking of E. coli OD600 measurements based on the diazotrophic strain source of the E. coli growth media to determine high ammonia producers.

To prepare the biosensor, the following is done: A cryostock vial of E. coli K12 MG1655 is inoculated into 110 mL of LB media and incubated overnight at 37° C. with shaking at 250 rpm. The culture is then centrifuged at 4122×g for 5 minutes, washed twice with 50 ml of PBS to remove any trace sources of nitrogen, and resuspended in 50 mL of PBS. The optical density at 600 nm (OD600) is measured using a plate reader, and the cell suspension is adjusted to an OD600 of 6.1 by adding PBS or removing supernatant and resuspending the pellet in the appropriate volume. An equal volume of 50% glycerol is added to the resuspended cells, and 15 μL aliquots were dispensed into ~50 PCR plates using a Hamilton liquid handling system with a 96-tip CORE head. The plates are then sealed and stored at −80° C.

The assay is run as follows: First, nitrogen-fixing microbes are grown in the manner described in Example 7. Strains are struck out on TSA plates and grown for 3 days at 30° C. Colonies are picked into 15 mL culture tubes filled with 3 ml of the same medium in liquid form and incubated overnight at 30° C. with shaking at 225 RPM for initial seed cultures. The optical density at 600 nm (OD600) is measured for the initial seed cultures, and then 25 mL of TSB in a 125 mL baffled Erlenmeyer flask was inoculated at an OD600 of 0.08 and incubated until mid log phase (approximately 4-5 hours) at 30° C. with shaking at 225 RPM for second seed cultures. The second seed cultures are centrifuged and washed twice in phosphate buffered saline (PBS) solution to remove residual nitrogenous compounds before resuspending in PBS solution at an OD600 of 5.0. A 96 well plate (2 mL, square wells, v-bottom, polypropylene) is filled with 1 mL fresh Seldin per well and wells are inoculated with 20 uL of washed seed culture for a starting OD600 of 0.1 at a minimum of six replicates per strain. The plates are sealed with gas-permeable membranes and transferred into an anaerobic chamber with a 97% nitrogen/3% hydrogen atmosphere and incubated statically at 30° C. for 1 to 6 days. Cultures are sterile filtered such that only the liquid supernatant is collected.

150 μL of sterile M9 minimal media without ammonia with 0.5% (W/V) glucoseis added to 96-well plates (Axygen® 96-well Clear Round Bottom 1.1 mL Polypropylene Deep Well Plate), and 150 μL of filtered supernatants is stamped onto these plates along with NH₄Cl standards to specific wells (40 mM and no NH₄Cl controls). 5 μL of thawed E. coli cells are then added to each well except for one (coordinate H12), and the plates were incubated at 37° C. with shaking at 1000 rpm for approximately 20 hours. Following incubation, 100 μL of culture from each well is transferred to a new flat-bottom plate (Corning® 96-well Clear Flat Bottom Polystyrene Not Treated Microplate) containing 100 μL of PBS. The OD600 of each well was measured using a plate reader, and the reading of the blank well (H12) is subtracted from all the raw measurements.

Data analysis methods: Edited strain performance is compared against the respective wild-type parent (PB172 or NRRL B-68085) and the percent change from their wild-type parent is calculated. Effect sizes are calculated using a mixed linear model on all available data for each strain within each assay, however some strains have more available data than others. Significance is calculated using a post-hoc Dunnett's Test on the results of the mixed linear models, compared to the relevant wild-type strain.

### Example 10. Construction of Additional Engineered Strains with Altered Expression of Ferredoxin

Additional engineering of wild-type Paenibacillus peoriae strain NRRL B-68085 was conducted to obtain strains overexpressing ferredoxin. Ferredoxins used were: Pl_fer from Candidatus Pristimantibacillus lignocellulolyticus; Fp_fer from Fontibacillus panacisegetis strain DSM 28129; Sb_fer from Saccharibacillus brassicae; Fs_fer from Fontibacillus solani; WB17_fer from Saccharibacillus sp. WB 17; Nm_fer from Closest match: Paenibacillus sp. 453mf; Sk_fer from Saccharibacillus kuerlensis; and Ss_fer from Saccharibacillus sacchari (see Table 15). The description of construction of these strains is presented here in Example 10, and the results of experiments using these strains is presented in Example 11.

Overexpression of ferredoxin was achieved by either expressing additional copies of ferredoxin, as in strains Pp5-Pp12, or by insertion of the promoter PtrnQ and its associated 5′ UTR (PtrnQ-5′ UTR) downstream of the native promoter sequence and upstream of the native ferredoxin gene, as in strain Pp13. In the case of strains Pp5-Pp12, a PtrnQ-5′ UTR cassette is inserted upstream of the additional ferredoxin copy. A synthetic spacer and ribosomal binding site, the sequence of which is given by SEQ ID NO: 86, is inserted downstream of every inserted PtrnQ-5′ UTR. The strains were constructed using the multi-plasmid system described in Example 5. Details of the strains are listed in Table 15. Details of the genetic construct components are listed in Table 16. For Strain IDs Pp5-Pp12 the homology pairs used for targeting integrations were SEQ ID NOs: 36 and 37. Strains Pp5-Pp12 are the result of engineering NRRL B-68085. The homology pair used for targeting integration for Strain ID Pp13 is given by SEQ ID NOs: 59 and 60 and also listed in Table 13. Strain Pp13 is the result of engineering NRRL B-68085.

TABLE 15
List of Paenibacillus strain IDs, genotype, purpose
of genetic modification, and SEQ ID NOs for cassettes.

| | | Enzyme | | |
|---|---|---|---|---|
| | | Commission | Purpose | SEQ ID |
| Strain | Expression cassette | Number/protein | of genetic | NO: for |
| ID | integrated in strain | class Gene | modification | cassette |
| Pp5 | czcB::PtrnQ-5′ UTR:PI_fer- | Protein electron | Increase flux of | 50 |
| | Tfer | carrier | electrons to nitrogen | |
| | | (ferredoxin) | fixation reaction | |
| Pp6 | czcB::PtrnQ-5′ UTR:Fp_fer- | Protein electron | Increase flux of | 51 |
| | Tfer | carrier | electrons to nitrogen | |
| | | (ferredoxin) | fixation reaction | |
| Pp7 | czcB::PtrnQ-5′ UTR:Sb_fer- | Protein electron | Increase flux of | 52 |
| | Tfer | carrier | electrons to nitrogen | |
| | | (ferredoxin) | fixation reaction | |
| Pp8 | czcB::PtrnQ-5′ UTR:Fs_fer- | Protein electron | Increase flux of | 53 |
| | Tfer | carrier | electrons to nitrogen | |
| | | (ferredoxin) | fixation reaction | |
| Pp9 | czcB::PtrnQ-5′ | Protein electron | Increase flux of | 54 |
| | UTR:WB17_fer-Tfer | carrier | electrons to nitrogen | |
| | | (ferredoxin) | fixation reaction | |
| Pp10 | czcB::PtrnQ-5′ UTR:Nm_fer- | Protein electron | Increase flux of | 55 |
| | Tfer | carrier | electrons to nitrogen | |
| | | (ferredoxin) | fixation reaction | |
| Pp11 | czcB::PtrnQ-5′ UTR:Sk_fer- | Protein electron | Increase flux of | 56 |
| | Tfer | carrier | electrons to nitrogen | |
| | | (ferredoxin) | fixation reaction | |
| Pp12 | czcB::PtrnQ-5′ UTR:Ss_fer- | Protein electron | Increase flux of | 57 |
| | Tfer | carrier | electrons to nitrogen | |
| | | (ferredoxin) | fixation reaction | |
| Pp13 | PtrnQ-5′ UTR downstream | Protein electron | Increase flux of | 58 |
| | of Pfer | carrier | electrons to nitrogen | |
| | | (ferredoxin) | fixation reaction | |

TABLE 16
Components of genetic constructs and component source.

| | | | GenBank | GenBank |
|---|---|---|---|---|
| | | | accession # | accession # |
| | | | of source | of introduced |
| Name | Description | Source | genome | component |
| PtrnQ-5′ | Strong | Bacillus subtilis | CP053102.1 | Locus tag |
| UTR | constitutive | | | HIR77_19330 (nt |
| | promoter and | | | 3646432-3646592 of |
| | associated 5′ | | | CP053102.1) |
| | UTR | | | |
| Synthetic | Synthetic spacer | Synthetic | N/A | Sequence given by |
| spacer | and ribosomal | | | SEQ ID NO: 86 |
| and RBS | binding site | | | |
| Tfer | Transcriptional | Paenibacillus | GCF_000520715.1 | WP_03673731 |
| | terminator | zanthoxyli JH29 | | |
| TgyrB | Transcriptional | Paenibacillus | CP000154.2 | Locus tag |
| | terminator | polymyxa | | ADM67911.1 (nt |
| | | | | 6761-6861) |
| Pl_fer | Ferredoxin | Candidatus | CP097899.1 | Amino-acid (AA) |
| | | Pristimantibacillus | | sequence source is |
| | | lignocellulolyticus | | URN94381.1. |
| | | | | The amino-acid |
| | | | | sequence is |
| | | | | provided as SEQ ID |
| | | | | NO: 61. |
| | | | | An exemplary |
| | | | | genomic source for |
| | | | | DNA encoding the |
| | | | | protein of SEQ ID |
| | | | | NO: 61 can be |
| | | | | CP097899.1 (locus |
| | | | | tag: NAG76_21585). |
| | | | | Alternatively, DNA |
| | | | | encoding the protein |
| | | | | can be designed |
| | | | | through reverse |
| | | | | translation using a |
| | | | | codon table to obtain |
| | | | | a DNA sequence. |
| | | | | For example, SEQ |
| | | | | ID NO: 62. |
| Fp_fer | Ferredoxin | Fontibacillus | NZ_FNBG01000014.1 | Amino-acid (AA) |
| | | panacisegetis | | sequence source is |
| | | strain DSM 28129 | | WP_091231084.1. |
| | | | | The amino-acid |
| | | | | sequence is |
| | | | | provided as SEQ ID |
| | | | | NO: 63. |
| | | | | An exemplary |
| | | | | genomic source for |
| | | | | DNA encoding the |
| | | | | protein of SEQ ID |
| | | | | NO: 63 can be |
| | | | | NZ_FNBG01000014.1 |
| | | | | (locus tag: |
| | | | | BLS49_RS13285). |
| | | | | Alternatively, DNA |
| | | | | encoding the protein |
| | | | | can be designed |
| | | | | through reverse |
| | | | | translation using a |
| | | | | codon table to obtain |
| | | | | a DNA sequence. |
| | | | | For example, SEQ |
| | | | | ID NO: 64 |
| Sb_fer | Ferredoxin | Saccharibacillus | NZ_CP041217.1 | Amino-acid (AA) |
| | | brassicae | | sequence source is |
| | | | | WP_141449228. |
| | | | | The amino-acid |
| | | | | sequence is |
| | | | | provided as SEQ ID |
| | | | | NO: 65. |
| | | | | An exemplary |
| | | | | genomic source for |
| | | | | DNA encoding the |
| | | | | protein of SEQ ID |
| | | | | NO: 65 can be |
| | | | | NZ_CP041217.1 |
| | | | | (locus tag: |
| | | | | FFV09_RS18680). |
| | | | | Alternatively, DNA |
| | | | | encoding the protein |
| | | | | can be designed |
| | | | | through reverse |
| | | | | translation using a |
| | | | | codon table to obtain |
| | | | | a DNA sequence. |
| | | | | For example, SEQ |
| | | | | ID NO: 66. |
| Fs_fer | Ferredoxin | Fontibacillus | NZ_JACJIP010000017.1 | Amino-acid (AA) |
| | | solani | | sequence source is |
| | | | | WP_091231084.1. |
| | | | | The amino-acid |
| | | | | sequence is |
| | | | | provided as SEQ ID |
| | | | | NO: 67. |
| | | | | An exemplary |
| | | | | genomic source for |
| | | | | DNA encoding the |
| | | | | protein of SEQ ID |
| | | | | NO: 67 can be |
| | | | | NZ_JACJIP010000017.1 |
| | | | | (locus tag: |
| | | | | FHR92_RS13660). |
| | | | | Alternatively, DNA |
| | | | | encoding the protein |
| | | | | can be designed |
| | | | | through reverse |
| | | | | translation using a |
| | | | | codon table to obtain |
| | | | | a DNA sequence. |
| | | | | For example, SEQ |
| | | | | ID NO: 68. |
| WB17_fer | Ferredoxin | Saccharibacillus | NZ_VPFK02000045.1 | Amino-acid (AA) |
| | | sp. WB 17 | | sequence source is |
| | | | | WP_150231153.1. |
| | | | | The amino-acid |
| | | | | sequence is |
| | | | | provided as SEQ ID |
| | | | | NO: 69. |
| | | | | An exemplary |
| | | | | genomic source for |
| | | | | DNA encoding the |
| | | | | protein of SEQ ID |
| | | | | NO: 69 can be |
| | | | | NZ_VPFK02000045.1 |
| | | | | (locus tag: |
| | | | | FTE24_RS20400). |
| | | | | Alternatively, DNA |
| | | | | encoding the protein |
| | | | | can be designed |
| | | | | through reverse |
| | | | | translation using a |
| | | | | codon table to obtain |
| | | | | a DNA sequence. |
| | | | | For example, SEQ |
| | | | | ID NO: 70. |
| Nm_fer | Ferredoxin | Closest match: | Closest match: | The amino-acid |
| | | Paenibacillus sp. | NZ_FPAC01000001.1 | sequence is |
| | | 453mf | | provided as SEQ ID |
| | | | | NO: 71. |
| | | | | DNA encoding the |
| | | | | protein can be |
| | | | | designed through |
| | | | | reverse translation |
| | | | | using a codon table |
| | | | | to obtain a DNA |
| | | | | sequence. For |
| | | | | example, SEQ ID |
| | | | | NO: 72. |
| Sk_fer | Ferredoxin | Saccharibacillus | NZ_KB899277.1 | Amino-acid (AA) |
| | | kuerlensis | | sequence source is |
| | | | | WP_018975213.1. |
| | | | | The amino-acid |
| | | | | sequence is |
| | | | | provided as SEQ ID |
| | | | | NO: 73 |
| | | | | An exemplary |
| | | | | genomic source for |
| | | | | DNA encoding the |
| | | | | protein of SEQ ID |
| | | | | NO: 73 can be |
| | | | | NZ_KB899277.1 |
| | | | | (locus tag: |
| | | | | F456_RS0101400). |
| | | | | Alternatively, DNA |
| | | | | encoding the protein |
| | | | | can be designed |
| | | | | through reverse |
| | | | | translation using a |
| | | | | codon table to obtain |
| | | | | a DNA sequence. |
| | | | | example, SEQ ID |
| | | | | NO: 74. |
| Ss_fer | Ferredoxin | Saccharibacillus | NZ_KK073875.1 | Amino-acid (AA) |
| | | sacchari | | sequence source is |
| | | | | WP_037282750.1. |
| | | | | The amino-acid |
| | | | | sequence is |
| | | | | provided as SEQ ID |
| | | | | NO: 75 |
| | | | | An exemplary |
| | | | | genomic source for |
| | | | | DNA encoding the |
| | | | | protein of SEQ ID |
| | | | | NO: 75 can be |
| | | | | NZ_KK073875.1 |
| | | | | (locus tag: |
| | | | | SACSAC_RS01545). |
| | | | | Alternatively, DNA |
| | | | | encoding the protein |
| | | | | can be designed |
| | | | | through reverse |
| | | | | translation using a |
| | | | | codon table to obtain |
| | | | | a DNA sequence. |
| | | | | example, SEQ ID |
| | | | | NO: 76. |

Amino acid sequence identities of the various ferredoxin proteins overexpressed in the Paenibacillus strains were compared using Geneious (which is a bioinformatics software) Multiple align feature. The mode used was “Geneious Alignment”, the alignment type was “Global alignment with free end gaps”, the Cost Matrix was “Blosum62”, the Gap open penalty was “12”, the Gap extension penalty was “3” & the Refinement Iterations were “2”. Results of comparisons are shown in Table 17.

TABLE 17
Amino acid sequence alignments for various ferredoxin proteins.

| | fer (P. | fer (P. | fer | fer | fer | fer | fer | fer | fer | fer | fer | fer |
|---|---|---|---|---|---|---|---|---|---|---|---|---|
| | brasilensis) | peoriae) | (Fp) | (Fs) | (Nm) | (Pl) | (Pm) | (Pz) | (Sb) | (Sk) | (Ss) | (WB17) |
| fer (P. | | 97.5 | 85 | 85 | 87.5 | 84.62 | 91.25 | 85.9 | 85 | 85 | 83.8 | 86.3 |
| brasilensis) | | | | | | | | | | | | |
| fer (P. | 97.5 | | 87.5 | 87.5 | 88.75 | 87.18 | 93.75 | 87.18 | 86.25 | 86.3 | 85 | 87.5 |
| peoriae) | | | | | | | | | | | | |
| fer(Fp) | 85 | 87.5 | | 100 | 88.75 | 87.18 | 85 | 92.31 | 80 | 80 | 78.8 | 78.8 |
| fer(Fs) | 85 | 87.5 | 100 | | 88.75 | 87.18 | 85 | 92.31 | 80 | 80 | 78.8 | 78.8 |
| fer(Nm) | 87.5 | 88.8 | 88.8 | 88.8 | | 84.62 | 87.5 | 85.9 | 82.5 | 85 | 82.5 | 81.3 |
| fer(Pl) | 84.6 | 87.2 | 87.2 | 87.2 | 84.62 | | 84.62 | 85.9 | 84.62 | 84.6 | 85.9 | 83.3 |
| fer(Pm) | 91.3 | 93.8 | 85 | 85 | 87.5 | 84.62 | | 82.05 | 86.25 | 83.8 | 85 | 87.5 |
| fer(Pz) | 85.9 | 87.2 | 92.3 | 92.3 | 85.9 | 85.9 | 82.05 | | 80.77 | 80.8 | 79.5 | 79.5 |
| fer(Sb) | 85 | 86.3 | 80 | 80 | 82.5 | 84.62 | 86.25 | 80.77 | | 96.3 | 95 | 98.8 |
| fer(Sk) | 85 | 86.3 | 80 | 80 | 85 | 84.62 | 83.75 | 80.77 | 96.25 | | 95 | 95 |
| fer(Ss) | 83.8 | 85 | 78.8 | 78.8 | 82.5 | 85.9 | 85 | 79.49 | 95 | 95 | | 93.8 |
| fer(WB17) | 86.3 | 87.5 | 78.8 | 78.8 | 81.25 | 83.33 | 87.5 | 79.49 | 98.75 | 95 | 93.8 | |

### Example 11. Performance of Additional Engineered Strains Overexpressing Ferredoxin

A number of the strains from Example 10 were assayed using the growth-based ammonia excretion assay described in Example 9, the acetylene reduction assay (ARA) described in Example 6, and the in planta acetylene reduction assay (in planta ARA) described in Example 9. The results were analyzed using the data analysis methods described in Example 9. Table 18 shows effects of overexpression of ferredoxin in genetically engineered strains compared to the performance of the parent strain NRRL B-68085. In each case, effects are presented as % differences compared to the performance of the wild-type strain (e.g., X % better than wild-type), with a higher number indicating a better performance. Positive values with a p-value of 0.1 or less are considered statistically significant increases.

TABLE 18
Mean delta % compared to wild-type in strains
engineered to overexpress ferredoxin

| | | growth- | | |
|---|---|---|---|---|
| | | based | | in |
| Strain | | ammonia | | planta |
| ID | Edit | excretion | ARA | ARA |
| Pp1 | czcB::PtrnQ:Pz_fer-Tfer | 2.71* | −29.97 | −6.75* |
| Pp10 | czcB::PtrnQ-5′ UTR:Nm_fer-Tfer | 14.9 | | |
| Pp11 | czcB::PtrnQ-5′ UTR:Sk_fer-Tfer | 10.05 | | |
| Pp12 | czcB::PtrnQ-5′ UTR:Ss_fer-Tfer | 19.39 | | |
| Pp13 | PtrnQ-5′ UTR downstream of Pfer | 16.52 | | |
| Pp2 | czcB::PtrnQ:Pm_fer-Tfer | | 15.36* | |
| Pp3 | czcB::PR′:Pm_fer-Tfer | | −19.38* | 39.42 |
| Pp4 | czcB::P43:Pm_fer-Tfer | | −36.15* | |
| Pp5 | czcB::PtrnQ-5′ UTR:Pl_fer-Tfer | 3.05* | | |
| Pp6 | czcB::PtrnQ-5′ UTR:Fp_fer-Tfer | 12.43 | | |
| Pp7 | czcB::PtrnQ-5′ UTR:Sb_fer-Tfer | 12.21 | | |
| Pp8 | czcB::PtrnQ-5′ UTR:Fs_fer-Tfer | 10.77 | | |
| Pp9 | czcB::PtrnQ-5′ UTR:WB17_fer- | 2.33* | | |
| | Tfer | | | |

*Asterisked results have a p-value greater than 0.1.

Table 18 shows that overexpression of different ferredoxins sourced from a variety of organisms has a positive effect on performance, as measured by the growth-based ammonia excretion assay. As can also be seen in Table 18, in one instance of ferredoxin overexpression (strain Pp1), the edit appeared to result in a small, nonsignificant decrease in nitrogenase activity as measured by in planta ARA, whereas in another instance (strain Pp3) a ferredoxin overexpression edit resulted in a significant increase. However, it is noted that strain Pp1 showed beneficial effects in a longer term greenhouse assay (see Example 20), indicating that over longer periods of time the improved performance of nitrogenase overcame any additional resource expenditure over the longer term.

### Example 12. Construction of Additional Engineered Strains Overexpressing Pyruvate Flavodoxin Oxidoreductase

Additional engineering of wild-type Paenibacillus brasilensis (Pb) strain PB172 or NRRL B-68085 was conducted to obtain strains overexpressing native (Paenibacillus brasilensis) pyruvate flavodoxin oxidoreductase, as described in this Example 12. Results of experiments with these strains are described in Example 13.

Overexpressing the native (Paenibacillus brasilensis) pyruvate flavodoxin oxidoreductase was achieved by inserting a cassette downstream of the native promoter sequence and upstream of the native pyruvate flavodoxin oxidoreductase gene cluster. The cassette can be the promoter PtrnQ and its associated 5′ UTR (PtrnQ-5′ UTR), as in strain Pb49, the promoter PR′ and the 5′ UTR associated with trnQ (PR′-5′ UTR), as in strain Pb48, or the terminator TR2 and the 5′ UTR associated with trnQ (TR2-5′ UTR), as in strain Pb50. A synthetic spacer and ribosomal binding site, the sequence of which is given by SEQ ID NO: 86, is inserted downstream of every inserted PtrnQ-5′ UTR, PR′-5′ UTR or TR2-5′ UTR cassette. The strains were constructed using the multi-plasmid system described in Example 5. Details of the strains are listed in Table 19. Details of the genetic construct components are listed in Table 20. For Strain IDs Pb48-Pb50 the homology pairs used for targeting integrations were SEQ ID NOs: 82 and 83. Strains Pb48-Pb50 are the result of engineering PB172. For Strain IDs Pp14-Pp15 the homology pairs used for targeting integrations were SEQ ID NOs: 80 and 81. Strains Pp14-Pp15 are the result of engineering NRRL B-68085.

TABLE 19
List of Paenibacillus strain IDs, genotype, purpose
of genetic modification, and SEQ ID NOs for cassettes.

| | | Enzyme | | |
|---|---|---|---|---|
| | | Commission | Purpose | SEQ ID |
| Strain | Expression cassette | Number/protein | of genetic | NO: for |
| ID | integrated in strain | class Gene | modification | cassette |
| Pb48 | PR′-5′ UTR | EC 1.2.7.1 | Increase flux of | 77 |
| | downstream of PporD | | electrons to nitrogen | |
| | | | fixation reaction | |
| Pb49 | PtrnQ-5′ UTR | EC 1.2.7.1 | Increase flux of | 78 |
| | downstream of PporD | | electrons to nitrogen | |
| | | | fixation reaction | |
| Pb50 | TR2-5′ UTR | EC 1.2.7.1 | Increase flux of | 79 |
| | downstream of PporD | | electrons to nitrogen | |
| | | | fixation reaction | |
| Pp14 | PR′-5′ UTR | EC 1.2.7.1 | Increase flux of | 77 |
| | downstream of PporD | | electrons to nitrogen | |
| | | | fixation reaction | |
| Pp15 | TR2-5′ UTR | EC 1.2.7.1 | Increase flux of | 79 |
| | downstream of PporD | | electrons to nitrogen | |
| | | | fixation reaction | |

TABLE 20
Components of genetic constructs and component source.

| | | | GenBank | GenBank |
|---|---|---|---|---|
| | | | accession # | accession # |
| | | | of source | of introduced |
| Name | Description | Source | genome | component |
| Synthetic | Synthetic spacer | Synthetic | N/A | Sequence given by |
| spacer and | and ribosomal | | | SEQ ID NO: 86 |
| RBS | binding site | | | |
| 5′ UTR | 5′ Untranslated | Bacillus | CP053102.1 | Nucleotides |
| | region | subtilis | | 3646432-3646459 |
| | | | | of CP053102.1 |
| PtrnQ | Strong | Bacillus | CP053102.1 | Locus tag |
| | constitutive | subtilis | | HIR77_19330 (nt |
| | promoter and | | | 3646432-3646592 |
| | associated 5′ | | | of CP053102.1) |
| | UTR | | | |
| PR′ | Medium-strength | Bacteriophage | NC_001416.1 | Locus tag |
| | constitutive | lambda | | lambdap72 Gene |
| | promoter | | | ID: 2703521 (nt |
| | | | | 44479-44603 of |
| | | | | NC_001416.1) |
| TR2 | Terminator | Bacteriophage | NC_001416.1 | Locus tag |
| | | lambda | | lambdap54 Gene |
| | | | | ID: 2703490 (nt |
| | | | | 40581-40629 of |
| | | | | NC_001416.1) |

### Example 13. Performance of Additional Engineered Strains with Altered Expression of Pyruvate Flavodoxin Oxidoreductase

A number of the strains of Example 12, descended from wild-type Paenibacillus brasilensis PB172 or wild-type Paenibacillus peoriae NRRL B-68085 and overexpressing native (Paenibacillus brasilensis) pyruvate flavodoxin oxidoreductase, were assayed using the acetylene reduction assay (ARA) described in Example 6 and the results were analyzed using the data analysis methods described in Example 9. Effects are presented as % differences compared to the performance of the wild-type strain (e.g., X % better than wild-type), with a higher number indicating a better performance. P-values less than 0.1 indicated a significant effect. The results are shown in Table 21.

TABLE 21
Mean delta % compared to wild-type in strains engineered
to overexpress pyruvate flavodoxin oxidoreductase.

| | | | | | OPP |
|---|---|---|---|---|---|
| Strain | | WT control | | in planta | ammonia |
| ID | Edit | Strain ID | ARA | ARA | excretion |
| Pb48 | PR′-5′ UTR downstream of PporD | PB172 | 16.2 | 54.95 | 34.88* |
| Pb49 | PtrnQ-5′ UTR downstream of PporD | PB172 | 44.4 | 50.18 | −29.91* |
| Pb50 | TR2-5′ UTR downstream of PporD | PB172 | −68.1 | 7.82* | −45.99* |
| Pp14 | PR′-5′ UTR downstream of PporD | NRRL B-68085 | −7.61* | −21.17* | −46.41 |
| Pp15 | TR2-5′ UTR downstream of PporD | NRRL B-68085 | | 5.97* | −85.57 |

*Asterisked results had a p-value of greater than 0.1.

### Example 14. Construction of Additional Engineered Strains Overexpressing DUF269

Additional engineering was conducted to obtain strains overexpressing DUF269. This was achieved by expressing additional copies of DUF269, as in strains Pp16-Pp22 and Pb40-Pb47. In the case of strains Pp16-Pp22 and Pb40-Pb47, a PR′-5′ UTR cassette (i.e. promoter PR′ and the 5′ UTR associated with PtrnQ) is inserted upstream of the additional DUF269 copy. A synthetic spacer and ribosomal binding site, the sequence of which is given by SEQ ID NO: 86, is inserted downstream of every inserted PR′-5′ UTR cassette. The strains were constructed using the multi-plasmid system described in Example 5. Details of the strains are listed in Table 14 (for strains derived from wild-type PB172) and Table 22 (for strains derived from NRRL B-68085). Details of the genetic construct components are listed in Table 23. For Strain IDs Pb40-Pb47 the homology pairs used for targeting integrations were SEQ ID NOs: 84 and 85. Strains Pb40-Pb47 are the result of engineering PB172. For Strain IDs Pp16-Pp22 the homology pairs used for targeting integrations were SEQ ID NOs: 36 and 37. Strains Pp16-Pp22 are the result of engineering NRRL B-68085.

TABLE 22
List of Paenibacillus strain IDs, genotype, purpose
of genetic modification, and SEQ ID NOs for cassettes.

| | | Enzyme | | |
|---|---|---|---|---|
| | | Commission | Purpose | SEQ ID |
| Strain | Expression cassette | Number/protein | of genetic | NO: for |
| ID | integrated in strain | class Gene | modification | cassette |
| Pp16 | czcB::PR′-5′ UTR- | Protein involved in | Increase | 38 |
| | DUF269(Pd) | protection of | oxygen | |
| | | nitrogenase from | protection | |
| | | oxidative damage | | |
| Pp17 | czcB::PR′-5′ UTR- | Protein involved in | Increase | 39 |
| | DUF269(Pf) | protection of | oxygen | |
| | | nitrogenase from | protection | |
| | | oxidative damage | | |
| Pp18 | czcB::PR′-5′ UTR- | Protein involved in | Increase | 40 |
| | DUF269(Pg) | protection of | oxygen | |
| | | nitrogenase from | protection | |
| | | oxidative damage | | |
| Pp19 | czcB::PR′-5′ UTR- | Protein involved in | Increase | 41 |
| | DUF269(Pj) | protection of | oxygen | |
| | | nitrogenase from | protection | |
| | | oxidative damage | | |
| Pp20 | czcB::PR′-5′ UTR- | Protein involved in | Increase | 42 |
| | DUF269(Pr) | protection of | oxygen | |
| | | nitrogenase from | protection | |
| | | oxidative damage | | |
| Pp21 | czcB::PR′-5′ UTR- | Protein involved in | Increase | 44 |
| | DUF269(Pstel) | protection of | oxygen | |
| | | nitrogenase from | protection | |
| | | oxidative damage | | |
| Pp22 | czcB::PR′-5′ UTR- | Protein involved in | Increase | 45 |
| | DUF269(Pz) | protection of | oxygen | |
| | | nitrogenase from | protection | |
| | | oxidative damage | | |

TABLE 23
Components of genetic constructs and component source.

| | | | GenBank | GenBank |
|---|---|---|---|---|
| | | | accession # | accession # |
| | | | of source | of introduced |
| Name | Description | Source | genome | component |
| DUF269 | protein involved | Paenibacillus | NZ_CP011114.1 | Amino-acid (AA) |
| (Pd) | in protection of | durus | | sequence source is |
| | nitrogenase from | | | WP_025698314.1. |
| | oxidative damage | | | The amino-acid |
| | | | | sequence is |
| | | | | provided as SEQ ID |
| | | | | NO: 88. |
| | | | | An exemplary |
| | | | | genomic source for |
| | | | | DNA encoding the |
| | | | | protein of SEQ ID |
| | | | | NO: 88 can be |
| | | | | NZ_CP011114.1. |
| | | | | Alternatively, DNA |
| | | | | encoding the protein |
| | | | | can be designed |
| | | | | through reverse |
| | | | | translation using a |
| | | | | codon table to |
| | | | | obtain a DNA |
| | | | | sequence. example, |
| | | | | SEQ ID NO: 89. |
| DUF269 | protein involved | Paenibacillus | NZ_JAUSUY010000035.1 | Amino-acid (AA) |
| (Pf) | in protection of | forsythiae | | sequence source is |
| | nitrogenase from | | | WP_025697219.1. |
| | oxidative damage | | | The amino-acid |
| | | | | sequence is |
| | | | | provided as SEQ ID |
| | | | | NO: 90. |
| | | | | An exemplary |
| | | | | genomic source for |
| | | | | DNA encoding the |
| | | | | protein of SEQ ID |
| | | | | NO: ZZZ can be |
| | | | | NZ_JAUSUY010000035.1. |
| | | | | Alternatively, DNA |
| | | | | encoding the protein |
| | | | | can be designed |
| | | | | through reverse |
| | | | | translation using a |
| | | | | codon table to |
| | | | | obtain a DNA |
| | | | | sequence. example, |
| | | | | SEQ ID NO: 91. |
| DUF269 | protein involved | Paenibacillus | NZ_JARLKP010000054.1 | Amino-acid (AA) |
| (Pg) | in protection of | graminis | | sequence source is |
| | nitrogenase from | | | WP_340457365.1. |
| | oxidative damage | | | The amino-acid |
| | | | | sequence is |
| | | | | provided as SEQ ID |
| | | | | NO: 92. |
| | | | | An exemplary |
| | | | | genomic source for |
| | | | | DNA encoding the |
| | | | | protein of SEQ ID |
| | | | | NO: 92 can be |
| | | | | NZ_JARLKP010000054.1. |
| | | | | Alternatively, DNA |
| | | | | encoding the protein |
| | | | | can be designed |
| | | | | through reverse |
| | | | | translation using a |
| | | | | codon table to |
| | | | | obtain a DNA |
| | | | | sequence. example, |
| | | | | SEQ ID NO: 93. |
| DUF269 | protein involved | Paenibacillus | NZ_CP048429.1 | Amino-acid (AA) |
| (Pj) | in protection of | jilunlii | | sequence source is |
| | nitrogenase from | | | WP_062523379.1. |
| | oxidative damage | | | The amino-acid |
| | | | | sequence is |
| | | | | provided as SEQ ID |
| | | | | NO: 94. |
| | | | | An exemplary |
| | | | | genomic source for |
| | | | | DNA encoding the |
| | | | | protein of SEQ ID |
| | | | | NO: 94 can be |
| | | | | NZ_CP048429.1. |
| | | | | Alternatively, DNA |
| | | | | encoding the protein |
| | | | | can be designed |
| | | | | through reverse |
| | | | | translation using a |
| | | | | codon table to |
| | | | | obtain a DNA |
| | | | | sequence. example, |
| | | | | SEQ ID NO: 95. |
| DUF269 | protein involved | Paenibacillus | NZ_LN831776.1 | Amino-acid (AA) |
| (Pr) | in protection of | riograndensis | | sequence source is |
| | nitrogenase from | | | WP_020426658.1. |
| | oxidative damage | | | The amino-acid |
| | | | | sequence is |
| | | | | provided as SEQ ID |
| | | | | NO: 96. |
| | | | | An exemplary |
| | | | | genomic source for |
| | | | | DNA encoding the |
| | | | | protein of SEQ ID |
| | | | | NO: 96 can be |
| | | | | NZ_LN831776.1. |
| | | | | Alternatively, DNA |
| | | | | encoding the protein |
| | | | | can be designed |
| | | | | through reverse |
| | | | | translation using a |
| | | | | codon table to |
| | | | | obtain a DNA |
| | | | | sequence. example, |
| | | | | SEQ ID NO: 97. |
| DUF269 | protein involved | Paenibacillus | NZ_CP004078.1 | Amino-acid (AA) |
| (Psab) | in protection of | sabinae | | sequence source is |
| | nitrogenase from | | | WP_025336123.1. |
| | oxidative damage | | | The amino-acid |
| | | | | sequence is |
| | | | | provided as SEQ ID |
| | | | | NO: 98. |
| | | | | An exemplary |
| | | | | genomic source for |
| | | | | DNA encoding the |
| | | | | protein of SEQ ID |
| | | | | NO: 98 can be |
| | | | | NZ_CP004078.1. |
| | | | | Alternatively, DNA |
| | | | | encoding the protein |
| | | | | can be designed |
| | | | | through reverse |
| | | | | translation using a |
| | | | | codon table to |
| | | | | obtain a DNA |
| | | | | sequence. example, |
| | | | | SEQ ID NO: 99. |
| DUF269 | protein involved | Paenibacillus | NZ_CP009286.1 | Amino-acid (AA) |
| (Pstel) | in protection of | stellifer | | sequence source is |
| | nitrogenase from | | | WP_038698114.1. |
| | oxidative damage | | | The amino-acid |
| | | | | sequence is |
| | | | | provided as SEQ ID |
| | | | | NO: 100. |
| | | | | An exemplary |
| | | | | genomic source for |
| | | | | DNA encoding the |
| | | | | protein of SEQ ID |
| | | | | NO: 100 can be |
| | | | | NZ_CP009286.1. |
| | | | | Alternatively, DNA |
| | | | | encoding the protein |
| | | | | can be designed |
| | | | | through reverse |
| | | | | translation using a |
| | | | | codon table to |
| | | | | obtain a DNA |
| | | | | sequence. example, |
| | | | | SEQ ID NO: 101. |
| Synthetic | Synthetic spacer | Synthetic | N/A | Sequence given by |
| spacer and | and ribosomal | | | SEQ ID NO: 86 |
| RBS | binding site | | | |
| 5′ UTR | 5′ Untranslated | Bacillus | CP053102.1 | Nucleotides |
| | region | subtilis | | 3646432-3646459 |
| | | | | of CP053102.1 |
| PR′ | Medium-strength | Bacteriophage | NC_001416.1 | Locus tag |
| | constitutive | lambda | | lambdap72 Gene |
| | promoter | | | ID: 2703521 (nt |
| | | | | 44479-44603 of |
| | | | | NC_001416.1) |
| TR2 | Terminator | Bacteriophage | NC_001416.1 | Locus tag |
| | | lambda | | lambdap54 Gene |
| | | | | ID: 2703490 (nt |
| | | | | 40581-40629 of |
| | | | | NC_001416.1) |

### Example 15. Performance of Additional Engineered Strains Overexpressing DUF269

A number of the strains of Example 5 (Table 14), descended from wild-type parent PB172 or wild-type parent strain NRRL B-68085, were assayed using the acetylene reduction assay (ARA) described in Example 6, the in planta acetylene reduction assay (in planta ARA) described in Example 9, and the OPP ammonia excretion assay described in Example 7 and the results were analyzed using the data analysis methods described in Example 9. Effects are presented in Table 24 as % differences compared to the performance of the wild-type strain (e.g., X % better than wild-type), with a higher number indicating a better performance. Results were not statistically significant at a p-value of 0.2 or less except in two cases. In the acetylene reduction assay (ARA), PB43 showed a significant decrease in activity compared to the wild-type Pb172 (p-value 0.00), while Pp21 showed a significant increase in performance in the OPP ammonia excretion assay (p-value 0.20). Some positive trends may be observed in the data based on numerical differences. In particular, the DUF269 genes originating from Paenibacillus stellifer, Paenibacillus riograndensis and Paenibacillus durus were the top 3 performing heterologous DUF269 gene insertions in PB172-derived strains and in NRRL B-68085-derived strains.

TABLE 24
Mean delta % compared to wild-type in strains
engineered to overexpress DUF269

| | | | Mean | |
|---|---|---|---|---|
| | | | delta (%) | |
| Strain | WT control | | relative | P- |
| ID | Strain ID | Assay | to WT | value |
| Pb40 | PB172 | OPP ammonia | 57.01 | 0.48 |
| | | excretion | | |
| Pb42 | PB172 | OPP ammonia | −0.44 | 1.00 |
| | | excretion | | |
| Pb43 | PB172 | ARA | −21.66 | 0.00 |
| Pb43 | PB172 | in planta ARA | 8.78 | 0.83 |
| Pb43 | PB172 | OPP ammonia | 2.63 | 1.00 |
| | | excretion | | |
| Pb44 | PB172 | ARA | −1.85 | 1.00 |
| Pb44 | PB172 | in planta ARA | 13.99 | 0.72 |
| Pb44 | PB172 | OPP ammonia | 43.09 | 0.79 |
| | | excretion | | |
| Pb45 | PB172 | OPP ammonia | 27.09 | 0.98 |
| | | excretion | | |
| Pb46 | PB172 | ARA | −15.82 | 0.33 |
| Pb46 | PB172 | in planta ARA | 15.68 | 0.68 |
| Pb46 | PB172 | OPP ammonia | 51.01 | 0.62 |
| | | excretion | | |
| Pp16 | NRRL B-68085 | OPP ammonia | 10.13 | 0.78 |
| | | excretion | | |
| Pp17 | NRRL B-68085 | OPP ammonia | −1.97 | 1.00 |
| | | excretion | | |
| Pp18 | NRRL B-68085 | OPP ammonia excretion | 1.81 | 1.00 |
| Pp19 | NRRL B-68085 | OPP ammonia | −5.6 | 0.99 |
| | | excretion | | |
| Pp20 | NRRL B-68085 | OPP ammonia excretion | 14.8 | 0.39 |
| Pp21 | NRRL B-68085 | OPP ammonia | 18.03 | 0.20 |
| | | excretion | | |

### Example 16. Construction of Additional Engineered Strains Overexpressing Certain Genes Responsible for Biogenesis of the Fe—S Clusters of the Nitrogenase Enzyme

Additional engineering was conducted to obtain strains overexpressing certain genes responsible for biogenesis of the Fe—S clusters of the nitrogenase enzyme. Genes heterologously expressed in wild-type Paenibacillus brasilensis PB172 include: Scaffold for Fe—S cluster assembly genes: nifU (Ps) from Stutzerimonas stutzeri, nifU (Kv) from Klebsiella variicola, nifU (Rt) from Rhizobium tropici, nifU (Av) from Azotobacter vinelandii, nifU (Fc) from Frankia canadensis, nifU (Bd) from Bradyrhizobium diazoefficiens, and nifU (Rc) from Rhodobacter capsulatus; Ferredoxin-like protein nifU (Tv) from Trichormus variabilis; Cysteine desulfurase genes: nifS (Ps) from Stutzerimonas stutzeri, nifS (Kv) from Klebsiella variicola, nifS (Rt) from Rhizobium tropici, nifS (Av) from Azotobacter vinelandii, nifS (Fc) from Frankia canadensis, nifS (Bd) from Bradyrhizobium diazoefficiens, nifS (Rc) from Rhodobacter capsulatus, and nifS (Tv) from Trichormus variabilis. Experimental results with these strains are shown in Example 17.

The names nifS and nifU are alternative names for sufS and sufU genes when such genes are located in close proximity to or inside the nitrogenase operon. Accordingly, under the present disclosure the terms nifS and sufS are used interchangeably, as are the terms nifU and sufU. In the case of Pp33, nifU is replaced with a ferredoxin-like protein. Upregulation is achieved either by insertion of the promoter PR′ and the 5′ UTR associated with trnQ (PR′-5′ UTR) downstream of the native promoter sequence and upstream of the native sufCDSUB operon, as in strain Pb23 or by expressing additional copies of genes responsible for biogenesis of the Fe—S clusters, as in strains Pp16-Pp22. In the case of strains Pp16-Pp22, a PR′-5′ UTR cassette or a PtrnQ-5′ UTR cassette (i.e. promoter PtrnQ and its associated 5′ UTR) was inserted upstream of the additional nifS and nifU copies. A synthetic spacer and ribosomal binding site, the sequence of which is given by SEQ ID NO: 86, is inserted downstream of every inserted PR′-5′ UTR or PtrnQ-5′ UTR cassette. The strains were constructed using the multi-plasmid system described in Example 5. Details of the strains are listed in Table 25. Details of the genetic construct components are listed in Table 26. For Strain ID Pb51 the homology pairs used for targeting integrations were SEQ ID NOs: 113 and 114. Strain Pb51 is the result of engineering wild-type parent strain PB172. For Strain IDs Pp23-Pp33 the homology pairs used for targeting integrations were SEQ ID NOs: 36 and 37. Strains Pp23-Pp33 are the result of engineering NRRL B-68085.

TABLE 25
List of Paenibacillus strain IDs, genotype, purpose
of genetic modification, and SEQ ID NOs for cassettes.

| | | Enzyme | | |
|---|---|---|---|---|
| | | Commission | | SEQ ID |
| Strain | Expression cassette | Number/protein | Purpose of genetic | NO: for |
| ID | integrated in strain | class Gene | modification | cassette |
| Pb51 | PsufC(Pb)::PR′-5′ UTR | Fe—S cluster | Increase availability of | 102 |
| | | assembly | the [4Fe—4S] metallo- | |
| | | | cluster to nitrogenase | |
| Pp23 | czcB::PtrnQ-5′ UTR- | Fe—S cluster | Increase availability of | 103 |
| | nifUS(Ps) | assembly | the [4Fe—4S] metallo- | |
| | | | cluster to nitrogenase | |
| Pp24 | czcB::PtrnQ-5′ UTR- | Fe—S cluster | Increase availability of | 104 |
| | nifUS(Kv) | assembly | the [4Fe—4S] metallo- | |
| | | | cluster to nitrogenase | |
| Pp25 | czcB::PtrnQ-5′ UTR- | Fe—S cluster | Increase availability of | 105 |
| | nifUS(Rt) | assembly | the [4Fe—4S] metallo- | |
| | | | cluster to nitrogenase | |
| Pp26 | czcB::PR′-5′ UTR- | Fe—S cluster | Increase availability of | 106 |
| | nifUS(Kv) | assembly | the [4Fe—4S] metallo- | |
| | | | cluster to nitrogenase | |
| Pp27 | czcB::PR′-5′ UTR- | Fe—S cluster | Increase availability of | 107 |
| | nifUS(Av) | assembly | the [4Fe—4S] metallo- | |
| | | | cluster to nitrogenase | |
| Pp28 | czcB::PR′-5′ UTR- | Fe—S cluster | Increase availability of | 108 |
| | nifUS(Ps) | assembly | the [4Fe—4S] metallo- | |
| | | | cluster to nitrogenase | |
| Pp29 | czcB::PR′-5′ UTR- | Fe—S cluster | Increase availability of | 109 |
| | nifUS(Fc) | assembly | the [4Fe—4S] metallo- | |
| | | | cluster to nitrogenase | |
| Pp30 | czcB::PtrnQ-5′ UTR- | Fe—S cluster | Increase availability of | 110 |
| | nifUS(Fc) | assembly | the [4Fe—4S] metallo- | |
| | | | cluster to nitrogenase | |
| Pp31 | czcB::PR′-5′ UTR- | Fe—S cluster | Increase availability of | 111 |
| | nifUS(Bd) | assembly | the [4Fe—4S] metallo- | |
| | | | cluster to nitrogenase | |
| Pp32 | czcB::PR′-5′ UTR- | Fe—S cluster | Increase availability of | 112 |
| | nifUS(Rc) | assembly | the [4Fe—4S] metallo- | |
| | | | cluster to nitrogenase | |
| Pp33 | czcB::PtrnQ-5′ UTR- | Fe—S cluster | Increase availability of | 87 |
| | nifUS(Tv) | assembly, protein | the [4Fe—4S] metallo- | |
| | | electron carrier | cluster to nitrogenase, | |
| | | (ferredoxin) | Increase flux of | |
| | | | electrons to nitrogen | |
| | | | fixation reaction | |

TABLE 26
Components of genetic constructs and component source.

| | | | GenBank | GenBank accession |
|---|---|---|---|---|
| | | | accession # of | # of introduced |
| Name | Description | Source | source genome | component |
| Synthetic | Synthetic spacer | Synthetic | N/A | Sequence given by |
| spacer and | and ribosomal | | | SEQ ID NO: 86 |
| RBS | binding site | | | |
| 5′ UTR | 5′ Untranslated | Bacillus | CP053102.1 | Nucleotides 3646432- |
| | region | subtilis | | 3646459 of |
| | | | | CP053102.1 |
| PR′ | Medium-strength | Bacteriophage | NC_001416.1 | Locus tag lambdap72 |
| | constitutive | lambda | | Gene ID: 2703521 (nt |
| | promoter | | | 44479-44603 of |
| | | | | NC_001416.1) |
| PtrnQ | Strong | Bacillus | CP053102.1 | Locus tag |
| | constitutive | subtilis | | HIR77_19330 (nt |
| | promoter and | | | 3646432-3646592 of |
| | associated 5′ | | | CP053102.1) |
| | UTR | | | |
| nifU(Ps) | Scaffold for | Stutzerimonas | NZ_CP098731.1 | Amino-acid (AA) |
| | Fe—S cluster | stutzeri | | sequence source is |
| | assembly | | | WP_011912527.1. |
| | | | | The amino-acid |
| | | | | sequence is provided |
| | | | | as SEQ ID NO: 115. |
| | | | | An exemplary genomic |
| | | | | source for DNA |
| | | | | encoding the protein of |
| | | | | SEQ ID NO: 115 can |
| | | | | be NZ_CP098731.1. |
| | | | | Alternatively, DNA |
| | | | | encoding the protein |
| | | | | can be designed |
| | | | | through reverse |
| | | | | translation using a |
| | | | | codon table to obtain a |
| | | | | DNA sequence. |
| | | | | example, SEQ ID NO: |
| | | | | 116. |
| nifU(Kv) | Scaffold for | Klebsiella | NZ_CP142520.1 | Amino-acid (AA) |
| | Fe—S cluster | variicola | | sequence source is |
| | assembly | | | WP_023340488.1. |
| | | | | The amino-acid |
| | | | | sequence is provided |
| | | | | as SEQ ID NO: 117. |
| | | | | An exemplary genomic |
| | | | | source for DNA |
| | | | | encoding the protein of |
| | | | | SEQ ID NO: 117 can |
| | | | | be NZ_CP142520.1. |
| | | | | Alternatively, DNA |
| | | | | encoding the protein |
| | | | | can be designed |
| | | | | through reverse |
| | | | | translation using a |
| | | | | codon table to obtain a |
| | | | | DNA sequence. |
| | | | | example, SEQ ID NO: |
| | | | | 118. |
| nifU(Rt) | Scaffold for | Rhizobium | NC_020061.1 | Amino-acid (AA) |
| | Fe—S cluster | tropici | | sequence source is |
| | assembly | | | WP_004126062.1. |
| | | | | The amino-acid |
| | | | | sequence is provided |
| | | | | as SEQ ID NO: 119. |
| | | | | An exemplary genomic |
| | | | | source for DNA |
| | | | | encoding the protein of |
| | | | | SEQ ID NO: 119 can |
| | | | | be NC_020061.1. |
| | | | | Alternatively, DNA |
| | | | | encoding the protein |
| | | | | can be designed |
| | | | | through reverse |
| | | | | translation using a |
| | | | | codon table to obtain a |
| | | | | DNA sequence. |
| | | | | example, SEQ ID NO: |
| | | | | 120. |
| nifU(Av) | Scaffold for | Azotobacter | NZ_CP092752.2 | Amino-acid (AA) |
| | Fe—S cluster | vinelandii | | sequence source is |
| | assembly | | | WP_012698853.1. |
| | | | | The amino-acid |
| | | | | sequence is provided |
| | | | | as SEQ ID NO: 121. |
| | | | | An exemplary genomic |
| | | | | source for DNA |
| | | | | encoding the protein of |
| | | | | SEQ ID NO: 121 can |
| | | | | be NZ_CP092752.2. |
| | | | | Alternatively, DNA |
| | | | | encoding the protein |
| | | | | can be designed |
| | | | | through reverse |
| | | | | translation using a |
| | | | | codon table to obtain a |
| | | | | DNA sequence. |
| | | | | example, SEQ ID NO: |
| | | | | 122. |
| nifU(Fc) | Scaffold for | Frankia | NZ_OESX01000479.1 | Amino-acid (AA) |
| | Fe—S cluster | canadensis | | sequence source is |
| | assembly | | | WP_101834302.1. |
| | | | | The amino-acid |
| | | | | sequence is provided |
| | | | | as SEQ ID NO: 123. |
| | | | | An exemplary genomic |
| | | | | source for DNA |
| | | | | encoding the protein of |
| | | | | SEQ ID NO: 123 can |
| | | | | be |
| | | | | NZ_OESX01000479.1. |
| | | | | Alternatively, DNA |
| | | | | encoding the protein |
| | | | | can be designed |
| | | | | through reverse |
| | | | | translation using a |
| | | | | codon table to obtain a |
| | | | | DNA sequence. |
| | | | | example, SEQ ID NO: |
| | | | | 124. |
| nifU(Bd) | Scaffold for | Bradyrhizobium | NZ_CP181400.1 | Amino-acid (AA) |
| | Fe—S cluster | diazoefficiens | | sequence source is |
| | assembly | | | WP_011084564.1. |
| | | | | The amino-acid |
| | | | | sequence is provided |
| | | | | as SEQ ID NO: 125. |
| | | | | An exemplary genomic |
| | | | | source for DNA |
| | | | | encoding the protein of |
| | | | | SEQ ID NO: 125 can |
| | | | | be NZ_CP181400.1. |
| | | | | Alternatively, DNA |
| | | | | encoding the protein |
| | | | | can be designed |
| | | | | through reverse |
| | | | | translation using a |
| | | | | codon table to obtain a |
| | | | | DNA sequence. |
| | | | | example, SEQ ID NO: |
| | | | | 126. |
| nifU(Rc) | Scaffold for | Rhodobacter | NZ_CP198940.1 | Amino-acid (AA) |
| | Fe—S cluster | capsulatus | | sequence source is |
| | assembly | | | WP_141516648.1. |
| | | | | The amino-acid |
| | | | | sequence is provided |
| | | | | as SEQ ID NO: 127. |
| | | | | An exemplary genomic |
| | | | | source for DNA |
| | | | | encoding the protein of |
| | | | | SEQ ID NO: 127 can |
| | | | | be NZ_CP198940.1. |
| | | | | Alternatively, DNA |
| | | | | encoding the protein |
| | | | | can be designed |
| | | | | through reverse |
| | | | | translation using a |
| | | | | codon table to obtain a |
| | | | | DNA sequence. |
| | | | | example, SEQ ID NO: |
| | | | | 128. |
| nifU(Tv) | Ferredoxin-like | Trichormus | CP047242.1 | Amino-acid (AA) |
| | protein | variabilis | | sequence source is |
| | | | | QHD82901.1. |
| | | | | The amino-acid |
| | | | | sequence is provided |
| | | | | as SEQ ID NO: 129. |
| | | | | An exemplary genomic |
| | | | | source for DNA |
| | | | | encoding the protein of |
| | | | | SEQ ID NO: 129 can |
| | | | | be CP047242.1. |
| | | | | Alternatively, DNA |
| | | | | encoding the protein |
| | | | | can be designed |
| | | | | through reverse |
| | | | | translation using a |
| | | | | codon table to obtain a |
| | | | | DNA sequence. |
| | | | | example, SEQ ID NO: |
| | | | | 130. |
| nifS(Ps) | Cysteine | Stutzerimonas | NZ_WIFO01000005.1 | Amino-acid (AA) |
| | desulfurase | stutzeri | | sequence source is |
| | | | | WP_200587152.1. |
| | | | | The amino-acid |
| | | | | sequence is provided |
| | | | | as SEQ ID NO: 131. |
| | | | | An exemplary genomic |
| | | | | source for DNA |
| | | | | encoding the protein of |
| | | | | SEQ ID NO: 131 can |
| | | | | be |
| | | | | NZ_WIFO01000005.1. |
| | | | | Alternatively, DNA |
| | | | | encoding the protein |
| | | | | can be designed |
| | | | | through reverse |
| | | | | translation using a |
| | | | | codon table to obtain a |
| | | | | DNA sequence. |
| | | | | example, SEQ ID NO: |
| | | | | 132. |
| nifS(Kv) | Cysteine | Klebsiella | NZ_CP196321.1 | Amino-acid (AA) |
| | desulfurase | variicola | | sequence source is |
| | | | | WP_023297938.1. |
| | | | | The amino-acid |
| | | | | sequence is provided |
| | | | | as SEQ ID NO: 133. |
| | | | | An exemplary genomic |
| | | | | source for DNA |
| | | | | encoding the protein of |
| | | | | SEQ ID NO: 133 can |
| | | | | be NZ_CP196321.1. |
| | | | | Alternatively, DNA |
| | | | | encoding the protein |
| | | | | can be designed |
| | | | | through reverse |
| | | | | translation using a |
| | | | | codon table to obtain a |
| | | | | DNA sequence. |
| | | | | example, SEQ ID NO: |
| | | | | 134. |
| nifS(Rt) | Cysteine | Rhizobium | NZ_JACHBH010000027.1 | Amino-acid (AA) |
| | desulfurase | tropici | | sequence source is |
| | | | | WP_004126059.1. |
| | | | | The amino-acid |
| | | | | sequence is provided |
| | | | | as SEQ ID NO: 135. |
| | | | | An exemplary genomic |
| | | | | source for DNA |
| | | | | encoding the protein of |
| | | | | SEQ ID NO: 135 can |
| | | | | be |
| | | | | NZ_JACHBH0100000 |
| | | | | 27.1. Alternatively, |
| | | | | DNA encoding the |
| | | | | protein can be |
| | | | | designed through |
| | | | | reverse translation |
| | | | | using a codon table to |
| | | | | obtain a DNA |
| | | | | sequence. example, |
| | | | | SEQ ID NO: 136. |
| nifS(Av) | Cysteine | Azotobacter | NZ_CP092752.2 | Amino-acid (AA) |
| | desulfurase | vinelandii | | sequence source is |
| | | | | WP_012698854.1. |
| | | | | The amino-acid |
| | | | | sequence is provided |
| | | | | as SEQ ID NO: 137. |
| | | | | An exemplary genomic |
| | | | | source for DNA |
| | | | | encoding the protein of |
| | | | | SEQ ID NO: 137 can |
| | | | | be NZ_CP092752.2. |
| | | | | Alternatively, DNA |
| | | | | encoding the protein |
| | | | | can be designed |
| | | | | through reverse |
| | | | | translation using a |
| | | | | codon table to obtain a |
| | | | | DNA sequence. |
| | | | | example, SEQ ID NO: |
| | | | | 138. |
| nifS(Fc) | Cysteine | Frankia | NZ_OESX01000479.1 | Amino-acid (AA) |
| | desulfurase | canadensis | | sequence source is |
| | | | | WP_101834330.1. |
| | | | | The amino-acid |
| | | | | sequence is provided |
| | | | | as SEQ ID NO: 139. |
| | | | | An exemplary genomic |
| | | | | source for DNA |
| | | | | encoding the protein of |
| | | | | SEQ ID NO: 139 can |
| | | | | be |
| | | | | NZ_OESX01000479.1. |
| | | | | Alternatively, DNA |
| | | | | encoding the protein |
| | | | | can be designed |
| | | | | through reverse |
| | | | | translation using a |
| | | | | codon table to obtain a |
| | | | | DNA sequence. |
| | | | | example, SEQ ID NO: |
| | | | | 140. |
| nifS(Bd) | Cysteine | Bradyrhizobium | NZ_CP172424.1 | Amino-acid (AA) |
| | desulfurase | diazoefficiens | | sequence source is |
| | | | | WP_060909242.1. |
| | | | | The amino-acid |
| | | | | sequence is provided |
| | | | | as SEQ ID NO: 141. |
| | | | | An exemplary genomic |
| | | | | source for DNA |
| | | | | encoding the protein of |
| | | | | SEQ ID NO: 141 can |
| | | | | be NZ_CP172424.1. |
| | | | | Alternatively, DNA |
| | | | | encoding the protein |
| | | | | can be designed |
| | | | | through reverse |
| | | | | translation using a |
| | | | | codon table to obtain a |
| | | | | DNA sequence. |
| | | | | example, SEQ ID NO: |
| | | | | 142. |
| nifS(Rc) | Cysteine | Rhodobacter | NZ_CP198940.1 | Amino-acid (AA) |
| | desulfurase | capsulatus | | sequence source is |
| | | | | WP_141516649.1. |
| | | | | The amino-acid |
| | | | | sequence is provided |
| | | | | as SEQ ID NO: 143. |
| | | | | An exemplary genomic |
| | | | | source for DNA |
| | | | | encoding the protein of |
| | | | | SEQ ID NO: 143 can |
| | | | | be NZ_CP198940.1. |
| | | | | Alternatively, DNA |
| | | | | encoding the protein |
| | | | | can be designed |
| | | | | through reverse |
| | | | | translation using a |
| | | | | codon table to obtain a |
| | | | | DNA sequence. |
| | | | | example, SEQ ID NO: |
| | | | | 144. |
| nifS(Tv) | Cysteine | Trichormus | CP047242.1 | Amino-acid (AA) |
| | desulfurase | variabilis | | sequence source is |
| | | | | QHD82902.1. |
| | | | | The amino-acid |
| | | | | sequence is provided |
| | | | | as SEQ ID NO: 145. |
| | | | | An exemplary genomic |
| | | | | source for DNA |
| | | | | encoding the protein of |
| | | | | SEQ ID NO: 145 can |
| | | | | be CP047242.1. |
| | | | | Alternatively, DNA |
| | | | | encoding the protein |
| | | | | can be designed |
| | | | | through reverse |
| | | | | translation using a |
| | | | | codon table to obtain a |
| | | | | DNA sequence. |
| | | | | example, SEQ ID NO: |
| | | | | 146. |

### Example 17. Performance of Additional Engineered Strains Overexpressing Certain Genes Responsible for Biogenesis of the Fe—S Clusters of the Nitrogenase Enzyme

A number of the strains of Example 16, descended from wild-type parent strains PB172 or NRRL B-68085, were assayed using the OPP ammonia excretion assay described in Example 7 and the results were analyzed using the data analysis methods described in Example 9. Table 27 shows effects of overexpression of certain genes responsible for biogenesis of the Fe—S clusters in genetically engineered strains in the OPP ammonia excretion assay. In each case, effects are presented as % differences compared to the performance of the wild-type strain (e.g., X % better than wild-type), with a higher number indicating a better performance. Results having a p-value less than 0.1 were considered statistically significant.

TABLE 27
Mean delta % compared to wild-type in strains
engineered to overexpress Fe—S clusters

| | WT control Strain | | Mean delta (%) | |
|---|---|---|---|---|
| Strain ID | ID | Edit | relative to WT | P-value |
| Pb51 | PB172 | PsufC(Pb)::PR′-5′ UTR | −8.39* | 0.98 |
| Pp23 | NRRL B-68085 | czcB::PtrnQ-5′ UTR- | 2436.48 | 0.00 |
| | | nifUS(Ps) | | |
| Pp24 | NRRL B-68085 | czcB::PtrnQ-5′ UTR- | −346.41* | 0.94 |
| | | nifUS(Kv) | | |
| Pp25 | NRRL B-68085 | czcB::PtrnQ-5′ UTR- | 3069.3 | 0.00 |
| | | nifUS(Rt) | | |
| Pp26 | NRRL B-68085 | czcB::PR′-5′ UTR-nifUS(Kv) | 2055.23 | 0.00 |
| Pp27 | NRRL B-68085 | czcB::PR′-5′ UTR-nifUS(Av) | −352.21* | 0.93 |
| Pp28 | NRRL B-68085 | czcB::PR′-5′ UTR-nifUS(Ps) | −241.91* | 1.00 |
| Pp29 | NRRL B-68085 | czcB::PR′-5′ UTR-nifUS(Fc) | 7946.12 | 0.00 |
| Pp30 | NRRL B-68085 | czcB::PtrnQ-5′ UTR- | −269* | 0.99 |
| | | nifUS(Fc) | | |
| Pp31 | NRRL B-68085 | czcB::PR′-5′ UTR-nifUS(Bd) | 2355.2 | 0.00 |
| Pp32 | NRRL B-68085 | czcB::PR′-5′ UTR-nifUS(Rc) | 7.74* | 1.00 |
| Pp33 | NRRL B-68085 | czcB::PtrnQ-5′ UTR- | −261.26* | 0.99 |
| | | nifUS(Tv) | | |

### Example 18. Construction of Additional Engineered Strains with Altered Expression of Flavodoxin

Engineering was conducted to obtain strains overexpressing flavodoxin. Flavodoxin genes heterologously expressed include: nifF (Ss) from Synechococcus sp. MIT S9508; nifF (Ps) from Pseudomonas sp. OF001; fldA (Bs) from Bacillus sp. FJAT-27264; nifF (Km) from Klebsiella michiganensis; nifF (Rc) from Rhodobacter capsulatus; fldA (Pt) from Paenibacillus tundrae; fldA (Cf) from Cohnella fermenti; fldA (Bm) from Brevibacillus marinus; nifF (Ac) from Azotobacter chroococcum; nifF (Mc) from Methylococcus capsulatus; and fldA (Ta) from Thermolongibacillus altinsuensis. The performance of these strains is described in Example 20.

Engineering was achieved either by the insertion of terminator TR2 and the 5′ UTR associated with trnQ (TR2-5′ UTR) downstream of the native promoter sequence and upstream of the flavodoxin gene, as in strain Pp34, or by expressing additional copies of the flavodoxin gene, as in strains Pp35-Pp49. In the case of strains Pp35-Pp49, a PR′-5′ UTR cassette (i.e. promoter PR′ and the 5′ UTR associated with trnQ) or a PtrnQ-5′ UTR cassette (i.e. promoter PtrnQ and its associated 5′ UTR) was inserted upstream of the additional flavodoxin copies. A synthetic spacer and ribosomal binding site, the sequence of which is given by SEQ ID NO: 86, is inserted downstream of every inserted TR2-5′ UTR, PR′-5′ UTR or PtrnQ-5′ UTR cassette. A flavodoxin can be encoded by a fldA or nifF gene. Accordingly, under the present disclosure the terms fldA gene and nifF gene are used interchangeably. Said nifF genes tend to be located close to or inside the nitrogenase operon. The strains were constructed using the multi-plasmid system described in Example 5. Details of the strains are listed in Table 28. Details of the genetic construct components are listed in Table 29. For Strain ID Pp34 the homology pairs used for targeting integrations were SEQ ID NOs: 185 and 186. For Strain IDs Pp35-Pp49 the homology pairs used for targeting integrations were SEQ ID NOs: 36 and 37. Strains Pp35-Pp49 are the result of engineering NRRL B-68085.

TABLE 28
List of Paenibacillus strain IDs, genotype, purpose
of genetic modification, and SEQ ID NOs for cassettes.

| | | Enzyme | | |
|---|---|---|---|---|
| | | Commission | | SEQ ID |
| Strain | Expression cassette | Number/protein | Purpose of genetic | NO: for |
| ID | integrated in strain | class Gene | modification | cassette |
| Pp34 | PfldA(Ppe)::TR2-5′ UTR | Protein electron | Negative control: | 147 |
| | | carrier (flavodoxin) | Decrease flux of | |
| | | | electrons to nitrogen | |
| | | | fixation reaction | |
| Pp35 | czcB::PtrnQ-5′ UTR- | Protein electron | Increase flux of | 148 |
| | nifF(Ss) | carrier (flavodoxin) | electrons to nitrogen | |
| | | | fixation reaction | |
| Pp36 | czcB::PtrnQ-5′ UTR- | Protein electron | Increase flux of | 149 |
| | nifF(Ps) | carrier (flavodoxin) | electrons to nitrogen | |
| | | | fixation reaction | |
| Pp37 | czcB::PtrnQ-5′ UTR- | Protein electron | Increase flux of | 150 |
| | fldA(Bs) | carrier (flavodoxin) | electrons to nitrogen | |
| | | | fixation reaction | |
| Pp38 | czcB::PtrnQ-5′ UTR- | Protein electron | Increase flux of | 151 |
| | nifF(Km) | carrier (flavodoxin) | electrons to nitrogen | |
| | | | fixation reaction | |
| Pp39 | czcB::PR′-5′ UTR-nifF(Rc) | Protein electron | Increase flux of | 152 |
| | | carrier (flavodoxin) | electrons to nitrogen | |
| | | | fixation reaction | |
| Pp40 | czcB::PR′-5′ UTR-fldA(Pt) | Protein electron | Increase flux of | 153 |
| | | carrier (flavodoxin) | electrons to nitrogen | |
| | | | fixation reaction | |
| Pp41 | czcB::PR′-5′ UTR-fldA(Cf) | Protein electron | Increase flux of | 154 |
| | | carrier (flavodoxin) | electrons to nitrogen | |
| | | | fixation reaction | |
| Pp42 | CzcB::PR′-5′ UTR- | Protein electron | Increase flux of | 155 |
| | fldA(Bm) | carrier (flavodoxin) | electrons to nitrogen | |
| | | | fixation reaction | |
| Pp43 | czcB::PR′-5′ UTR-nifF(Ac) | Protein electron | Increase flux of | 156 |
| | | carrier (flavodoxin) | electrons to nitrogen | |
| | | | fixation reaction | |
| Pp44 | czcB::PR′-5′ UTR-nifF(Ss) | Protein electron | Increase flux of | 157 |
| | | carrier (flavodoxin) | electrons to nitrogen | |
| | | | fixation reaction | |
| Pp45 | czcB::PR′-5′ UTR-nifF(Ps) | Protein electron | Increase flux of | 158 |
| | | carrier (flavodoxin) | electrons to nitrogen | |
| | | | fixation reaction | |
| Pp46 | czcB::PR′-5′ UTR-fldA(Bs) | Protein electron | Increase flux of | 159 |
| | | carrier (flavodoxin) | electrons to nitrogen | |
| | | | fixation reaction | |
| Pp47 | czcB::PR′-5′ UTR-nifF(Mc) | Protein electron | Increase flux of | 160 |
| | | carrier (flavodoxin) | electrons to nitrogen | |
| | | | fixation reaction | |
| Pp48 | czcB::PR′-5′ UTR-fldA(Ta) | Protein electron | Increase flux of | 161 |
| | | carrier (flavodoxin) | electrons to nitrogen | |
| | | | fixation reaction | |
| Pp49 | czcB::PR′-5′ UTR- | Protein electron | Increase flux of | 162 |
| | nifF(Km) | carrier (flavodoxin) | electrons to nitrogen | |
| | | | fixation reaction | |

TABLE 29
Components of genetic constructs and component source.

| | | | GenBank | GenBank |
|---|---|---|---|---|
| | | | accession # | accession # |
| | | | of source | of introduced |
| Name | Description | Source | genome | component |
| Synthetic | Synthetic | Synthetic | N/A | Sequence given by |
| spacer | spacer and | | | SEQ ID NO: 86 |
| and RBS | ribosomal | | | |
| | binding site | | | |
| 5′ UTR | 5′ Untranslated | Bacillus subtilis | CP053102.1 | Nucleotides |
| | region | | | 3646432-3646459 |
| | | | | of CP053102.1 |
| PR′ | Medium- | Bacteriophage | NC_001416.1 | Locus tag |
| | strength | lambda | | lambdap72 Gene |
| | constitutive | | | ID: 2703521 (nt |
| | promoter | | | 44479-44603 of |
| | | | | NC_001416.1) |
| PtrnQ | Strong | Bacillus subtilis | CP053102.1 | Locus tag |
| | constitutive | | | HIR77_19330 (nt |
| | promoter and | | | 3646432-3646592 |
| | associated 5′ | | | of CP053102.1) |
| | UTR | | | |
| TR2 | Terminator | Bacteriophage | NC_001416.1 | Locus tag |
| | | lambda | | lambdap54 Gene |
| | | | | ID: 2703490 (nt |
| | | | | 40581-40629 of |
| | | | | NC_001416.1) |
| nifF(Ss) | Flavodoxin | Synechococcus | NZ_LVHU01000002.1 | Amino-acid (AA) |
| | | sp. MIT S9508 | | sequence source is |
| | | | | WP_369818416.1. |
| | | | | The amino-acid |
| | | | | sequence is |
| | | | | provided as SEQ ID |
| | | | | NO: 163. |
| | | | | An exemplary |
| | | | | genomic source for |
| | | | | DNA encoding the |
| | | | | protein of SEQ ID |
| | | | | NO: 163 can be |
| | | | | NZ_LVHU01000002.1. |
| | | | | Alternatively, |
| | | | | DNA encoding the |
| | | | | protein can be |
| | | | | designed through |
| | | | | reverse translation |
| | | | | using a codon table |
| | | | | to obtain a DNA |
| | | | | sequence. |
| | | | | example, SEQ ID |
| | | | | NO: 164. |
| nifF(Ps) | Flavodoxin | Pseudomonas | NZ_CAJGBM010000007.1 | Amino-acid (AA) |
| | | sp. OF001 | | sequence source is |
| | | | | WP_201485681.1. |
| | | | | The amino-acid |
| | | | | sequence is |
| | | | | provided as SEQ ID |
| | | | | NO: 165. |
| | | | | An exemplary |
| | | | | genomic source for |
| | | | | DNA encoding the |
| | | | | protein of SEQ ID |
| | | | | NO: 165 can be |
| | | | | NZ_CAJGBM010000007.1. |
| | | | | Alternatively, DNA |
| | | | | encoding the |
| | | | | protein can be |
| | | | | designed through |
| | | | | reverse translation |
| | | | | using a codon table |
| | | | | to obtain a DNA |
| | | | | sequence. |
| | | | | example, SEQ ID |
| | | | | NO: 166. |
| fldA(Bs) | Flavodoxin | Bacillus sp. | NZ_MAQW01000001.1 | Amino-acid (AA) |
| | | FJAT-27264 | | sequence source is |
| | | | | WP_066362945.1. |
| | | | | The amino-acid |
| | | | | sequence is |
| | | | | provided as SEQ ID |
| | | | | NO: 167. |
| | | | | An exemplary |
| | | | | genomic source for |
| | | | | DNA encoding the |
| | | | | protein of SEQ ID |
| | | | | NO: 167 can be |
| | | | | NZ_MAQW01000001.1. |
| | | | | Alternatively, |
| | | | | DNA encoding the |
| | | | | protein can be |
| | | | | designed through |
| | | | | reverse translation |
| | | | | using a codon table |
| | | | | to obtain a DNA |
| | | | | sequence. |
| | | | | example, SEQ ID |
| | | | | NO: 168. |
| nifF(Km) | Flavodoxin | Klebsiella | NZ_CABGHZ010000001.1 | Amino-acid (AA) |
| | | michiganensis | | sequence source is |
| | | | | WP_048260848.1. |
| | | | | The amino-acid |
| | | | | sequence is |
| | | | | provided as SEQ ID |
| | | | | NO: 169. |
| | | | | An exemplary |
| | | | | genomic source for |
| | | | | DNA encoding the |
| | | | | protein of SEQ ID |
| | | | | NO: 169 can be |
| | | | | GENEACCESSION. |
| | | | | Alternatively, DNA |
| | | | | encoding the |
| | | | | protein can be |
| | | | | designed through |
| | | | | reverse translation |
| | | | | using a codon table |
| | | | | to obtain a DNA |
| | | | | sequence. |
| | | | | example, SEQ ID |
| | | | | NO: 170. |
| nifF(Rc) | Flavodoxin | Rhodobacter | NC_014034.1 | Amino-acid (AA) |
| | | capsulatus | | sequence source is |
| | | | | WP_013067145.1. |
| | | | | The amino-acid |
| | | | | sequence is |
| | | | | provided as SEQ ID |
| | | | | NO: 171. |
| | | | | An exemplary |
| | | | | genomic source for |
| | | | | DNA encoding the |
| | | | | protein of SEQ ID |
| | | | | NO: 171 can be |
| | | | | NC_014034.1. |
| | | | | Alternatively, DNA |
| | | | | encoding the |
| | | | | protein can be |
| | | | | designed through |
| | | | | reverse translation |
| | | | | using a codon table |
| | | | | to obtain a DNA |
| | | | | sequence. |
| | | | | example, SEQ ID |
| | | | | NO: 172. |
| fldA(Pt) | Flavodoxin | Paenibacillus | NZ_CP145605.1 | Amino-acid (AA) |
| | | tundrae | | sequence source is |
| | | | | WP_056689646.1. |
| | | | | The amino-acid |
| | | | | sequence is |
| | | | | provided as SEQ ID |
| | | | | NO: 173. |
| | | | | An exemplary |
| | | | | genomic source for |
| | | | | DNA encoding the |
| | | | | protein of SEQ ID |
| | | | | NO: 173 can be |
| | | | | NZ_CP145605.1. |
| | | | | Alternatively, DNA |
| | | | | encoding the |
| | | | | protein can be |
| | | | | designed through |
| | | | | reverse translation |
| | | | | using a codon table |
| | | | | to obtain a DNA |
| | | | | sequence. |
| | | | | example, SEQ ID |
| | | | | NO: 174. |
| fldA(Cf) | Flavodoxin | Cohnella | NZ_SSOB01000008.1 | Amino-acid (AA) |
| | | fermenti | | sequence source is |
| | | | | WP_136369241.1. |
| | | | | The amino-acid |
| | | | | sequence is |
| | | | | provided as SEQ ID |
| | | | | NO: 175. |
| | | | | An exemplary |
| | | | | genomic source for |
| | | | | DNA encoding the |
| | | | | protein of SEQ ID |
| | | | | NO: 175 can be |
| | | | | NZ_SSOB01000008.1. |
| | | | | Alternatively, |
| | | | | DNA encoding the |
| | | | | protein can be |
| | | | | designed through |
| | | | | reverse translation |
| | | | | using a codon table |
| | | | | to obtain a DNA |
| | | | | sequence. |
| | | | | example, SEQ ID |
| | | | | NO: 176. |
| fldA(Bm) | Flavodoxin | Brevibacillus | NZ_CP034541.1 | Amino-acid (AA) |
| | | marinus | | sequence source is |
| | | | | WP_126428096.1. |
| | | | | The amino-acid |
| | | | | sequence is |
| | | | | provided as SEQ ID |
| | | | | NO: 177. |
| | | | | An exemplary |
| | | | | genomic source for |
| | | | | DNA encoding the |
| | | | | protein of SEQ ID |
| | | | | NO: 177 can be |
| | | | | NZ_CP034541.1. |
| | | | | Alternatively, DNA |
| | | | | encoding the |
| | | | | protein can be |
| | | | | designed through |
| | | | | reverse translation |
| | | | | using a codon table |
| | | | | to obtain a DNA |
| | | | | sequence. |
| | | | | example, SEQ ID |
| | | | | NO: 178. |
| nifF(Ac) | Flavodoxin | Azotobacter | NZ_CP066310.1 | Amino-acid (AA) |
| | | chroococcum | | sequence source is |
| | | | | WP_039800902.1. |
| | | | | The amino-acid |
| | | | | sequence is |
| | | | | provided as SEQ ID |
| | | | | NO: 179. |
| | | | | An exemplary |
| | | | | genomic source for |
| | | | | DNA encoding the |
| | | | | protein of SEQ ID |
| | | | | NO: 179 can be |
| | | | | NZ_CP066310.1. |
| | | | | Alternatively, DNA |
| | | | | encoding the |
| | | | | protein can be |
| | | | | designed through |
| | | | | reverse translation |
| | | | | using a codon table |
| | | | | to obtain a DNA |
| | | | | sequence. |
| | | | | example, SEQ ID |
| | | | | NO: 180. |
| nifF(Mc) | Flavodoxin | Methylococcus | NZ_DAIONT010000004.1 | Amino-acid (AA) |
| | | capsulatus | | sequence source is |
| | | | | WP_333687284.1. |
| | | | | The amino-acid |
| | | | | sequence is |
| | | | | provided as SEQ ID |
| | | | | NO: 181. |
| | | | | An exemplary |
| | | | | genomic source for |
| | | | | DNA encoding the |
| | | | | protein of SEQ ID |
| | | | | NO: 181 can be |
| | | | | NZ_DAIONT010000004.1. |
| | | | | Alternatively, DNA |
| | | | | encoding the |
| | | | | protein can be |
| | | | | designed through |
| | | | | reverse translation |
| | | | | using a codon table |
| | | | | to obtain a DNA |
| | | | | sequence. |
| | | | | example, SEQ ID |
| | | | | NO: 182. |
| fldA(Ta) | Flavodoxin | Thermolongibacillus | NZ_BSVG01000009.1 | Amino-acid (AA) |
| | | altinsuensis | | sequence source is |
| | | | | WP_285518104.1. |
| | | | | The amino-acid |
| | | | | sequence is |
| | | | | provided as SEQ ID |
| | | | | NO: 183. |
| | | | | An exemplary |
| | | | | genomic source for |
| | | | | DNA encoding the |
| | | | | protein of SEQ ID |
| | | | | NO: 183 can be |
| | | | | NZ_BSVG01000009.1. |
| | | | | Alternatively, |
| | | | | DNA encoding the |
| | | | | protein can be |
| | | | | designed through |
| | | | | reverse translation |
| | | | | using a codon table |
| | | | | to obtain a DNA |
| | | | | sequence. |
| | | | | example, SEQ ID |
| | | | | NO: 184. |

### Example 19. Performance of Additional Engineered Strains with Altered Expression of Flavodoxin

A number of the strains of Example 18, descended from NRRL B-68085, were assayed using the OPP ammonia excretion assay described in Example 7 and the results were analyzed using the data analysis methods described in Example 9. Table 30 shows effects of overexpression of flavodoxin in genetically engineered strains. In each case, effects are presented as % differences compared to the performance of the wild-type strain (e.g., X % better than wild-type), with a higher number indicating a better performance. Results having a p-value less than 0.1 were considered statistically significant.

TABLE 30
Mean delta % compared to wild-type in strains
engineered to overexpress flavodoxin.

| Strain | Mean delta (%) | P- |
|---|---|---|
| ID | relative to WT | value |
| Pp35 | −149.77 | 1.00 |
| Pp36 | −56.16 | 1.00 |
| Pp37 | 4333.89 | 0.00 |
| Pp38 | 20.59 | 1.00 |
| Pp39 | 387.52 | 1.00 |
| Pp40 | 3862.12 | 0.00 |
| Pp41 | 2182.86 | 0.00 |
| Pp42 | 310.77 | 1.00 |
| Pp43 | 3465.24 | 0.00 |
| Pp44 | 7915.2 | 0.00 |
| Pp45 | −824.42 | 0.09 |
| Pp46 | −536.06 | 0.54 |
| Pp47 | 1476.59 | 0.00 |
| Pp48 | 6761.75 | 0.00 |
| Pp49 | 2418.74 | 0.00 |

### Example 20. Performance of Selected Engineered Strains in 28-Day Nitrogen-Stress Greenhouse Assay

Strain Pb39 (descended from PB172, but in which Pz fer ferredoxin has been upregulated) and strain Pp1 (descended from NRRL B-68085, but also in which Pz fer ferredoxin has been upregulated), and their wild-type parents PB172 and NRRL B-68085 were assayed using the 28-day nitrogen-stress greenhouse assay described in Example 9 and the results were analyzed using the data analysis methods described in Example 9. Table 31 shows the performance of entries, including chemical controls, certain engineered strains and their wild-type parents as measured by plant digital biomass in the 28-day nitrogen-stress greenhouse assay. Effects are presented as % differences compared to the performance of the seed not treated with nitrogen-fixing strains at suboptimal/low nitrogen (e.g., X % better than baseline), with a higher number indicating a better performance. Seed not treated with nitrogen-fixing strains at optimal nitrogen levels is marked as UTC High. P-values are provided.

TABLE 31
28-day nitrogen-stress greenhouse assay
for strains over-expressing ferredoxin

| | | Mean delta | |
|---|---|---|---|
| | | (%) relative | P- |
| Entry ID | Control ID | to UTC LOW | value |
| NRRL B- | UTC Low | 3.78 | 0.13 |
| 68085 | | | |
| PB172 | UTC Low | 6.28 | 0.00 |
| Pb39 | UTC Low | 7.43 | 0.21 |
| Pp1 | UTC Low | 8.62 | 0.01 |
| UTC HIGH | UTC Low | 29.85 | 0.00 |

Optimal nitrogen conditions in the absence of nitrogen-fixing strains, as shown in UTC High, lead to a significant increase in plant biomass compared to suboptimal nitrogen conditions in the absence of nitrogen-fixing strains, i.e. the baseline shown in Table 31. Every nitrogen-fixing strain tested had a positive effect on plant biomass compared to baseline. Furthermore, it is apparent that ferredoxin upregulation leads to improved nitrogen performance in the engineered strain compared to that of the wild-type parent. As can be seen in Table 31, Pb39, overexpressing the fer gene from Paenibacillus zanthoxyli (Pz_fer) encoding ferredoxin, had better performance than its wild-type parent PB172; and Pp1, also overexpressing the fer gene from Paenibacillus zanthoxyli (Pz_fer) encoding ferredoxin, performed better than its wild-type parent NRRL B-68085.

Strains Pp1, Pp8, Pp10, Pp12 and Pp13 (in each of which ferredoxin is upregulated, each of them descended from NRRL B-68085) and their wild-type parent NRRL B-68085 were assayed using the 28-day nitrogen-stress greenhouse assay described in Example 9 and the results were analyzed using the data analysis methods described in Example 9. None of the results showed statistically significant improvement over wild-type.

Strains Pp34, Pp37, Pp43 and Pp48, in which flavodoxin is upregulated, each of them descended from NRRL B-68085, and their wild-type parent NRRL B-68085 were assayed using the 28-day nitrogen-stress greenhouse assay described in Example 9 and the results were analyzed using the data analysis methods described in Example 9. None of the strains showed statistically significant results: Pp43, Pp48 and Pp37 showed numerical increases in plant biomass while Pp34 showed a decrease.

Strain Pp23 in which certain genes responsible for biogenesis of the Fe—S clusters are upregulated, descended from NRRL B-68085, and its wild-type parent NRRL B-68085 were assayed using the 28-day nitrogen-stress greenhouse assay described in Example 9 and the results were analyzed using the data analysis methods described in Example 9. Pp23 showed a 2.59% increase in plant biomass compared to the wild-type parent strain at a p-value of 0.57.

Strain Pp21 in which DUF269 is upregulated, descended from NRRL B-68085, and its wild-type parent NRRL B-68085 were assayed using the 28-day nitrogen-stress greenhouse assay described in Example 9 and the results were analyzed using the data analysis methods described in Example 9. The strain showed a 6.4% increase in plant biomass compared to the wild-type parent strain at a p-value of 0.08. It is noted that overexpressing DUF269 in strain Pp21 to improve nitrogenase oxygen tolerance leads to increase in plant biomass indicating a positive effect conferred by the genetic edit.

### Example 21. Performance of Selected Engineered Strains in 49-Day Nitrogen-Stressed Greenhouse Experiments

NRRLB-68085 (wild-type parent Paenibacillus peoriae strain) and various genetically engineered strains described in the examples above were tested in greenhouse assays as follows. A fermentation broth of each strain was prepared as follows: The Paenibacillus brasilensis wild-type and edited strains were each cultured in shake flasks containing a soluble complex seed medium, such as TSB. Briefly, a seed culture was created by inoculating 50 mL of the medium in a 250 mL baffled flask with 400 uL of an archived glycerol stock of each strain. This seed culture was incubated 16-20 hours on a rotary shaker at 200 rpm at 30° C. For spore production, 5 mL of the seed culture was used to inoculate 250 ml of a soy-based medium in a 1000 mL flask, and incubated on a rotary shaker at 200 rpm at 30° C. for 5 days to facilitate sporulation. A sample of the culture was streaked on TSA and incubated for 3 days at 30° C. to check for purity and contamination. Spore enumeration was performed by plating serial dilutions of heat-treated sporulation culture onto TSA. Corn seeds were treated first with a fungicide seed treatment. The microbial fermentation broths described above were concentrated by centrifugation and then applied as an overtreatment with 5.9 mL of fermented product/lb of seed. Seeds were gently spun until dry and stored at 4° C. until planting-Immediately prior to planting an aliquot of seeds were taken, washed with 30 ml of PBS buffer, the effluent plated and CFUs were determined.

Thirteen strains, including the wild-type strain, were tested in June 2025. There were twenty replicants blocked across the greenhouse to reduce variability. The entry list within each replicant was randomized and mapped before planting.

Seeds were planted the day of or the day after treating into 1.5 gallon pots containing Berger BM2 germination mix (Hummert International). Irrigation was applied daily through a drip irrigation system to bring the soil to field capacity. Ten days after planting all macro and micro nutrients, except nitrogen, were applied daily with the same irrigation system. Nitrogen, in the form of ammonium nitrate and potassium nitrate dissolved into water, was applied to reach 120 lbs N per plant at a commercial seeding density, which is a reduced rate of N compared to standard practices. 40% of total nitrogen was applied at V3 and the remaining 60% of N was applied at V7. Plants were grown 46 or 43 days, respectively, and then were destructively harvested. Harvest was done just prior to tassel; plants were V14 and V16 respectively. At harvest the plant shoot was cut at 1 inch above the soil surface to avoid aerial roots. All above ground biomass was bagged and then dried at 80° C. for at least 14 days. After drying the material was weighed and recorded as grams and plant nitrogen concentration was measured. A one-sided statistical analysis was run to determine if entries were significantly greater than wild-type parent strain for dry biomass and nitrogen concentration within a trial. A p-value of 0.2 was used to determine significant difference between the test entry and respective control. Results are shown in Table 32. An initial greenhouse study with the wild-type strain and Pp1 was conducted in April 2025 but did not yield statistically significant differences at a p-value of 0.2.

TABLE 32
Percent difference biomass of plants from seed treated with engineered
strains compared to those treated with wild-type parent strain.

| | | | Biomass (g) | |
|---|---|---|---|---|
| | | | % | |
| | | | difference | P |
| Strain ID | Edits | CFU | WT | value |
| Pp1 | czcB::PtrnQ:Pz_fer | 1.00E+05 | 1.34 | 0.27* |
| Pp34 | PfldA(Ppe)::TR2-5′ UTR | 8.00E+04 | 3.99 | 0.04 |
| Pp12 | czcB::PtrnQ-5′ UTR:Ss_fer- | 3.00E+05 | 1.73 | 0.22* |
| | Tfer | | | |
| Pp13 | PtrnQ-5′ UTR downstream | 1.63E+05 | −1.17 | 0.70* |
| | of Pfer | | | |
| Pp10 | czcB::PtrnQ-5′ | 2.83E+05 | 0.99 | 0.33* |
| | UTR:Nm_fer-Tfer | | | |
| Pp48 | czcB::PR′-5′ UTR-fldA(Ta) | 1.56E+06 | 2.36 | 0.15 |
| Pp15 | TR2-5′ UTR downstream of | 2.13E+05 | 2.62 | 0.12 |
| | PporD | | | |
| Pp21 | czcB::PR′-5′ UTR- | 1.87E+05 | 1.86 | 0.20 |
| | DUF269(Pstel) | | | |
| Pp43 | czcB::PR′-5′ UTR-nifF(Ac) | 7.80E+05 | 2.62 | 0.12 |
| Pp20 | czcB::PR′-5′ UTR- | 1.43E+05 | −0.95 | 0.67* |
| | DUF269(Pr) | | | |
| Pp16 | czcB::PR′-5′ UTR- | 1.67E+04 | 3.74 | 0.05 |
| | DUF269(Pd) | | | |
| Pp6 | czcB::PtrnQ-5′ UTR:Fp_fer- | 1.87E+05 | 2.15 | 0.16 |
| | Tfer | | | |

### (vii) VARIANTS

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of similar amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

As indicated herein, variants of gene sequences can include codon optimized variants, sequence polymorphisms, splice variants, and/or mutations that do not affect the function of an encoded product to a statistically significant degree.

Variants of the protein, nucleic acid, and gene sequences also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including (but not limited to) those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y., Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridize under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

### (viii) EMBODIMENTS

For further illustration, additional non-limiting Embodiments of the present disclosure are set forth below.

Embodiment 1 is a genetically modified Paenibacillus microbe comprising at least one heterologous or native gene associated with an electron transport chain operably linked to a heterologous promoter.

Embodiment 2 is the genetically modified Paenibacillus microbe of Embodiment 1, wherein the at least one heterologous or native gene associated with an electron transport chain is or comprises: Paenibacillus pyruvate ferredoxin-oxidoreductase porCDAB operon; Paenibacillus fldA gene; Paenibacillus fer gene; or combinations thereof.

Embodiment 3 is the genetically modified Paenibacillus microbe of Embodiment 1 or Embodiment 2, wherein the at least one heterologous or native gene associated with an electron transport chain is from Paenibacillus zanthoxyli (Pz), Bacillus sp. FJAT-27264 (Bs), Synechococcus sp. MIT S9508 (Ss), Thermolongibacillus altinsuensis (Ta), Azotobacter chroococcum (Ac), Pseudomonas sp. OF001 (Ps), Klebsiella michiganensis (Km), Rhodobacter capsulatus (Rc), Paenibacillus tundrae (Pt), Cohnella fermenti (Cf), Brevibacillus marinus (Bm), Methylococcus capsulatus (Mc), Candidatus Pristimantibacillus lignocellulolyticus (PI), Fontibacillus panacisegetis strain DSM 28129 (Fp), Saccharibacillus brassicae (Sb), Fontibacillus solani (Fs), Saccharibacillus sp. WB 17 (WB17), Paenibacillus sp. 453mf (Nm), Saccharibacillus kuerlensis (Sk), Saccharibacillus sacchari (Ss), Paenibacillus zanthoxyli JH29 (Pz), Paenibacillus (multispecies) (Pm) or Paenibacillus forsythiae (Pf).

Embodiment 4 is the genetically modified Paenibacillus microbe of Embodiment 1, wherein the at least one heterologous or native gene associated with an electron transport chain is or comprises a gene encoding a ferredoxin.

Embodiment 5 is the genetically modified Paenibacillus microbe of Embodiment 4 wherein the gene encoding a ferredoxin is Pz fer, Pm fer, Ss_fer, Nm_fer, Pl_fer, Fp_fer, Sb_fer, Fs_fer, WB17_fer, or Sk_fer.

Embodiment 6 is the genetically modified Paenibacillus microbe of any one of Embodiments 1-4, wherein the genetically modified Paenibacillus microbe has increased pyruvate oxidoreductase activity as compared to a control Paenibacillus microbe that does not comprise the at least one heterologous or native gene associated with an electron transport chain, or has increased protein electron carrier activity compared to a control Paenibacillus microbe that does not comprise the at least one heterologous or native gene associated with an electron transport chain.

Embodiment 7 is the genetically modified Paenibacillus microbe of Embodiment 6, wherein the control Paenibacillus microbe is from the same species as the genetically modified Paenibacillus microbe but does not comprise the at least one heterologous or native gene associated with an electron transport chain.

Embodiment 8 is the genetically modified Paenibacillus microbe of Embodiment 6, wherein the control Paenibacillus microbe is a Paenibacillus microbe that is not genetically modified.

Embodiment 9 is a genetically modified Paenibacillus microbe comprising at least one heterologous or native gene associated with iron-sulfur cluster assembly or with oxygen protection, wherein the at least one gene is operably linked to a heterologous promoter.

Embodiment 10 is the genetically modified Paenibacillus microbe of Embodiment 9, wherein the at least one heterologous or native gene associated with iron-sulfur cluster assembly or with oxygen protection is selected from the group consisting of: Paenibacillus stellifer (Pstel), Paenibacillus durus (Pd), Paenibacillus riograndensis (Pr), Paenibacillus zanthoxyli (Pz), Paenibacillus forsythiae (Pf), Paenibacillus brasilensis (Pb), Paenbacillus graminis (Pg), Paenibacillus jilunlii (Pj), and Paenibacillus sabinae (Psab).

Embodiment 11 is the genetically modified Paenibacillus microbe of any one of Embodiments 1-3, wherein the at least one heterologous or native gene associated with an electron transport chain is or comprises a Paenibacillus porCDAB operon encoding an enzyme of E.C. 1.2.7.1.

Embodiment 12 is the genetically modified Paenibacillus microbe of Embodiment 9 or Embodiment 10, wherein the at least one heterologous or native gene is associated with oxygen protection and comprises a DUF269 gene.

Embodiment 13 is the genetically modified Paenibacillus microbe of Embodiment 12, wherein the DUF269 gene is selected from the group consisting of: DUF269 (PStel), DUF269 (Pr), DUF269 (Pd), DUF269 (Pf), DUF269 (Pg), DUF269 (Pj), DUF269 (Psab), and DUF269 (Pz).

Embodiment 14 is the genetically modified Paenibacillus microbe of Embodiment 9, wherein the at least one heterologous or native gene is associated with iron-sulfur assembly and comprises sufA, sufB, sufC, sufD, sufS, sufE, and/or sufU genes of the Paenibacillus suf operon.

Embodiment 15 is the genetically modified Paenibacillus microbe of Embodiment 9 or Embodiment 14, wherein the genetically modified Paenibacillus microbe has increased iron-sulfur cluster assembly as compared to a control Paenibacillus microbe which does not comprise the at least one heterologous or native gene associated with iron-sulfur cluster assembly or with oxygen protection.

Embodiment 16 is the genetically modified Paenibacillus microbe of Embodiment 9, wherein the genetically modified Paenibacillus microbe has increased oxygen protection as compared to a control Paenibacillus microbe which does not comprise the at least one heterologous or native gene associated with iron-sulfur cluster assembly or with oxygen protection.

Embodiment 17 is the genetically modified Paenibacillus microbe of Embodiment 15 or Embodiment 16, wherein the control Paenibacillus microbe is from the same species as the genetically modified Paenibacillus microbe but does not comprise the at least one heterologous gene associated with iron-sulfur assembly or with oxygen protection, wherein the oxygen protection is oxidoreductase activity.

Embodiment 18 is the genetically modified Paenibacillus microbe of Embodiment 15 or Embodiment 16, wherein the control Paenibacillus microbe is a Paenibacillus microbe that is not genetically modified.

Embodiment 19 is the genetically modified Paenibacillus microbe of any one of Embodiments 1-18, wherein the heterologous promoter is a heterologous constitutive promoter.

Embodiment 20 is the genetically modified Paenibacillus microbe of Embodiment 19, wherein (a) the at least one heterologous gene associated with an electron transport chain is integrated into the genome of the genetically modified Paenibacillus microbe, and/or (b) at least one heterologous gene associated with nitrogen fixation or nitrogen regulation is operably linked to a heterologous promoter and is integrated into the genome of the genetically modified Paenibacillus microbe.

Embodiment 21 is the genetically modified Paenibacillus microbe of Embodiment 19, wherein the heterologous constitutive promoter is selected from the group consisting of: P43, PR′, PSigX, PtrnQ, PclpE, PywbO, PypuA, PywrK, PsigV, PydaH, PyjoB, PyqeZ, PyacL, PyceG, PyrhK, PradA, PywaC, PmurB, PfabHA, PhtrB, PaprE, PycbR, PhtrA, PyrhH, PpbpE, PybfP, PywnJ, PytpA, PoatA, PmreBH, PyeaA, PybfO, PmurF, PyuaF, PypbG, PbcrC, PmetA, PylxX, PybgB, Pyusl, PcsbB, PyjbC, PclpC, PythP, PhtpG, Pyxil, PtilS, PspoOM, PdivlB, PypuD, PcssR, PyxzE, PdnaJ, PybfQ, PxpaC, PyngC, PyvlA, PpssA, PyoaF, PminC, PdltE, ProdA, PpspA, PclpP, PdivIC, PydjO, PydbS, PysdB, PyoaG, Pddl, PfosB, Pabh, Pspa, PyceE, PyknW, or PmreB.

Embodiment 22 is the genetically modified Paenibacillus microbe of Embodiment 19, wherein the heterologous constitutive promoter is PsigX.

Embodiment 23 is the genetically modified Paenibacillus microbe of Embodiment 19, wherein the heterologous constitutive promoter is PtrnQ or PR′.

Embodiment 24 is the genetically modified Paenibacillus microbe of Embodiment 19, wherein the heterologous constitutive promoter is PmreB.

Embodiment 25 is the genetically modified Paenibacillus microbe of any one of Embodiments 1-18, wherein the heterologous promoter is an inducible promoter selected from the group consisting of: PgroES, pLac, pTac, pBad, pL, pR, cspA, Ptrp, phoA, recA, proU, tetA, cst, cadA, cadR, and nar.

Embodiment 26 is the genetically modified Paenibacillus microbe of Embodiment 19, wherein the heterologous promoter is derived from B. subtilis.

Embodiment 27 is the genetically modified Paenibacillus microbe of Embodiment 19, wherein the heterologous promoter is derived from Paenibacillus genus.

Embodiment 28a is the genetically modified Paenibacillus microbe of Embodiment 19, wherein the at least one heterologous gene associated with an electron transport chain is part of an expression cassette integrated at a thymine-adenine (TA) dinucleotide site in the Paenibacillus genome, and/or the at least one heterologous gene associated with iron-sulfur cluster assembly or with oxygen protection operably linked to a heterologous promoter is part of an expression cassette integrated at a thymine-adenine (TA) dinucleotide site in the Paenibacillus genome.

Embodiment 28b is the genetically modified Paenibacillus microbe of Embodiment 19, wherein the at least one heterologous gene associated with an electron transport chain is part of an expression cassette integrated at a czcB locus in the Paenibacillus microbe genome, and/or the at least one heterologous gene associated with iron-sulfur cluster assembly or with oxygen protection operably linked to a heterologous promoter is part of an expression cassette integrated at a czcB locus in the Paenibacillus microbe genome.

Embodiment 28c is the genetically modified Paenibacillus microbe of Embodiment 19, wherein the at least one heterologous gene associated with an electron transport chain is part of an expression cassette integrated at a PROKKA_03723 locus in the Paenibacillus microbe genome, and/or the at least one heterologous gene associated with iron-sulfur cluster assembly or with oxygen protection operably linked to a heterologous promoter is part of an expression cassette integrated at a PROKKA_03723 locus in the Paenibacillus microbe genome.

Embodiment 29 is the genetically modified Paenibacillus microbe of any of embodiments 1-28, wherein the genetically modified Paenibacillus microbe comprises a selectable marker.

Embodiment 30 is the genetically modified Paenibacillus microbe of Embodiment 29, wherein the selectable marker comprises chloramphenicol acetyl transferase.

Embodiment 31a is the genetically modified Paenibacillus microbe of any one of Embodiments 1-30, wherein the genetically modified Paenibacillus microbe is selected from the group consisting of: P. abyssi, P. aestuarii, P. agarexedens, P. agaridevorans, P. alba, P. algeriensis, P. alginolyticus, P. algorifonticola, P. alkaliterrae, P. alvei, P. amylolyticus, P. anaericanus, P. antarcticus, P. antibioticophila, P. apiarius, P. assamensis, P. azoreducens, P. barcinonensis, P. barengoltzii, P. beijingensis, P. borealis, P. bovis, P. brasilensis, P. brassicae, P. camelliae, P. camerounensis, P. campinasensis, P. castaneae, P. catalpa, P. cathormii, P. cavernae, P. cellulositrophicus, P. cellulosilyticus, P. chartarius, P. chibensis, P. chinensis, P. chinjuensis, P. chitinolyticus, P. chondroitinus, P. chungangensis, P. cineris, P. contaminans, P. cookii, P. cucumis, P. curdlanotyticus, P. daejeonensis, P. dakarensis, P. darwinianus, P. dauci, P. dendritiformis, P. dongdonensis, P. donghaensis, P. doosanensis, P. durus, P. edaphicus, P. ehimensis, P. elgii, P. endophyticus, P. enshidis, P. etheri, P. faecis, P. favisporus, P. ferrarius, P. filicis, P. fonticola, P. forsythiae, P. frigoriresistens, P. gansuensis, P. gelatinilyticus, P. ginsengarvi, P. ginsengihumi, P. ginsengiterrae, P. glacialis, P. glucanolyticus, P. glycanilyticus, P. gorillae, P. graminis, P. granivorans, P. guangzhouensis, P. harenae, P. hemerocallicola, P. herberti, P. hodogayensis, P. hongkongensis, P. hordei, P. humi, P. humicus, P. hunanensis, P. ihumii, P. illinoisensis, P. insulae, P. jamilae, P. jilunlii, P. kobensis, P. koleovorans, P. konsidensis, P. koreensis, P. kribbensis, P. kyungheensis, P. lactis, P. larvae, P. lautus, P. lemnae, P. lentimorbus, P. lentus, P. lupini, P. macerans, P. macquariensis, P. marchantiophytorum, P. marinisediminis, P. marinum, P. massiliensis, P. medicaginis, P. mendelii, P. montaniterrae, P. motobuensis, P. mucilaginosus, P. nanensis, P. naphthalenovorans, P. nasutitermitis, P. nematophilus, P. nicotianae, P. oceanisediminis, P. odorifer, P. oenotherae, P. pabuli, P. panacisoli, P. panaciterrae, P. pasadenensis, P. pectinilyticus, P. peoriae, P. periandrae, P. phoenicis, P. phyllosphaerae, P. physcomitrellae, P. pinesoli, P. pini, P. pinihumi, P. pocheonensis, P. polymyxa, P. popilliae, P. populi, P. profundus, P. prosopidis, P. provencensis, P. pueri, P. puldeungensis, P. purispatii, P. qingshengii, P. quercus, P. radicis, P. relictisesami, P. residui, P. rhizoryzae, P. rhizosphaerae, P. rigui, P. riograndensis, P. ripae, P. sabinae, P. sacheonensis, P. sanguinis, P. sediminis, P. selenii, P. selenitireducens, P. senegalensis, P. septentrionalis, P. sepulcri, P. shenyangensis, P. shirakamiensis, P. siamensis, P. soli, P. sonchi, P. sophorae, P. sputi, P. stellifer, P. susongensis, P. swuensis, P. taichungensis, P. taihuensis, P. taiwanensis, P. taohuashanense, P. tarimensis, P. telluris, P. terrae, P. terreus, P. terrigena, P. tezpurensis, P. thailandensis, P. thermoaerophilus, P. thermophilus, P. thiaminolyticus, P. tianmuensis, P. tibetensis, P. timonensis, P. tundrae, P. turicensis, P. tylopili, P. typhae, P. tyraminigenes, P. uliginis, P. urinalis, P. validus, P. vini, P. vortex, P. vulneris, P. wenxiniae, P. wooponensis, P. woosongensis, P. wulumuqiensis, P. wynnii, P. xanthinilyticus, P. xinjiangensis, P. xylanexedens, P. xylaniclasticus, P. xylanisolvens, P. xylanilyticus, P. yonginensis, P. yunnanensis, P. zanthoxyli, and P. zeae.

Embodiment 31b is the genetically modified Paenibacillus microbe of Embodiment 31a wherein the genetically modified microbe is Paenibacillus brasilensis PB172 or Paenibacillus peoriae NRRL B-68085.

Embodiment 32 is the genetically modified Paenibacillus microbe of any one of Embodiment 1-31, wherein nitrogenase activity of the genetically modified Paenibacillus microbe is increased by up to 10%, 20%, 30%, 40%, or 50%, as compared to nitrogenase activity of a control Paenibacillus microbe as measured by an acetylene reduction assay.

Embodiment 33 is the genetically modified Paenibacillus microbe of Embodiment 32, wherein the control Paenibacillus microbe is a parent Paenibacillus microbe that is not genetically modified.

Embodiment 34 is the genetically modified Paenibacillus microbe of Embodiment 32, wherein the control Paenibacillus microbe is from the same species as the genetically modified Paenibacillus microbe but does not comprise the at least one heterologous gene associated with an electron transport chain and/or does not comprise the at least one heterologous gene associated with nitrogen fixation or nitrogen regulation.

Embodiment 35 is a formulation comprising a genetically modified Paenibacillus microbe of any one of Embodiments 1-34, and a carrier.

Embodiment 36 is the formulation of Embodiment 35, wherein the formulation comprises a buffer, a tackifier, a plant growth regulator, a stabilizer, a surfactant, an adherent, a desiccant, a fungicide, a nematicide, a rodenticide, an insecticide, an herbicide, a virucide, a nutrient, or a combination thereof.

Embodiment 37 is the formulation of Embodiment 35, wherein the formulation is a seed coating.

Embodiment 38 is a method of inoculating a plant or plant part, comprising contacting a plant or plant part with a formulation of any one of Embodiments 35-37.

Embodiment 39 is the method of Embodiment 38, further comprising growing the inoculated plant or plant part.

Embodiment 40 is a plant or plant part produced by the method of Embodiment 39.

Embodiment 41 is a method of improving an agronomic trait in a plant, comprising growing the plant from a plant or plant part that has been contacted with a formulation of any one of Embodiments 35-37.

Embodiment 42 is the method of Embodiment 41 further comprising administering nitrogen fertilizer to the plant.

Embodiment 43 is a method of reducing nitrogen fertilizer application, comprising:
inoculating a plant or plant part with a formulation of any one of Embodiments 35-37, and
growing a plant from the inoculated plant or plant part,
wherein application of nitrogen to the grown plant is reduced as compared to application of nitrogen to a corresponding plant grown from a plant or plant part that has not been contacted with the formulation.

Embodiment 44 is the method of Embodiment 43, wherein the reduction in the application of nitrogen (N) is measured as N replacement per application, and wherein the N replacement is at least 5 ppm of N, at least 6 ppm of N, at least 7 ppm of N, at least 8 ppm of N, at least 9 ppm of N, at least 10 ppm of N, at least 11 ppm of N, or at least 12 ppm of N, and up to 13 ppm of N.

Embodiment 45 is a method for preparing a composition, comprising contacting the surface of a plant or plant part with a formulation of any one of Embodiments 35-37 to produce an inoculated plant or plant part comprising the genetically modified Paenibacillus microbe, wherein the genetically modified Paenibacillus microbe is present in the formulation in an amount capable of improving an agronomic trait of a plant grown from the inoculated plant or plant part.

Embodiment 46 is the method of Embodiment 45, wherein the plant part comprises a root, a stem, or a leaf.

Embodiment 47 is a composition comprising a plant or plant part and a genetically modified Paenibacillus microbe of any one of Embodiments 1-34.

Embodiment 48 is the composition of Embodiment 47, wherein the plant part is a seed and the seed is coated with the genetically modified Paenibacillus microbe.

Embodiment 49 is the composition of Embodiment 47, wherein the genetically modified Paenibacillus microbe is present in an amount of 10⁵-10⁹ CFU per plant or plant part.

Embodiment 50 is the composition of Embodiment 47, wherein the plant or plant part is selected from the group consisting of: a whole plant, a seedling, meristematic tissue, ground tissue, vascular tissue, dermal tissue, seed, leaf, root, shoot, stem, flower, fruit, stolon, bulb, tuber, corm, keikis, bud, pod, tiller, sprig, pistil, ovaries, pollen, stamen, phloem, xylem, and blade.

Embodiment 51 is the composition of Embodiment 47, wherein the plant or plant part is selected from the group consisting of: wheat, soybean, maize, barley, millet, rice, turfgrass, cotton, canola, rapeseed, alfalfa, tomato, sugarbeet, oats, rye, sorghum, almond, walnut, apple, cannabis, peanut, strawberry, lettuce, orange, potato, banana, sugarcane, cassava, mango, guava, palm, onions, olives, peppers, tea, yams, cacao, sunflower, asparagus, carrot, coconut, lemon, lime, watermelon, cabbage, cucumber, and grape.

Embodiment 52 is a composition of any of Embodiments 47-51, further comprising a medium that promotes plant growth.

Embodiment 53 is a plant grown from the composition of any of Embodiments 47-52, wherein the plant exhibits an improved agronomic trait as compared to a plant not grown from the composition, and wherein the improved agronomic trait is selected from the group consisting of: increased nitrogen fixation, reduced nitrogen usage, increased plant yield, increased plant biomass, increased shoot biomass, increased shoot length, increased dry shoot weight, increased fresh shoot weight, increased seedling shoot length, increased dry seedling weight, increased fresh seedling weight, increased leaf surface area, increased root biomass, increased root length, increased root surface area, increased germination rate, increased emergence rate, increased photosynthetic capability, increased chlorophyll content, increased vigor, increased seed yield, increased dry weight of mature seeds, increased fresh weight of mature seeds, increased number of mature seeds per plant, increased number of pods per plant, increased length of pods per plant, increased plant height, increased pathogen resistance, increased pest resistance, earlier or increased flowering, increased protein content, increased carbohydrate content, increased antioxidant content, increased ammonium production, or a combination thereof.

Embodiment 54 is the plant of Embodiment 53, wherein the plant has a 3 to 20% increase in dry shoot weight as compared to the plant not grown from the composition.

Embodiment 55 is a seed coating comprising a genetically modified Paenibacillus microbe of any one of Embodiments 1-34.

Embodiment 56 is a genetic construct comprising, in 5′ to 3′ order:
(a) a heterologous promoter; and
(b) (i) at least one heterologous or native gene associated with an electron transport chain, and/or (ii) at least one heterologous or native gene associated with iron-sulfur cluster assembly, and/or (iii) at least one heterologous or native gene associated with oxygen protection.

Embodiment 57 is the genetic construct of Embodiment 56, wherein the at least one heterologous gene associated with an electron transport chain comprises: Paenibacillus pyruvate ferredoxin-oxidoreductase porCDAB operon; Paenibacillus fldA gene; Bacillus fldA gene; Synechococcus fldA gene; Thermolongibacillus fldA gene; Azotobacter fldA gene; Pseudomonas fldA gene; Klebsiella fldA gene; Rhodobacter fldA gene; Cohnella fldA gene; Brevibacillus fldA gene; Methylococcus fldA gene; Paenibacillus fer gene; Candidatus Pristimantibacillus fer gene; Fontibacillus fer gene; Saccharibacillus fer gene; Fontibacillus fer gene; or combinations thereof.

Embodiment 58 is the genetic construct of Embodiment 56 or Embodiment 57, wherein the heterologous promoter is a heterologous constitutive promoter.

Embodiment 59 is the genetic construct of Embodiment 58, wherein the heterologous constitutive promoter is selected from the group consisting of: P43, PR′, PSigX, PtrnQ, PclpE, PywbO, PypuA, PywrK, PsigV, PydaH, PyjoB, PyqeZ, PyacL, PyceG, PyrhK, PradA, PywaC, PmurB, PfabHA, PhtrB, PaprE, PycbR, PhtrA, PyrhH, PpbpE, PybfP, PywnJ, PytpA, PoatA, PmreBH, PyeaA, PybfO, PmurF, PyuaF, PypbG, PbcrC, PmetA, PylxX, PybgB, Pyusl, PcsbB, PyjbC, PclpC, PythP, PhtpG, Pyxil, PtilS, PspoOM, PdivlB, PypuD, PcssR, PyxzE, PdnaJ, PybfQ, PxpaC, PyngC, PyvlA, PpssA, PyoaF, PminC, PdltE, ProdA, PpspA, PclpP, PdivIC, PydjO, PydbS, PysdB, PyoaG, Pddl, PfosB, Pabh, Pspa, PyceE, PyknW, or PmreB.

Embodiment 60 is the genetic construct of Embodiment 58, wherein the heterologous constitutive promoter is PsigX.

Embodiment 61 is the genetic construct of Embodiment 58, wherein the heterologous constitutive promoter is PtrnQ.

Embodiment 62 is the genetic construct of Embodiment 58, wherein the heterologous constitutive promoter is PmreB.

Embodiment 63 is the genetic construct of Embodiment 56, wherein the heterologous promoter is a heterologous inducible promoter.

Embodiment 64 is the genetic construct of Embodiment 63, wherein the heterologous inducible promoter is selected from the group consisting of: PgroES, pLac, pTac, pBad, pL, pR, cspA, Ptrp, phoA, recA, proU, tetA, cst, cadA, cadR, or nar.

Embodiment 65 is the genetic construct of Embodiment 56, wherein the heterologous promoter is derived from Paenibacillus or Bacillus subtilis.

Embodiment 66 is the genetic construct of Embodiment 56, wherein the at least one heterologous gene associated with an electron transport chain, and/or the at least one heterologous gene associated with iron-sulfur cluster assembly and/or oxygen protection is from Paenibacillus zanthoxyli, Paenibacillus forsythia, or Paenibacillus brasilensis.

Embodiment 67 is the genetic construct of Embodiment 57, wherein the at least one heterologous gene associated with an electron transport chain comprises the Paenibacillus porCDAB operon encoding an enzyme of E.C. 1.2.7.1.

Embodiment 68 is the genetic construct of Embodiment 67, wherein the genetic construct comprises a sequence having at least 90% sequence identity to SEQ ID NO: 2 or comprising the sequence set forth in SEQ ID NO: 2.

Embodiment 69 is the genetic construct of Embodiment 67, wherein the genetic construct comprises a sequence having at least 90% sequence identity to SEQ ID NO: 6 or comprising the sequence set forth in SEQ ID NO: 6.

Embodiment 70 is the genetic construct of Embodiment 67, wherein the genetic construct comprises a sequence having at least 90% sequence identity to SEQ ID NO: 16 or comprising the sequence set forth in SEQ ID NO: 16.

Embodiment 71 is the genetic construct of Embodiment 67, wherein the genetic construct comprises a sequence having at least 90% sequence identity to SEQ ID NO: 11 or comprising the sequence set forth in SEQ ID NO: 11.

Embodiment 72 is the genetic construct of Embodiment 67, wherein the genetic construct comprises a sequence having at least 90% sequence identity to SEQ ID NO: 13 or comprising the sequence set forth in SEQ ID NO: 13.

Embodiment 73 is the genetic construct of Embodiment 57, wherein the at least one heterologous gene associated with an electron transport chain comprises the Paenibacillus fldA gene encoding a flavodoxin.

Embodiment 74 is the genetic construct of Embodiment 73, wherein the genetic construct comprises a sequence having at least 90% sequence identity to SEQ ID NO: 4 or comprising the sequence set forth in SEQ ID NO: 4.

Embodiment 75 is the genetic construct of Embodiment 73, wherein the genetic construct comprises a sequence having at least 90% sequence identity to SEQ ID NO: 9 or comprising the sequence set forth in SEQ ID NO: 9.

Embodiment 76 is the genetic construct of Embodiment 73, wherein the genetic construct comprises a sequence having at least 90% sequence identity to SEQ ID NO: 14 or comprising the sequence set forth in SEQ ID NO: 14.

Embodiment 77 is the genetic construct of Embodiment 73, wherein the genetic construct comprises a sequence having at least 90% sequence identity to SEQ ID NO: 12 or comprising the sequence set forth in SEQ ID NO: 12.

Embodiment 78 is the genetic construct of Embodiment 73, wherein the genetic construct comprises a sequence having at least 90% sequence identity to SEQ ID NO: 17 or comprising the sequence set forth in SEQ ID NO: 17.

Embodiment 79 is the genetic construct of Embodiment 56, wherein the at least one heterologous gene associated with an electron transport chain comprises a gene encoding a ferredoxin.

Embodiment 80 is the genetic construct of Embodiment 79, wherein the genetic construct comprises a sequence having at least 80% sequence identity to SEQ ID NO: 5 or comprising the sequence set forth in SEQ ID NO: 5.

Embodiment 81 is the genetic construct of Embodiment 79, wherein the genetic construct comprises a sequence having at least 80% sequence identity to SEQ ID NO: 8 or comprising the sequence set forth in SEQ ID NO: 8.

Embodiment 82 is the genetic construct of Embodiment 79, wherein the genetic construct comprises a sequence having at least 80% sequence identity to SEQ ID NO: 18 or comprising the sequence set forth in SEQ ID NO: 18.

Embodiment 83 is the genetic construct of Embodiment 79, wherein the genetic construct comprises a sequence having at least 80% sequence identity to SEQ ID NO: 29 or comprising the sequence set forth in SEQ ID NO: 29.

Embodiment 84 is the genetic construct of Embodiment 79, wherein the genetic construct comprises a sequence having at least 80% sequence identity to SEQ ID NO: 30 or comprising the sequence set forth in SEQ ID NO: 30.

Embodiment 85 is the genetic construct of Embodiment 79, wherein the genetic construct comprises a sequence having at least 80% sequence identity to SEQ ID NO: 31 or comprising the sequence set forth in SEQ ID NO: 31.

Embodiment 86 is the genetic construct of Embodiment 79, wherein the genetic construct comprises a sequence having at least 80% sequence identity to SEQ ID NO: 32 or comprising the sequence set forth in SEQ ID NO: 32.

Embodiment 87 is the genetic construct of Embodiment 79, wherein the genetic construct comprises a sequence having at least 80% sequence identity to SEQ ID NO: 33 or comprising the sequence set forth in SEQ ID NO: 33.

Embodiment 88 is the genetic construct of Embodiment 56, wherein the at least one heterologous gene associated with iron-sulfur cluster assembly comprises a gene from the sufCDSUB operon, the sufCDSUB operon comprising sufB, sufC, sufD, sufS, and sufU genes.

Embodiment 89 is the genetic construct of Embodiment 88, wherein the genetic construct comprises a sequence having at least 90% sequence identity to SEQ ID NO: 3 or comprising the sequence set forth in SEQ ID NO: 3.

Embodiment 90 is the genetic construct of Embodiment 88, wherein the genetic construct comprises a sequence having at least 90% sequence identity to SEQ ID NO: 7 or comprising the sequence set forth in SEQ ID NO: 7.

Embodiment 91 is the genetic construct of Embodiment 88, wherein the genetic construct comprises a sequence having at least 90% sequence identity to SEQ ID NO: 10 or comprising the sequence set forth in SEQ ID NO: 10.

Embodiment 92 is the genetic construct of Embodiment 88, wherein the genetic construct comprises a sequence having at least 90% sequence identity to SEQ ID NO: 15 or comprising the sequence set forth in SEQ ID NO: 15.

Embodiment 93 is the genetic construct of Embodiment 56, wherein the at least one heterologous gene associated with oxygen protection comprises a DUF269 gene.

Embodiment 94 is the genetic construct of Embodiment 93, wherein the genetic construct comprises a sequence having at least 90% sequence identity to SEQ ID NO: 19 or comprising the sequence set forth in SEQ ID NO: 19.

Embodiment 95 is the genetic construct of Embodiment 93, wherein the genetic construct comprises a sequence having at least 90% sequence identity to SEQ ID NO: 20 or comprising the sequence set forth in SEQ ID NO: 20.

Embodiment 96 is the genetic construct of Embodiment 93, wherein the genetic construct comprises a sequence having at least 90% sequence identity to SEQ ID NO: 38 or comprising the sequence set forth in SEQ ID NO: 38.

Embodiment 97 is the genetic construct of Embodiment 93, wherein the genetic construct comprises a sequence having at least 90% sequence identity to SEQ ID NO: 39 or comprising the sequence set forth in SEQ ID NO: 39.

Embodiment 98 is the genetic construct of Embodiment 93, wherein the genetic construct comprises a sequence having at least 90% sequence identity to SEQ ID NO: 40 or comprising the sequence set forth in SEQ ID NO: 40.

Embodiment 99 is the genetic construct of Embodiment 93, wherein the genetic construct comprises a sequence having at least 90% sequence identity to SEQ ID NO: 41 or comprising the sequence set forth in SEQ ID NO: 41.

Embodiment 100 is the genetic construct of Embodiment 93, wherein the genetic construct comprises a sequence having at least 90% sequence identity to SEQ ID NO: 42 or comprising the sequence set forth in SEQ ID NO: 42.

Embodiment 101 is the genetic construct of Embodiment 93, wherein the genetic construct comprises a sequence having at least 90% sequence identity to SEQ ID NO: 43 or comprising the sequence set forth in SEQ ID NO: 43.

Embodiment 102 is the genetic construct of Embodiment 93, wherein the genetic construct comprises a sequence having at least 90% sequence identity to SEQ ID NO: 44 or comprising the sequence set forth in SEQ ID NO: 44.

Embodiment 103 is the genetic construct of Embodiment 93, wherein the genetic construct comprises a sequence having at least 90% sequence identity to SEQ ID NO: 45 or comprising the sequence set forth in SEQ ID NO: 45.

Embodiment 104 is the genetic construct of Embodiment 56, wherein the genetic construct comprises a selectable marker.

Embodiment 105 is the genetic construct of Embodiment 104, wherein the selectable marker comprises a chloramphenicol acetyl transferase resistance gene.

Embodiment 106 is the genetic construct of Embodiment 56, wherein the genetic construct comprises a first pair of inverted mariner terminal repeat sequences from Haematobia irritans at a position 5′ of the heterologous constitutive promoter and a second pair of inverted mariner terminal repeat sequences from Haematobia irritans at a position 3′ of the at least one heterologous gene associated with an electron transport chain and/or the at least one heterologous gene associated with nitrogen fixation or nitrogen regulation.

Embodiment 107 is the genetic construct of Embodiment 56 wherein the gene encoding a ferredoxin is a Paenibacillus fer gene.

Embodiment 108 is the genetic construct of Embodiment 56 wherein the ferredoxin is encoded by the nucleic acid sequence of SEQ. ID NO: 47 or the nucleic acid sequence of SEQ. ID NO: 49.

Embodiment 109 is the genetic construct of Embodiment 58 wherein the heterologous constitutive promoter is PR′ or P43.

Embodiment 110 is a cell comprising the genetic construct of any one of Embodiments 56-106 or 106-109.

Embodiment 111 is the genetically modified Paenibacillus microbe of Embodiment 1, wherein the at least one heterologous gene associated with an electron transport chain is Pz_fer or Pm_fer.

Embodiment 112 is the genetically modified Paenibacillus microbe of Embodiment 1, wherein the at least one heterologous gene associated with an electron transport chain comprises the nucleic acid sequence of SEQ ID NO: 47 or the nucleic acid sequence of SEQ ID NO: 49.

Embodiment 113 is the genetically modified Paenibacillus microbe of any one of Embodiments 1-34 and 111-112, wherein the heterologous gene associated with an electron transport chain is operably linked to a heterologous constitutive promoter.

Embodiment 114 is the genetically modified Paenibacillus microbe of Embodiment 113, wherein the heterologous constitutive promoter is selected from: PtrnQ, PR′, or P43.

Embodiment 115 is the genetically modified Paenibacillus microbe of Embodiment 4 wherein the gene encoding a ferredoxin is a Paenibacillus fer gene.

Embodiment 116 is the genetically modified Paenibacillus microbe of Embodiment 4 wherein the ferredoxin is encoded by the nucleic acid sequence of SEQ. ID NO: 47 or the nucleic acid sequence of SEQ. ID NO: 49.

Embodiment 117 is the genetically modified Paenibacillus microbe of Embodiment 13 wherein the DUF269 gene is operably linked to a heterologous constitutive promoter.

Embodiment 118 is the genetically modified Paenibacillus microbe of Embodiment 117 wherein the heterologous constitutive promoter is PR′.

Embodiment 119 is the genetically modified Paenibacillus microbe of Embodiment 21, wherein the heterologous constitutive promoter is PR′.

Embodiment 120 is the genetically modified Paenibacillus microbe of Embodiment 21, wherein the heterologous constitutive promoter is P43.

Embodiment 121 is the genetically modified Paenibacillus microbe of Embodiment 88, wherein the sufCDSUB operon gene is from Stutzerimonas stutzeri (Ps), Rhizobium tropici (Rt), Frankia canadensis (Fc), Klebsiella variicola (Kv), Azotobacter vinelandii (Av), Bradyrhizobium diazoefficiens (Bd), Rhodobacter capsulatus (Rc), or Trichormus variabilis (Tv).

Embodiment 122 is the genetic construct of Embodiment 56, wherein the at least one heterologous or native gene associated with iron-sulfur cluster assembly comprises an sufCDSUB operon gene.

Embodiment 123 is the genetic construct of Embodiment 122, wherein the genetic construct comprises a sequence having at least 90% sequence identity to SEQ ID NO: 87 or comprising the sequence set forth in SEQ ID NO: 87.

Embodiment 124 is the genetic construct of Embodiment 122, wherein the genetic construct comprises a sequence having at least 90% sequence identity to SEQ ID NO: 103 or comprising the sequence set forth in SEQ ID NO: 103.

Embodiment 125 is the genetic construct of Embodiment 122, wherein the genetic construct comprises a sequence having at least 90% sequence identity to SEQ ID NO: 104 or comprising the sequence set forth in SEQ ID NO: 104.

Embodiment 126 is the genetic construct of Embodiment 122, wherein the genetic construct comprises a sequence having at least 90% sequence identity to SEQ ID NO: 105 or comprising the sequence set forth in SEQ ID NO: 105.

Embodiment 127 is the genetic construct of Embodiment 122, wherein the genetic construct comprises a sequence having at least 90% sequence identity to SEQ ID NO: 106 or comprising the sequence set forth in SEQ ID NO: 106.

Embodiment 128 is the genetic construct of Embodiment 122, wherein the genetic construct comprises a sequence having at least 90% sequence identity to SEQ ID NO: 107 or comprising the sequence set forth in SEQ ID NO: 107.

Embodiment 129 is the genetic construct of Embodiment 122, wherein the genetic construct comprises a sequence having at least 90% sequence identity to SEQ ID NO: 108 or comprising the sequence set forth in SEQ ID NO: 108.

Embodiment 130 is the genetic construct of Embodiment 122, wherein the genetic construct comprises a sequence having at least 90% sequence identity to SEQ ID NO: 109 or comprising the sequence set forth in SEQ ID NO: 109.

Embodiment 131 is the genetic construct of Embodiment 122, wherein the genetic construct comprises a sequence having at least 90% sequence identity to SEQ ID NO: 110 or comprising the sequence set forth in SEQ ID NO: 110.

Embodiment 132 is the genetic construct of Embodiment 122, wherein the genetic construct comprises a sequence having at least 90% sequence identity to SEQ ID NO: 111 or comprising the sequence set forth in SEQ ID NO: 111.

Embodiment 133 is the genetic construct of Embodiment 122, wherein the genetic construct comprises a sequence having at least 90% sequence identity to SEQ ID NO: 112 or comprising the sequence set forth in SEQ ID NO: 112.

Embodiment 134 is the genetic construct of Embodiment 122, wherein the genetic construct comprises a sequence having at least 90% sequence identity to SEQ ID NO: 87 or comprising the sequence set forth in SEQ ID NO: 87.

Embodiment 135 is the genetic construct of Embodiment 56, wherein the one heterologous or native gene associated with an electron transport chain comprises a ferredoxin (fer) gene.

Embodiment 136 is the genetic construct of Embodiment 135, wherein the genetic construct comprises a sequence having at least 90% sequence identity to SEQ ID NO: 50 or comprising the sequence set forth in SEQ ID NO: 50.

Embodiment 137 is the genetic construct of Embodiment 135, wherein the genetic construct comprises a sequence having at least 90% sequence identity to SEQ ID NO: 51 or comprising the sequence set forth in SEQ ID NO: 51.

Embodiment 138 is the genetic construct of Embodiment 135, wherein the genetic construct comprises a sequence having at least 90% sequence identity to SEQ ID NO: 52 or comprising the sequence set forth in SEQ ID NO: 52.

Embodiment 139 is the genetic construct of Embodiment 135, wherein the genetic construct comprises a sequence having at least 90% sequence identity to SEQ ID NO: 53 or comprising the sequence set forth in SEQ ID NO: 53.

Embodiment 140 is the genetic construct of Embodiment 135, wherein the genetic construct comprises a sequence having at least 90% sequence identity to SEQ ID NO: 54 or comprising the sequence set forth in SEQ ID NO: 54.

Embodiment 141 is the genetic construct of Embodiment 135, wherein the genetic construct comprises a sequence having at least 90% sequence identity to SEQ ID NO: 55 or comprising the sequence set forth in SEQ ID NO: 55.

Embodiment 142 is the genetic construct of Embodiment 135, wherein the genetic construct comprises a sequence having at least 90% sequence identity to SEQ ID NO: 56 or comprising the sequence set forth in SEQ ID NO: 56.

Embodiment 143 is the genetic construct of Embodiment 135, wherein the genetic construct comprises a sequence having at least 90% sequence identity to SEQ ID NO: 57 or comprising the sequence set forth in SEQ ID NO: 57.

Embodiment 144 is the genetic construct of Embodiment 56, wherein the one heterologous or native gene associated with an electron transport chain comprises a flavodoxin (fldA) gene.

Embodiment 145 is the genetic construct of Embodiment 144, wherein the genetic construct comprises a sequence having at least 90% sequence identity to SEQ ID NO: 147 or comprising the sequence set forth in SEQ ID NO: 147.

Embodiment 146 is the genetic construct of Embodiment 144, wherein the genetic construct comprises a sequence having at least 90% sequence identity to SEQ ID NO: 148or comprising the sequence set forth in SEQ ID NO: 148.

Embodiment 147 is the genetic construct of Embodiment 144, wherein the genetic construct comprises a sequence having at least 90% sequence identity to SEQ ID NO: 149 or comprising the sequence set forth in SEQ ID NO: 149.

Embodiment 148 is the genetic construct of Embodiment 144, wherein the genetic construct comprises a sequence having at least 90% sequence identity to SEQ ID NO: 150 or comprising the sequence set forth in SEQ ID NO: 150.

Embodiment 149 is the genetic construct of Embodiment 144, wherein the genetic construct comprises a sequence having at least 90% sequence identity to SEQ ID NO: 151 or comprising the sequence set forth in SEQ ID NO: 151.

Embodiment 150 is the genetic construct of Embodiment 144, wherein the genetic construct comprises a sequence having at least 90% sequence identity to SEQ ID NO: 152 or comprising the sequence set forth in SEQ ID NO: 152.

Embodiment 151 is the genetic construct of Embodiment 144, wherein the genetic construct comprises a sequence having at least 90% sequence identity to SEQ ID NO: 153 or comprising the sequence set forth in SEQ ID NO: 153.

Embodiment 152 is the genetic construct of Embodiment 144, wherein the genetic construct comprises a sequence having at least 90% sequence identity to SEQ ID NO: 154 or comprising the sequence set forth in SEQ ID NO: 154.

Embodiment 153 is the genetic construct of Embodiment 144, wherein the genetic construct comprises a sequence having at least 90% sequence identity to SEQ ID NO: 155 or comprising the sequence set forth in SEQ ID NO: 155.

Embodiment 154 is the genetic construct of Embodiment 144, wherein the genetic construct comprises a sequence having at least 90% sequence identity to SEQ ID NO: 156 or comprising the sequence set forth in SEQ ID NO: 156.

Embodiment 155 is the genetic construct of Embodiment 144, wherein the genetic construct comprises a sequence having at least 90% sequence identity to SEQ ID NO: 157 or comprising the sequence set forth in SEQ ID NO: 157.

Embodiment 156 is the genetic construct of Embodiment 144, wherein the genetic construct comprises a sequence having at least 90% sequence identity to SEQ ID NO: 158 or comprising the sequence set forth in SEQ ID NO: 158.

Embodiment 157 is the genetic construct of Embodiment 144, wherein the genetic construct comprises a sequence having at least 90% sequence identity to SEQ ID NO: 159 or comprising the sequence set forth in SEQ ID NO: 159.

Embodiment 158 is the genetic construct of Embodiment 144, wherein the genetic construct comprises a sequence having at least 90% sequence identity to SEQ ID NO: 160 or comprising the sequence set forth in SEQ ID NO: 160.

Embodiment 159 is the genetic construct of Embodiment 144, wherein the genetic construct comprises a sequence having at least 90% sequence identity to SEQ ID NO: 161 or comprising the sequence set forth in SEQ ID NO: 161.

Embodiment 160 is the genetic construct of Embodiment 144, wherein the genetic construct comprises a sequence having at least 90% sequence identity to SEQ ID NO: 162 or comprising the sequence set forth in SEQ ID NO: 162.

Embodiment 161 is the genetic construct of Embodiment 144, wherein the genetic construct comprises a sequence having at least 90% sequence identity to SEQ ID NO: 163 or comprising the sequence set forth in SEQ ID NO: 163.

Embodiment 162 is the genetic construct of Embodiment 144, wherein the genetic construct comprises a sequence having at least 90% sequence identity to SEQ ID NO: 164 or comprising the sequence set forth in SEQ ID NO: 164.

Embodiment 163 is a genetically modified Paenibacillus microbe comprising a heterologous or native fldA gene encoding flavodoxin operably linked to a heterologous promoter, wherein the fldA gene operably linked to the heterologous promoter is integrated into the genome of the genetically modified Paenibacillus microbe.

Embodiment 164 is the genetically modified Paenibacillus microbe of Embodiment 163, wherein the fldA gene is from Paenibacillus zanthoxyli (Pz), Bacillus sp. FJAT-27264 (Bs), Synechococcus sp. MIT S9508 (Ss), Thermolongibacillus altinsuensis (Ta), Azotobacter chroococcum (Ac), Pseudomonas sp. OF001 (Ps), Klebsiella michiganensis (Km), Rhodobacter capsulatus (Rc), Paenibacillus tundrae (Pt), Cohnella fermenti (Cf), Brevibacillus marinus (Bm), Methylococcus capsulatus (Mc), or Paenibacillus forsythiae (Pf).

Embodiment 165 is the genetically modified Paenibacillus microbe of Embodiment 163, wherein the heterologous promoter is constitutive.

Embodiment 166 is the genetically modified Paenibacillus microbe of Embodiment 165, wherein the heterologous constitutive promoter is selected from the group consisting of: P43, PR′, PSigX, PtrnQ, PclpE, PywbO, PypuA, PywrK, PsigV, PydaH, PyjoB, PyqeZ, PyacL, PyceG, PyrhK, PradA, PywaC, PmurB, PfabHA, PhtrB, PaprE, PycbR, PhtrA, PyrhH, PpbpE, PybfP, PywnJ, PytpA, PoatA, PmreBH, PyeaA, PybfO, PmurF, PyuaF, PypbG, PbcrC, PmetA, PylxX, PybgB, Pyusl, PcsbB, PyjbC, PclpC, PythP, PhtpG, Pyxil, PtilS, PspoOM, PdivlB, PypuD, PcssR, PyxzE, PdnaJ, PybfQ, PxpaC, PyngC, PyvlA, PpssA, PyoaF, PminC, PdltE, ProdA, PpspA, PclpP, PdivIC, PydjO, PydbS, PysdB, PyoaG, Pddl, PfosB, Pabh, Pspa, PyceE, PyknW, and PmreB.

Embodiment 167 is the genetically modified Paenibacillus microbe of Embodiment 165, wherein the heterologous constitutive promoter is PsigX.

Embodiment 168 is the genetically modified Paenibacillus microbe of Embodiment 167, wherein PsigX is from B. subtilis.

Embodiment 169 is the genetically modified Paenibacillus microbe of Embodiment 165, wherein the heterologous constitutive promoter is derived from Paenibacillus genus.

Embodiment 170 is the genetically modified Paenibacillus microbe of any one of Embodiments 163-169, wherein the fldA gene operably linked to a heterologous promoter is part of an expression cassette integrated at a thymine-adenine (TA) dinucleotide site in the Paenibacillus genome, a czcB locus in the Paenibacillus microbe genome or a PROKKA_03723 locus in the Paenibacillus microbe genome.

Embodiment 171 is the genetically modified Paenibacillus microbe of any one of Embodiments 163-170, wherein the genetically modified Paenibacillus microbe comprises a selectable marker.

Embodiment 172 is the genetically modified Paenibacillus microbe of Embodiment 163, wherein the genetically modified Paenibacillus microbe is selected from the group consisting of: P. abyssi, P. aestuarii, P. agarexedens, P. agaridevorans, P. alba, P. algeriensis, P. alginolyticus, P. algorifonticola, P. alkaliterrae, P. alvei, P. amylolyticus, P. anaericanus, P. antarcticus, P. antibioticophila, P. apiarius, P. assamensis, P. azoreducens, P. barcinonensis, P. barengoltzii, P. beijingensis, P. borealis, P. bovis, P. brasilensis, P. brassicae, P. camelliae, P. camerounensis, P. campinasensis, P. castaneae, P. catalpa, P. cathormii, P. cavernae, P. cellulositrophicus, P. cellulosilyticus, P. chartarius, P. chibensis, P. chinensis, P. chinjuensis, P. chitinolyticus, P. chondroitinus, P. chungangensis, P. cineris, P. contaminans, P. cookii, P. cucumis, P. curdlanotyticus, P. daejeonensis, P. dakarensis, P. darwinianus, P. dauci, P. dendritiformis, P. dongdonensis, P. donghaensis, P. doosanensis, P. durus, P. edaphicus, P. ehimensis, P. elgii, P. endophyticus, P. enshidis, P. etheri, P. faecis, P. favisporus, P. ferrarius, P. filicis, P. fonticola, P. forsythiae, P. frigoriresistens, P. gansuensis, P. gelatinilyticus, P. ginsengarvi, P. ginsengihumi, P. ginsengiterrae, P. glacialis, P. glucanolyticus, P. glycanilyticus, P. gorillae, P. graminis, P. granivorans, P. guangzhouensis, P. harenae, P. hemerocallicola, P. herberti, P. hodogayensis, P. hongkongensis, P. hordei, P. humi, P. humicus, P. hunanensis, P. ihumii, P. illinoisensis, P. insulae, P. jamilae, P. jilunlii, P. kobensis, P. koleovorans, P. konsidensis, P. koreensis, P. kribbensis, P. kyungheensis, P. lactis, P. larvae, P. lautus, P. lemnae, P. lentimorbus, P. lentus, P. lupini, P. macerans, P. macquariensis, P. marchantiophytorum, P. marinisediminis, P. marinum, P. massiliensis, P. medicaginis, P. mendelii, P. montaniterrae, P. motobuensis, P. mucilaginosus, P. nanensis, P. naphthalenovorans, P. nasutitermitis, P. nematophilus, P. nicotianae, P. oceanisediminis, P. odorifer, P. oenotherae, P. pabuli, P. panacisoli, P. panaciterrae, P. pasadenensis, P. pectinilyticus, P. peoriae, P. periandrae, P. phoenicis, P. phyllosphaerae, P. physcomitrellae, P. pinesoli, P. pini, P. pinihumi, P. pocheonensis, P. polymyxa, P. popilliae, P. populi, P. profundus, P. prosopidis, P. provencensis, P. pueri, P. puldeungensis, P. purispatii, P. qingshengii, P. quercus, P. radicis, P. relictisesami, P. residui, P. rhizoryzae, P. rhizosphaerae, P. rigui, P. riograndensis, P. ripae, P. sabinae, P. sacheonensis, P. sanguinis, P. sediminis, P. selenii, P. selenitireducens, P. senegalensis, P. septentrionalis, P. sepulcri, P. shenyangensis, P. shirakamiensis, P. siamensis, P. soli, P. sonchi, P. sophorae, P. sputi, P. stellifer, P. susongensis, P. swuensis, P. taichungensis, P. taihuensis, P. taiwanensis, P. taohuashanense, P. tarimensis, P. telluris, P. terrae, P. terreus, P. terrigena, P. tezpurensis, P. thailandensis, P. thermoaerophilus, P. thermophilus, P. thiaminolyticus, P. tianmuensis, P. tibetensis, P. timonensis, P. tundrae, P. turicensis, P. tylopili, P. typhae, P. tyraminigenes, P. uliginis, P. urinalis, P. validus, P. vini, P. vortex, P. vulneris, P. wenxiniae, P. wooponensis, P. woosongensis, P. wulumuqiensis, P. wynnii, P. xanthinilyticus, P. xinjiangensis, P. xylanexedens, P. xylaniclasticus, P. xylanisolvens, P. xylanilyticus, P. yonginensis, P. yunnanensis, P. zanthoxyli, and P. zeae.

Embodiment 173 is the genetically modified Paenibacillus microbe of any one of Embodiments 163-172, wherein nitrogenase activity of the genetically modified Paenibacillus microbe is increased by up to 10%, 20%, 30%, 40%, or 50%, as compared to nitrogenase activity of a wild-type control Paenibacillus microbe of the same species as measured by an acetylene reduction assay.

Embodiment 174 is the genetically modified Paenibacillus microbe of any one of Embodiments 163-173, wherein ammonium production of the genetically modified Paenibacillus microbe is increased by up to 10×, 20×, or 30×, as compared to ammonium production of a wild-type control Paenibacillus microbe of the same species as measured by an ammonia excretion assay.

Embodiment 175 is a formulation comprising a genetically modified Paenibacillus microbe of any one of Embodiments 163-174 and a carrier.

Embodiment 176 is the formulation of Embodiment 175, wherein the formulation comprises a buffer, a tackifier, a plant growth regulator, a stabilizer, a surfactant, an adherent, a desiccant, a fungicide, a nematicide, a rodenticide, an insecticide, an herbicide, a virucide, or a nutrient, or a combination thereof.

Embodiment 177 is the formulation of Embodiment 175, wherein the formulation is a seed coating.

Embodiment 178 is a method of inoculating a plant or plant part, comprising contacting a plant or plant part with a formulation of any one of Embodiments 175-177.

Embodiment 179 is the method of Embodiment 178, further comprising growing the inoculated plant or plant part.

Embodiment 180 is a plant or plant part produced by the method of Embodiment 179.

Embodiment 181 is a method of improving an agronomic trait in a plant, comprising growing the plant from a plant or plant part that has been contacted with a formulation of any one of Embodiments 175-177.

Embodiment 182 is the method of Embodiment 181, further comprising administering fertilizer to the plant.

Embodiment 183 is a method of reducing nitrogen fertilizer application, comprising
inoculating a plant or plant part with a formulation of any one of Embodiments 175-177, and
growing a plant from the inoculated plant or plant part,
wherein application of nitrogen to the grown plant is reduced as compared to application of nitrogen to a corresponding plant grown from a plant or plant part that has not been contacted with the formulation.

Embodiment 184 is the method of Embodiment 183, wherein the reduction in the application of nitrogen (N) is measured as N replacement per application, and wherein the N replacement is up to: 5 ppm of N, 6 ppm of N, 7 ppm of N, 8 ppm of N, 9 ppm of N, 10 ppm of N, 11 ppm of N, 12 ppm of N, 13 ppm of N.

Embodiment 185 is a method for preparing a composition, comprising contacting the surface of a plant or plant part with a formulation of any one of Embodiments 175-177 to produce an inoculated plant or plant part comprising the genetically modified Paenibacillus microbe, wherein the genetically modified Paenibacillus microbe is present in the formulation in an amount capable of improving an agronomic trait of a plant grown from the inoculated plant or plant part.

Embodiment 186 is the method of Embodiment 185, wherein the plant part comprises a root, a stem, or a leaf.

Embodiment 187 is a composition comprising a plant or plant part and a genetically modified Paenibacillus microbe of any one of Embodiments 163-174.

Embodiment 188 is the composition of Embodiment 187, wherein the plant part is a seed and the seed is coated with the genetically modified Paenibacillus microbe.

Embodiment 189 is the composition of Embodiment 187 or Embodiment 188, wherein the genetically modified Paenibacillus microbe is present in an amount of 10⁵-10⁹ CFU per plant or plant part.

Embodiment 190 is the composition of Embodiment 187 or 189, wherein the plant or plant part is selected from the group consisting of: a whole plant, a seedling, meristematic tissue, ground tissue, vascular tissue, dermal tissue, seed, leaf, root, shoot, stem, flower, fruit, stolon, bulb, tuber, corm, keikis, bud, pod, tiller, sprig, pistil, ovaries, pollen, stamen, phloem, xylem, and blade.

Embodiment 191 is the composition of any one of Embodiments 187-190, wherein the plant or plant part is selected from the group consisting of: wheat, soybean, maize, barley, millet, rice, turfgrass, cotton, canola, rapeseed, alfalfa, tomato, sugarbeet, oats, rye, sorghum, almond, walnut, apple, cannabis, peanut, strawberry, lettuce, orange, potato, banana, sugarcane, cassava, mango, guava, palm, onions, olives, peppers, tea, yams, cacao, sunflower, asparagus, carrot, coconut, lemon, lime, watermelon, cabbage, cucumber, and grape.

Embodiment 192 is the composition of any one of Embodiments 187-191, further comprising a medium that promotes plant growth.

Embodiment 193 is a plant grown from the composition of any of Embodiments 187-192, wherein the plant exhibits an improved agronomic trait as compared to a plant not grown from the composition, and wherein the improved agronomic trait is selected from the group consisting of: increased nitrogen fixation, reduced nitrogen usage, increased plant yield, increased plant biomass, increased shoot biomass, increased shoot length, increased dry shoot weight, increased fresh shoot weight, increased seedling shoot length, increased dry seedling weight, increased fresh seedling weight, increased leaf surface area, increased root biomass, increased root length, increased root surface area, increased germination rate, increased emergence rate, increased photosynthetic capability, increased chlorophyll content, increased vigor, increased seed yield, increased dry weight of mature seeds, increased fresh weight of mature seeds, increased number of mature seeds per plant, increased number of pods per plant, increased length of pods per plant, increased plant height, increased pathogen resistance, increased pest resistance, earlier or increased flowering, increased protein content, increased carbohydrate content, or increased antioxidant content, increased ammonium production, or a combination thereof.

Embodiment 194 is the plant of Embodiment 193, wherein the plant has a 3 to 20% increase in dry shoot weight as compared to the plant not grown from the composition.

Embodiment 195 is a seed coating comprising a genetically modified Paenibacillus microbe of any one of Embodiments 163-174.

Embodiment 196 is a genetic construct comprising, in 5′ to 3′ order:
a heterologous constitutive promoter; and
an fldA gene encoding a flavodoxin.

Embodiment 197 is the genetic construct of Embodiment 196, wherein the heterologous constitutive promoter is B. subtilis PsigX.

Embodiment 198 is the genetic construct of Embodiment 196 or 197, wherein the fldA gene is from Pz.

Embodiment 199 is the genetic construct of Embodiment 196, wherein the genetic construct comprises a sequence having at least 90% sequence identity to SEQ ID NO: 4 or comprising the sequence set forth in SEQ ID NO: 4.

Embodiment 200 is the genetic construct of any one of Embodiments 196-199, wherein the genetic construct comprises a selectable marker.

Embodiment 201 is the genetic construct of Embodiment 200, wherein the selectable marker comprises a chloramphenicol acetyl transferase resistance gene.

Embodiment 202 is the genetic construct of any one of Embodiments 196-201, wherein the genetic construct comprises a first pair of inverted mariner terminal repeat sequences from Haematobia irritans at a position 5′ of the heterologous constitutive promoter and a second pair of inverted mariner terminal repeat sequences from Haematobia irritans at a position 3′ of the gene.

Embodiment 203 is a cell comprising the genetic construct of any one of Embodiments 196-202.

Embodiment 204 is a genetically modified microorganism as described herein.

Embodiment 205 is a method of making the genetically modified microorganism of Embodiment 204.

Embodiment 206 is a method of using the genetically modified microorganism of Embodiment 204.

Embodiment 207 is the genetically modified Paenibacillus microbe of Embodiment 4 or Embodiment 5, wherein the fer gene operably linked to the heterologous promoter is integrated into the genome of the genetically modified Paenibacillus microbe.

Embodiment 208 is the genetically modified Paenibacillus microbe of Embodiment 4, Embodiment 5, or Embodiment 207, wherein the fer gene encodes a ferredoxin protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to a ferredoxin protein selected from the following group: Pz fer (SEQ ID NO: 46), Pm fer (SEQ ID NO: 48), Ss_fer (SEQ ID NO: 75), Nm_fer (SEQ ID NO: 71), Pl_fer (SEQ ID NO: 61), Fp_fer (SEQ ID NO: 63), Sb_fer (SEQ ID NO: 65), Fs_fer (SEQ ID NO: 67), WB17_fer (SEQ ID NO: 69), and Sk_fer (SEQ ID NO: 73).

Embodiment 209 is the genetically modified Paenibacillus microbe of Embodiment 4, Embodiment 5, Embodiment 207 or Embodiment 208, wherein the heterologous promoter is constitutive.

Embodiment 210 is the genetically modified Paenibacillus microbe of Embodiment 209, wherein the heterologous constitutive promoter is selected from the group consisting of: P43, PR′, PSigX, PtrnQ, PclpE, PywbO, PypuA, PywrK, PsigV, PydaH, PyjoB, PyqeZ, PyacL, PyceG, PyrhK, PradA, PywaC, PmurB, PfabHA, PhtrB, PaprE, PycbR, PhtrA, PyrhH, PpbpE, PybfP, PywnJ, PytpA, PoatA, PmreBH, PyeaA, PybfO, PmurF, PyuaF, PypbG, PbcrC, PmetA, PylxX, PybgB, Pyusl, PcsbB, PyjbC, PclpC, PythP, PhtpG, Pyxil, PtilS, PspoOM, PdivlB, PypuD, PcssR, PyxzE, PdnaJ, PybfQ, PxpaC, PyngC, PyvlA, PpssA, PyoaF, PminC, PdltE, ProdA, PpspA, PclpP, PdivIC, PydjO, PydbS, PysdB, PyoaG, Pddl, PfosB, Pabh, Pspa, PyceE, PyknW, and PmreB.

Embodiment 210b is the genetically modified Paenibacillus microbe of Embodiment 210, wherein the heterologous constitutive promoter is PR′ or PtrnQ.

Embodiment 210c is the genetically modified Paenibacillus microbe of Embodiment 210, wherein the heterologous constitutive promoter is PR′ from Bacillus subtilis or PtrnQ form bacteriophage lambda.

Embodiment 211 is the genetically modified Paenibacillus microbe of Embodiment 210, wherein the heterologous constitutive promoter is PsigX.

Embodiment 212 is the genetically modified Paenibacillus microbe of Embodiment 211, wherein PsigX is from B. subtilis.

Embodiment 213 is the genetically modified Paenibacillus microbe of Embodiment 210, wherein the heterologous constitutive promoter is derived from Paenibacillus genus.

Embodiment 214 is the genetically modified Paenibacillus microbe of any one of Embodiments 4-5 and Embodiments 207-214, wherein the fer gene operably linked to a heterologous promoter is part of an expression cassette integrated at a thymine-adenine (TA) dinucleotide site in the Paenibacillus genome, a czcB locus in the Paenibacillus microbe genome or a PROKKA_03723 locus in the Paenibacillus microbe genome.

Embodiment 214b is the genetically modified Paenibacillus microbe of any one of Embodiments 4-5 and Embodiments 207-214, wherein the fer gene operably linked to a heterologous promoter is part of an expression cassette integrated at a czcB locus in the Paenibacillus microbe genome.

Embodiment 215 is the genetically modified Paenibacillus microbe of any one of Embodiments 4-5 and Embodiments 207-214b, wherein the genetically modified Paenibacillus microbe comprises a selectable marker.

Embodiment 216 is the genetically modified Paenibacillus microbe of any one of Embodiments 4-5 and Embodiments 207-214b, wherein the genetically modified Paenibacillus microbe is selected from the group consisting of: P. abyssi, P. aestuarii, P. agarexedens, P. agaridevorans, P. alba, P. algeriensis, P. alginolyticus, P. algorifonticola, P. alkaliterrae, P. alvei, P. amylolyticus, P. anaericanus, P. antarcticus, P. antibioticophila, P. apiarius, P. assamensis, P. azoreducens, P. barcinonensis, P. barengoltzii, P. beijingensis, P. borealis, P. bovis, P. brasilensis, P. brassicae, P. camelliae, P. camerounensis, P. campinasensis, P. castaneae, P. catalpa, P. cathormii, P. cavernae, P. cellulositrophicus, P. cellulosilyticus, P. chartarius, P. chibensis, P. chinensis, P. chinjuensis, P. chitinolyticus, P. chondroitinus, P. chungangensis, P. cineris, P. contaminans, P. cookii, P. cucumis, P. curdlanotyticus, P. daejeonensis, P. dakarensis, P. darwinianus, P. dauci, P. dendritiformis, P. dongdonensis, P. donghaensis, P. doosanensis, P. durus, P. edaphicus, P. ehimensis, P. elgii, P. endophyticus, P. enshidis, P. etheri, P. faecis, P. favisporus, P. ferrarius, P. filicis, P. fonticola, P. forsythiae, P. frigoriresistens, P. gansuensis, P. gelatinilyticus, P. ginsengarvi, P. ginsengihumi, P. ginsengiterrae, P. glacialis, P. glucanolyticus, P. glycanilyticus, P. gorillae, P. graminis, P. granivorans, P. guangzhouensis, P. harenae, P. hemerocallicola, P. herberti, P. hodogayensis, P. hongkongensis, P. hordei, P. humi, P. humicus, P. hunanensis, P. ihumii, P. illinoisensis, P. insulae, P. jamilae, P. jilunlii, P. kobensis, P. koleovorans, P. konsidensis, P. koreensis, P. kribbensis, P. kyungheensis, P. lactis, P. larvae, P. lautus, P. lemnae, P. lentimorbus, P. lentus, P. lupini, P. macerans, P. macquariensis, P. marchantiophytorum, P. marinisediminis, P. marinum, P. massiliensis, P. medicaginis, P. mendelii, P. montaniterrae, P. motobuensis, P. mucilaginosus, P. nanensis, P. naphthalenovorans, P. nasutitermitis, P. nematophilus, P. nicotianae, P. oceanisediminis, P. odorifer, P. oenotherae, P. pabuli, P. panacisoli, P. panaciterrae, P. pasadenensis, P. pectinilyticus, P. peoriae, P. periandrae, P. phoenicis, P. phyllosphaerae, P. physcomitrellae, P. pinesoli, P. pini, P. pinihumi, P. pocheonensis, P. polymyxa, P. popilliae, P. populi, P. profundus, P. prosopidis, P. provencensis, P. pueri, P. puldeungensis, P. purispatii, P. qingshengii, P. quercus, P. radicis, P. relictisesami, P. residui, P. rhizoryzae, P. rhizosphaerae, P. rigui, P. riograndensis, P. ripae, P. sabinae, P. sacheonensis, P. sanguinis, P. sediminis, P. selenii, P. selenitireducens, P. senegalensis, P. septentrionalis, P. sepulcri, P. shenyangensis, P. shirakamiensis, P. siamensis, P. soli, P. sonchi, P. sophorae, P. sputi, P. stellifer, P. susongensis, P. swuensis, P. taichungensis, P. taihuensis, P. taiwanensis, P. taohuashanense, P. tarimensis, P. telluris, P. terrae, P. terreus, P. terrigena, P. tezpurensis, P. thailandensis, P. thermoaerophilus, P. thermophilus, P. thiaminolyticus, P. tianmuensis, P. tibetensis, P. timonensis, P. tundrae, P. turicensis, P. tylopili, P. typhae, P. tyraminigenes, P. uliginis, P. urinalis, P. validus, P. vini, P. vortex, P. vulneris, P. wenxiniae, P. wooponensis, P. woosongensis, P. wulumuqiensis, P. wynnii, P. xanthinilyticus, P. xinjiangensis, P. xylanexedens, P. xylaniclasticus, P. xylanisolvens, P. xylanilyticus, P. yonginensis, P. yunnanensis, P. zanthoxyli, and P. zeae.

Embodiment 216b is the genetically modified Paenibacillus microbe of Embodiment 216, wherein the genetically modified Paenibacillus microbe is Paenibacillus brasilensis PB172 or Paenibacillus peoriae NRRL B-68085.

Embodiment 217 is the genetically modified Paenibacillus microbe of any one of Embodiments 4-5 and Embodiments 207-216b, wherein nitrogenase activity of the genetically modified Paenibacillus microbe is increased by up to 10%, 20%, 30%, 40%, or 50%, as compared to nitrogenase activity of a wild-type control Paenibacillus microbe of the same species as measured by an acetylene reduction assay.

Embodiment 218 is the genetically modified Paenibacillus microbe of any one of Embodiments 4-5 and Embodiments 207-216b, wherein ammonium production of the genetically modified Paenibacillus microbe is increased by up to 10×, 20×, or 30×, as compared to nitrogenase activity of a wild-type control Paenibacillus microbe of the same species as measured by an ammonia excretion assay.

Embodiment 219 is a formulation comprising a genetically modified Paenibacillus microbe of any one of Embodiments 4-5 and Embodiments 207-218 and a carrier.

Embodiment 220 is the formulation of Embodiment 219, wherein the formulation comprises a buffer, a tackifier, a plant growth regulator, a stabilizer, a surfactant, an adherent, a desiccant, a fungicide, a nematicide, a rodenticide, an insecticide, an herbicide, a virucide, or a nutrient, or a combination thereof.

Embodiment 221 is the formulation of Embodiment 219, wherein the formulation is a seed coating.

Embodiment 222 is a method of inoculating a plant or plant part, comprising contacting a plant or plant part with a formulation of any one of Embodiments 219-221.

Embodiment 223 is the method of Embodiment 222, further comprising growing the inoculated plant or plant part.

Embodiment 224 is a plant or plant part produced by the method of Embodiment 223.

Embodiment 225 is a method of improving an agronomic trait in a plant, comprising growing the plant from a plant or plant part that has been contacted with a formulation of any one of Embodiments 219-221.

Embodiment 226 is the method of Embodiment 225, further comprising administering fertilizer to the plant.

Embodiment 227 is a method of reducing nitrogen fertilizer application, comprising:
inoculating a plant or plant part with a formulation of any one of Embodiments 219-221, and growing a plant from the inoculated plant or plant part,
wherein application of nitrogen to the grown plant is reduced as compared to application of nitrogen to a corresponding plant grown from a plant or plant part that has not been contacted with the formulation.

Embodiment 228 is the method of Embodiment 227, wherein the reduction in the application of nitrogen (N) is measured as N replacement per application, and wherein the N replacement is up to: 5 ppm of N, 6 ppm of N, 7 ppm of N, 8 ppm of N, 9 ppm of N, 10 ppm of N, 11 ppm of N, 12 ppm of N, 13 ppm of N.

Embodiment 229 is a method for preparing a composition, comprising contacting the surface of a plant or plant part with a formulation of any one of Embodiments 219-221 to produce an inoculated plant or plant part comprising the genetically modified Paenibacillus microbe, wherein the genetically modified Paenibacillus microbe is present in the formulation in an amount capable of improving an agronomic trait of a plant grown from the inoculated plant or plant part.

Embodiment 230 is the method of Embodiment 229, wherein the plant part comprises a root, a stem, or a leaf.

Embodiment 231 is a composition comprising a plant or plant part and a genetically modified Paenibacillus microbe of any one of Embodiments 4-5 and Embodiments 207-218.

Embodiment 232 is the composition of Embodiment 231, wherein the plant part is a seed and the seed is coated with the genetically modified Paenibacillus microbe.

Embodiment 233 is the composition of Embodiment 231 or Embodiment 232, wherein the genetically modified Paenibacillus microbe is present in an amount of 10⁵-10⁹ CFU per plant or plant part.

Embodiment 234 is the composition of Embodiment 231 or 233, wherein the plant or plant part is selected from the group consisting of: a whole plant, a seedling, meristematic tissue, ground tissue, vascular tissue, dermal tissue, seed, leaf, root, shoot, stem, flower, fruit, stolon, bulb, tuber, corm, keikis, bud, pod, tiller, sprig, pistil, ovaries, pollen, stamen, phloem, xylem, and blade.

Embodiment 235 is the composition of any one of Embodiments 231-234, wherein the plant or plant part is selected from the group consisting of: wheat, soybean, maize, barley, millet, rice, turfgrass, cotton, canola, rapeseed, alfalfa, tomato, sugarbeet, oats, rye, sorghum, almond, walnut, apple, cannabis, peanut, strawberry, lettuce, orange, potato, banana, sugarcane, cassava, mango, guava, palm, onions, olives, peppers, tea, yams, cacao, sunflower, asparagus, carrot, coconut, lemon, lime, watermelon, cabbage, cucumber, and grape.

Embodiment 236 is the composition of any one of Embodiments 231-235, further comprising a medium that promotes plant growth.

Embodiment 237 is a plant grown from the composition of any of Embodiments 231-236, wherein the plant exhibits an improved agronomic trait as compared to a plant not grown from the composition, and wherein the improved agronomic trait is selected from the group consisting of: increased nitrogen fixation, reduced nitrogen usage, increased plant yield, increased plant biomass, increased shoot biomass, increased shoot length, increased dry shoot weight, increased fresh shoot weight, increased seedling shoot length, increased dry seedling weight, increased fresh seedling weight, increased leaf surface area, increased root biomass, increased root length, increased root surface area, increased germination rate, increased emergence rate, increased photosynthetic capability, increased chlorophyll content, increased vigor, increased seed yield, increased dry weight of mature seeds, increased fresh weight of mature seeds, increased number of mature seeds per plant, increased number of pods per plant, increased length of pods per plant, increased plant height, increased pathogen resistance, increased pest resistance, earlier or increased flowering, increased protein content, increased carbohydrate content, or increased antioxidant content, or a combination thereof.

Embodiment 238 is the plant of Embodiment 237, wherein the plant has a 3 to 20% increase in dry shoot weight as compared to the plant not grown from the composition.

Embodiment 239 is a seed coating comprising a genetically modified Paenibacillus microbe of any one of Embodiments 4-5 and Embodiments 207-218.

Embodiment 240 is a genetic construct comprising 5′ to 3′:
a heterologous constitutive promoter; and
a fer gene encoding a ferredoxin.

Embodiment 241 is the genetic construct of Embodiment 240, wherein the heterologous constitutive promoter is PtrnQ or PR′.

Embodiment 242 is the genetic construct of Embodiment 240 or 241, wherein the fer gene is from Pz fer, Pm fer, Ss_fer, Nm_fer, Pl_fer, Fp_fer, Sb_fer, Fs_fer, WB17_fer, or Sk_fer.

Embodiment 242b is the genetic construct of Embodiment 242, wherein the fer gene encodes a ferredoxin protein having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to a ferredoxin protein selected from the following group: Pz fer (SEQ ID NO: 46), Pm fer (SEQ ID NO: 48), Ss_fer (SEQ ID NO: 75), Nm_fer (SEQ ID NO: 71), Pl_fer (SEQ ID NO: 61), Fp_fer (SEQ ID NO: 63), Sb_fer (SEQ ID NO: 65), Fs_fer (SEQ ID NO: 67), WB17_fer (SEQ ID NO: 69), and Sk_fer (SEQ ID NO: 73).

Embodiment 243 is the genetic construct of Embodiment 196, wherein the genetic construct comprises a sequence having at least 90% sequence identity to any one of SEQ ID NOs: 29-33 or comprising the sequence set forth in any one of SEQ ID NOs: 29-33.

Embodiment 244 is the genetic construct of any one of Embodiments 240-243, wherein the genetic construct comprises a selectable marker.

Embodiment 245 is the genetic construct of Embodiment 244, wherein the selectable marker comprises a chloramphenicol acetyl transferase resistance gene.

Embodiment 246 is the genetic construct of any one of Embodiments 240-245, wherein the genetic construct comprises inverted mariner terminal repeat sequences from Haematobia irritans 5′ of the promoter and 3′ of the gene.

### (ix) CLOSING PARAGRAPHS

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” mean: have, but are not limited to, and allow for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability of a genetically modified Paenibacillus microbe of the disclosure to reduce the amount of nitrogen application for an agricultural plant when the genetically modified Paenibacillus microbe is associated with the plant.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

1. A genetically modified Paenibacillus microbe comprising at least one heterologous or native gene associated with an electron transport chain operably linked to a heterologous promoter, wherein the at least one heterologous or native gene associated with an electron transport chain is or comprises porCDAB operon, fldA gene, fer gene or combinations thereof.

2. The genetically modified Paenibacillus microbe of claim 1, wherein the at least one heterologous or native gene associated with an electron transport chain is or comprises: Paenibacillus pyruvate ferredoxin-oxidoreductase porCDAB operon; Paenibacillus fldA gene; Paenibacillus fer gene; or combinations thereof.

3. The genetically modified Paenibacillus microbe of claim 1, wherein the at least one heterologous or native gene associated with an electron transport chain is from Paenibacillus zanthoxyli (Pz), Bacillus sp. FJAT-27264 (Bs), Synechococcus sp. MIT S9508 (Ss), Thermolongibacillus altinsuensis (Ta), Azotobacter chroococcum (Ac), Pseudomonas sp. OF001 (Ps), Klebsiella michiganensis (Km), Rhodobacter capsulatus (Rc), Paenibacillus tundrae (Pt), Cohnella fermenti (Cf), Brevibacillus marinus (Bm), Methylococcus capsulatus (Mc), Candidatus Pristimantibacillus lignocellulolyticus (PI), Fontibacillus panacisegetis strain DSM 28129 (Fp), Saccharibacillus brassicae (Sb), Fontibacillus solani (Fs), Saccharibacillus sp. WB 17 (WB17), Paenibacillus sp. 453mf (Nm), Saccharibacillus kuerlensis (Sk), Saccharibacillus sacchari (Ss), Paenibacillus zanthoxyli JH29 (Pz), Paenibacillus (multispecies) (Pm), Paenibacillus forsythiae (Pf) or Paenibacillus brasilensis (Pb).

4. The genetically modified Paenibacillus microbe of claim 1, wherein the at least one heterologous or native gene associated with an electron transport chain is or comprises a gene encoding a ferredoxin.

5. The genetically modified Paenibacillus microbe of claim 4 wherein the gene encoding a ferredoxin is Pz fer, Pm fer, Ss_fer, Nm_fer, Pl_fer, Fp_fer, Sb_fer, Fs_fer, WB17_fer, or Sk_fer.

6-10. (canceled)

11. The genetically modified Paenibacillus microbe of claim 1, wherein the at least one heterologous or native gene associated with an electron transport chain is or comprises a Paenibacillus porCDAB operon encoding an enzyme of E.C. 1.2.7.1.

12-18. (canceled)

19. The genetically modified Paenibacillus microbe of claim 1, wherein the heterologous promoter is a heterologous constitutive promoter.

20. The genetically modified Paenibacillus microbe of claim 19, wherein (a) the at least one heterologous gene associated with an electron transport chain is integrated into the genome of the genetically modified Paenibacillus microbe, and/or (b) at least one heterologous gene associated with nitrogen fixation or nitrogen regulation is operably linked to a heterologous promoter and is integrated into the genome of the genetically modified Paenibacillus microbe.

21. The genetically modified Paenibacillus microbe of claim 19, wherein the heterologous constitutive promoter is selected from the group consisting of: P43, PR′, PSigX, PtrnQ, PclpE, PywbO, PypuA, PywrK, PsigV, PydaH, PyjoB, PyqeZ, PyacL, PyceG, PyrhK, PradA, PywaC, PmurB, PfabHA, PhtrB, PaprE, PycbR, PhtrA, PyrhH, PpbpE, PybfP, PywnJ, PytpA, PoatA, PmreBH, PyeaA, PybfO, PmurF, PyuaF, PypbG, PbcrC, PmetA, PylxX, PybgB, PyusI, PcsbB, PyjbC, PclpC, PythP, PhtpG, Pyxil, PtilS, PspoOM, PdivlB, PypuD, PcssR, PyxzE, PdnaJ, PybfQ, PxpaC, PyngC, PyvlA, PpssA, PyoaF, PminC, PdltE, ProdA, PpspA, PclpP, PdivlC, PydjO, PydbS, PysdB, PyoaG, Pddl, PfosB, Pabh, Pspa, PyceE, PyknW, or PmreB.

22-24. (canceled)

25. The genetically modified Paenibacillus microbe of claim 1, wherein the heterologous promoter is an inducible promoter selected from the group consisting of: PgroES, pLac, pTac, pBad, pL, pR, cspA, Ptrp, phoA, recA, proU, tetA, cst, cadA, cadR, and nar.

26-30. (canceled)

31. The genetically modified Paenibacillus microbe of claim 1, wherein the genetically modified Paenibacillus microbe is selected from the group consisting of: P. abyssi, P. aestuarii, P. agarexedens, P. agaridevorans, P. alba, P. algeriensis, P. alginolyticus, P. algorifonticola, P. alkaliterrae, P. alvei, P. amylolyticus, P. anaericanus, P. antarcticus, P. antibioticophila, P. apiarius, P. assamensis, P. azoreducens, P. barcinonensis, P. barengoltzii, P. beijingensis, P. borealis, P. bovis, P. brasilensis, P. brassicae, P. camelliae, P. camerounensis, P. campinasensis, P. castaneae, P. catalpa, P. cathormii, P. cavernae, P. cellulositrophicus, P. cellulosilyticus, P. chartarius, P. chibensis, P. chinensis, P. chinjuensis, P. chitinolyticus, P. chondroitinus, P. chungangensis, P. cineris, P. contaminans, P. cookii, P. cucumis, P. curdlanotyticus, P. daejeonensis, P. dakarensis, P. darwinianus, P. dauci, P. dendritiformis, P. dongdonensis, P. donghaensis, P. doosanensis, P. durus, P. edaphicus, P. ehimensis, P. elgii, P. endophyticus, P. enshidis, P. etheri, P. faecis, P. favisporus, P. ferrarius, P. filicis, P. fonticola, P. forsythiae, P. frigoriresistens, P. gansuensis, P. gelatinilyticus, P. ginsengarvi, P. ginsengihumi, P. ginsengiterrae, P. glacialis, P. glucanolyticus, P. glycanilyticus, P. gorillae, P. graminis, P. granivorans, P. guangzhouensis, P. harenae, P. hemerocallicola, P. herberti, P. hodogayensis, P. hongkongensis, P. hordei, P. humi, P. humicus, P. hunanensis, P. ihumii, P. illinoisensis, P. insulae, P. jamilae, P. jilunlii, P. kobensis, P. koleovorans, P. konsidensis, P. koreensis, P. kribbensis, P. kyungheensis, P. lactis, P. larvae, P. lautus, P. lemnae, P. lentimorbus, P. lentus, P. lupini, P. macerans, P. macquariensis, P. marchantiophytorum, P. marinisediminis, P. marinum, P. massiliensis, P. medicaginis, P. mendelii, P. montaniterrae, P. motobuensis, P. mucilaginosus, P. nanensis, P. naphthalenovorans, P. nasutitermitis, P. nematophilus, P. nicotianae, P. oceanisediminis, P. odorifer, P. oenotherae, P. pabuli, P. panacisoli, P. panaciterrae, P. pasadenensis, P. pectinilyticus, P. peoriae, P. periandrae, P. phoenicis, P. phyllosphaerae, P. physcomitrellae, P. pinesoli, P. pini, P. pinihumi, P. pocheonensis, P. polymyxa, P. popilliae, P. populi, P. profundus, P. prosopidis, P. provencensis, P. pueri, P. puldeungensis, P. purispatii, P. qingshengii, P. quercus, P. radicis, P. relictisesami, P. residui, P. rhizoryzae, P. rhizosphaerae, P. rigui, P. riograndensis, P. ripae, P. sabinae, P. sacheonensis, P. sanguinis, P. sediminis, P. selenii, P. selenitireducens, P. senegalensis, P. septentrionalis, P. sepulcri, P. shenyangensis, P. shirakamiensis, P. siamensis, P. soli, P. sonchi, P. sophorae, P. sputi, P. stellifer, P. susongensis, P. swuensis, P. taichungensis, P. taihuensis, P. taiwanensis, P. taohuashanense, P. tarimensis, P. telluris, P. terrae, P. terreus, P. terrigena, P. tezpurensis, P. thailandensis, P. thermoaerophilus, P. thermophilus, P. thiaminolyticus, P. tianmuensis, P. tibetensis, P. timonensis, P. tundrae, P. turicensis, P. tylopili, P. typhae, P. tyraminigenes, P. uliginis, P. urinalis, P. validus, P. vini, P. vortex, P. vulneris, P. wenxiniae, P. wooponensis, P. woosongensis, P. wulumuqiensis, P. wynnii, P. xanthinilyticus, P. xinjiangensis, P. xylanexedens, P. xylaniclasticus, P. xylanisolvens, P. xylanilyticus, P. yonginensis, P. yunnanensis, P. zanthoxyli, and P. zeae.

32. The genetically modified Paenibacillus microbe of claim 1, wherein nitrogenase activity of the genetically modified Paenibacillus microbe is increased by up to 10%, 20%, 30%, 40%, or 50%, as compared to nitrogenase activity of a control Paenibacillus microbe as measured by an acetylene reduction assay.

33. The genetically modified Paenibacillus microbe of claim 32, wherein the control Paenibacillus microbe is a parent Paenibacillus microbe that is not genetically modified.

34. (canceled)

35. A formulation comprising a genetically modified Paenibacillus microbe of claim 1, and a carrier.

36-37. (canceled)

38. A method of inoculating a plant or plant part, comprising contacting a plant or plant part with a formulation of claim 35.

39-42. (canceled)

43. A method of reducing nitrogen fertilizer application, comprising inoculating a plant or plant part with the formulation of claim 35, and
growing a plant from the inoculated plant or plant part,
wherein application of nitrogen to the grown plant is reduced as compared to application of nitrogen to a corresponding plant grown from a plant or plant part that has not been contacted with the formulation.

44-46. (canceled)

47. A composition comprising a plant or plant part and a genetically modified Paenibacillus microbe of claim 1.

48. The composition of claim 47, wherein the plant part is a seed and the seed is coated with the genetically modified Paenibacillus microbe.

49-55. (canceled)

56. A genetic construct comprising, in 5′ to 3′ order:
(a) a heterologous promoter; and
(b) at least one heterologous or native gene associated with an electron transport chain, wherein the at least one heterologous or native gene associated with an electron transport chain is or comprises porCDAB operon, fldA gene, fer gene or combinations thereof.

57-109. (canceled)

110. A cell comprising the genetic construct of claim 56.

111-206. (canceled)
